SEQUENCE LISTING (94 sequences)

SEQ ID NO: 1 - DNA, 69 bp, Solanum lycopersicum
  source 1..69: mol_type = genomic DNA
gcaacagataggaggggaagaccaccatcaagacctaaagttggtagtggacctcctcctcagaacaat

SEQ ID NO: 2 - protein, 23 aa, Solanum lycopersicum
ATDRRGRPPSRPKVGSGPPPQNN

SEQ ID NO: 3 - DNA, 69 bp, Capsicum annuum
  source 1..69: mol_type = genomic DNA
acaggccggaggaggggaagaccaccatcaagacctggagttggaaggggacctcctcctgagaacaat

SEQ ID NO: 4 - protein, 23 aa, Capsicum annuum
TGRRRGRPPSRPGVGRGPPPENN

SEQ ID NO: 5 - DNA, 69 bp, Glycine max
  source 1..69: mol_type = genomic DNA
ggaagtgtagtagttctacaaacgaaaagaaaacatgatggaggaaaaggacgtgatccacaaactaat

SEQ ID NO: 6 - protein, 23 aa, Glycine max
GSVVVLQTKRKHDGGKGRDPQTN

SEQ ID NO: 7 - DNA, 70 bp, Helianthus annuus
  source 1..70: mol_type = genomic DNA
aggagactcacaagaagaccccctccgccaagggggccgattagctctggaggaggcggtcaaaccaact

SEQ ID NO: 8 - protein, 23 aa, Helianthus annuus
RRLTRRPPPPRGPISSGGGGQTN

SEQ ID NO: 9 - DNA, 69 bp, Gossypium arboreum
  source 1..69: mol_type = genomic DNA
cttcccatggtttctctctttactcctaaaaggccagggacaagcgccggcagtggacctcagattaat

SEQ ID NO: 10 - protein, 23 aa, Gossypium arboreum
LPMVSLFTPKRPGTSAGSGPQIN

SEQ ID NO: 11 - DNA, 69 bp, Oryza sativa
  source 1..69: mol_type = genomic DNA
gatgattccaagccaacgcggcctggagcgccggcggagggctccggcggcaacggcggagcaatccac

SEQ ID NO: 12 - protein, 23 aa, Oryza sativa
DDSKPTGPGAPAEGSGGDGGAIH

SEQ ID NO: 13 - DNA, 69 bp, Zea mays
  source 1..69: mol_type = genomic DNA
aggaggccgcggccgcggccgccggatcacgcgagagaagggagcggtggcaatggaggcgtacaccac

SEQ ID NO: 14 - protein, 23 aa, Zea mays
RRPRPRPPDHAREGSGGNGGVHH

SEQ ID NO: 15 - DNA, 69 bp, Triticum aestivum
  source 1..69: mol_type = genomic DNA
gcggtgaggaggccgcggccgcccggcaacccgagggaagggcgcggcggcggtggaggaagccacaac

SEQ ID NO: 16 - protein, 23 aa, Triticum aestivum
AVRRPRPPGNPREGRGGGGGSHN

SEQ ID NO: 17 - DNA, 87 bp, Cucumis sativus
  source 1..87: mol_type = genomic DNA
tataaattggaaggagagctacaaaagagattcgaggatttattaaaaacaaccacggataatgatattcctcgtggtggaaactat

SEQ ID NO: 18 - protein, 29 aa, Cucumis sativus
YKLEGELQKRFEDLLKTTTDNDIPRGGNY

SEQ ID NO: 19 - DNA, 69 bp, Prunus persica
  source 1..69: mol_type = genomic DNA
aaaagaccgagaattactcttcgtgcggcaaggccagaaataagcacagggagtggtgctcagactaat

SEQ ID NO: 20 - protein, 23 aa, Prunus persica
KRPRITLRAARPEISTGSGAQTN

SEQ ID NO: 21 - DNA, 429 bp, Solanum lycopersicum
  source 1..429: mol_type = genomic DNA
atggaagagaaaaaagagagtagatcatcagtttatgatgaagttatcaccaagaatccatttttttacttacaagaaggcattaaagctattctaaaatgccttggttttgaatcttcaaaacttgttcatcaagcatcctcttcatcctcgtcgtcctcatcatcatcgtcttcttcttcttcgagtatgttaggtactaataataaaaaagaagaagaagagagtgaaaaacaagaacaagagtgtgttttgttccatgaagatggtaagaagcaaggttcagattcaacaaatgataattacgaaaatgaccctccagctgaaactaatgatgaagatccaactcttatattagcaacagataggaggggaagaccaccatcaagacctaaagttggtagtggacctcctcctcagaacaattaa

SEQ ID NO: 22 - protein, 142 aa, Solanum lycopersicum
MEEKKESRSSVYDEVITKNPFFYLQEGIKAILKCLGFESSKLVHQASSSSSSSSSSSSSSSSSMLGTNNKKEEEESEKQEQECVLFHEDGKKQGSDSTNDNYENDPPAETNDEDPTLILATDRRGRPPSRPKVGSGPPPQNN

SEQ ID NO: 23 - DNA, 408 bp, Capsicum annuum
  source 1..408: mol_type = genomic DNA
atggaagagggtgcagataccagagagaaaagagagagtggcataacaacaggttatgatgttacaaagaatccaatgttttgcttacaagaagccataagaactattctcaaatgtcttggttttgaatcttcaaaacctgatcacccttcttcttcttcttcttcgtcaagtatgtcaaatgatactaaaaaagaagaggagaataatattatagaagaacaaagtgaacaacaaaagtgtgtgttccaacaagatgatggtcaagggaatcagaaacaggatgatgaccctccagctgaagtagatgatccaccaccagttatagaagcagcaacaggccggaggaggggaagaccaccatcaagacctggagttggaaggggacctcctcctgagaacaattaa

SEQ ID NO: 24 - protein, 135 aa, Capsicum annuum
MEEGADTREKRESGITTGYDVTKNPMFCLQEAIRTILKCLGFESSKPDHPSSSSSSSSMSNDTKKEEENNIIEEQSEQQKCVFQQDDGQGNQKQDDDPPAEVDDPPPVIEAATGRRRGRPPSRPGVGRGPPPENN

SEQ ID NO: 25 - DNA, 375 bp, Glycine max
  source 1..375: mol_type = genomic DNA
atgatggctctagctactccaggtagccctagctccatgcacatgccactggtgcaaacgtccaaccctagctcacaaaattctatgacgacaattaccgaaaggagaggaagtgtagtagttctacaaacgaaaagaaaacatgatggaggaaaaggacgtgatccacaaactaatagcatagggtcactcaaaagcacaacaaccaaaaggagagggagtgttctacaaacgaaaagaagacatgacggagggaaaggacgtgatccgcaaacgaatagcgtagggctactcaaaagcacagaaaatggtccaaaaactaatagtgtagggctactcgaaaccacaacaaataatcgatcttcagaaaattga

SEQ ID NO: 26 - protein, 124 aa, Glycine max
MMALATPGSPSSMHMPLVQTSNPSSQNSMTTITERRGSVVVLQTKRKHDGGKGRDPQTNSIGSLKSTTTKRRGSVLQTKRRHDGGKGRDPQTNSVGLLKSTENGPKTNSVGLLETTTNNRSSEN

SEQ ID NO: 27 - DNA, 345 bp, Helianthus annuus
  source 1..345: mol_type = genomic DNA
atggtggaggaaagggctgaagttgtgtatgatattggatatggttatggtaacccttgtaggtatttacaagaagtttttagaagctttttaaggtgtttggggttagagaaacaagaaggaaaagaaagaaacactagtggtggtgttggtggccgcgacacaggcggtggtgacggtgacggtgacggtgacggtgacggaggtggaggtgaggtggatccacctacaacttctcctattatggatcctactgatgaacctatgtctacaaggagactcacaagaagaccccctccgccaagggggccgattagctctggaggaggcggtcaaaccaactaa

SEQ ID NO: 28 - protein, 114 aa, Helianthus annuus
MVEERAEVVYDIGYGYGNPCRYLQEVFRSFLRCLGLEKQEGKERNTSGGVGGRDTGGGDGDGDGDGDGGGGEVDPPTTSPIMDPTDEPMSTRRLTRRPPPPRGPISSGGGGQTN

SEQ ID NO: 29 - DNA, 381 bp, Gossypium arboreum
  source 1..381: mol_type = genomic DNA
atggaagatacttcatcagcaaaggaagtaacgttgcaaggaactccagctaattacttccccaggtccttccatgaagttgtaggtgccattctcaggtgtttgggacttgaaactggattccaacaaaaccctaatccatgtccaaagaaagaagatgacagtaaagccaatcataatcaatctgtttctcagaaggaaagtccagatccaccttcatcaacagacaattcagatccatcaaccactgtgatcgacccaccagctgatcctcctccttccaccactggagacacgaacgatggcgaacttcccatggtttctctctttactcctaaaaggccagggacaagcgccggcagtggacctcagattaattaa

SEQ ID NO: 30 - protein, 126 aa, Gossypium arboreum
MEDTSSAKEVTLQGTPANYFPRSFHEVVGAILRCLGLETGFQQNPNPCPKKEDDSKANHNQSVSQKESPDPPSSTDNSDPSTTVIDPPADPPPSTTGDTNDGELPMVSLFTPKRPGTSAGSGPQIN

SEQ ID NO: 31 - DNA, 282 bp, Oryza sativa
  source 1..282: mol_type = genomic DNA
atggcgatgtcgtcgtctcccgcctcgccgccgccatccttcctgatcggcggcgcccaggcccagctcctgcgccaccgggaggaaatgctgcttgttcttccctcccctccctccggccggcagctgccatcggaggaggaagaagcagcaccatgcgccgtcaatggccggagtacgatccttgccgccgccgatgattccaagccaacgcggcctggagcgccggcggagggctccggcggcaacggcggagcaatccacaccgctgcttcttcctga

SEQ ID NO: 32 - protein, 93 aa, Oryza sativa
MAMSSSPASPPPSFLIGGAQAQLLRHREEMLLVLPSPPSGRQLPSEEEEAAPCAVNGRSTILAAADDSKPTRPGAPAEGSGGNGGAIHTAASS

SEQ ID NO: 33 - DNA, 516 bp, Zea mays
  source 1..516: mol_type = genomic DNA
atggatgagcacggggaaaaggaagagaacaagtctcaagattcggctttggcggcggagcagcgcgaggagacggcggcggcggagggagaggacacgtctgaggaatccacggaccagcgcgaggacgggcacgggtataaagcggacgaatcggcgggcctgctgcccgaggacgacgtgggctctggagaagcctcggcggcgccacacttcgggcacccgtgctcgttgttgcgcgcctgcgccggattcctgggcctgcacggctgcggcggcgatcagaagccggcggcggctgccgttgctgcatctgcagctgaagccgccacggcggcggcggcgagctcgtcccaggatgaagaagacggcggcgtcgacatggcgaaggctaacggtttctacatgcatgaggtgatcacccgcgtatgggcggtcgcggtgaggaggccgcggccgcggccgccggatcacgcgagagaagggagcggtggcaatggaggcgtacaccactag

SEQ ID NO: 34 - protein, 171 aa, Zea mays
MDEHGEKEENKSQDSALAAEQREETAAAEGEDTSEESTDQREDGHGYKADESAGLLPEDDVGSGEASAAPHFGHPCSLLRACAGFLGLHGCGGDQKPAAAAVAASAAEAATAAAASSSQDEEDGGVDMAKANGFYMHEVITRVWAVAVRRPRPRPPDHAREGSGGNGGVHH

SEQ ID NO: 35 - DNA, 720 bp, Triticum aestivum
  source 1..720: mol_type = genomic DNA
atggaccagcaccaggagctggaggtggatgtggagggagagccaacggatcagcgcaaggaggaggaggcggacgcggacgcgggcaagcctgaagagccgacggatcagcgcaaggaggaggaggaggagcgggaggagggagacaacctcaaagagccaacggatcagcgcaaggaggacaagcctgacgggctgacggagcagcacaaggcggaggcggtggcggttgcggtggtggtggcggaggtggacaagtctgaagagctgacggaccagcgcaaggcgggggcggtggtggaggtggaggtggacaagtttgaagagctcaccgaccagcgcaaggaggaggcggcggaggacaaggacaaggagacggagaggagctccagcgaagagtcgccgtcgctcttcgtgcacccgtgctcgctgctgctgcggtacctcgtccgtgcctacgcctggtgcatgggcatggccgactggttcggcggcggcggcacaaggccgagcgccgctcctgccgcgtcccttaactcgtcacgggaagaagcaggcgaggcggcgggcgtcaggaccagggagatctccagctccaagaccactgacagagggttctacgtgcgggagatgatcgtgagcatgtgggcggtgaggaggccgcggccgcccggcaacccgagggaagggcgcggcggcggtggaggaagccacaactag

SEQ ID NO: 36 - protein, 237 aa, Triticum aestivum
MDQHQELEVDVEGEPTDQRKEEEADADAGKPEEPTDQRKEEEEREEGDNLKEPTDQRKEDKPDGLTEQHKAEAVAVVVVAEVDKSEELTDQRKAGAVVEVEVDKFEELTDQRKEEAAEDKDKETERSSSEESPSLFVHPCSLLLRYLVRAYAWCMGMADWFGGGGTRPSAAPAASLNSSREEAGEAAGVRTREISSSKTTDRGFYVREMIVSMWAVRRPRPPGNPREGRGGGGGSHN

SEQ ID NO: 37 - DNA, 258 bp, Cucumis sativus
  source 1..258: mol_type = genomic DNA
atggcttccaacagggtttcgatgattttgtatgtgctgttcttcattattatgctttcaatttcatctaataataattataatgcttctgcaagaaaatttcttgacgttaatttccctcttcttgacgatgccttcgaccttcaaatgggtgcatcttcattattttataaattggaaggagagctacaaaagagattcgaggatttattaaaaacaaccacggataatgatattcctcgtggtggaaactattaa

SEQ ID NO: 38 - protein, 85 aa, Cucumis sativus
MASNRVSMILYVLFFIIMLSISSNNNYNASARKFLDVNFPLLDDAFDLQMGASSLFYKLEGELQKRFEDLLKTTTDNDIPRGGNY

SEQ ID NO: 39 - DNA, 351 bp, Prunus persica
  source 1..351: mol_type = genomic DNA
atggagaaggcatcatcaggaaaggaggtcatcatcactactgagaagatggttgctgtttcttatgatcgttaccaattccttggagcagctgttaggtctcttttcaagtgtttgggtgttggggattcttcaacttcaaccacacaacaagtgcttacagatttttcggttgaagggacaccagccatgtcagctacggcagcagagcaggttgttccaccgccagcggcagtagtcgcccctgaagctgaccctgctgctgcttctaacgcccctaaaagaccgagaattactcttcgtgcggcaaggccagaaataagcacagggagtggtgctcagactaattga

SEQ ID NO: 40 - protein, 116 aa, Prunus persica
MEKASSGKEVIITTEKMVAVSYDRYQFLGAAVRSLFKCLGVGDSSTSTTQQVLTDFSVEGTPAMSATAAEQVVPPPAAVVAPEADPAAASNAPKRPRITLRAARPEISTGSGAQTN

SEQ ID NO: 41 - DNA, 3315 bp, Solanum lycopersicum
  source 1..3315: mol_type = genomic DNA
atgaagatagctgttcataatttgatctttttctactgctgctatttctcagtgtcggcttttgctgtatgtggtctcacttctgatggcaccgctttagtttccctttctagtgactggattggtgtaccctcttcttggaatgcttcagataccaatccatgttcttgggttggcgttgagtgtgatgacaaccactttgtcacctctttgaacctctctggctatgatatctctggccaattgggacctgaaattgcttatttgaaacacttgcttaccatggacttgagttataatgctttctctgcctccattccttctcaactcactaactgcactcttcttcgttacttggatctatcctacaacacctttactggagaaatcccttcaaacattggaaatttacacaagttaacctacataagcctcttctctaattcactcactggtaatattcctcattccttgttttctatcccacatttggaaactatctactttaaccaaaacagtttgaatggttccatcccgtccggcattgctaacttgactcacctgttgtctctctatctttatcagaatgacttgtccggtccaattccctcttccataggaaactgtacaaacttgcaagaattgtatttgaatgataatcatttagttggtagtttgcctgagagtttacaaaagctacaacaccttgtttatttggacttaagcaataatagtctacaaggaagtatccctttcagtttaggcaactacaaacatctcgacactctggtcttgtcatccaatagtttcaatggggaacttccaccaactttgatgaattctacgaacctaaaagttcttgctgctttttcttcgggtttaagtggccctattcctgctactctaggccagctcacaaagttggagaaactttaccttactgacaacaatttctctggaaaaataccacctgagctgggaaagtgccaggccttgatggaattacatttacccggaaaccaactggaaggtgaaattcctagtgaactaggatcactcacccagttgcagtatctctctctatattccaacaaattgagtggtgagattccccccactatttggaagattcaaagtcttcaacatattcttgtctatcggaacaacctgactggggagttacctcttgaaatgactgagttgaaacaattgaagaacatatccttatttgagaaccagtttaccggagttatacctcaaggtttggggattaacagcagtctaacgctgctggatttcacaaacaacaccttcacaggtcctgttccacctaatctttgttttggaaagaaactggagaagcttcttttaggttataatcatcttgaaggtggcattccttctcaattaggacaatgtcatactttgacgagagtaattctcaaaaaaaataatctgtcaggtgccattccagattttgttaagaatataaatcctattttcttggatctcagtgagaatggcttcagtgggaaaatatctccaagtttggcaaatcttgaaaatgctacatcaattgacttatcagtgaataagctttcaggatttatgccaccagagctggcaaaccttgccaacctccagggtttaaatctgtcgtacaatggtttggaaggtgtactgccatctcaactttcaaactggcagagactgttgaagtttgatgcaagtcataatttgctgagtggatcgatcccatccgcgtttggaagcttggaggagctatccattttgagtctgtgtgagaataatctttcaggaggtattcccacctccttgtttgcactcaagaagctgtccaagctgcagcttggtggaaatgcacttggtggggaaattcattcagcgattgcgactgcatcaagagaaacactgaggtgtttaaatctgagtagtaacaggttgacaggtgaacttcctgcggagctaggaaaatttactttcctggaggagttagatatagctggcaataacatttctggaactttaagagttcttgatggaatgcattcattgttattcatcaatgtatcagacaacctcttttctggtccagtacctgcacatctgatgaagttcctcaactcaactcctacctctttctcgggaaacttgggcctttgtgtgcactgtgatccagaagaaggctcaaattgccctgagaatatcacgttaaggccctgtgatcttcaatcaaacaatggaaggcacctaagtgtagcagaaacggcaatgattgcacttggggcactgatatttaccatttccttactcctagtaattgcttacatgcttctgtggcgcaaaagttctgggaaaggagttgcaatctctgctcaagaaggtgcatcgtccctgcttaataaagtattggaagctactggaaacctaaacgataagtatgtcattgggaggggagctcatggagttgtatataaggccatcttgggtccagggaaagtgtatgctgtgaaaaagctggtgtttgttggtatgaaggatggaagcagaagtatggttagagaaattcaaacaattggaaaggttaggcaccgcaatcttgtcaaattagaagacttctggctgagaaaggattatgggttgattctttataattacatggagaacgggagccttcatgatattctccatgagactaagccacctgtaacattagaatggagcgttcggtaccaaattgctattggagttgctcaagggttgtcatatctccatttcgactgtgatcctgccatcgtgcatcgagacatcaagcccatgaatatcttgttggactctgatctggagcctcacatatcagattttggtattgccaagcttctggatcagtctgcagcaacttccgcctccaatgcccttcagggcacagttgggtacatggctccagaaactgcatttgcagctacaaagagcaaggagtcggatgtttatagttatggcattgtcctattagaacttataactcgaaagaaggtgttggaccgttcactctatggggaaacagatattgtatgttgggttaggtctgtttggacagaaactgaagaaattgagaagattgtggatccaagacttttggacgaattcatagactcaagtgtcatggaacaagtaattgaagtgctttcactagctcttagatgtacagagaaggaagtcagcaaaagaccctcaatgaaagaggtagtcaagctattaacaaggtcaagttcgagtatacgaagcaagtactag

SEQ ID NO: 42 - protein, 1104 aa, Solanum lycopersicum
MKIAVHNLIFFYCCYFSVSAFAVCGLTSDGTALVSLSSDWIGVPSSWNASDTNPCSWVGVECDDNHFVTSLNLSGYDISGQLGPEIAYLKHLLTMDLSYNAFSASIPSQLTNCTLLRYLDLSYNTFTGEIPSNIGNLHKLTYISLFSNSLTGNIPHSLFSIPHLETIYFNQNSLNGSIPSGIANLTHLLSLYLYQNDLSGPIPSSIGNCTNLQELYLNDNHLVGSLPESLQKLQHLVYLDLSNNSLQGSIPFSLGNYKHLDTLVLSSNSFNGELPPTLMNSTNLKVLAAFSSGLSGPIPATLGQLTKLEKLYLTDNNFSGKIPPELGKCQALMELHLPGNQLEGEIPSELGSLTQLQYLSLYSNKLSGEIPPTIWKIQSLQHILVYRNNLTGELPLEMTELKQLKNISLFENQFTGVIPQGLGINSSLTLLDFTNNTFTGPVPPNLCFGKKLEKLLLGYNHLEGGIPSQLGQCHTLTRVILKKNNLSGAIPDFVKNINPIFLDLSENGFSGKISPSLANLENATSIDLSVNKLSGFMPPELANLANLQGLNLSYNGLEGVLPSQLSNWQRLLKFDASHNLLSGSIPSAFGSLEELSILSLCENNLSGGIPTSLFALKKLSKLQLGGNALGGEIHSAIATASRETLRCLNLSSNRLTGELPAELGKFTFLEELDIAGNNISGTLRVLDGMHSLLFINVSDNLFSGPVPAHLMKFLNSTPTSFSGNLGLCVHCDPEEGSNCPENITLRPCDLQSNNGRHLSVAETAMIALGALIFTISLLLVIAYMLLWRKSSGKGVAISAQEGASSLLNKVLEATGNLNDKYVIGRGAHGVVYKAILGPGKVYAVKKLVFVGMKDGSRSMVREIQTIGKVRHRNLVKLEDFWLRKDYGLILYNYMENGSLHDILHETKPPVTLEWSVRYQIAIGVAQGLSYLHFDCDPAIVHRDIKPMNILLDSDLEPHISDFGIAKLLDQSAATSASNALQGTVGYMAPETAFAATKSKESDVYSYGIVLLELITRKKVLDRSLYGETDIVCWVRSVWTETEEIEKIVDPRLLDEFIDSSVMEQVIEVLSLALRCTEKEVSKRPSMKEVVKLLTRSSSSIRSKY

SEQ ID NO: 43 - DNA, 3321 bp, Capsicum annuum
  source 1..3321: mol_type = genomic DNA
atgaagattgatgtcaaaaatgtcatctttttctacctttgctgcttctctgtgtcggtttgtgctctcaattcccatggcacagttctactttcccttttcaggcactggactgctgtgccctcctctgttaagtcttcttggaatgcttcagattccactccctgttcttgggttggtgtggaatgtgataccactcaccttgtcacttctttgaacctctctggctatggaatctctggccaattgggtcctgaaattgcttatttggagcacttgcgtaccattgatctgagctataatgctttctttggccctattccttctcaactcgtaaattgcactcttcttgattacttggacctgtcctacaacacctttactggagaaatcccttcaaagattggaaatttacacaagttgacctacataagcctctacgctaattcattgactggtaatattcctgattccttgttttctatcccacatttggactccatttaccttttccaaaacaggttaaatggttccatcccatctcgcattggcaacttgactaagcttgtgtctctctttctttatgacaatgagttgttcggtccaattccgtcttccattagtaactgtactaacttgcaagaactgtatttgaatgaaaacaatgtagttggtagtttgcctgagaatttagataagctagaacaccttgtttatttggacttaagcagtaatagactacaaggaagtatccctttcagtttaggtgggaactgcaaagatcttgacactttggtcttgtcatccaacaatttaaatggaaatcttccaccaagtttgagcaattgtactaacctacgagttcttgctgctttctcttctagtttaagtggccctatacctgcttctctaggccagctcacaaagttagagaagctttaccttgctgacaacaatttctctggaaaaataccacctgagctggggaagtgccagtccttgctggaattacttttaccagaaaaccaactggaaggtgaaattcctagtgaactagggtcactcagccagttgcagtatctcgctctatattccaacaaattgagcggcgagattccccgtgccatttggaagattcaaagtcttcaacattttcttgtctatcggaacaacctgaccggggaggtacctcttgaaatgaccgagttgaaacaactgaagaacatatctttatttgacaaccggttcactggagttatacctcaaggtttggggattaacagcagtctaacgctgctggatttcacgaacaacgccttcacaggtcctgttccacctaatctttgttttggcaagaaactacagaaacttatgctaggttataatcattttgaaggtggcattccttctcaattaggtaagtgtgctactttggcgagagtaattctcaaaaagaataaactctcaggtgccattccagattttggtaagaatataaatcctattttcttggatctcagtgaaaatggcttcagtgggagaatacccccaagtttggcaaatcttggaaatgttacattaattgacttatcagtgaataagctctcggggtttataccaccagagctggcaaaccttgtcaacctccaggttcttgatttgtcgtacaatagtttggaaggtgtactgccatctcaactttcaaactggcagagactgttgcagtttgatgcaagccataatttgttgagtggatcgatcccatccacgtttggaagcttgggagagctatccattttgagtctgagtgagaataatctttcaggaggtattccaacctccttgtttgaactcaagaagctgtctgagctgcagcttggcggaaatgcacttggtggtgaaattcattcagcaattgcgacttcatcaagagaaacactgaggttgttgaatctgagtagtaacgggttgacaggtgaaattcctgcagagctagggaaatttactttcctggagaagttagatgtagctggaaacaatattactggaactttaagaatttttgaggggatgcgttcattgatcttcgtcaatgtgtcacacaacctcttttctggtccagtacctgcaaatttgatgaagttcctaaactcaacacctacctctttctcgggaaactttggcctttgcgtgcactgtgatccagaaaaaggctcaaattgccctgagaatggcactctaaggccttgtgatctccaatcaaaaaatggaagacacctaagtggagcagaaacgacaatgattgcacttggggtactgttatttaccatttgcttactcctagtaattgcttacatgcttctgtggcgcaaaaattctgggaaaggagttgcaatctgtgctcaagaaggtgcttcatccctgcttaataaagtattggaagctacagagaacctaaatgataagtatgtcattgggaggggagcacatggaattgtatttaaggccatcttgggtccagggaaagtgtatgccgtgaagaagctggtgtttgttggtataaaggatggaagcacaagtatggttagggaaattcagacaattggaaaggttagacaccgcaatcttgtcaaattagaagacttctggctgagaaaggattatggactgattctttataactacatggagaacgggagccttcatgatattctccatgagattaatccacctgtagcattagaatggagtgttcggtaccgaattgctattggaactgctcaagggttgtcatatctccactttgactgtgatcctgcaatcgttcatcgagatatcaagcccatgaatatcttgttggactcagatctggagcctcacatatctgattttggcattgccaagcttcttgatcagtctggagcaacttccacctccaataccctccagggtacagttggatacatggctccagaaactgcatttgcagcttcaaagagcaaggagtcagatgtttatagttatggtgttgtcctgttagaacttataactcgaaagaaggccttggatccttcattgtatggggatacagatattgtcagttgggttaggtctatttggacagaaattgaagaaattgaggagattgtggatccaagccttttggacgaattcatggactcaagtgtcatggaacaagtaattgaagtgctttcactagctcttagatgtacagagaaggatgtaagcagaagaccctctatgaaagaggtggtgaagctattaacaaggtccagttcgagtataagaatcaaatactag

SEQ ID NO: 44 - protein, 1106 aa, Capsicum annuum
MKIDVKNVIFFYLCCFSVSVCALNSHGTVLLSLFRHWTAVPSSVKSSWNASDSTPCSWVGVECDTTHLVTSLNLSGYGISGQLGPEIAYLEHLRTIDLSYNAFFGPIPSQLVNCTLLDYLDLSYNTFTGEIPSKIGNLHKLTYISLYANSLTGNIPDSLFSIPHLDSIYLFQNRLNGSIPSRIGNLTKLVSLFLYDNELFGPIPSSISNCTNLQELYLNENNVVGSLPENLDKLEHLVYLDLSSNRLQGSIPFSLGGNCKDLDTLVLSSNNLNGNLPPSLSNCTNLRVLAAFSSSLSGPIPASLGQLTKLEKLYLADNNFSGKIPPELGKCQSLLELLLPENQLEGEIPSELGSLSQLQYLALYSNKLSGEIPRAIWKIQSLQHFLVYRNNLTGEVPLEMTELKQLKNISLFDNRFTGVIPQGLGINSSLTLLDFTNNAFTGPVPPNLCFGKKLQKLMLGYNHFEGGIPSQLGKCATLARVILKKNKLSGAIPDFGKNINPIFLDLSENGFSGRIPPSLANLGNVTLIDLSVNKLSGFIPPELANLVNLQVLDLSYNSLEGVLPSQLSNWQRLLQFDASHNLLSGSIPSTFGSLGELSILSLSENNLSGGIPTSLFELKKLSELQLGGNALGGEIHSAIATSSRETLRLLNLSSNGLTGEIPAELGKFTFLEKLDVAGNNITGTLRIFEGMRSLIFVNVSHNLFSGPVPANLMKFLNSTPTSFSGNFGLCVHCDPEKGSNCPENGTLRPCDLQSKNGRHLSGAETTMIALGVLLFTICLLLVIAYMLLWRKNSGKGVAICAQEGASSLLNKVLEATENLNDKYVIGRGAHGIVFKAILGPGKVYAVKKLVFVGIKDGSTSMVREIQTIGKVRHRNLVKLEDFWLRKDYGLILYNYMENGSLHDILHEINPPVALEWSVRYRIAIGTAQGLSYLHFDCDPAIVHRDIKPMNILLDSDLEPHISDFGIAKLLDQSGATSTSNTLQGTVGYMAPETAFAASKSKESDVYSYGVVLLELITRKKALDPSLYGDTDIVSWVRSIWTEIEEIEEIVDPSLLDEFMDSSVMEQVIEVLSLALRCTEKDVSRRPSMKEVVKLLTRSSSSIRIKY

SEQ ID NO: 45 - DNA, 3252 bp, Glycine max
  source 1..3252: mol_type = genomic DNA
atggggtatctgtatctcttgctgcttctatgtttttcttccttgttatatgctgcttctgcattgaactctgatggtttggctttgttgtccctcttgagggattggactactgtgcctagtgacataaactccacatggaggttgtctgattccactccatgctcatcttgggcaggagtgcattgtgataatgccaacaatgtggtttctctaaacctcactagttattcgattttgggtcaattaggacctgatcttggacgtttggttcacttgcaaaccatagacttatcatataatgatttctttgggaaaatccccccagaattagagaactgtagcatgcttgagtacttgaacctttctgtaaacaactttagcggaggaatacctgagagcttcaaaagcttgcaaaatttgaagcatatataccttttatctaatcaccttaatggtgaaattcctgaatccttgtttgaaatttctcacctggaagaagtggatcttagcagaaacagtttaactggttcaatccccttaagtgttgggaatatcactaagcttgtcactctggatctttcttataatcagttgtcagggacaattccaatatccattggaaattgtagtaacttagagaatctgtatttggaaaggaatcaattagagggagttattcctgagagtctaaataatctcaaaaatcttcaagagttatatctcaattataataaccttggaggtactgttcaattgggatctggatattgcaaaaagttgtctattttgagtatttcttacaataactttagtgggggtataccatcaagcttggggaattgtagtggtctaatagagttttatgcttcagggaataacttagttggcactataccatcaaccttcggcctcctgcccaacctttctatgttattcattccggagaacctattgtcagggaaaatacctccacagattggtaattgcaaatcactgaaagagttgagtttgaattccaatcaacttgaaggagaaattccaagcgaattaggaaacttgagtaaattgcgtgatcttagattgtttgaaaaccatttgacaggagaaattccacttggcatatggaaaattcaaagccttgagcagatccatatgtacattaataacctctcgggcgagctaccacttgagatgacagagcttaaacatcttaagaatgtctccttgtttaacaaccagttctccggagtcatacctcaaagcttaggaatcaatagcagtttggtggtgttagattttatgtataataatttcactggtaccctcccaccaaatctttgttttggaaagcacctggtcaggctgaatatgggtggcaatcaatttattggcagcatacctcctgatgtaggaaggtgtacaactcttacaaggttgagacttgaagataataatttaactggggcacttcctgattttgaaactaatccaaacctctcttacatgagcatcaacaacaacaatatcagtggagcaattccatcaagtttgggaaactgcacaaatctctctcttttagatttgtccatgaacagcttgacgggtcttgtaccttcagagctaggaaaccttgtgaatcttcagactttggatctttctcacaataacttgcaaggtcctttgccacatcagctgtcaaactgtgccaaaatgatcaagtttaatgtcggattcaattctttgaatggttcggttccttcaagttttcagagctggacaacattaacaactttaattctctcagagaatcgttttaatggtggtattccagctttcttgtcagaatttaaaaagctcaacgagttacgacttggtggaaatacgtttggaggaaacattcctagatcaattggagaattggtgaatttgatatatgagctaaatttaagtgctaatgggctgataggtgaacttcctagggagattggaaacctgaagaatctgctaagcctggatctatcttggaacaatttgacaggaagtatacaagttcttgatgagctcagttcattatctgaattcaacatctcatttaattcttttgaaggtcctgtgccacaacagctaacaacattaccaaattcttctttatcatttttgggcaatcctggtctgtgtgactcgaatttcactgtgagcagctatttacagccatgtagcacaaattcaaaaaagtcaaaaaagctgagtaaagttgaagctgtgatgatagcacttggatccttagtatttgttgttctgctgctggggttaatctgtatattctttatcagaaaaattaagcaggaagccataatcattgaggaggatgattttccaacacttcttaatgaagtaatggaagcgacagaaaatctaaatgatcaatatattattggcagaggagctcaaggagtggtttacaaagcagcaataggtccagacaaaattttggctataaagaaatttgtatttgctcatgatgaagggaaaagctcaagcatgaccagagaaattcaaaccattggaaagattaggcatcgaaatttggtcaaattggaagggtgctggttgagagaaaactatggtctaattgcatacaaatacatgccaaatggaagccttcatggtgctttgcatgagaggaatccaccatactccttagaatggaatgtccggaataggatagctcttggaattgctcatggattggcttatctccattatgactgtgatcctgtcatagtgcacagagacatcaaaaccagcaatatacttctagattctgacatggagcctcatattgcagattttggtatttccaagcttttagatcagccttctacctcaacacagtcgtcatctgttactggtacacttggatatatagcaccagagaaatcctatacaacaacaaagggtaaggaatctgatgtatacagttatggggtagttttgctggaactgatatccagaaagaagccattggatgcatcatttatggaagggacggatatagttaattgggctagatctgtctgggaggaaacaggagttattgacgaaattgttgatccagagatggctgatgaaatttcaaattctgatgtgatgaaacaagttgccaaggtgcttttggtggctttgagatgcacattaaaggatccacgcaagagacctacgatgagggatgttatcaagcatttgtag

SEQ ID NO: 46 - protein, 1083 aa, Glycine max
MGYLYLLLLLCFSSLLYAASALNSDGLALLSLLRDWTTVPSDINSTWRLSDSTPCSSWAGVHCDNANNVVSLNLTSYSILGQLGPDLGRLVHLQTIDLSYNDFFGKIPPELENCSMLEYLNLSVNNFSGGIPESFKSLQNLKHIYLLSNHLNGEIPESLFEISHLEEVDLSRNSLTGSIPLSVGNITKLVTLDLSYNQLSGTIPISIGNCSNLENLYLERNQLEGVIPESLNNLKNLQELYLNYNNLGGTVQLGSGYCKKLSILSISYNNFSGGIPSSLGNCSGLIEFYASGNNLVGTIPSTFGLLPNLSMLFIPENLLSGKIPPQIGNCKSLKELSLNSNQLEGEIPSELGNLSKLRDLRLFENHLTGEIPLGIWKIQSLEQIHMYINNLSGELPLEMTELKHLKNVSLFNNQFSGVIPQSLGINSSLVVLDFMYNNFTGTLPPNLCFGKHLVRLNMGGNQFIGSIPPDVGRCTTLTRLRLEDNNLTGALPDFETNPNLSYMSINNNNISGAIPSSLGNCTNLSLLDLSMNSLTGLVPSELGNLVNLQTLDLSHNNLQGPLPHQLSNCAKMIKFNVGFNSLNGSVPSSFQSWTTLTTLILSENRFNGGIPAFLSEFKKLNELRLGGNTFGGNIPRSIGELVNLIYELNLSANGLIGELPREIGNLKNLLSLDLSWNNLTGSIQVLDELSSLSEFNISFNSFEGPVPQQLTTLPNSSLSFLGNPGLCDSNFTVSSYLQPCSTNSKKSKKLSKVEAVMIALGSLVFVVLLLGLICIFFIRKIKQEAIIIEEDDFPTLLNEVMEATENLNDQYIIGRGAQGVVYKAAIGPDKILAIKKFVFAHDEGKSSSMTREIQTIGKIRHRNLVKLEGCWLRENYGLIAYKYMPNGSLHGALHERNPPYSLEWNVRNRIALGIAHGLAYLHYDCDPVIVHRDIKTSNILLDSDMEPHIADFGISKLLDQPSTSTQSSSVTGTLGYIAPEKSYTTTKGKESDVYSYGVVLLELISRKKPLDASFMEGTDIVNWARSVWEETGVIDEIVDPEMADEISNSDVMKQVAKVLLVALRCTLKDPRKRPTMRDVIKHL

SEQ ID NO: 47 - DNA, 3261 bp, Glycine max
  source 1..3261: mol_type = genomic DNA
atgtccatgatttggattgttttcttttccttgtcttgcatgtcttgtgctgttgtttcttcactcacctccgatggggtgactctcttgtcactcttgaggcactggacatccgtgcctccttccataaacgccacctggcttgcctccgataccactccatgctcctcctgggtaggagtacaatgtgaccattcccaccatgtcgtcaaccttaccctcccagattatggtattgctggtcaattaggacctgaaattggaaatttaagtcgcctagagtacttagaacttgctagcaacaaccttactggtcaaatacctgacgccttcaaaaacatgcacaacctcaatttactcagccttccatataatcaactgtctggtgaaattccagattccttgactcatgctccccaactaaatcttgttgatctttctcataacactttaagtggatccatccccacaagtattgggaacatgactcagctcttgcagttgtatcttcagagtaaccagttgtctgggacaattccctcatccattgggaactgcagcaaattacaagaattgtttttggataaaaatcacttggagggtatcctgcctcagagtcttaacaatctcaatgatcttgcttattttgatgttgccagcaatagacttaagggtaccattccttttggttctgctgccagttgtaaaaatttgaagaatttggatctctcattcaatgactttagtggaggccttccctcaagcttggggaactgtagcgctttatctgaattttctgccgtgaactgcaatttggatggcaatattcctccctcctttggtctactgaccaagctttctattctataccttcccgagaaccacttatctggaaaagtacctccagaaattggcaactgcatgtctttgacagagttacatctgtattccaatcaacttgagggaaacattccaagtgaactgggaaaactaagaaaactagtggatcttgaattgttttcaaatcaattgacgggtgaaattccactcagcatctggaagattaaatctctgaagcatctccttgtgtataataacagtctttctggggaacttcctttggagatgacagagctcaagcaactgaaaaacatctcattgtttagcaaccagttctccggagtcataccgcaaagcttgggaattaacagcagtttagttcttttggattttacaaataataaattcactggcaacatcccaccaaatctgtgttttggcaagaaattaaacatcctgaatttgggcatcaaccaacttcaaggcagcattcctcctgatgttgggagatgtacaacccttagaaggttaattcttcaacaaaataactttactgggcctcttcctgatttcaaaagcaatccaaatctcgaacatatggatatcagcagcaacaaaatccatggtgaaattccatcaagtttgcgaaattgcagacatatcactcacctaattttgtccatgaacaaatttaatgggcctataccctcagagctagggaacattgtcaatcttcaaactttgaatctcgctcacaacaacttagaaggtcctttgccctctcagctgtcaaagtgtaccaaaatggacaggtttgatgttggatttaatttcctaaatggttcattgccatcaggtctgcagagctggacaaggctaaccacattaattttgagtgagaatcactttagtgggggcctcccagctttcttgtcggaatataaaatgctttctgaactacaacttggtggcaatatgtttggtggcagaattcctagatcggttggagcattgcagagtttgaggtatggtatgaatctgagttcaaatgggctgataggagacattcctgttgagattggaaacttgaactttttggaaagactggatctgtctcagaacaatttgaccggaagcatagaagttcttggtgaactcctctccttagttgaagtcaatatttcatacaattcttttcatggtcgtgtaccaaagaagctaatgaaattgctcaagtctcccttgtcatcatttttgggcaatcctggcctatgtaccaccaccaggtgttcagcatctgatggcttggcttgcactgcaagaagctctataaaaccatgtgatgacaaatctactaaacagaaaggcctcagtaaagttgaaattgtgatgatagctctcgggtcctcaatacttgttgttttgctgttgctgggattagtttatattttttattttggaagaaaagcttaccaggaagtccatatctttgctgaagggggttcttcttcccttcttaacgaagtcatggaggctacagcaaacctaaatgatcggtatattattggcagaggagcctatggagttgtttataaagccctggtgggtccagacaaagcctttgctgcgaagaagataggatttgctgcgagcaaaggtaagaacttgagcatggccagagaaattgaaacccttgggaaaattcggcatcgaaatctggtcaaattggaagacttttggttgagagaagattatggtataattttgtacagctacatggcaaatggaagtcttcatgatgttttgcacgaaaagacaccaccactaaccttagagtggaatgtccggaataagatagctgttggaattgctcatggattggcttatctccattatgactgtgatcctcccatagtgcaccgagacatcaagccaagcaatatacttctagactctgatatggagcctcacattgctgactttggtattgccaaacttctggatcagtcttctgcttcaaatccttccatttctgttccgggtacaattggttatattgcaccagagaatgcttatacaacaacaaatagtagggagtctgatgtatacagttacggggtagttttgcttgagctgataaccagaaagaaggcagcagaatcagatccttccttcatggagggtactatagtagtggattgggttaggtctgtgtggagggaaacaggagacattaatcaaattgttgattcaagccttgcagaggaatttctagatatccatataatggaaaatattaccaaagtgcttatggtggctctgagatgtactgagaaggatccacacaagagacccacaatgagagatgttaccaagcagttagcagatgctaatccacgggcaagaagtacaaagggctag

SEQ ID NO: 48 - protein, 1086 aa, Glycine max
MSMIWIVFFSLSCMSCAVVSSLTSDGVTLLSLLRHWTSVPPSINATWLASDTTPCSSWVGVQCDHSHHVVNLTLPDYGIAGQLGPEIGNLSRLEYLELASNNLTGQIPDAFKNMHNLNLLSLPYNQLSGEIPDSLTHAPQLNLVDLSHNTLSGSIPTSIGNMTQLLQLYLQSNQLSGTIPSSIGNCSKLQELFLDKNHLEGILPQSLNNLNDLAYFDVASNRLKGTIPFGSAASCKNLKNLDLSFNDFSGGLPSSLGNCSALSEFSAVNCNLDGNIPPSFGLLTKLSILYLPENHLSGKVPPEIGNCMSLTELHLYSNQLEGNIPSELGKLRKLVDLELFSNQLTGEIPLSIWKIKSLKHLLVYNNSLSGELPLEMTELKQLKNISLFSNQFSGVIPQSLGINSSLVLLDFTNNKFTGNIPPNLCFGKKLNILNLGINQLQGSIPPDVGRCTTLRRLILQQNNFTGPLPDFKSNPNLEHMDISSNKIHGEIPSSLRNCRHITHLILSMNKFNGPIPSELGNIVNLQTLNLAHNNLEGPLPSQLSKCTKMDRFDVGFNFLNGSLPSGLQSWTRLTTLILSENHFSGGLPAFLSEYKMLSELQLGGNMFGGRIPRSVGALQSLRYGMNLSSNGLIGDIPVEIGNLNFLERLDLSQNNLTGSIEVLGELLSLVEVNISYNSFHGRVPKKLMKLLKSPLSSFLGNPGLCTTTRCSASDGLACTARSSIKPCDDKSTKQKGLSKVEIVMIALGSSILVVLLLLGLVYIFYFGRKAYQEVHIFAEGGSSSLLNEVMEATANLNDRYIIGRGAYGVVYKALVGPDKAFAAKKIGFAASKGKNLSMAREIETLGKIRHRNLVKLEDFWLREDYGIILYSYMANGSLHDVLHEKTPPLTLEWNVRNKIAVGIAHGLAYLHYDCDPPIVHRDIKPSNILLDSDMEPHIADFGIAKLLDQSSASNPSISVPGTIGYIAPENAYTTTNSRESDVYSYGVVLLELITRKKAAESDPSFMEGTIVVDWVRSVWRETGDINQIVDSSLAEEFLDIHIMENITKVLMVALRCTEKDPHKRPTMRDVTKQLADANPRARSTKG

SEQ ID NO: 49 - DNA, 3249 bp, Glycine max
  source 1..3249: mol_type = genomic DNA
atggggtatttgtatctcttgctgatttcatatttgtctgccttgttgtatgctgcttctgcattgaactctgatgggttggctttgttgtccctcttgagggactggactattgtgcctagtgacataaactccacatggaagttgtctgattccactccgtgctcatcttgggcaggagtgcattgtgataatgccaataatgtggtttctctaaacctcactagttattctatttttggtcaattaggacctgatcttggacgtatggttcacttgcaaaccatagacttatcatataatgatctatttggaaaaattcccccagaattagacaactgtaccatgcttgagtacttggacctttctgtaaacaactttagtggaggaatacctcagagcttcaaaaacttgcaaaatttgaagcatatagacctttcatctaatccgctgaatggtgaaattcctgaacccttgtttgacatttatcacctggaagaagtgtatcttagcaacaacagtttgactggttcaatttcctcaagtgttgggaatatcactaagcttgtcacactggatctttcttataatcagctgtcagggacaattcccatgtccattggaaattgtagtaacttagagaatctatatttggaaaggaatcaattagagggagttattcctgagagtctaaataatctcaaaaatcttcaggagttatttctcaattataataaccttggaggcactgttcaattgggaactggaaattgcaaaaagttgtctagtttaagtctttcttacaataacttcagtgggggtataccatcaagcttggggaattgtagcggtctaatggagttttatgctgcacggagtaacttagttggcagtataccatcaaccttgggcctcatgcccaacctttctcttctaatcattccagagaacctattgtctgggaaaatacctccacagattggtaattgcaaagcactggaagagttgcgtttgaattccaatgaacttgagggagaaattcccagtgaattgggaaacttgagtaaattacgcgaccttagattgtatgaaaaccttttgacaggagaaattccacttggcatatggaaaattcaaagccttgagcagatctatctgtacattaataacctttcgggggagctaccttttgagatgacagagctcaaacatcttaagaatatctccttgtttaacaaccagttctccggagtcatacctcaaagtttaggaatcaatagcagtttggtggtgttagacttcatgtataataatttcactggtacccttccaccaaatctttgttttggaaagcaactggtgaagctgaatatgggtgtcaatcaattttatggtaacatacctccagatgtgggaaggtgtacaactcttacaagggtgagacttgaagaaaatcatttcactgggtctcttcctgatttttatattaatccaaatctctcttacatgagcatcaacaacaacaatatcagtggagcaattccatcaagtttgggaaaatgcacaaatctctctcttttaaatttgtccatgaacagcttgacgggtcttgtaccttcagagcttggaaaccttgagaatctccagactttggatctttctcacaataacttggaaggtcctttgccacatcagctgtcaaactgtgccaaaatgatcaagtttgatgtcagattcaattccttgaatggttcggttccatcaagttttcggagctggacaacattaacagctttaattctctcagagaatcattttaatggtggtatcccagctttcttgtcagaatttaaaaagctcaacgagttacaacttggtggaaacatgtttggaggaaacattcctagatcaatcggagagctggtgaatttgatatatgaactaaatctaagtgctactgggctgataggtgagcttcctagggagattggaaacctgaagagtctgctaagcctggatctatcttggaacaatttgacaggaagtatacaagttcttgatgggctcagttcattatctgaattcaacatctcatataattcttttgaaggtcctgtgccacaacagctaacaacattaccaaactcttctttatcatttttgggcaatcctggcctgtgtggctcgaatttcactgagagcagctatttaaagccttgtgacacaaattcaaaaaagtcaaaaaagctcagtaaagttgcaactgtgatgatagcacttggatctgcaatatttgttgttctgctgctgtggttagtatatatattctttatcagaaaaattaagcaagaagccataatcattaaggaagatgattctccaacccttcttaacgaagtgatggaagctacagaaaatctaaatgatgagtatattattggcagaggagctcaaggagttgtttataaagcagcaataggtccagacaaaacattggctataaagaagtttgtattttctcatgaagggaaaagctcaagcatgaccagagaaattcaaacccttggaaagattaggcatcgaaatttagtcaaattggaagggtgctggttgagagaaaactatggtctaattgcatacaaatacatgccaaatggaagcctacatgatgctttgcatgagaagaatccaccatactccttagaatggattgttcggaataacatagcacttggaattgctcacggattgacttatctccattatgactgtgatcctgtcatagtgcacagagatatcaaaacaagcaacatacttctagattcagaaatggagcctcatattgcagattttggtattgctaaacttatagatcagccttctacctcaacacagttatcatctgttgctggtacacttggttatatagcaccagagaatgcttatacaacaacaaagggtaaggaatctgatgtatacagttatggggtagttttgctggagctgatatccagaaagaagccattggatgcatcatttatggaaggaacggatatagttaattgggcaagatctgtctgggaggaaacgggagttgttgatgaaattgttgatccagagctggctgatgaaatttcaaattctgaagtgatgaaacaagttaccaaggtgcttttggtggctttgagatgcactgaaaaggatccacgtaagagacctacgatgagggatgttatcaggcatttgtag

SEQ ID NO: 50 - protein, 1061 aa, Glycine max
MGYLYLLLISYLSALLYAASALNSDGLALLSLLRDWTIVPSDINSTWKLSDSTPCSSWAGVHCDNANNVVSLNLTNLSYNDLFGKIPPELDNCTMLEYLDLSVNNFSGGIPQSFKNLQNLKHIDLSSNPLNGEIPEPLFDIYHLEEVYLSNNSLTGSISSSVGNITKLVTLDLSYNQLSGTIPMSIGNCSNLENLYLERNQLEGVIPESLNNLKNLQELFLNYNNLGGTVQLGTGNCKKLSSLSLSYNNFSGGIPSSLGNCSGLMEFYAARSNLVGSIPSTLGLMPNLSLLIIPENLLSGKIPPQIGNCKALEELRLNSNELEGEIPSELGNLSKLRDLRLYENLLTGEIPLGIWKIQSLEQIYLYINNLSGELPFEMTELKHLKNISLFNNQFSGVIPQSLGINSSLVVLDFMYNNFTGTLPPNLCFGKQLVKLNMGVNQFYGNIPPDVGRCTTLTRVRLEENHFTGSLPDFYINPNLSYMSINNNNISGAIPSSLGKCTNLSLLNLSMNSLTGLVPSELGNLENLQTLDLSHNNLEGPLPHQLSNCAKMIKFDVRFNSLNGSVPSSFRSWTTLTALILSENHFNGGIPAFLSEFKKLNELQLGGNMFGGNIPRSIGELVNLIYELNLSATGLIGELPREIGNLKSLLSLDLSWNNLTGSIQVLDGLSSLSEFNISYNSFEGPVPQQLTTLPNSSLSFLGNPGLCGSNFTESSYLKPCDTNSKKSKKLSKVATVMIALGSAIFVVLLLWLVYIFFIRKIKQEAIIIKEDDSPTLLNEVMEATENLNDEYIIGRGAQGVVYKAAIGPDKTLAIKKFVFSHEGKSSSMTREIQTLGKIRHRNLVKLEGCWLRENYGLIAYKYMPNGSLHDALHEKNPPYSLEWIVRNNIALGIAHGLTYLHYDCDPVIVHRDIKTSNILLDSEMEPHIADFGIAKLIDQPSTSTQLSSVAGTLGYIAPENAYTTTKGKESDVYSYGVVLLELISRKKPLDASFMEGTDIVNWARSVWEETGVVDEIVDPELADEISNSEVMKQVTKVLLVALRCTEKDPRKRPTMRDVIRHL

SEQ ID NO: 51 - DNA, 3399 bp, Helianthus annuus
  source 1..3399: mol_type = genomic DNA
atggaaatggaaacaacaagaagaagaagaagtcttttgtttttgttttctcttctagttctaccattctatcttgtctctgggttgagctctgatgggttaaacctattatcattgatgacccactggacttcagtacctccttccatctattctacctggaatgcttcacacccaacaccatgctcatgggttggagttcactgtaaccctaacaaccatcatgtccactctctcaacctctcctcttacctcatttccggtcatataggaggcccagagcttgcaaacctcacccatctcacatcccttgacttgagcttcaataacctctctggacccttacctccacaacttggtaactgtacccgtcttcgttatctggacctttcctacaacatcctctctggacccattccacacactctagggaacctcatccacttaacgcagatcattctttacagtaacaaacttaccggttctatcccttaccagattggcaacttgagtaatcttgaggaacttgatttctctttaaaccacctcacaggaacaataccagaaagcctatggaagttgcaaaatcttagggtcttaagtctttatggcaactctcttctaggttctgtacccttcttagttaacgctccctatttggaagctgtttatctcacttctaatcagcttagtggtcccatttcatcgaccataggtaaccttagcaacctagttgacttgcagttagacagcaatcagttctggggtcctattccctcatccattgggaagtgcagcaggctgcagaccctcatgcttggtaacaatcgcttcactggtgaattgcctaataccctcaatcatctttctcagcttactaaattagacgtccataacaacagtttaaaaggtagcattcgctttggcggtggtaactgtttgaatttggtctacttggatatttcatttaaccagtttcatggcctcctgcctcatgaactgggaaactgcagcagcttaaaccaagttgctgctgttaactctgggttaggtggctctattccttcttctattggtaaacttactgcactcaccactctttacctttctattaatcatttttctggaaatataccacctgagcttggcggttgtaagtccttggttgatctacaactccatgctaaccaactggagggccttatcccgaacgaactcgggatgttaagtcaactgcaagtacttgaagcgtttgataaccgtctgacaggagaggtgcctctgggaatctggaagattaagggcctcaagaagctcaacatatacaacaataatctttcaggggaactgcccgatgaggttgctggaatgaagcaactaagggagattacgttgtacaacaatcgcttcactggagctatacctcaggggttgggtatcaatagcagtttgactgtgatcgatttcaccaataactcattcaccgggaaaattccaccaagcctctgctttcgaaagcagctgcaaaggctacttttgggttataattatttacaaggcagtgtgcctcatgatgttggaagctgttctagtctttcaagattaattctgcaacataacaacctcacaggagtccttccagagtttatggagaaccgcaacatgctatacatgaaccttaggggcaacttttttagcggaaaaattccagccagcttcggaaagcttaccaatatcacagaaattgacctctcaatgaataaactctctggtaacatacctctagagttgggaaatcttgtgcagattcaagctctgaatatttcctacaatgaactccaaggtcctttgccatcccagctaggaaattgtagcaggttgttggaattcgatgcaagtcataattcggttagtggttccatcccagccgcatttgggaagttgactcagctttcaactttaagtctaagcgataatcatttctctggggaaattccgcattttatatctcaactccaagatttaatagatctccaattaggtgggaattcattcagtggtcatatcccgtcatcgatagtagagcttcatagtctaaacaggttgaatctcagcagcaacaaactgattggtggcattccatcaaattttaggaagatggtaatgctagagcatttggatgtttcacacaacaatctaactggggatttagcatcacttggcgacaccgtcgggctggtcaccttagatgtttcttttaatcatttcacaggcccgataccagcaactttattgaagtctatcagctcgtcgtccttcgtggggaacacaggtctttgtgtagattgttgcattgaaaacagcaatatcaaaccatgtgttgttcgatcacggggcctgaagaaacgacattatataatgatagcagttggaatgtgtctgtttgtgatggttgtctatctagtactctatctggcggttatctgtcgcagaaaacaaaaacaggaaatggacatgcttgctgaagggtgctgttcatctttggtcataaaggcgctggaagccactgagaatttgaatgataagtatattattgggaggggggcccatggtacagtgtacaaggcttgcttgggtcatggtagggtctatgctgtgaagaggcttggttttggaggggggcaaacaagtatgaaaagagaagttgagacggttgggaaggtgagacacaggaatctggtgacgatacaagatgtgttgataaagaaggattatagtttgattttgtacaagtatatggaaaacggtagcctatatgatgttttgtatgaaaaagaaccaacattagtcttaagttggagcgttcggtacaggatagctttaggaactgcccagggtttggcatatctccattttgactgtgatcctgttatcatacaccgggatataaaacccatgaacattcttttggatggtgatatggaagctcatatctcggactttggcattgccaagcttctagatcagtctgctactggtgcactagcaaccagcacacttatgggcacaataggttacatcgctccagaaaacgcgttcacgaggtcagcaagcagggagttggacgtatatagctatggagttgtgttgctggagcttataagtaggcagaagcctgtgttgatagaaaatgtggacatggtgtcgtgggtaaggtcgagatcggaagaaatcgagaagattgtagacagagggctgttagaggaaattgaagaagataagagggtgagggaacaagtgagaaaagtggtgttggtggccttgagatgtacagagtgggaaccaaccagaagaccaacaatgagggaggtggtcaagtgtcttcaagattcagtttag

SEQ ID NO: 52 - protein, 1132 aa, Helianthus annuus
MEMETTRRRRSLLFLFSLLVLPFYLVSGLSSDGLNLLSLMTHWTSVPPSIYSTWNASHPTPCSWVGVHCNPNNHHVHSLNLSSYLISGHIGGPELANLTHLTSLDLSFNNLSGPLPPQLGNCTRLRYLDLSYNILSGPIPHTLGNLIHLTQIILYSNKLTGSIPYQIGNLSNLEELDFSLNHLTGTIPESLWKLQNLRVLSLYGNSLLGSVPFLVNAPYLEAVYLTSNQLSGPISSTIGNLSNLVDLQLDSNQFWGPIPSSIGKCSRLQTLMLGNNRFTGELPNTLNHLSQLTKLDVHNNSLKGSIRFGGGNCLNLVYLDISFNQFHGLLPHELGNCSSLNQVAAVNSGLGGSIPSSIGKLTALTTLYLSINHFSGNIPPELGGCKSLVDLQLHANQLEGLIPNELGMLSQLQVLEAFDNRLTGEVPLGIWKIKGLKKLNIYNNNLSGELPDEVAGMKQLREITLYNNRFTGAIPQGLGINSSLTVIDFTNNSFTGKIPPSLCFRKQLQRLLLGYNYLQGSVPHDVGSCSSLSRLILQHNNLTGVLPEFMENRNMLYMNLRGNFFSGKIPASFGKLTNITEIDLSMNKLSGNIPLELGNLVQIQALNISYNELQGPLPSQLGNCSRLLEFDASHNSVSGSIPAAFGKLTQLSTLSLSDNHFSGEIPHFISQLQDLIDLQLGGNSFSGHIPSSIVELHSLNRLNLSSNKLIGGIPSNFRKMVMLEHLDVSHNNLTGDLASLGDTVGLVTLDVSFNHFTGPIPATLLKSISSSSFVGNTGLCVDCCIENSNIKPCVVRSRGLKKRHYIMIAVGMCLFVMVVYLVLYLAVICRRKQKQEMDMLAEGCCSSLVIKALEATENLNDKYIIGRGAHGTVYKACLGHGRVYAVKRLGFGGGQTSMKREVETVGKVRHRNLVTIQDVLIKKDYSLILYKYMENGSLYDVLYEKEPTLVLSWSVRYRIALGTAQGLAYLHFDCDPVIIHRDIKPMNILLDGDMEAHISDFGIAKLLDQSATGALATSTLMGTIGYIAPENAFTRSASRELDVYSYGVVLLELISRQKPVLIENVDMVSWVRSRSEEIEKIVDRGLLEEIEEDKRVREQVRKVVLVALRCTEWEPTRRPTMREVVKCLQDSV

SEQ ID NO: 53 - DNA, 3354 bp, Gossypium arboreum
  source 1..3354: mol_type = genomic DNA
atgaaaggtaactttctctttccattaaccttgctcttgtgtttcttgtttttagaacaaaacactggtctttttgtttatgctttgaattctgatggggaaaccctgttttctttgttacctcactggtcatccttaccttcttctttaacctcttcatggaatgcttctgatccaaatccatgcaaatgggttggtgttgaatgtgattctgaaacccataatgtccttactcttaaccttacaaacttagccatttcaggccaattgccacctcaaattggtgctttacaccatttgaacacccttgatttgagcaacaataggttctttggtccaataccctcagctttggccaattgtagctcccttcaacacttggatttgtctaacaatgaactcattggaccaatgcctaatactttcaattccttgcaaaagctgaattacttgaacttgttttccaattccttgagtggtgaaataccagaaacattgtttcatgttactggtttggtgtccgtgtatttgaataataacaacttgagtggttcaatacctatgaatgttggtaacttgagcgagcttgtagctttaaatttgtatgataatgggttgataggtaccataccagagtcaatagggaattgtagtaaactacaagaactttttttagatgggaactattttgttgggggtttgccttccagtgttatgaatctccaaaacttaatgtatttgtatttgcgtcacaatagtttcaatggtgaaatcccaagtcctaccaagtgtaaaaatttgagtgttttggatttatcattcaatagtttcaatggggggataccaccagggttagccaattgtagcaccttgactgagctggttattgtacatagtaacttaacaggttatattccttcttcattaggcttattagatcagctttcaaagcttgatctttctgaaaaccatttatatggtgaaatcccatttcagcttgggaaatgtaagtctttgaaacagctattattgtatgataatcaactcaaaggtgaaatccctaatgaattagggatgttaagtgagttgaatgatcttgagttgtttatgaaccatttgagtggtgagattcctattagtatttggaggattccaagcttagagtatttacttgtttataagaacaacctaacaggggaattgcctttagagatcactgagttcaagcaattgaaaaacatttcgttgtacgataatggtttcttcggggtcatacctcggaatttggggattaatgctagtttacagcagttggatttcacaaacaatatgttcagtggtacaatccctccatttctttgctttggaaagaagttaagggtgttgaatttgggtcaaaatcagcttaccggcagtgtaaccgatgatatcggaggctgtaaaactctgtggagattgatccttaagcagaataacctcaacggtgtgctgcccgagtttgcagagaataaaaatcttgtacacatggatattagtgaaaatgacatctccggtccaattccatccagcttggggaactgtagaaaccttacttctatcaatttgtccatgaatcgcttcacgggatttataccttcggagttaagcaacctagcagatcttcagactttgaatgtttctcacaaccttttacaaggttctttgccatctcagttgtcaaattgtagcagattggtagagttcgacgtggggtttaattcgctgaatggtttgattccgaatgctttgacgagctggaaacagctgtcaactcttattctgagtgaaaaccgatttactggtggcattccttctttcttgttggaactcgaaatgctttcggacctgcagctcgggggaaatccatttggtggtagtattccttcgtcgattggagctatgaagaatctgatttatggcttgaatttgagcagcaatagtttgactggagagattccctcggagctaagaaatctgttcaagctggttagattggatttatctaataacaatctaacagggactttaacagttcttgatggaatggattccttggttgaggtcaatgtttcctacaatcacttcactggtccgataccgacaacaatgatgcgtttcatgaacttgtcctcatcatctttcctcggcaatcccgggttatgcattgattgtctttcatcaggtgaggaaacttgtcctacaagaaattaccttaatccttgtaatgaaatgaaaaatcaaaggggccttaccaaattggaagttgcaatgatagccctcggttcatctctacttgttgtggcactcctagttgtggcttcgatattcattttctgcaggataaaaaagcaagaacatgaggtctgtgtcgaagaaggcgcatcgaccttattgaacaaagtgatggaagctaccgagaatttaaatgagaggtatattatcggcagaggagcacatggagtcgtttatagagcatcattgagtccagggagtgattttgctgtaaaaaggatcaatgtagcaaagcataaaggaggaaaccaaagcatggttcgagagatcgaaactattgggaaagttaagcatcgaaacctggtgagattagaagatttttggttgagaaaagattatggattactcttatacaggtacatgcaaaatgggagcctacatgatgttttacataataataatgatcatacaacaaatgaggtacaaatcctgaaatggagtgctcggtatagaatagcactaggaacggctcatgggttggaatatctccattatgattgtgatcccggtatagttcatcgagacatcaagccagaaaacatcctccttgactccgatatggaaccacacatctccgattttggaattgctaagctccttgatgagtctgtggcttccgaaccgtctatggtagttgcgggcacggttggatatatagctccagaaaatgcattcagaacaacatggagcaaagaagcggatgtgtatagctatggggtggttttgctcgagctgataaccgggaagagggcattggatccatcatttatgggggagactgatatcgtggggtgggtcagatcagttttgagcggcgaaatcgaaaccgaaatcgagaggatcgtggattcgaggattgtggatgagttgatggaatgggaagtgagggagcaggttattaatgtggttttggtagctttgagatgtactgaaaaggaaccaagcagaagacctgcaatgagagatgttgtgaggcagctcttaaacaccaaactccctagaaaatctaaacatcggagctaa

SEQ ID NO: 54 - protein, 126 aa, Gossypium arboreum; identical to SEQ ID NO: 30

SEQ ID NO: 55 - DNA, 3033 bp, Oryza sativa
  source 1..3033: mol_type = genomic DNA
atgggactgcacatatggtgttggttggttgtcttgttcagcttggccccattgtgttgtagtttgagcgcagatggcctggctcttctggatctagccaagactctgatactgcccagctccataagctcgaattggagtgctgatgatgcaactccgtgtacatggaaaggagttgattgtgatgaaatgagcaatgtggtttctcttaacttatcatattctggattgtctggttctctaggtcctcagataggactcatgaagcacctgaaagtcattgatttatcaggtaatggtatatcaggaccaatgcccagttccattggcaactgcaccaaactggaggtgctccatctactacgtaatcgattgagtgggatccttccagatacattgagcaatattgaagcattaagggtttttgatctctcccgcaatagcttcacaggcaaggtcaatttcagatttgagaactgcaagcttgaggagttcatcttgtcattcaattatctcagaggcgaaatcccggtgtggatagggaattgcagcagcttgacacagcttgcatttgtcaacaatagtatcaccggtcaaataccaagttcaatcggtttattgagaaacctttcttaccttgtactttcccagaactccttgtctggcacaatccctcctgagattggtaactgccaattgctgatatggctgcatctagatgcaaaccagctcgagggcactataccaaaagaactagcaaacctgaggaacttgcagaagctctatctttttgagaattgcctcactggggagtttcctgaagatatatggggaatccaaagcctactatctgtcgacatctataaaaacaatttcactgggcagctgcctatagtgttggctgagatgaagcagctccagcaaattacgctattcaataattcattcactggtgtcataccacaggggttgggtgtaaatagcagtttgtccgtaattgatttcataaacaatagttttgttggcacaatccctccaaaaatttgttcagggggaagattggaagttttgaacttgggttcaaatcttctcaatggtagcatcccctctggtatcgctgactgcccaactttgagacgagtaattctcaaccaaaataatctcattggatcaattccacaatttgtaaattgtagcagtcttaattatattgatctcagctataatttattaagtggggacattcctgctagcttgagcaaatgtatcaatgttacatttgtgaactggtcatggaacaagcttgctggtctaataccatcagaaattgggaacttagggaacttaagtagtcttaacctctcaggaaacagactatatggtgaactccctgtggaaatttctggatgctccaagttatataagcttgatttgagctacaactctttgaacggttcggcactcacaactgtaagtagccttaaatttctgtcacagctacggttgcaggagaataaattcagtggaggtatacctgattctttatctcagttggatatgcttattgaactgcaacttggtggcaacattcttgggggtagtatcccttcatcgttaggaaagttagttaaactgggcattgcattaaacctcagtagaaatggactagttggtgacattccaccactaggcaatttggtggagctgcagagtttagatttgtcatttaataacctcaccggaggtcttgcttcattaggaaacctacagtttttgtatttcttgaatgtttcctacaacatgtttagtggaccagtaccaaaaaatcttgtgaggtttctgaattccactccaagttcatttagtggaaatgcagatctatgtatctcttgccatgaaaatgattcatcttgcacaggttctaatgttttgagaccttgtggttcaatgagtaaaaaaagtgcactcacaccactcaaggttgctatgatagttcttggttcggtttttgctggtgcatttctgatactctgtgtccttctaaaatataatttcaagcctaagattaacagtgatttaggtatattatttcaaggatcttcttctaaattaaatgaggctgtagaagtgactgaaaacttcaataacaagtacattatcggttccggggcccatggaattgtctacaaggcagtactgaggtcaggagaagtatatgctgtaaagaagcttgtacatgctgctcacaagggctcaaatgcaagcatgatccgcgagctgcagacgcttggtcaaattaggcacaggaacctgataagacttaatgaattcttgtttaagcatgagtatggtttgatcctatatgattttatggagaatggtagcctgtatgatgtgttgcatgggactgagcccactccaactttggactggagcatccgctacagcatagctcttggaacagcccatggtctagcatatctccataatgactgtcaccctgctatcatacatcgagatattaaaccaaaaaatatattgctggacaacgacatggtaccgcatatctcagattttggcattgcaaagctcatggatcaatatcctgctgctttacagaccacaggaatcgttggtactattggatatatggccccagaaatggccttttcaaccaaggctaccacagaattcgatgtgtacagttacggtgtggtattacttgagttgatcaccagaaagatggctgtggattcctcattccctggcaacatggacatagttagctgggtatcctccaagttgaatgagactaatcagatcgaaactatttgcgacccagctctcattactgaagtatatggaacacatgaaatggaagaagtgcgcaagctgttgtcattagctcttagatgcacagcaaaggaggcaagccaaaggccttccatggccgttgttgtcaaagagctgacagatgcaagacatgttgctggctcatactcgaagcagaattcaggccccagcaattcttga

SEQ ID NO: 56 - protein, 1010 aa, Oryza sativa
MGLHIWCWLVVLFSLAPLCCSLSADGLALLDLAKTLILPSSISSNWSADDATPCTWKGVDCDEMSNVVSLNLSYSGLSGSLGPQIGLMKHLKVIDLSGNGISGPMPSSIGNCTKLEVLHLLRNRLSGILPDTLSNIEALRVFDLSRNSFTGKVNFRFENCKLEEFILSFNYLRGEIPVWIGNCSSLTQLAFVNNSITGQIPSSIGLLRNLSYLVLSQNSLSGTIPPEIGNCQLLIWLHLDANQLEGTIPKELANLRNLQKLYLFENCLTGEFPEDIWGIQSLLSVDIYKNNFTGQLPIVLAEMKQLQQITLFNNSFTGVIPQGLGVNSSLSVIDFINNSFVGTIPPKICSGGRLEVLNLGSNLLNGSIPSGIADCPTLRRVILNQNNLIGSIPQFVNCSSLNYIDLSYNLLSGDIPASLSKCINVTFVNWSWNKLAGLIPSEIGNLGNLSSLNLSGNRLYGELPVEISGCSKLYKLDLSYNSLNGSALTTVSSLKFLSQLRLQENKFSGGIPDSLSQLDMLIELQLGGNILGGSIPSSLGKLVKLGIALNLSRNGLVGDIPPLGNLVELQSLDLSFNNLTGGLASLGNLQFLYFLNVSYNMFSGPVPKNLVRFLNSTPSSFSGNADLCISCHENDSSCTGSNVLRPCGSMSKKSALTPLKVAMIVLGSVFAGAFLILCVLLKYNFKPKINSDLGILFQGSSSKLNEAVEVTENFNNKYIIGSGAHGIVYKAVLRSGEVYAVKKLVHAAHKGSNASMIRELQTLGQIRHRNLIRLNEFLFKHEYGLILYDFMENGSLYDVLHGTEPTPTLDWSIRYSIALGTAHGLAYLHNDCHPAIIHRDIKPKNILLDNDMVPHISDFGIAKLMDQYPAALQTTGIVGTIGYMAPEMAFSTKATTEFDVYSYGVVLLELITRKMAVDSSFPGNMDIVSWVSSKLNETNQIETICDPALITEVYGTHEMEEVRKLLSLALRCTAKEASQRPSMAVVVKELTDARHVAGSYSKQNSGPSNS

SEQ ID NO: 57 - DNA, 3339 bp, Oryza sativa
  source 1..3339: mol_type = genomic DNA
atgaggctggttgtgtggcactggtttttcttcttcttcttcacttctgtttcatcgtcttggagtttgacttcagatggtctagcccttctttctctgtctagggatctcatattacctcattccataagctccacttggaaagcttctgatacaactccttgtaattgggatggggtttcctgcaacaaaaagaatagtgtggtttctcttgacctgtcatcttctggagtttctggttctcttggaccccaaataggacttatgaagagcctacaagtactcagtttgtcaaataacagcatatctggttcaatccctcaagaattgggcaattgtagcatgcttgatcaattggatttgtccagtaacagtttttctggtgagataccagcatcccttggtgacatcaaaaagctttcgtctctctctttgtacagtaactccctcactggtgaaataccagaggggttgttcaagaatcagtttctggagcaagtgtacctccattacaataaactcagtggttctatccccttgacagttggagaaatgactagccttaggtacctgtggctgcatggcaataaattatctggagttctaccagattcaattggcaactgcaccaagttggaggagctctatctactagataatcaattgagtgggagtcttccgaaaaccttgagctatatcaaaggactgaagattttcgatattaccgcaaatagtttcacaggtgagatcacatttagttttgaggattgcaagcttgaggtattcatattgtcattcaatcagattagcaacgaaattccatcatggctagggaattgtagtagcttgacacagcttgcatttgtcaacaataatatatctggccagattccatcgtctctgggtttattgagaaacctctctcaacttttactttctgagaactcactttctgggccaattcctcctgagataggtaactgccagttgctggtgtggctggagttggatgcaaaccagctcaatggcactgttcctaaagagctggcaaatctgagaaaattggagaaactctttctgtttgaaaaccgcctcattggggagttccctgaggatatttggagcatcaagagcctgcaaagtgtccttatctatgaaaacagttttactgggaggctacctccagtgctagctgagctgaagttcctgaagaacattacacttttcaacaatttcttcactggagttataccaccagatttgggtgttaatagtcgtttaacccaaattgatttcacaaacaacagttttgttggtggaatcccgcctaacatttgttcagggaaaagattgagaattttggacttggggcttaatcttctcaatggtagcatcccatccaatgttatggactgcccaagtttggaacgatttattctccaaaacaacaatctaagtgggcccattccacaatttaggaactgtgcaaatctgagctatatagatctgagtcataattccttaagtggcaacattccagcaagcttggggagatgtgtaaatattacgatgataaaatggtcagaaaacaagttggttggtccaataccatctgaaattagagacttggtgaatttgagagtgctaaacctctcgcaaaacagcctgcaaggtgttcttccagtgcagatttctagttgctccaagctgtacttgcttgacttgagtttcaactctttgaatggttcggcactcacaaccgtaagcaaccttaagtttctgtcacaactacggttacaagagaataaattcagtggaggcatacctgattccctctcgcagttggatatgcttattgagctgcaacttggtggcaacgttcttgggggcagtatcccttcatcgttaggaaggttggtaaaactgggcattgcattgaatatttgtagcaatggactcgttggtggcattccgccattattgagcaatttggtggagctgcaaagtttagatttgtcacttaatggcctcactggagacctagacatgttaggaaacttacaattactgcatgtattgaatgtttcctacaatagattcagtggtccggtcccagaaaatcttctgaattttctggtttcctcaccgagctcctttaatggcaatccagacctctgtatctcttgccataccaatggttcttattgcaaggggtctaatgttttgaaaccttgtggagagaccaaaaaactacacaaacacgtcaagattgctgttatagttattggttcattgttcgttggagcagtttccatacttatactgagttgcatccttttaaagttttatcatccaaagacaaaaaatttagaatcagtcagtactctgtttgaaggttcttcttctaaattaaatgaggttatagaggctactgaaaactttgatgacaagtatatcatcggtactggtgctcatggaactgtttacaaggcaacactgaggtcaggagaagtatatgctgtaaagaagcttgcaatatctgcacagaaaggttcgtacaaaagcatgatcagagaactgaagacattaggcaaaatcaagcatcggaacttgataaagctgaaagagttttggttaagaagtgagtatgggttcatgctttatgtttatatggagcaaggtagccttcaagatgttctgcatgggatccaacctcctccaagtttggactggagtgtgcgctataccatagctcttggtactgcccatgggctagcgtatcttcatgatgactgtcaacctgcaattattcaccgagatattaagcccagtaatatacttctgaatggggacatggttccacatatagcagattttggcattgcaaagctcatggaccagtcttcttctgctccacagactactggagttattggcacctttggatatatggccccagagttggcattttccaccaggagtagtatcgagtccgatgtatacagctacggcgtcatactccttgagctgctaacaaaaaaacaggtggtggatccctcgttccccgacaacatggacattgtcggttgggtgaccgcaacgctcaacggcaccgaccaaatcgaactcgtctgcgactcgacgctgatggaggaagtctatggcacggtggaaatagaggaagtcagcaaggtcctgtccttggctcttaggtgcgcagcgaaggaagcgagccgaaggccgcccatggccgatgttgtgaaggagctgactgatgtcaggaaatccgccgggaagttgtccaagccggagaagacggcctcccggagctcgtcctga

SEQ ID NO: 58 - protein, 1112 aa, Oryza sativa
MRLVVWHWFFFFFFTSVSSSWSLTSDGLALLSLSRDLILPHSISSTWKASDTTPCNWDGVSCNKKNSVVSLDLSSSGVSGSLGPQIGLMKSLQVLSLSNNSISGSIPQELGNCSMLDQLDLSSNSFSGEIPASLGDIKKLSSLSLYSNSLTGEIPEGLFKNQFLEQVYLHYNKLSGSIPLTVGEMTSLRYLWLHGNKLSGVLPDSIGNCTKLEELYLLDNQLSGSLPKTLSYIKGLKIFDITANSFTGEITFSFEDCKLEVFILSFNQISNEIPSWLGNCSSLTQLAFVNNNISGQIPSSLGLLRNLSQLLLSENSLSGPIPPEIGNCQLLVWLELDANQLNGTVPKELANLRKLEKLFLFENRLIGEFPEDIWSIKSLQSVLIYENSFTGRLPPVLAELKFLKNITLFNNFFTGVIPPDLGVNSRLTQIDFTNNSFVGGIPPNICSGKRLRILDLGLNLLNGSIPSNVMDCPSLERFILQNNNLSGPIPQFRNCANLSYIDLSHNSLSGNIPASLGRCVNITMIKWSENKLVGPIPSEIRDLVNLRVLNLSQNSLQGVLPVQISSCSKLYLLDLSFNSLNGSALTTVSNLKFLSQLRLQENKFSGGIPDSLSQLDMLIELQLGGNVLGGSIPSSLGRLVKLGIALNICSNGLVGGIPPLLSNLVELQSLDLSLNGLTGDLDMLGNLQLLHVLNVSYNRFSGPVPENLLNFLVSSPSSFNGNPDLCISCHTNGSYCKGSNVLKPCGETKKLHKHVKIAVIVIGSLFVGAVSILILSCILLKFYHPKTKNLESVSTLFEGSSSKLNEVIEATENFDDKYIIGTGAHGTVYKATLRSGEVYAVKKLAISAQKGSYKSMIRELKTLGKIKHRNLIKLKEFWLRSEYGFMLYVYMEQGSLQDVLHGIQPPPSLDWSVRYTIALGTAHGLAYLHDDCQPAIIHRDIKPSNILLNGDMVPHIADFGIAKLMDQSSSAPQTTGVIGTFGYMAPELAFSTRSSIESDVYSYGVILLELLTKKQVVDPSFPDNMDIVGWVTATLNGTDQIELVCDSTLMEEVYGTVEIEEVSKVLSLALRCAAKEASRRPPMADVVKELTDVRKSAGKLSKPEKTASRSSS

SEQ ID NO: 59 - DNA, 3372 bp, Zea mays
  source 1..3372: mol_type = genomic DNA
atgaagctggttttctggcattggatttttctattcttcgtgttgctttcaacatcacagggtatgagttcagatggcctagctcttcttgctctgtccaaaaccctaatactaccaagtttcataaggaccaactggagtgcttctgatgcaactccttgtacatggaacggtgttggctgcaatggaaggaacagagtgatttctctcgacctatcgtcatcagaggtctcaggttttataggacctgaaatagggcgtctgaaatacctgcaggttctcattttatctgctaacaacatatctggtttgatccctctagaattgggcaactgcagtatgcttgaacaattggatctgtcccaaaacttgctttctggcaatataccggcatcaatgggcagcctcaagaaattgtcatcactgtcgctgtactacaactctttccatggaacaataccagaggagttgttcaagaaccagtttctggagcaagtgtacctacatggaaatcagctcagtggttggatacccttctcggttggtgaaatgacaagccttaagtcattgtggttgcacgaaaatatgttgtccggagttttgcccagttcaattggcaactgcaccaagttggaggagctgtatctactccataatcaactgagtggcagtattccagaaaccttgagtaagatcgaaggcctcaaggtttttgatgccactgccaatagtttcacgggcgagatctctttcagttttgagaactgcaagctggaaatattcatcttgtcattcaataatataaagggtgaaattccgtcatggctagggaattgcaggagcttgcaacaacttggatttgtcaataatagtctgtctggcaaaattccaaattttataggcttattcagcaacctcacgtatcttttactttcacagaactccctgactgggctgatcccacctgagattggtaactgtcggttgctgcagtggctagagctagatgcaaatcagctggagggcactgttcctgaagaatttgcaaatttaaggtatttgtcaaagctctttcttttcgagaatcacctcatgggagacttccctgagagtatttggagtatccaaaccctcgagagtgtccttctttatagcaacaaattcacagggaggctaccttcagtgttagctgagctgaagtccctaaagaacatcacactgtttgataatttcttcactggagtcataccacaggagctgggtgttaatagccccttggtccagatagatttcacaaataacagttttgttggtggtatcccaccaaacatttgttcaggaaaagcattgagaattttggacttggggtttaatcatctcaacggtagcatcccatccagtgttctggactgcccaagtctggagcgagtcattgtcgaaaacaataaccttgttgggtctattccgcaatttataaactgtgcaaatctaagttatatggatctgagccacaattccttgagtggtaacataccatcaagtttcagcaggtgtgtaaaaattgctgagataaactggtcagagaacaatatttttggggcaataccaccagaaattggaaagttggtgaatctgaaaaggcttgacctctcacacaatctattgcatggttcgatccctgtgcaaatttctagttgctccaagttgtattcacttgatttgggttttaactctttgaatggttcggccctcagcacagtaagcagcctgaagtttctgacacagctacgattgcaagagaatagattcagcggaggtttgcctgatcctttctcacaattggaaatgcttattgagctgcaacttggtggaaatattcttgggggcagtatcccttcatcattaggacagctggtgaaactgggtacaaccttgaaccttagtagcaatggtctagtgggtgacattccatcacaattcggtaatttggtggagttgcaaaacttagatttgtcatttaataatctcacaggaggccttgctacattgcgaagtctacgctttttgcaggccttgaatgtttcttacaaccaatttagtggaccagttccagataatcttgtgaagtttctgagttccacaacaaattcttttgatggaaacccaggcctctgtatctcttgcagcaccagtgattcttcttgcatgggagctaatgttctgaaaccttgtggcgggtcaaagaaaagagcagtgcatggccgattcaaaattgttctcatagttcttggctcattatttgtgggagcagttctggtactcatactctggtgcatccttctgaaatctcgagatcagaagaagaatagtgaggaagcagtcagtcatatgtttgaaggttcctcatctaaattaaatgaggttatagaggcaactgaatgttttgatgacaagtatatcattggtaaaggtggtcacggaaccgtttacaaggcaacactgaggtcaggggatgtttatgctataaagaaacttgtgatttctgcacacaaaggttcatacaaaagcatggttggagaactgaagacactaggtaaaatcaagcacaggaacttgattaagctgaaagaatcttggttgagaaatgacaatggattcatactgtatgattttatggaaaaaggtagccttcatgatgttctacatgtagttcagccagcaccagccttagactggtgtgtgcggtatgacatagccctcggcactgcccatgggttagcatatctacatgatgactgccgccctgcgatcattcatcgcgacatcaagccaagtaatatactgctggacaaggacatggtgccacatatttcagattttggcattgcaaagctcttggagcagccttctactgctcctcagaccactggtgttgttggcaccattggatatatggccccagagttagcgttctccaccaagagcagcatggagtccgacgtgtacagctacggcgtggtgctgctggagctgctcacgaggagggcggcggtggatccctcgtttcccgacggcacggacatagtcagctgggcgtcgtccgccctgaacggcactgacaaaatcgaggccgtctgcgacccggccctcatggaggaagtcttcggcacggtggagatggaggaggtgagtaaggtcctgtcagtggcgctgcggtgcgcggccagggaggcgagccaaaggccctccatgaccgcggtcgtgaaggagctgacggatgcacggcctgccactggcggcggccggtcgttgtccaagtcgaagcaggggaaaccaggatcgcaatccaacagcagcgcctaccggcagtag

SEQ ID NO: 60 - protein, 1123 aa, Zea mays
MKLVFWHWIFLFFVLLSTSQGMSSDGLALLALSKTLILPSFIRTNWSASDATPCTWNGVGCNGRNRVISLDLSSSEVSGFIGPEIGRLKYLQVLILSANNISGLIPLELGNCSMLEQLDLSQNLLSGNIPASMGSLKKLSSLSLYYNSFHGTIPEELFKNQFLEQVYLHGNQLSGWIPFSVGEMTSLKSLWLHENMLSGVLPSSIGNCTKLEELYLLHNQLSGSIPETLSKIEGLKVFDATANSFTGEISFSFENCKLEIFILSFNNIKGEIPSWLGNCRSLQQLGFVNNSLSGKIPNFIGLFSNLTYLLLSQNSLTGLIPPEIGNCRLLQWLELDANQLEGTVPEEFANLRYLSKLFLFENHLMGDFPESIWSIQTLESVLLYSNKFTGRLPSVLAELKSLKNITLFDNFFTGVIPQELGVNSPLVQIDFTNNSFVGGIPPNICSGKALRILDLGFNHLNGSIPSSVLDCPSLERVIVENNNLVGSIPQFINCANLSYMDLSHNSLSGNIPSSFSRCVKIAEINWSENNIFGAIPPEIGKLVNLKRLDLSHNLLHGSIPVQISSCSKLYSLDLGFNSLNGSALSTVSSLKFLTQLRLQENRFSGGLPDPFSQLEMLIELQLGGNILGGSIPSSLGQLVKLGTTLNLSSNGLVGDIPSQFGNLVELQNLDLSFNNLTGGLATLRSLRFLQALNVSYNQFSGPVPDNLVKFLSSTTNSFDGNPGLCISCSTSDSSCMGANVLKPCGGSKKRAVHGRFKIVLIVLGSLFVGAVLVLILWCILLKSRDQKKNSEEAVSHMFEGSSSKLNEVIEATECFDDKYIIGKGGHGTVYKATLRSGDVYAIKKLVISAHKGSYKSMVGELKTLGKIKHRNLIKLKESWLRNDNGFILYDFMEKGSLHDVLHVVQPAPALDWCVRYDIALGTAHGLAYLHDDCRPAIIHRDIKPSNILLDKDMVPHISDFGIAKLLEQPSTAPQTTGVVGTIGYMAPELAFSTKSSMESDVYSYGVVLLELLTRRAAVDPSFPDGTDIVSWASSALNGTDKIEAVCDPALMEEVFGTVEMEEVSKVLSVALRCAAREASQRPSMTAVVKELTDARPATGGGRSLSKSKQGKPGSQSNSSAYRQ

SEQ ID NO: 61 - DNA, 3372 bp, Zea mays
  source 1..3372: mol_type = genomic DNA
atgaagctggttttatggcatcagttttttctcttcttcgtgttagtttcaacatcacagggtatgagttcagatggcctagctcttcttgctctgtccaaaagcctcatactaccaagtcccataagaaccaactggagtgattctgatgcaactccctgtacatggagcggtgttggttgcaatggaaggaacagagtcatctctctcgacctatcatcgtcaggtgtttcgggttctataggacctgcaatagggcgtctgaaatacctgcggattctcatcttatcagctaacaacatatctggtttgatccctctagaattgggagactgcaatatgcttgaagaactggatttgtcccaaaacctgttttctggcaatataccagcatcattgggcaacctcaagaaattgtcatcactgtcactgtaccgcaactccttcaatggaacaataccagaggagttgttcaagaaccagtttctggagcaagtgtacctacatgacaatcagctcagtggttcggtgcccttatcggttggtgaaatgacaagccttaagtcactgtggttacaggaaaatatgttgtctggagttttgcccagttcaattggaaactgcaccaagttggaggatctgtatctactcgataatcaactgagtggcagtattcctgaaaccttgggtatgatcaaaggtctcaaggtttttgatgctactaccaatagtttcacaggtgagatctctttcagttttgaggactgcaagctagaaatattcatcttgtctttcaataatataaaaggtgaaattccatcatggctggggaactgcatgagcttgcaacaacttggatttgtcaataatagtttgtatggcaaaattccaaattctcttggcttattgagcaacctcacatatcttttactttcacagaactccctttctgggccgatcccacctgagattggtaactgtcagtcgctgcagtggctagagttagatgcaaaccagctggatggcactgttcctgaagaatttgcaaatttacggagtttgtcaaagctctttctttttgagaatcgcctcatgggagacttccctgagaatatttggagtatccaaaccctcgagagtgtcctactttatagcaacagattcacagggaagctaccttcagtgttagctgagctgaagttcctaaagaacatcacactgtttgataatttcttcactggagtcataccacaggagctaggtgttaatagccccttggtccagatagatttcacaaacaacagttttgttggtagtatcccaccaaacatctgttcaagaaaagcattgagaattttggacttagggtttaatcatctcaacggtagcatcccatccagtgttgtggactgcccaagtttgaagcgagtcattttacaaaacaataaccttaacgggtctattccacaatttgtaaactgtgcaaatctaagttatatggacctaagccacaattccttgagtggtaacattccagcaagcttcagcagatgtgtaaacattactgagataaactggtcagagaacaagctttttggagcaataccacctgaaattggaaacttagtgaatctgaaaagacttgacctctcacacaacatattgcatggttcgatccctgtgcaaatttccagttgctccaagttgtactcacttgatttgagttttaactcattgaatggttcggccctccgcaccgtaagcaacttgaagtttctgacacagctacgattgcaagagaatagattcagcggaggcttgcctgattctctctcacaattggaaatgcttattgagctgcaacttggtggaaatattcttgggggtagtatcccttcgtcattaggacagctggtgaaactgggcacagccttgaaccttagtagcaatggcctaatgggtgacattccaacacaattgggtaatttggtggagttgcaaaacttagatttttcatttaataatctcacaggaggccttgctacattgagaagtctaggctttttgcaggccttgaatgtttcttacaaccaatttagtggaccagtcccagataatcttctgaagtttctgagttccacaccatattcttttgatggaaacccaggcctctgtatctcctgcagcaccagtggctcttcttgcatgggagctaatgttttgaaaccttgtggtgggtcgaagaaaagaggagtacatggccaattgaaaattgttctcatagttctcggttcattatttgtgggaggagttcttgtacttgtactgtgttgcatccttctgaaatctcgagattggaagaaaaataaagtcagtaacatgtttgaaggttcctcatctaaattaaatgaggttacagaggccactgaaaatttcgatgacaagtatatcatcggtacaggtgctcacggaactgtttacaaggcaacactgaggtcaggggatgtttatgctataaagaagcttgcgatttctgcacacaaaggttcatacaaaagcatggttagagaactgaagacactgggtgaaattaagcacagaaacttgataaagctgaaagaattttggttgagaagtgataatggattcatactgtatgattttatggaaaagggcagcctccatgatattctgcatgtaattcagccagcaccagctttggactggtgtgtgaggtatgacatagctcttggcaccgcccatgggttagcatatcttcatgatgactgccgccctgcgatcattcaccgtgatattaaaccaagaaatatactgctcgacaaggacatggtgccacatatttcagattttggcattgcaaagcacatggaccagtcttctactactgctccacagaccactggaatcgttggcactattggatatatggccccagaattggcgttttccaccaagagcagcatggagtctgacgtgtacagctacggtgtggtgctactggagctgttgaccaggaggacggcggtggatcctttgttccccgacagcgcggacatagtcggctgggtgtcgtccgtgctggacggcaccgacaaaatcgaggccgtctgtgacccggccctcatggaggaagtcttcggcacggtggagatggaggaggtgcgtaaggtcctgtcggtggcgctccggtgcgcggccagggaggtgagccaaaggccctccatgactgccgtcgtgaaggagctgacggatgcgcggccagcctctgccagcagcggcagccggtcgttgtccaagtcgagggaagggaaaccgggattgcaatccagcagcagcgcgtactggcagtag

SEQ ID NO: 62 - protein, 1123 aa, Zea mays
MKLVLWHQFFLFFVLVSTSQGMSSDGLALLALSKSLILPSPIRTNWSDSDATPCTWSGVGCNGRNRVISLDLSSSGVSGSIGPAIGRLKYLRILILSANNISGLIPLELGDCNMLEELDLSQNLFSGNIPASLGNLKKLSSLSLYRNSFNGTIPEELFKNQFLEQVYLHDNQLSGSVPLSVGEMTSLKSLWLQENMLSGVLPSSIGNCTKLEDLYLLDNQLSGSIPETLGMIKGLKVFDATTNSFTGEISFSFEDCKLEIFILSFNNIKGEIPSWLGNCMSLQQLGFVNNSLYGKIPNSLGLLSNLTYLLLSQNSLSGPIPPEIGNCQSLQWLELDANQLDGTVPEEFANLRSLSKLFLFENRLMGDFPENIWSIQTLESVLLYSNRFTGKLPSVLAELKFLKNITLFDNFFTGVIPQELGVNSPLVQIDFTNNSFVGSIPPNICSRKALRILDLGFNHLNGSIPSSVVDCPSLKRVILQNNNLNGSIPQFVNCANLSYMDLSHNSLSGNIPASFSRCVNITEINWSENKLFGAIPPEIGNLVNLKRLDLSHNILHGSIPVQISSCSKLYSLDLSFNSLNGSALRTVSNLKFLTQLRLQENRFSGGLPDSLSQLEMLIELQLGGNILGGSIPSSLGQLVKLGTALNLSSNGLMGDIPTQLGNLVELQNLDFSFNNLTGGLATLRSLGFLQALNVSYNQFSGPVPDNLLKFLSSTPYSFDGNPGLCISCSTSGSSCMGANVLKPCGGSKKRGVHGQLKIVLIVLGSLFVGGVLVLVLCCILLKSRDWKKNKVSNMFEGSSSKLNEVTEATENFDDKYIIGTGAHGTVYKATLRSGDVYAIKKLAISAHKGSYKSMVRELKTLGEIKHRNLIKLKEFWLRSDNGFILYDFMEKGSLHDILHVIQPAPALDWCVRYDIALGTAHGLAYLHDDCRPAIIHRDIKPRNILLDKDMVPHISDFGIAKHMDQSSTTAPQTTGIVGTIGYMAPELAFSTKSSMESDVYSYGVVLLELLTRRTAVDPLFPDSADIVGWVSSVLDGTDKIEAVCDPALMEEVFGTVEMEEVRKVLSVALRCAAREVSQRPSMTAVVKELTDARPASASSGSRSLSKSREGKPGLQSSSSAYWQ

SEQ ID NO: 63 - DNA, 3265 bp, Triticum aestivum
  source 1..3265: mol_type = genomic DNA
atggggcttgtattgtggcattcattgtttctcttcttaagtttggtttccacgtcatggagtttgaattcagatggtcgcgcccttcttgctctgtccaaaaatctcatattgcctagttccataaagtcaagttggaatgcttctgatacaaccccatgtaactggactggaattagttgtgatgaaaggaacaatgtggtttctcttgacctaacatcgtctggagtttctggttcattaggagttcaaataggtcttctaaagtacatacaagtcatcattttgctaaacaatagcatatctggtccaatccctcaagaactgggcaattgtagcatgcttgaacagttggatctttccatgaacttcctttctggtgaaataccagaatcactgtccaacctaaaaaaactatcatcattctcgttgtacactaactccctcagtggggaaataccagaggggttgttcaagaaccagtttctgcaggacgtgtacctccatgggaataatctcagtggttctatcccttcatcagttggtgaaatgacaagccttagatcattttggttgacgaagaatgcattatctggaggtctgccagattcaattgcaactgcaccaagttggaggagctctatctactggataatcagttgagcgggagccttccaaaaaccttgagctatgtcaaaggactgaaagttttagatgccactggaaatagctttaccggagagattgatttcagttttgagaactgcaagttggagatattcatattgtcgttcaatcatatgagtggcggtattccatcatggctagggaattgcactagcttgacacaacttgcacttgtcgacaaccgtttcactggccaaattccggcttctcttggcctattgagcaacctcaccttgcttatgctttctcaaaactccttgtctggctcaatccctcctgagattggtaactgttggttgctggagtggctagagttggatcataacatgctcgagggcactgttcctaaagagctggctaatctgagacacttgcagaagctctttcttttcgagaatcgcctgaccggggagtttcctgagggtatttggagcatccggtaccttcaaagtgttcttatttacagcaatggttttactgggaagctacctcttaagttagctgaattgaagttattggagaacatcacattgttcgataatttcttcaccggagtcatacccccgggtttgggtgttaatagccctttacagcaaattgatttcaccaacaacagttttaccggtggaatacctccgtacatttgttcaaggaaaagactcagggtgttggttttgggtctcaatcatctgaatggtagcatcccatccaatgttgcagattgcccaggtttggaacgaatcattctcaaaaacaatgatcttactgggcccattccacattttagaaactgtgcagcgctgggctatatggatttcagtcataattctttaagtggagatattccagcaagcttgggaaaatgcttaaatactacaatgataaactggtcagcaaacaaacttgttggtccaataccacctgaagttagaaacttggtgaatttggaagttcttaacctctcacaaaacagtctacagggtgcacttccagctcaggtttctagttgctccaagctgcttattttggatttgagttttaactctctgcatggtccggcactcacgacattggttatgcttcttgagctgcaacttggtggcaacattcttggaggcagaatcccttcgtcgttaggaaagttgatcaaactcattgcattcaatctcagcagcaacggactggtgggtgatattccaacaccattgggcatttggtggaactgcaaagtttagatttgtcagttaataacctcactggagctctcggcgcattagggagtctacattcattgcacgccttgaatctttcctataataggttcagtggaccagtgccagaatatcttctgaaatttctgaactccacaccaagctcttttaatggaaattcgggtctctgtatctcttgccgtgacaatgattcttcttgcaagagatctaatgtgctgaaaccttgtggaggatcagggaaaaaaggaataaagcgtcgattcaaggttgctcttattattcttggttcattgttcattggagcagtagcggtacttatcctctgctgtatccttctaaagaatcgagattcgaagacaaaaagtgaggagacaatcagtaatttgcttgaaggctcttcttctaaattaaatgagatcatagaaaagacagaaaactttgatgacaagtatatcataggcgcaggtgcgcatgggactgtgtacaaggcaatattgaattcaggggaggtctttgccataaagaagcttgcgatttcagcgcgcagcagctcctacaaaagcatgatcagagagctgaagacacttggtaaagttcggcacaggaacttgataaagctgaaggaattttgggtgagaggcgactctgggttcatactgtatgacttcatggagcatggtagcctctatgatgtcctgcacaggatccggacgccgagtctggactggagcatgcgctataacatagctcttggaactgcccatggtctggcatatcttcaccatgactctgtccctgcgatcattcatcgagatattaagccaagcaatatattgttaaacaaggacatggtgccgcgcatcgcggatttcggcatagcaaagatcatggatcagtgttctgctgctccacagtccaccggggtcgtcggcaccactggatatatggcaccagagctggcattttccaccagaaacagtatcaagaccgacgtgtacagctacggcgtcgtcctgcttgagcttataacaggaaagacggcggtggatccctcattcccggagaacatggacattgtcgcctgggtgccccacgccctgaacggcgctgagcagatcgggcccgtctgcgacccggccctcctggacgaagtgtacagcactgtcgaaatggaggaggtgcgcaaggtcctgcgcttggcccttaggtgcacagcgaaggagccgagtcaacgaccgtccatggtcgacgtcgtgaaggagctgactgacgcgaggttcgcgggcatcccttcgtcgtcgaagcaggggaagccaggctcctcctccggcggcggctcttcttga

SEQ ID NO: 64 - protein, 1088 aa, Triticum aestivum
MGLVLWHSLFLFLSLVSTSWSLNSDGRALLALSKNLILPSSIKSSWNASDTTPCNWTGISCDERNNVVSLDLTSSGVSGSLGVQIGLLKYIQVIILLNNSISGPIPQELGNCSMLEQLDLSMNFLSGEIPESLSNLKKLSSFSLYTNSLSGEIPEGLFKNQFLQDVYLHGNNLSGSIPSSVGEMTSLRSFWLTKNALSGGLPDSIGNCTKLEELYLLDNQLSGSLPKTLSYVKGLKVLDATGNSFTGEIDFSFENCKLEIFILSFNHMSGGIPSWLGNCTSLTQLALVDNRFTGQIPASLGLLSNLTLLMLSQNSLSGSIPPEIGNCWLLEWLELDHNMLEGTVPKELANLRHLQKLFLFENRLTGEFPEGIWSIRYLQSVLIYSNGFTGKLPLKLAELKLLENITLFDNFFTGVIPPGLGVNSPLQQIDFTNNSFTGGIPPYICSRKRLRVLVLGLNHLNGSIPSNVADCPGLERIILKNNDLTGPIPHFRNCAALGYMDFSHNSLSGDIPASLGKCINTTMINWSANKLVGPIPPEVRNLVNLEVLNLSQNSLQGALPAQVSSCSKLLILDLSFNSLHGPALTTLVMLLELQLGGNILGGRIPSSLGKLIKLIAFNLSSNGLVGDIPTPLGNLVELQSLDLSVNNLTGALGALGSLHSLHALNLSYNRFSGPVPEYLLKFLNSTPSSFNGNSGLCISCRDNDSSCKRSNVLKPCGGSGKKGIKRRFKVALIILGSLFIGAVAVLILCCILLKNRDSKTKSEETISNLLEGSSSKLNEIIEKTENFDDKYIIGAGAHGTVYKAILNSGEVFAIKKLAISARSSSYKSMIRELKTLGKVRHRNLIKLKEFWVRGDSGFILYDFMEHGSLYDVLHRIRTPSLDWSMRYNIALGTAHGLAYLHHDSVPAIIHRDIKPSNILLNKDMVPRIADFGIAKIMDQCSAAPQSTGVVGTTGYMAPELAFSTRNSIKTDVYSYGVVLLELITGKTAVDPSFPENMDIVAWVPHALNGAEQIGPVCDPALLDEVYSTVEMEEVRKVLRLALRCTAKEPSQRPSMVDVVKELTDARFAGIPSSSKQGKPGSSSGGGSS

SEQ ID NO: 65 - DNA, 3345 bp, Triticum aestivum
  source 1..3345: mol_type = genomic DNA
atggggctaattttatggcatcatttgcttctcttctccaacctagtctcgctctgctgcggtttgagttcagatggccatgcccttctggctctctccaggcggctcatactgcctgatatcataagctcgaactggagttcttctgatacaactccctgtggatggaagggagttcagtgtgagatgaacattgtggttcacctcaatctatcatactccaaagtttctggttcaattggtcctgaggtagggcgtatgaagtacctgcggcaactcgatctgtcaagcaacaacatctccggtccgatcccttatgaattgggaaattgtcttctgcttgacctgctggatctgtcagggaacagcctttccggtggaattccgacatccctcatgaacctaaagaagctatcacagctcggcttgtacagcaactccttcagtggagagatacctgaggggctgttcaagaaccggtttctggagcgggtgtatctccaagacaataaactcagtggctccatccctccatcggttggtgaaatgaagagtctgaaatactttaggttggatgggaacatgttgtccggagctctgccggactcaattggcaactgcaccaagttggagaatctatatctatatgataataaactgaatgggagtcttccgagatcattgagcaacatcaaagggctcgtgctctttgaggcctacaacaacagttttacaggcgacatctctttcagattcaagagctgcaagcttgaagtgtttgtgctatcttggaatcagatcagtggggaaatcccggggtggctgggaaattgtagcagcttaatcagacttgcatttctccacaaccgtctctctggccagataccaacttcactcggtttattgaaaaagctatcaatccttatacttactcagaattctttgtctgggctgatccctcctgagattggtagctgtcggtcgctggtgtggctgcagctggacgcaaaccagcttgagggcaccgttccgaaacagctggctaatctgcgtaacttgcagcagctatttctgtttgagaatcgcctcagtggggagttccctcaggatatttggggcatccaaggcctcgaatatgtccttctgtacaacaacagcttatctgggggcctacctccaatgtcggccgagttgaagcaccttaagttcgtcaaactacaggataatctgttcactggagtcataccaccaggatttgggattaacagccctttaatagagatcgacttcacaaataataggtttgttggtggaatcccaccgaacatttgttcgggtaaaagattaacagtttggaatttggggcataattttctcaatggtaccatcccgttcactgttgctaactgcccaagtttagagcgagttcgactccataacaacaatctcagtgggcaagttccgcaattccgagactgtgcaaatctgcggtacatagatttgagtcacaattccttaagtggtcatattcctgcaagcttgggcaggtgtgctaacattacagcgataaactggtctgcaaacaagcttggtggtccaataccacccgaactcggacaattagtgaagttggaaagtcttgacctctcccacaacagcctagagggggcaattcctgcacagatttccagttgctcgaagttgcacttgtttgatttgagtttcaactctttgaatggttctgcgctcacaacagtatgcaagctggagtttatgttaaatctgcggctacaggggaatagattaagtggaggcattccagattgtatctcgcagttacatgggctagtcgagctacaacttggcggcaatgctcttggtggtcatctcccttcatcattaggaattttgaaaagattgagtactgcactaaaccttagcagcaatgggctggagggcagcattccatctcaattacgctacttggtggatcttgccagcttagatttgtctggtaataatctcagtggggatcttgctccgttaggaagtctacgtgcattgtatacattgaatctttccaataacagattcagcgggccagtgccagagaatcttttacggtttatgaattctacaccaagcccatttagtggaaattcagatctctgtgtgtcttgtcatgatggtgattcttcttgcaagggagctaatgttttggaaccttgtagttcattgaggagaagaggagtacatggccgagtcaagatagctatgatatgtcttggttcagtttttgttggtgtgtttctgatactctgtatctttctgaaatacagaggttccaagactaaacctgagggagagttaaatccattttttggggagtcatcttctaaattaaatgaggttttggaatcaactgaaaactttgatgacaagtacatcatcggcacaggtggccaaggaactgtatacaaggcaacattgaggtcaggagaagtatatgccgtgaagaagcttgtgggtcatgcacacaagattttgcatggaagcatgataagggagatgaatacgcttggtcaaattaggcataggaacttagtaaagctaaaggatgttttgttcaggcgtgagtatgggttgatcctctatgaatttatggacaatggtagcctttatgatgttctccatggggctgagccagctccaattctggagtggaggatacggtatgacatagctcttggtacagcacatggtttagcttatctccacaacgactgccacccagccattattcaccgcgacattaaactgaaaaatatactgttggacaaggacatggtgccacatatttcagactttggcattgcaaagcttatcgacctgtctcctgctgcttcagaaactaccggaatcattggcactgttggatatatggccccagagatggcattttcaaccaggagtaccattgagttcgatgtgtacagctacggagtggtattacttgaactgattaccagaaagatggccctggatccctcgttccctcacgatgtggacctagtcagctgggtatcctccaccctgaatgagggcaatgtgatcgagtccgtgtgcgaccctgccctaatgcgtgaggtatgtggcactgctgaactggaggaagtatgcagtgtgctgtcgatagcccttagatgcaccacggaggacgcaagacagaggccttccatgatggatgttgtgaaagagctgacacgtgctaggcgtgatgctgtatcgctaccgaagcaggccatatctggttccagcagttcctgtctaaacctggcaacctga

SEQ ID NO: 66 - protein, 1114 aa, Triticum aestivum
MGLILWHHLLLFSNLVSLCCGLSSDGHALLALSRRLILPDIISSNWSSSDTTPCGWKGVQCEMNIVVHLNLSYSKVSGSIGPEVGRMKYLRQLDLSSNNISGPIPYELGNCLLLDLLDLSGNSLSGGIPTSLMNLKKLSQLGLYSNSFSGEIPEGLFKNRFLERVYLQDNKLSGSIPPSVGEMKSLKYFRLDGNMLSGALPDSIGNCTKLENLYLYDNKLNGSLPRSLSNIKGLVLFEAYNNSFTGDISFRFKSCKLEVFVLSWNQISGEIPGWLGNCSSLIRLAFLHNRLSGQIPTSLGLLKKLSILILTQNSLSGLIPPEIGSCRSLVWLQLDANQLEGTVPKQLANLRNLQQLFLFENRLSGEFPQDIWGIQGLEYVLLYNNSLSGGLPPMSAELKHLKFVKLQDNLFTGVIPPGFGINSPLIEIDFTNNRFVGGIPPNICSGKRLTVWNLGHNFLNGTIPFTVANCPSLERVRLHNNNLSGQVPQFRDCANLRYIDLSHNSLSGHIPASLGRCANITAINWSANKLGGPIPPELGQLVKLESLDLSHNSLEGAIPAQISSCSKLHLFDLSFNSLNGSALTTVCKLEFMLNLRLQGNRLSGGIPDCISQLHGLVELQLGGNALGGHLPSSLGILKRLSTALNLSSNGLEGSIPSQLRYLVDLASLDLSGNNLSGDLAPLGSLRALYTLNLSNNRFSGPVPENLLRFMNSTPSPFSGNSDLCVSCHDGDSSCKGANVLEPCSSLRRRGVHGRVKIAMICLGSVFVGVFLILCIFLKYRGSKTKPEGELNPFFGESSSKLNEVLESTENFDDKYIIGTGGQGTVYKATLRSGEVYAVKKLVGHAHKILHGSMIREMNTLGQIRHRNLVKLKDVLFRREYGLILYEFMDNGSLYDVLHGAEPAPILEWRIRYDIALGTAHGLAYLHNDCHPAIIHRDIKLKNILLDKDMVPHISDFGIAKLIDLSPAASETTGIIGTVGYMAPEMAFSTRSTIEFDVYSYGVVLLELITRKMALDPSFPHDVDLVSWVSSTLNEGNVIESVCDPALMREVCGTAELEEVCSVLSIALRCTTEDARQRPSMMDVVKELTRARRDAVSLPKQAISGSSSSCLNLAT

SEQ ID NO: 67 - DNA, 3348 bp, Triticum aestivum
  source 1..3348: mol_type = genomic DNA
atggggcttgtattgtggcattcattgtttctcttcttatgtttggtttcctcgtcatggagtttgaattcagatggtcgtgcccttcttgctctgtccaaaaatctcatattgcctagttccataaagtcaagttggaatgcttctgatacaaccccatgtaactggactggaattagttgtgataaaaggaacaatgtggtttctcttgacctaacattgtctggagtttctggttcactaggagttcatatagggcttctaaagtacataaaagtcatcaatttgccgagcaataacatatgtggtccaatcccccaagaattgggcaattgtagcatgcttgaacagttggatgtttccgggaacttcctttctggtgaaataccagaatcgcttggcaacctcaaaaaactatcatacctctcgttgtacaataactccctcagtggggaaataccagaggggttgttcaagaaccagtttctgcaggacgtgttcctcaatgagaataaactcagtggttctatcccttcatcagttggtgaaatgacaagccttagatcattttggttgacgcagaatgcattatctggaggtctgccagattcaattggcaactgcaccaagttggaggagctctatctgctggataatcggttgagcgggagccttccgaaaaccttgagctatgtcaaaggactgaaagttttagatgccactggaaatagcttcaccggagagattgatttcagttttgagaactgcaagttggagaaattcatattctcgttcaatcagatgaggggcggtattccagcgtggctaggtaattgcagtagcttgacagaacttgcacttgtcaacaacagtttctctggccaaattccgccttctcttggcctattgagcaacctcaccttgcttatgctttctcaaaactccttgtctggcccaatccctcctgagattggtaattgtcggttgctggagtggctagagttggatcataacatgctggagggcactgttcctaaagagctggctaatctgagacacttgcagaagctctttcttttcgagaatcgcctgaccggggagtttcctgagggtatttggagcatccggtaccttcgaagtgttcttatttacagcaatggttttactgggaagctacctcttaagttagctgaattgaagttattggagaacatcacattgttcgataatttcttcactggagtcatacccctgggtttgggtgttaatagccctttacagcaaattgatttcactaacaacagttttaccggtggaatacctccgtacatttgttcaaggaaaagactcagggtgttggttttgggtttcaatcttctgaatggtagcatcccatccaatgttgcagactgcccaggcttgcaacggatcattctcagaaacaatgatcttactgggcccattccacattttagaaactgtgcagctctgggctatacagatttcagtcataattctttaagtggagatattccagcaagcttgggcaaatgcataaatactacaatgataaactggtcagcaaacaaacttgttggtccaataccacctgaaattggaaacttggtgaatttgggagttcttaacctctcacaaaacagtctccagggtgcacttccagcgcaggtttctagttgctccaagctgtatattctggatttgagtttcaactctctgcatggttcggcactcacgacagtaagcagcctgaagcttctcgcacaactacggttgcaggagaataaattcagtgggggcttacctgattctctttcgcagttggttatgcttcttgagctgcaacttggtggcaacattcttggaggcagcatcccttcgtcgttaggaaagttgatcaaactcattgcattgaatctcagcagcaacggactggtgggtgatcttcgaacaccattgggcaatttggtggaactgcaaagtttagatttgtcagttaataacctcactggaggtcttggcgcattaggaagtctacattcattgcatgccttgaatctttcctataataggttcagtggaccagtgccagaatatcttctgaaatttctgaactccgcaccaagctcttttaatggaaattcgggtctctgtatctcttgccgtgacagtgattcttcttgcaagagatctaatgtgctgaaaccttgtggaggatcagggaaaaaaggaataaagcgtcgattcaaggttgctcttattattcttggttcattgttcattggagcagtagcggtacttatcctctgctgtatccttctaaagaatcgagattcgaagacaaaaagtgaggagacaatcagtaatttgcttgaaggctcttcttctaaattgaatgagatcatagaaaagacagaaaactttgatgacaagtatatcataggcgcaggtgctcatgggactgtgtacaaggcaatattgaattcaggggaggtctttgctataaagaagcttgcgatttccgcgcgcagcagttcctacaaaagcatggtcagagaactgaagacgcttggtaaagttcggcacaggaacttgataaagctgaaagaattttgggtgagaggcgactctgggttcatactgtatgactttatggagcatggtagcctctatgatgtcctgcacaggatccggacgccgagtctggactggagcatgcgctataacatagctcttggaactgcccatggtctggcatatcttcaccatgactctgtccctgcgatcattcatcgagatattaagccgagcaacatattgctgaacaaggacttggtgccgcgcatcgcagatttcggcatagcgaagatcatggatcagtgttcagctgctccacagaccaccggggtcgtcggcacaactggatatatggcaccagagctggcattttccaccagaaacagtatcaagaccgacgtgtacagctacggcgttgtcctgctcgagctgataacgggaaagacggcagtggatccctcgttcccggagaacatggacattgtcggctgggtgccccacgccctgaacggcgctgagcagatcgggcccgtctgcgacccggccctcctggacgaagtgtacagcactgtcgaaatggaggaggtgcgcaaggtcctgcgcctggccctcaggtgcacggcgaaggagccgagtcaacgaccgtccatggtcgatgtcgtgaaggagttgactgacgcgaggtttgcgggcatcccttcatcgtcgaagcaggggaagcctggctcctcctccggcggcggctcttcttga

SEQ ID NO: 68 - protein, 1115 aa, Triticum aestivum
MGLVLWHSLFLFLSLVSSSWSLNSDGRALLALSKNLILPSSIKSSWNASDTTPCNWTGISCGKRNNVVSLDLTSSGVSGSLGVQIGLLKYIQVIILLNNSISGPIPQELGNCSMLEQLDLSENFLSGEIPESLSNLKKLSSLSLYTNSLSGEIPEGLFKNQFLQDVYLNENKLSGSIPLSVGETTSLRSFWLMHNALSGGLSDSIGNCTKLEELYLLDNRLSGSLPKTLSYVKGLKVLDATGNSFTGEIDFSFENCKLEKFILSFNQMRGGIPARLGNCSSLTELALVNNSFSGQIPASLGLLSNLTLLMLSQNSLSGSIPPEIGNCRLLEWLELDHNMLEGTVPKELANLRHLQKLFLFENRLTGEFPEDIWSIRYLRSVLIYSNGFTGKLPLKLAELKLLENITLFDNFFTGVIPPGLGVNSPLQQIDFTNNSFTGGIPPYICSRKRLRVLILGFNLLNGSIPSNVADCPGLERIILKNNDLTGPIPHFRNCARLGYMDFSHNSLSRDIPASLGKCINTTMINWSGNKLVGPIPPEIGNLVNLGVLNLSQNSLHGALPAQVSSCSKLYILDLSFNSLHGSALMTVSSLKLLAQLRLQENKFSGGLPDSLSQLVMLLELQLGGNILGGSIPSSLGKLIKLIALNLSSNGLVGDLPTPLGNLVELQSLDLSVNNLTGGLGALGTLHSLHALNLSYNRFSGPVPEYLLKFLNSTPSSFNGNSGLCVSCHDSDSSCKRSDVLKPCGGSGKKGIKRRFKVALIILGSLFIGAVAVLILCCILLQNRDSKTKSEETISNLLEGSSSKLNEIIEKTENFDDKYVIGTGAHGTVYKATLNSGEVFAIKKLAISARSSSYKSMIRELKTLGKVRHRNLIKLKEFWVRGDSGFILYDFMEHGSLYDVLHRIRTPSLDWSMRYNIALGTAHGLAYLHHDSVPAIIHRDIKPSNILLNKDMVPRIADFGIAKIMDQCSAAPQSTGVVGTTGYMAPELAFSTRNSIKTDVYSYGVVLLELITGKTAVDPSFPENMDIVGWVPHALNGTEQIGPVCDPALLDEVYSTVEMEEVRKVLRLALRCTANEPSRRPSMVDVVKELTDARFAGIPSSSKQGKPGSSSGGGSS

SEQ ID NO: 69 - DNA, 3348 bp, Triticum aestivum; identical to SEQ ID NO: 67
  source 1..3348: mol_type = genomic DNA

SEQ ID NO: 70 - protein, 1090 aa, Triticum aestivum
MGLVLWHSLFLFLCLVSSSWSLNSDGRALLALSKNLILPSSIKSSWNASDTTPCNWTGISCDKRNNVVSLDLTLSGVSGSLGVHIGLLKYIKVINLPSNNICGPIPQELGNCSMLEQLDVSGNFLSGEIPESLGNLKKLSYLSLYNNSLSGEIPEGLFKNQFLQDVFLNENKLSGSIPSSVGEMTSLRSFWLTQNALSGGLPDSIGNCTKLEELYLLDNRLSGSLPKTLSYVKGLKVLDATGNSFTGEIDFSFENCKLEKFIFSFNQMRGGIPAWLGNCSSLTELALVNNSFSGQIPPSLGLLSNLTLLMLSQNSLSGPIPPEIGNCRLLEWLELDHNMLEGTVPKELANLRHLQKLFLFENRLTGEFPEGIWSIRYLRSVLIYSNGFTGKLPLKLAELKLLENITLFDNFFTGVIPLGLGVNSPLQQIDFTNNSFTGGIPPYICSRKRLRVLVLGFNLLNGSIPSNVADCPGLQRIILRNNDLTGPIPHFRNCAALGYTDFSHNSLSGDIPASLGKCINTTMINWSANKLVGPIPPEIGNLVNLGVLNLSQNSLQGALPAQVSSCSKLYILDLSFNSLHGSALTTVSSLKLLAQLRLQENKFSGGLPDSLSQLVMLLELQLGGNILGGSIPSSLGKLIKLIALNLSSNGLVGDLRTPLGNLVELQSLDLSVNNLTGGLGALGSLHSLHALNLSYNRFSGPVPEYLLKFLNSAPSSFNGNSGLCISCRDSDSSCKRSNVLKPCGGSGKKGIKRRFKNRDSKTKSEETISNLLEGSSSKLNEIIEKTENFDDKYIIGAGAHGTVYKAILNSGEVFAIKKLAISARSSSYKSMVRELKTLGKVRHRNLIKLKEFWVRGDSGFILYDFMEHGSLYDVLHRIRTPSLDWSMRYNIALGTAHGLAYLHHDSVPAIIHRDIKPSNILLNKDLVPRIADFGIAKIMDQCSAAPQTTGVVGTTGYMAPELAFSTRNSIKTDVYSYGVVLLELITGKTAVDPSFPENMDIVGWVPHALNGAEQIGPVCDPALLDEVYSTVEMEEVRKVLRLALRCTAKEPSQRPSMVDVVKELTDARFAGIPSSSKQGKPGSSSGGGSS

SEQ ID NO: 71 - DNA, 3345 bp, Triticum aestivum
  source 1..3345: mol_type = genomic DNA
atggggctgatttcatggcatcgtttgcttctcttctccaacctagtctcgctctgttgcggtttgagttcagatggccatgcccttctggctctctccaggaggctcatactacctgataccataagctcgaactggagttcttctgatacaactccctgtggatggaagggagttcagtgtgagatgaacaatgtggttcacctcaatctatcatactacaaagtttctggttcaattggtcccgaggtagggcgtatgaagtacctgcggcaactcgatctgtcaagcaacaacatctccggtccgatccctcatgaattaggcaattgtgttctgcttgatctgcttgatctgtcaggtaacagcctttccggtggaattccgacatccctcatgaacctaaagaagctttcacagctcggtttgtacagcaactccctcagtggagagatacccgaggggctgttcaagaaccggtttctggagcgggtgtatctccaagacaataaactcagtggctctatcccttcatcagttggtgaaatgaagagtctgaagtactttaggttggatgggaacatgttgtccggagctctgccggactcaattggcaactgcaccaagttggagaatctatatctatacggtaataaactgaatgggagtcttccgagatcattgagcaacatcaaagggctcgtgctctttgaagccaacaacaacagttttacaggtgacatctctttcagattcaagagctgcaagcttgaagtgtttgtgctatcttggaatcagatcagtggggaaatcccggggtggctgggaaattgtagcagcttaatcagacttgcatttctccacaaccgtctctctggccagataccgacttcactcggtttattgaaaaaactatcaatccttatacttacccagaattctttgtctgggctgatccctcctgagattggtagctgtcggtcgctggtgtggctggagctggacgcaaaccagctcgagggcaccgtcccgaaacagctggctaatctgcgtaacttgcagcagctatttctgtttgagaatcgcctcagtggggagttccctcaggatatttggggcatccaaggcctcgaatccgtccttctgtacaacaacagcttatctgggggcctacctccaatgtcggccgagttgaagcacctaaagttcgtcaaactacaggataatttgttcactggagtcataccaccaggatttgggattaacagccctttagtagagattgacttcacaaataataggtttgttggtggaatcccgccgaacatttgttcgggtaaaagattgacagcttggaatttggggcataattttctcaatggtaccataccgttcactgttgctagctgcccaagtttagaacgagttcgactccataacaacaatctcagtgggcaagttccgcaattccgagactgtgcaaatctgcggtacatagatttgagtcacaattccttaagtggtcatattcctgcaagcttgggcaggtgtgctaacattacagcgataaactggtctcaaaacaagcttggtggtccaataccacctgaactcggacaattagtgaagttggaaagtcttgacctctcccacaacagcctagagggggcaattcctgcacagatttccagttgctcgaagttgcacttgtttgatttgagtttcaactctttgaatggttctgcactcacaacagtatgcaagctggagtttatgttaaatctgcggctacagaggaatagattaagtggaggcattccagattgtatctcgcagttacatgggctagtcgagctacaacttggcggcaatgttcttgggggtcatctcccttcagcattaggaactttgaaaagattgagtactgcactaaaccttagcagcaatgggctggagggcagcattccatctcaattacgcttcttggtggatcttgcaagcttagatttgtctggtaataatctaagtggggatcttgctccgttaggaagtctacatgcattgtataccttgaatctttcgaataacagattcagcgggccagtgccagagaatcttgtacaatttattaattccacaccaagtccattcagtggaaattcagatctctgtgtgtcttgccatgatgatgattcttcttgcaagggagctaatgttttggaaccttgtagttcattgaggagaagaggagtacatggccgcgtcaagatagctatgatatgtcttggttcagtttttgttggtgcgtttctgatactctgtatctttctgaaatacagaggttccaagactaaacctgagggagagttaaatccattttttggggagtcatcttctaaattaaatgaggttttggaatcaactgaaaactttgatgacaagtacatcatcggcacaggtggccaaggaactgtatacaaggcaacattgaggtcaggagaagtatatgccgtgaagaagcttgtgggtcatgcacacaagattttgcatggaagcatgataagggagatgaatacgcttggtcaaattaggcataggaacttagtaaaactaaaggatgttttgttcaggcgtgagtatgggttgatcctctatgaatttatggacaatggtagcctttatgatgttctgcatgggactgaggcagctccagttctagagtggaggacacggtatgacatagctcttggtacagcacatggtttagcttatctccacaatgactgccacccagccattattcaccgtgacattaaaccgaaaaatatactgttggataaggacatggtgccacatatttcagactttggcattgcaaagcttatcgacctgtctcctgctgcttcagaaactactggaatcgttggcactgttgggtatatggccccagagatggcattttcaaccaggagtaccattgagttcgacgtgtacagctacggagtcgtattacttgaattgattaccagaaagatggccctggatccctcgttccctcacgatgtggacctagtcagctgggtatcctccaccctgaatgagggcaacgtgatcgagtccgtgtgcgaccctgccctaatgcgtgaggtatgtggcactgctgaactggaggaagtatgcagtgtgctgtcgatagcccttagatgcaccgcggaggacgcaagacagaggccttccatgatggatgttgtgaaagagctgacacgtgctaggcatgatgttgtatcgctaccgaagcaggcggtgtctggttccagcagttcctgtcaaaacctggcaacctga

SEQ ID NO: 72 - protein, 1114 aa, Triticum aestivum
MGLISWHRLLLFSNLVSLCCGLSSDGHALLALSRRLILPDTISSNWSSSDTTPCGWKGVQCEMNNVVHLNLSYYKVSGSIGPEVGRMKYLRQLDLSSNNISGPIPHELGNCVLLDLLDLSGNSLSGGIPTSLMNLKKLSQLGLYSNSLSGEIPEGLFKNRFLERVYLQDNKLSGSIPSSVGEMKSLKYFRLDGNMLSGALPDSIGNCTKLENLYLYGNKLNGSLPRSLSNIKGLVLFEANNNSFTGDISFRFKSCKLEVFVLSWNQISGEIPGWLGNCSSLIRLAFLHNRLSGQIPTSLGLLKKLSILILTQNSLSGLIPPEIGSCRSLVWLELDANQLEGTVPKQLANLRNLQQLFLFENRLSGEFPQDIWGIQGLESVLLYNNSLSGGLPPMSAELKHLKFVKLQDNLFTGVIPPGFGINSPLVEIDFTNNRFVGGIPPNICSGKRLTAWNLGHNFLNGTIPFTVASCPSLERVRLHNNNLSGQVPQFRDCANLRYIDLSHNSLSGHIPASLGRCANITAINWSQNKLGGPIPPELGQLVKLESLDLSHNSLEGAIPAQISSCSKLHLFDLSFNSLNGSALTTVCKLEFMLNLRLQRNRLSGGIPDCISQLHGLVELQLGGNVLGGHLPSALGTLKRLSTALNLSSNGLEGSIPSQLRFLVDLASLDLSGNNLSGDLAPLGSLHALYTLNLSNNRFSGPVPENLVQFINSTPSPFSGNSDLCVSCHDDDSSCKGANVLEPCSSLRRRGVHGRVKIAMICLGSVFVGAFLILCIFLKYRGSKTKPEGELNPFFGESSSKLNEVLESTENFDDKYIIGTGGQGTVYKATLRSGEVYAVKKLVGHAHKILHGSMIREMNTLGQIRHRNLVKLKDVLFRREYGLILYEFMDNGSLYDVLHGTEAAPVLEWRTRYDIALGTAHGLAYLHNDCHPAIIHRDIKPKNILLDKDMVPHISDFGIAKLIDLSPAASETTGIVGTVGYMAPEMAFSTRSTIEFDVYSYGVVLLELITRKMALDPSFPHDVDLVSWVSSTLNEGNVIESVCDPALMREVCGTAELEEVCSVLSIALRCTAEDARQRPSMMDVVKELTRARHDVVSLPKQAVSGSSSSCQNLAT

SEQ ID NO: 73 - DNA, 3315 bp, Cucumis sativus
  source 1..3315: mol_type = genomic DNA
atgcagcttctcacccgccatttcttcttactggtatgcttctctttccatttcgttgttgttgtttttggtttaacttcagatgggttggccttgttatcactccaaagccgctggactactcatacctcctttgtccctgtttggaatgcctctcattccactccctgttcttgggctgggattgaatgtgatcaaaacctccgtgtcgtcaccttcaatctctctttctatggggtttcgggtcaccttggacctgaaatttcaagtttgactcagttgcgtactattgatttgaccaccaacgatttctctggtgaaattccttatgggattggtaactgtagccatttagagtacttggatctctccttcaaccaatttagtggacaaattcctcagtcattgaccctccttacgaacttgacgtttttgaacttccatgaaaatgttttaactggtccaatacccgactccttatttcagaatcttaatttccagtatgtgtatcttagtgaaaacaatctcaacggttctatcccttcaaatgtggggaattcgaatcagctgttgcatttgtatctgtatggaaatgagttttctggttccataccttcttccatagggaactgtagtcaattggaggatctttatttggatgggaaccagttagtgggaacattgcccgatagtcttaacaatcttgataatcttgttaatctgggtgtaagccgaaacaatctccagggtccaattcctttgggttcaggagtttgtcagagtttagaatatatagatttgtcattcaatggttatacaggaggtatacctgccgggttgggcaactgtagtgccctaaaaaccttacttattgtaaattccagtttaacaggtcatattccttcctcctttggccgcctaaggaagctttcacatattgatctctctagaaatcaactgtctgggaatatacctcctgaatttggggcttgcaaatccttgaaagaattggatttgtacgataaccaactcgagggacgtatccctagtgaactgggcttgctaagtagattagaggtccttcaattgttttcaaaccggttgactggtgaaattccaattagcatctggaagattgcaagtctccaacagattcttgtgtatgacaacaacctttttggggaattacccttgataataacagaactcaggcacctcaaaatcatttctgtgttcaacaatcatttttctggagtcatacctcaaagtttgggactcaacagtagcttagtgcaggtggagttcaccaataatcagttcactggtcaaattccacctaatttatgctctggaaagacattaagggtgctaaatttaggtttgaatcaatttcaaggaaatgtacctcttgatattggaacttgtttgactcttcagagattaattttaagaaggaataatctagcaggggttctgccagaatttacgataaatcacggtcttcgattcatggatgccagtgaaaataacctcaatggaacaattccctcaagtttgggaaattgcatcaatcttacctcaattaatcttcaaagcaacaggctttcaggccttatacctaatggattgagaaatcttgagaatcttcaaagtttgattttgtctcataacttcttggaaggacctttgccatcttccctctcaaattgcacgaagctggataaatttgacgtaggatttaatttattgaatggttctatacctcgtagtttagccagctggaaagttatatccacgtttataattaaagagaatcgatttgctggaggtatcccgaatgtattatcagaacttgaaagcctttcactactagatcttggtggcaatctgtttggaggtgaaatcccttcatctattggaaatttgaagagtctgttttactccctgaatcttagtaacaatgggttaagtggcacactaccttctgagctggcgaatcttgtcaagttacaggaattagatatatctcacaataatttgactggaagtttaactgtccttggcgaactaagttcgacattagttgagcttaacatttcatataattttttcaccggtccagtgccacaaacattaatgaagttattgaattctgatccctcatcgttcttaggtaaccctgggttgtgcattagttgtgatgtaccagatggtctaagttgcaatagaaatatcagtattagtccttgtgctgttcattcaagcgcccgtggtagctctcgacttggaaatgtacagattgcaatgatagctctggggtcttcactttttgttattcttttgcttcttgggttggtttataagtttgtttataacagaagaaacaagcaaaacattgagactgctgctcaagtcggaacaacttccttgctcaacaaggtaatggaggccaccgataatttagatgaacgtttcgtcattggaagaggagcacatggagttgtttataaggtctccctggattcaaataaagtttttgctgtaaagaagcttacatttttaggacataaaaggggaagccgggatatggttaaagaaattagaactgtcagcaacatcaagcaccggaacttgatctctttggaaagtttttggttgggaaaagattatggtctattgctttacaaatactatccaaatgggagcctttatgatgtgttgcacgagatgaatacaactccatctctcacatggaaagcccgctataatatagcgatcggtattgctcatgcattggcatatctccattacgattgtgatcctcccattatacaccgagacatcaaaccacagaatatacttctagattcggagatggaacctcatatcgccgactttggtcttgcaaagctattggatcaaactttcgaacccgcgacttcatcttcttttgcgggtacaattggctacattgcaccagagaatgcattttcagcagcaaagaccaaagcctcagatgtgtacagttatggggtggttttactggagctggtgacgggaaagaagccatcagatccatcatttatagaagtcgggaatatgacggcttggattcggtcggtttggaaggagagagatgaaatagatagaattgttgatccaaggcttgaggaagaacttgctaatttggatcatagggagcagatgaatcaggtggttcttgtggctttgagatgcacagaaaacgaggctaacaaaagacctataatgagagagattgtggatcacttgattgatttaaagatcagtcgttag

SEQ ID NO: 74 - protein, 1114 aa, Cucumis sativus; identical to SEQ ID NO: 72

SEQ ID NO: 75 - DNA, 3261 bp, Prunus persica
  source 1..3261: mol_type = genomic DNA
atgcagctttatctgttcaatttcttcttgttgttgctgttgctgtgcttctctgtgtccatatctactgtatctagtttgagctctgatgggttggctttattgtcactctccaagcattggacgtctgtccctgcttccatatcctcaagctggagtgcttctgatgccactccatgccaatgggttggaatagaatgtgacaatgcccacaatgtggttaccttgaacctcactggttatggaatttctggtcaattgggacctgaagttggcagctttaggcacttgcagactcttgatttgagtgtcaacaatttttctggcaaaatcccaaaagaattggccaattgtagtcttcttgaaaacttggacttgtataaaaatggcttttctggtgcaatacctgaatccttgtttgcaattcctgccttagcttatgtacatctgtatactaacaatttgaatggttccatccctggaaatgttgggaatctgagtgagcttgtgcatctgtatttgtatgagaatcagttttcaggagtgattccttcatccattggaaactgtagtaaattgcaggagttgtttttgggtcggaaccagttgacaggagagcttcctatgagcctgaacaaccttcagaacctggtatatctagatgtagctataaatagtcttgagggtagcattcctttgggttcaggcacttgcaagaatttaatttacttggatttgtcatacaataagtttagtggaggtattccaccaggacttggaaattgtagtaatttaacacaattttctgctgtgggtagtaatttagaggggactattccatcttcctttggccaactcaagtacctttcaaccctgtaccttcctctaaatcatttgtctggtaaaatacctcctgaacttggcaagtgtgaatccttgaagatactacgcttgtataagaaccaactagtgggagaaattcctagtgaattgggaatgctgactcaattagaggaccttgaattatttgagaaccggttaactggtgaaattccagttagcatatggaagattcaaagtcttcagcatatccttgtgtacaataatagcctcactggggagctgcctgaagtgatgactgagctgaagcaactaaaaaatatatcattgtacaacaacctattttttggagttatacctcaaagtttggggattaacagcagtttgtggcagttagattttatcaataacaagttcactggtaaaatccctccaaatctttgccatggaaagcagttaagggtgttgaatctgggtttcaatcgatttcaaggcaccataccttctgatgttggaaattgctctactctttggaggttgaaactggaacagaacagactcattggggctcttccacagtttgcaaaaaattcaagcctttcatatatggacatcagcaacaatgagattagtggagaaattccatcaagcttgggaaactgtagcaatctcacagccatcaatttgtccatgaacaatttaacaggagttataccccaggaactagggagtcttgcagaacttggttctttgattcttttcaaaaacaatttggttggtcctctgcctcctcacctatcaaattgtaccaaaatgtataagtttgatgtgggatcaaatttgttgaatggctccattccatcgagtttgagaagttggacaggtttatcaacgctgattttaagtgacaacagttttactggcggtgttccacctttcttgtcggagtttgaaaagctttcagagctacaacttggtggaaattttttgggaggtgccattccctcatcaattggagcattggttagtatgttttatgcattgaatcttagcaataatgcattgacaggtccgattccttcagagctggggaagttggcaagactacaacgactagatctatctcataacaatttgacagggactttaaaagctcttgattatatcaattcattgattgaggttgatgtttcagacaataacttcacaggtgcagtaccagagacattgatgaaccttttgaactcatctccattatcatttttgggcaatccctacctatgtgtcgattaccttccatcatgtggctcaacttgtgcaagaagaaacaacagttttaagccatgcaacagtcaatcaagcaagcatagaggccttagtaaagtggcaattgcatttatatccctaggttcttcattatttgttgtttttgtgcttcatgtactggtttatatgtttctcttgcgcaaaaagacaaagcaggaacttgagatctctgctcaagaggggccatctggcttactcaacaaagtattggaggctacagcgaaccttaatggtcagtatatcattgggaaaggagcccatggaactgtctataaggcctctttggctccagacaaagattatgctgtcaagaagcttctgtttgcggggcatgaaggaacgcgtttgagcatggttagagaaattcaaaccctcgggactattaggcatcggaatctggttaaattggaagacttctggttaagaaaggaccacggtttaatcttgtataggtacatgcaaaatgggagccttaatgatgttttgcatgaaattaaacccccaccaactcttgagtggagtgtccgctataggatagcacttggaactgcatacgggttggagtatctccattatgattgtgacccccctatagttcatcgagatgtcaaaccaatgaacatcctcttagacgctgatatggagcctcatattgctgattttggtattgctaaacttctggatcagtcttctgcatcaacgacgtccattgcagttgtgggaacaactggatatattgcaccagaaaatgcatttagaccagcaaagagcgtggaatctgatgtgtacagttatggggttgttctacttgagctgataactagaaagaaggcattggatccatcatttgtggagcaaactgacattgtaggatgggttaggtcagtgtggagcaacacagaagaaattcatcaaattgttgattcaagccttaaggaggaatttctggattcatgtattatggaccaagttgtcgatgtgcttatggtggcattcagatgtactgataaagatcctagaaagaggccgacgatgagagatgttgtgaagcaattattagatgcaaatccccaagtgagaagcataaaaggctag

SEQ ID NO: 76 - protein, 1086 aa, Prunus persica
MQLYLFNFFLLLLLLCFSVSISTVSSLSSDGLALLSLSKHWTSVPASISSSWSASDATPCQWVGIECDNAHNVVTLNLTGYGISGQLGPEVGSFRHLQTLDLSVNNFSGKIPKELANCSLLENLDLYKNGFSGAIPESLFAIPALAYVHLYTNNLNGSIPGNVGNLSELVHLYLYENQFSGVIPSSIGNCSKLQELFLGRNQLTGELPMSLNNLQNLVYLDVAINSLEGSIPLGSGTCKNLIYLDLSYNKFSGGIPPGLGNCSNLTQFSAVGSNLEGTIPSSFGQLKYLSTLYLPLNHLSGKIPPELGKCESLKILRLYKNQLVGEIPSELGMLTQLEDLELFENRLTGEIPVSIWKIQSLQHILVYNNSLTGELPEVMTELKQLKNISLYNNLFFGVIPQSLGINSSLWQLDFINNKFTGKIPPNLCHGKQLRVLNLGFNRFQGTIPSDVGNCSTLWRLKLEQNRLIGALPQFAKNSSLSYMDISNNEISGEIPSSLGNCSNLTAINLSMNNLTGVIPQELGSLAELGSLILFKNNLVGPLPPHLSNCTKMYKFDVGSNLLNGSIPSSLRSWTGLSTLILSDNSFTGGVPPFLSEFEKLSELQLGGNFLGGAIPSSIGALVSMFYALNLSNNALTGPIPSELGKLARLQRLDLSHNNLTGTLKALDYINSLIEVDVSDNNFTGAVPETLMNLLNSSPLSFLGNPYLCVDYLPSCGSTCARRNNSFKPCNSQSSKHRGLSKVAIAFISLGSSLFVVFVLHVLVYMFLLRKKTKQELEISAQEGPSGLLNKVLEATANLNGQYIIGKGAHGTVYKASLAPDKDYAVKKLLFAGHEGTRLSMVREIQTLGTIRHRNLVKLEDFWLRKDHGLILYRYMQNGSLNDVLHEIKPPPTLEWSVRYRIALGTAYGLEYLHYDCDPPIVHRDVKPMNILLDADMEPHIADFGIAKLLDQSSASTTSIAVVGTTGYIAPENAFRPAKSVESDVYSYGVVLLELITRKKALDPSFVEQTDIVGWVRSVWSNTEEIHQIVDSSLKEEFLDSCIMDQVVDVLMVAFRCTDKDPRKRPTMRDVVKQLLDANPQVRSIKG

SEQ ID NO: 77 - DNA, 3273 bp, Prunus persica
  source 1..3273: mol_type = genomic DNA
atgcagctttatctgttcaattttttgttgttgttgtgcttgtctgtgtccatatccactgtatctagtttgaactctgatggggtggctttgttgtcactctccaagcattggacctctgtccctgcttccatatcctcaagctggaatgcttctgattccactccatgccaatgggttgggatagaatgtgacaatgaccacaatgtggtttccctgaagctcactggttatggaatttctggtcaattgggacctgaaattagcagatttaggtacttgaagattcttgatttgagtgttaacaaattttctggcaaaattccaacagaattggccaattgtagtcttcttgaaaacttggacttgtatgaaaatggcttttctggtgaaattcctgaatccttctttgcaattcctgccttagcttatgtgcatctgtatagtaaccgtttgaatggttccatccctggaaatgttgggaatctgagtgagcttgtgcatctggatttatatgagaatcagttttcaggagtcattccttcatccgttggaaactgtagtaaattggaggacttgtatttggctgagaaccagttgataggagaacttcctaagagtctgaacaagcttgagaacctggtatatctagatgtggctaacaatagtcttgagggtagcattcccttgggttcaggtacttgcaagaatttaatttacttggatttttcatacaataagtttagtggaggtattccaccaggacttggaaattgtagtaatttaacacaattttctgctgtgggtagcaatttagaggggactattccatcttcctttggccaactcaagtacctttccatcctgtaccttcctctaaatcatttgtctggtaaaatacctcctgaacttggcaagtgtgaatccttgaaggaactacacttgtatacaaaccaactagtgggagaaattcctggtgaattggggatgctgactcaattacaggaccttaaattatttgagaatcggttaactggtgaaattccagttagcatatggaagattcaaagtcttcagcatatccttgtgtacaataatagcctcaccggggaactgcctgtagtgatgactgagttgaagcagctaaaaaatatatcattgttcaacaacctgttttttggagttatacctcaaactttggggattaacagcagtttgtggctgttagattttaccaataataagttcactggtaaaatccctccaagtctttgccgtggaaagcagttatggaagttgaatatgggtttcaatcgaattcaaggtaccattccttctgatgttggaaattgctctagtctaagcaggttgaaactgggacacaataacctcactggggttctaccacagtttgcaaaaaattcaagacttttatatatggacatcagcaacaatgagattagtggagaaatcccatctatcttgggaaattgtagcaatctcacaaccatcaatttgtccataaacaagttaacaggaggtataccccaggagctagggaatcttgaagagcttcgttctttgattcttttcaagaacaatttggttggtcctctacctcctcagctttcaaagtgtaccaaaatggataagtttgacgtgggatcaaatttgttgaatggttccattccatcgagtttgagaagttggacagatttatcaacactgattttaagtgacaatagttttactggagaaattccacgtttcttcacagagtttgaaaaacttatagagctacgacttggtggaaatttgtttgcaggtgcgattccctcatcaatcggagcattggttagtctatcatatgcattgaatcttagcaataatgcattgacaggtcggattccttcagagcttgggaagttgacaagcctacaacaactcgatctatctcataacaatttgacagggactttaaaagctcttgatcatatgatttcattgactgaggtggatgtttcagacaataacttcacaggttcagtaccagagacgtttatgaagctgttgaactcatcatcattatcatttttgggcaatccctacctatgtgtcagttaccttccattatgtggctcaacttgtggaagaaacaacagttttaagctgtgcaaccgtcaattaagcaatcataaaggccttagtaaagtagaaattgcatttacagccctggggtcttcattattcgttgtttttgtgctttatggactggtttatatgttcctcttacgcaaaaagaccaagcaggaacttgaggtctctgctcaagacaggctgtcttccctactcaaggaagtaatggaggccacagaaaatcttaatgatcaatatatcattgggaaaggagcccatggaactgtctataaggcctttttggctccagacaaagattatgccgtcaagaagcttgtatttgcagggcatgaaggaacgcgttcaagcatggttagagaaattcaaaccctggggacgataaggcatcgaaatctagttaaattggaagacttctggttgagaaaggaccatggtttaatcttgtacaggtacatggaaaatgggagccttcatgatgctctacatgaaattaaacccccaccaactcttgagtggattgtccgctacaggatagcacttggaactgcatacggtttggagtatctccattttgattgtgacccccgtatagttcatcgagatgtaaaaccaatgaacattctcttagactctgatatggagcctcatgtcgctgattttggtattgctaaacttttggatcagtcttctgcatcaacggcgtccgctgcagttgtgggtacaactggatatattgcaccagaaaatgcatctagaccatcaacgagtgtggaatctgatgtgtacagttatggggttgttttacttgagctgataactaggaagaaggcattggatccagcatttggggagcaaactgacattgtaggatgggctaggtcagcgtggagcaacacagaagacattgatcagatcgttgattcaagccttaaggaggaactcccgcattcaaatataatcgaccaagttgtggatgtgcttatggtggctttcagatgtactgataaaaatcctagaaagagaccgacgatgagagatgttatccagcaattattagatgcaaatccccaagtgagaaaaagaaggatattgaatcctcgtaatggtttgtcctaa

SEQ ID NO: 78 - protein, 1090 aa, Prunus persica
MQLYLFNFLLLLCLSVSISTVSSLNSDGVALLSLSKHWTSVPASISSSWNASDSTPCQWVGIECDNDHNVVSLKLTGYGISGQLGPEISRFRYLKILDLSVNKFSGKIPTELANCSLLENLDLYENGFSGEIPESFFAIPALAYVHLYSNRLNGSIPGNVGNLSELVHLDLYENQFSGVIPSSVGNCSKLEDLYLAENQLIGELPKSLNKLENLVYLDVANNSLEGSIPLGSGTCKNLIYLDFSYNKFSGGIPPGLGNCSNLTQFSAVGSNLEGTIPSSFGQLKYLSILYLPLNHLSGKIPPELGKCESLKELHLYTNQLVGEIPGELGMLTQLQDLKLFENRLTGEIPVSIWKIQSLQHILVYNNSLTGELPVVMTELKQLKNISLFNNLFFGVIPQTLGINSSLWLLDFTNNKFTGKIPPSLCRGKQLWKLNMGFNRIQGTIPSDVGNCSSLSRLKLGHNNLTGVLPQFAKNSRLLYMDISNNEISGEIPSILGNCSNLTTINLSINKLTGGIPQELGNLEELRSLILFKNNLVGPLPPQLSKCTKMDKFDVGSNLLNGSIPSSLRSWTDLSTLILSDNSFTGEIPRFFTEFEKLIELRLGGNLFAGAIPSSIGALVSLSYALNLSNNALTGRIPSELGKLTSLQQLDLSHNNLTGTLKALDHMISLTEVDVSDNNFTGSVPETFMKLLNSSSLSFLGNPYLCVSYLPLCGSTCGRNNSFKLCNRQLSNHKGLSKVEIAFTALGSSLFVVFVLYGLVYMFLLRKKTKQELEVSAQDRLSSLLKEVMEATENLNDQYIIGKGAHGTVYKAFLAPDKDYAVKKLVFAGHEGTRSSMVREIQTLGTIRHRNLVKLEDFWLRKDHGLILYRYMENGSLHDALHEIKPPPTLEWIVRYRIALGTAYGLEYLHFDCDPRIVHRDVKPMNILLDSDMEPHVADFGIAKLLDQSSASTASAAVVGTTGYIAPENASRPSTSVESDVYSYGVVLLELITRKKALDPAFGEQTDIVGWARSAWSNTEDIDQIVDSSLKEELPHSNIIDQVVDVLMVAFRCTDKNPRKRPTMRDVIQQLLDANPQVRKRRILNPRNGLS

SEQ ID NO: 79 - protein, 8 aa, Solanum lycopersicum
  VARIANT 2: note = X is any amino acid
  VARIANT 5..6: note = X is any amino acid
GXPPXXNN

SEQ ID NO: 80 - protein, 9 aa, Arabidopsis thaliana
  VARIANT 4..5: note = X is any amino acid
  VARIANT 8..9: note = X is any amino acid
SSGXXGXXN

SEQ ID NO: 81 - protein, 9 aa, Arabidopsis thaliana
  VARIANT 4: note = X is any amino acid
  VARIANT 7: note = X is any amino acid
  VARIANT 9: note = X is any amino acid
SGGXGGXHX

SEQ ID NO: 82 - protein, 23 aa, Solanum lycopersicum
ATDRRGRPPSRPKVGSGPPPQNN

SEQ ID NO: 83 - DNA, 68 bp, Solanum lycopersicum
  source 1..68: mol_type = genomic DNA
cttgttcatcaagcatcctcctcttcatcctcgtcgtcctcccatgaagatggtaagaagcaaggttc

SEQ ID NO: 84 - DNA, 67 bp, Solanum lycopersicum
  source 1..67: mol_type = genomic DNA
cttgttcatcaagcatcctcctcttatcctcgtcgtcctcccatgaagatggtaagaagcaaggttc

SEQ ID NO: 85 - DNA, 24 bp, Solanum lycopersicum
  source 1..24: mol_type = genomic DNA
cttgctcccatgaaagcaaggttc

SEQ ID NO: 86 - DNA, 80 bp, Solanum lycopersicum
  source 1..80: mol_type = genomic DNA
cattccttgttttctatcccacatttggaaactatctactttaaccaaaacagtttgaatggttccatcccgtccggcat

SEQ ID NO: 87 - DNA, 76 bp, Solanum lycopersicum
  source 1..76: mol_type = genomic DNA
cattccttgttttctatcccacatttggaaactatctactttaaccaaaacagtttgaatggttccagtccggcat

SEQ ID NO: 88 - DNA, 81 bp, Solanum lycopersicum
  source 1..81: mol_type = genomic DNA
cattccttgttttctatcccacatttggaaactatctactttaaccaaaacagtttgaatggttccatccccgtccggcat

SEQ ID NO: 89 - protein, 23 aa, Hordeum vulgare
AVRRPRPPGNPREGRGGGGGHHN

SEQ ID NO: 90 - DNA, 68 bp, Hordeum vulgare
  source 1..68: mol_type = genomic DNA
cggtgaggaggccgcggccgcccggcaacccgagggaagggcgcggcggcggcggaggacaccacaac

SEQ ID NO: 91 - protein, 225 aa, Hordeum vulgare
MDQHQVKEGGTMDQHKEEERQQEEGEPTDQRREEEVAGKPDEPTDRRKEEEEEPEEGDNLNEPTGQRKEEDKEDKSGELTDQRKADAVAEELTDQRKVEVEEGEGEKETERSSGEESMSASSLSLFVHPCSLLLRYLFRAYAWCLGMADSFGGGTRPSAVPAASTNSSREEPGEAADIGTREISKTTTDRGFYMREVIMRVRAVRRPRPPGNPREGRGGGGGHHN

SEQ ID NO: 92 - DNA, 678 bp, Hordeum vulgare
  source 1..678: mol_type = genomic DNA
atggatcagcaccaggtgaaggagggagggacaatggatcagcacaaggaggaggagcgtcagcaggaggagggagagccgacggatcagcgcagggaggaggaggtggcgggcaagcccgacgagccgacggatcggcgcaaggaggaggaggaggagccggaggagggagacaacctcaacgagccaacgggtcagcgcaaggaggaagacaaggaggacaagtctggagagctcacggaccagcgcaaggcggatgcggtggctgaagagctcacggaccagcgcaaggtggaggtggaggagggggagggggagaaggagacggagaggagctccggcgaagagtccatgtcggcctcgtcgctctcgctcttcgtgcacccgtgctcgctcctgctgcggtacctcttccgcgcctacgcgtggtgcctgggcatggccgactccttcggcggcggcacaaggccgagcgccgtccccgctgcatctaccaactcatcccgggaagaaccaggcgaggcggcggacatcgggaccagggagatctccaagaccaccactgacagagggttctacatgcgggaggtgatcatgcgcgtgagggcggtgaggaggccgcggccgcccggcaacccgagggaagggcgcggcggcggcggaggacaccacaactag

SEQ ID NO: 93 - protein, 1114 aa, Hordeum vulgare
MGLISWHRLLVFFNLVSLCCGLSSDGHALLALSRRLILPDIISSNWSSSDTTPCGWKGVQCEMNIVVHLNLSYSEVSGSIGPEVGRLKYLRQLDLSSNNISGPIPHELGNCVLLDLLDLSGNSLSGGIPASLVNLKKLSQLGLYSNSLSGEIPEGLFKNRFLERVYLQDNELSGSIPSSVGEMKSLKYFTLDGNMLSGALPDSIGNCTKLEILYLYDNKLNGSLPRSLSNIKGLVLFDASNNSFTGDISFRFRRCKLEVLVLSSNQISGEIPGWLGNCSSLTTLAFLHNRLSGQIPTSLGLLKKLSFLILTQNSLSGVIPPEIGSCRSLVWLQLGTNQLEGTVPKQLSNLSKLRRLFLFENRLTGEFPRDIWGIQGLEYILLYNNSLSGVLPPMSAELKHLQFVKLMDNLFTGVIPPGFGGNSPLVEIDFTNNGFVGGIPPNICLGKRLKVWNLGHNFLNGTIPSTVANCPSLERVRLHNNRLNGQVPQFRDCANLRYIDLSDNSLSGHIPASLGRCANITTINWSKNKLGGPIPHELGQLVKLESLDLSHNSLEGAIPAQISSCSKLHLFDLSFNFLNGSALTTVCKLEFMLNLRLQGNRLSGGIPDCILQLHGLVELQLGGNVLGGNLPSSLGALKRLSTALNLSSNGLEGSIPSELRYLVDLASLDLSGNNLSGDLAPLGSLRALYTLNLSNNRFSGPVPENLIQFINSTPSPFSGNSGLCVSCHDGDSSCKGANVLEPCSSLRKRGVHGRVKIAMICLGSVFVGAFLVLCIFLKYRGSKTKPEGELNPFFGESSSKLNEVLESTENFDDKYIIGTGGQGTVYKATLNSGEVYAVKKLVGHAHKILHGSMIREMNTLGQIRHRNLVKLKDVLFKREYGLILYEFMDNGSLYDVLHGTEAAPNLEWRIRYDIALGTAHGLAYLHNDCHPAIIHRDIKPKNILLDKDMVPHISDFGIAKLINLSPADSQTTGIVGTVGYMAPEMAFSTRSTIEFDVYSYGVVLLELITRKMALDPSLPEDLDLVSWVSSTLNEGNVIESVCDPALVREVCGTAELEEVCSVLSIALRCTAEDARHRPSMMDVVKELTHARRDVVSLPKQGISGSSSSCQNLST

SEQ ID NO: 94 - DNA, 3345 bp, Hordeum vulgare
  source 1..3345: mol_type = genomic DNA
atggggctgatttcatggcatcgtttacttgtcttcttcaacctggtctcgctctgttgcggtttgagttcagatggccatgcccttctggctctctccaggaggctcatactacccgacatcataagctcgaactggagttcttctgatacgactccctgtgggtggaagggagttcagtgcgagatgaacattgtagttcacctcaacctatcatactccgaagtttctggttccattggtcctgaggtagggcgtctgaagtacctgcggcaactcgatttgtcaagcaacaacatctccggtccgatccctcatgaattgggcaactgtgttctgcttgatctgctggatctgtcaggtaacagcctttccggtgggatcccggcatccctcgtgaatctgaagaagctatcgcagctcggtttgtacagcaactccctcagtggagagatacccgagggcctgttcaagaaccggtttctggagcgggtgtatctccaagacaatgaactcagtggttctatcccttcatcggttggtgagatgaagagtcttaaatactttacgttggatgggaacatgttgtctggagctctgccggattcgattggcaactgcacaaagttggagattctatatctatatgataataaactgaatgggagtcttccaagatcattgagcaacatcaaagggctcgtgctcttcgatgccagcaacaacagttttacaggtgacatctctttcagattcaggagatgcaaacttgaagtgttagtgctatcttcaaatcagatcagcggggaaatccctgggtggctgggaaattgtagcagcttaaccacgcttgcatttctccacaaccgtctctctggccagataccgacttcactgggtttattgaaaaagctatcattccttatacttactcagaattctttgtctggggtaatccctcctgagatcggtagctgccggtcgctggtgtggctgcagctgggcacaaaccagcttgagggcactgttccgaaacagctgtctaatctgagtaagttgcggcggctatttctgtttgagaatcgcctcacaggggagttccctcgggatatttggggtatccaaggcctcgaatacatccttctgtacaacaacagcctatctggggtactacctccaatgtcagccgagttgaaacacctacagttcgtcaaacttatggataatctgttcactggagtcataccaccaggatttgggggtaatagccctttagtagagattgacttcacaaataacggatttgttggtggaatcccgccgaacatttgtttgggtaaaagattgaaagtttggaacttggggcataattttcttaatggtaccatcccgtccactgttgctaactgcccaagtttggaacgagttcgtctccataacaaccgtctcaatgggcaagttccgcaattccgagactgtgcaaatctgcggtacatagatttgagtgacaattccttaagtggccatattcctgcaagtttgggcagatgtgctaacattacaacgataaactggtctaaaaacaagcttggtggaccaataccgcacgaacttggacaattggtgaagctggaaagtcttgacctctcccacaacagcctagagggggcaattcctgcacagatttccagttgctcgaagttgcacttgtttgatttgagtttcaactttttgaatggttctgcgctcacaacagtatgcaagctggagtttatgttaaatctgcggctacaggggaatagattaagtggaggcattccagattgcatcttgcagttacatgggctagtcgagctgcaacttggtggcaatgttcttgggggtaatctcccttcatcattaggagctttgaaaagattgagtactgcactaaaccttagcagcaacgggctggagggcagcattccatctgaattacgttacttggtggatcttgcaagcttagatttgtctggtaataatctcagtggggatcttgctccgttaggaagtctacgtgcattgtataccttgaatctttccaataacagattcagcgggccagtgccagagaatcttatacagtttataaattccacaccaagtccattcagtggaaattcaggtctctgtgtgtcttgccatgatggtgattcttcttgcaagggagctaatgttttggaaccttgtagttcattgaggaaaagaggagtacatggccgagtcaagatagctatgatatgtcttggttcggtttttgttggtgcatttctggtactctgtatctttctgaagtacagaggttccaagactaaacctgagggagagttaaatccattttttggggagtcatcttctaaattaaatgaggttttggaatcaactgaaaactttgatgacaagtacatcatcggcacaggtggccaaggaactgtatacaaggcaacactgaattcaggagaagtatatgccgtgaagaagcttgtgggtcatgcacacaagattttgcatggaagcatgataagggagatgaatacacttggtcaaattaggcacaggaacttagtaaaactaaaggatgttttgttcaagcgtgagtatgggttgatcctctatgaatttatggacaatggtagcctttatgatgttctccatgggaccgaggcagctccaaatctagagtggaggatacggtatgacatagctcttggtacagcacatggtttagcttatctccacaacgactgccacccagccattattcaccgtgacattaaacccaaaaatatactgttggataaggacatggtgccacatatttcagactttggcattgcgaaacttatcaacctgtctcctgctgattcacagactactggaattgttggcactgttggatatatggccccagagatggcattttcaaccaggagtaccatagagttcgacgtgtacagctacggagtggtattacttgaactgattaccagaaaaatggccctggacccctcattgcctgaagatctggacctagtcagttgggtgtcctccaccctgaatgagggcaacgtaatcgaatccgtgtgcgaccctgctctagtgcgtgaggtatgtggcactgctgaactggaggaagtatgcagtgtgctgtcgatagcccttagatgcaccgcggaggacgcaagacacaggccttccatgatggacgttgtgaaagagctgacgcatgctaggcgtgatgttgtatcgctacccaagcaggggatatctggttccagcagttcctgtcaaaacctgtcaacttga

(19) United States
(12) Patent Application Publication
(10) Pub. No.: US 2026/0265771 A1
(43) Pub. Date: Sep. 10, 2026

(54) METHODS OF INCREASING THE REGENERATION EFFICIENCY IN PLANTS

(71) Applicant: Institute of Genetics and Developmental Biology Chinese Academy of Sciences, Beijing (CN)

(72) Inventors: Chuanyou Li, Beijing (CN); Wentao Yang, Beijing (CN); Fangming Wu, Beijing (CN); Huawei Zhai, Beijing (CN); Chuanlong Sun, Beijing (CN); Lei Deng, Beijing (CN)

(21) Appl. No.: 18/869,564

(22) PCT Filed: May 26, 2023
(86) PCT No.: PCT/EP2023/064285

§ 371 Date: Nov. 26, 2024

(30) Foreign Application Priority Data
May 27, 2022 (WO) PCT/CN2022/095561

(51) Int. Cl. C12N 15/82; C07K 14/415

(52) U.S. Cl. CPC C12N 15/8291; C07K 14/415

(57) ABSTRACT

The invention relates to methods of increasing regenerative capacity in plants. In particular, the methods of the invention relate to increasing the levels and/or activity of peptides involved in local wound signalling and plant regeneration. Also described are plants expressing these peptides and well as methods of producing such plants.

### TECHNICAL FIELD

The present invention relates to methods for increasing the regeneration efficiency of a plant or a part thereof. Also described are tissue regeneration factors and plant tissue culture mediums that can be used to increase regeneration efficiencies, as well as genetically altered plants expressing the tissue regeneration factors and tissue regeneration factor receptors.

### BACKGROUND

Living organisms are often exposed to a wide range of biotic and abiotic stresses that cause severe wounding, leading to partial or complete organ loss. Upon injury, both plants and animals are able to regenerate new tissues or organs to minimize the impact of regular wear and tear (Birnbaum and Sanchez Alvarado, 2008; Mathew and Prasad, 2021; Pulianmackal et al., 2014; Sena and Birnbaum, 2010; Sugimoto et al., 2011; Sugimoto et al., 2019). Compared to their animal counterparts, plants encounter injuries more frequently and have evolved a remarkable regenerative capacity to repair wounded tissues and to regenerate new organs or whole plants, although the extent of this ability varies among species and tissue types (Birnbaum and Sanchez Alvarado, 2008; Ikeuchi et al., 2019; Ikeuchi et al., 2016; Mathew and Prasad, 2021; Sena and Birnbaum, 2010; Sugimoto et al., 2019). Thus, plants display diverse modes of regeneration upon loss or injury of body parts. For example, most plants reconstruct shoot apical meristem or root apical meristem when local damage occurs (Ikeuchi et al., 2019; Ikeuchi et al., 2016). More strikingly, new organs or whole plants can grow from small pieces of injured tissue or even from single cells through de novo organogenesis or somatic embryogenesis (Ikeuchi et al., 2019; Ikeuchi et al., 2016; Kareem et al., 2016; Mathew and Prasad, 2021; Mendez-Hernandez et al., 2019). A feature of de novo organogenesis is the reactivation of cell proliferation at wounding sites and the formation of a cell mass called callus, which is competent for regenerative responses (Ikeuchi et al., 2013).

The regenerative capacity of plant cells can be enhanced in vitro when cuttings and similar explants are cultured on nutrient media supplemented with auxin and cytokinins (George et al., 2008; Skoog and Miller, 1957). A routinely used protocol for in vitro regeneration involves pre-culture of explants on an auxin-rich medium to generate a callus that is competent for organ regeneration (Valvekens et al., 1988). The intrinsic regeneration capacity of plants provides the fundamental basis for various agricultural and biotechnological procedures for the in vitro propagation and production of many plant species under controlled conditions (Ikeuchi et al., 2019; Ikeuchi et al., 2016; Sugiyama, 2015).

Given that wounding is essential to start regeneration both in nature and in vitro (Birnbaum and Sanchez Alvarado, 2008; Ikeuchi et al., 2013; Iwase et al., 2015; Sugiyama, 2015), it is generally believed that wounding stimuli may in fact provide a primary inductive trigger for this phenomenon (Birnbaum and Sanchez Alvarado, 2008; Hoermayer and Friml, 2019; Ikeuchi et al., 2019; Ikeuchi et al., 2016; Ikeuchi et al., 2020; Mathew and Prasad, 2021). However, what plants exactly perceive as a wound signal and how they start regeneration has remained unknown for decades.

There is therefore a need to understand the mechanisms of plant regeneration. Further to this is there is a need to be able to modulate the regenerative capacity of plants for agricultural and biotechnological applications, particularly for plants that are recalcitrant to culturing. There is also a need to modulate the regenerative capacity of plants that are commercially important, as well as rare or endangered species and those with advantageous characteristics. Traditionally plant regeneration has been limited to particular genotypes, and regeneration is often more difficult in genetically modified plants. The present invention addresses the need to increase the regeneration efficiency in all plants, including transgenic plants.

### SUMMARY OF THE INVENTION

Here we report that a Plant Elicitor Peptide (PEP) is a local wound signal that is able to increase the regenerative capacity of plants. In contrast to systemin that mainly regulates systemic defense responses, we show that mutations of PEPs, PEP precursors (PROPEPs) and PEP receptors (PEPRs) impair both local and systemic defense responses, suggesting that PEP is a local wound signal. Of significant note, while depletion of PROPEP or PEPR abolished the regeneration capacity of tomato plants, overexpression of these genes led to an enhanced regeneration capacity, and exogenous application of PEP dramatically increased regeneration and transformation efficiency. These observations show that PEP is the long sought-after wound signal promoting plant regeneration. Thus, PEP was designated as REGENERATION FACTOR1 (REF1), the regeneration factor precursor was designated as PROPEP, PROSIPEP, PROREF1 PRR or PRP (PROREF1) and the REF1 receptor was designated as PEPR, SIPEPR1, RER or PORK1 (REF1 receptor); these terms are used interchangeably herein. In further contrast to systemin, which has only been found in Solanaceous plants, orthologues of REF1, PRR and RER can be identified in most, if not all, sequenced species of the angiosperms, demonstrating that the function of PEPs is highly conserved in the plant kingdom. As such, this invention is applicable to all plants. Further evidence of this is provided in the Examples. Here we show that exogenous application of REF1 or overexpression or PRR and/or RER significantly increases the regenerative capacity of all plants tested, which included testing a number of different cultivars and plants known to be recalcitrant to regeneration.

As used herein, a tissue regeneration factor may be used interchangeably with REF1. A tissue regeneration factor precursor or PROREF1 may be used interchangeably herein. A tissue regeneration factor receptor may be used interchangeably with RER herein.

In one aspect of the invention, there is provided a tissue regeneration factor comprising an amino acid sequence as defined in SEQ ID NO: 2 or a functional variant or homologue thereof, wherein the homologue is preferably selected from SEQ ID NO: 4, 6, 8, 10, 12, 14, 16, 18, 89 or 20 or a functional variant or homologue thereof of any of SEQ ID NOs 4, 6, 8, 10, 12, 14, 16, 18, 89 or 20.

In one embodiment, the homologue or functional variant comprises a conserved motif. The sequence of the conserved motif may be selected from (SEQ ID NO: 79) GXPPXXNN, (SEQ ID NO: 80) SSGXXGXXN and (SEQ ID NO: 81) SGGXGGXHX, where X is any amino acid.

In another aspect of the invention, there is provided a plant tissue culture medium comprising the tissue regeneration factor of the invention. In one embodiment, the tissue culture medium is a callus-inducing medium, a shoot-inducing medium or a root-inducing medium.

In a further embodiment, the plant tissue culture medium further comprises auxin and/or cytokinin.

In one embodiment, the plant tissue culture medium comprises the tissue regeneration factor at a concentration between 0.00001 and 100 nM. In a further embodiment, the plant tissue culture medium comprises the tissue regeneration factor at a concentration of around 0.M nM, 1 nM, 10 nM, 50 nM or 100 nM.

In another aspect of the invention, there is provided a nucleic acid construct comprising a nucleic acid sequence encoding at least one of a tissue regeneration factor, a tissue regeneration factor precursor and a tissue regeneration factor receptor;
wherein the tissue regeneration factor is selected from SEQ ID NO: 2 or a functional variant or homologue thereof, wherein the homologue is preferably selected from SEQ ID NO: 4, 6, 8, 10, 12, 14, 16, 18, 89 or 20 or a functional variant or homologue thereof; and
wherein the tissue regeneration factor precursor is selected from SEQ ID NO: 22 or a functional variant or homologue thereof, wherein the homologue is preferably selected from SEQ ID NO: 24, 26, 28, 30, 32, 34, 36, 38, 91 or 40 or a functional variant or homologue thereof; and
wherein the tissue regeneration factor receptor is selected from SEQ ID NO: 42 or a functional variant or homologue thereof, wherein the homologue is preferably selected from SEQ ID NO: 44, 46, 48, 50, 52, 54, 56, 58, 60, 62, 64, 66, 68, 70, 72, 74, 76, 93 or 78 or a functional variant or homologue thereof.

In one embodiment, the homologue or functional variant of the tissue regeneration factor or tissue regeneration factor precursor comprises a conserved motif, where the sequence of the conserved motif is selected from (SEQ ID NO: 79) GXPPXXNN, (SEQ ID NO: 80) SSGXXGXXN and (SEQ ID NO: 81) SGGXGGXHX, where X is any amino acid.

In another embodiment, the nucleic acid construct comprises a nucleic acid sequence encoding a tissue regeneration factor precursor or a tissue regeneration factor receptor.

In another embodiment, the nucleic acid construct comprises a nucleic acid sequence encoding a tissue regeneration factor precursor and a tissue regeneration factor receptor.

In a further embodiment, the nucleic acid sequence encoding at least one of a tissue regeneration factor, a tissue regeneration factor precursor and a tissue regeneration factor receptor is operably linked to one or more regulatory sequences. The regulatory sequence may be a constitutive or strong promoter or an inducible promoter, wherein the inducible promoter is preferably the nopaline synthase promoter.

In another aspect of the invention, there is provided a host cell comprising the nucleic acid construct of the invention.

In another aspect of the invention, there is provided a genetically altered plant, plant part thereof, or plant cell, wherein said plant, part thereof or plant cell is characterised by increased expression and/or levels at least one of
i. a tissue regeneration factor; and/or
ii. a tissue regeneration factor precursor; and/or
iii. a tissue regeneration factor receptor
wherein the tissue regeneration factor is selected from SEQ ID NO: 2 or a functional variant or homologue thereof, wherein the homologue is preferably selected from SEQ ID NO: 4, 6, 8, 10, 12, 14, 16, 18, 89 or 20 or a functional variant or homologue thereof; and
wherein the tissue regeneration factor precursor is selected from SEQ ID NO: 22 or a functional variant or homologue thereof, wherein the homologue is preferably selected from SEQ ID NO: 24, 26, 28, 30, 32, 34, 36, 38, 91 or 40 or a functional variant or homologue thereof; and
wherein the tissue regeneration factor receptor is selected from SEQ ID NO: 42 or a functional variant or homologue thereof, wherein the homologue is preferably selected from SEQ ID NO: 44, 46, 48, 50, 52, 54, 56, 58, 60, 62, 64, 66, 68, 70, 72, 74, 76, 93 or 78 or a functional variant or homologue thereof.

In one embodiment, the homologue or functional variant of the tissue regeneration factor or tissue regeneration factor precursor comprises a conserved motif, where the sequence of the conserved motif is selected from (SEQ ID NO: 79) GXPPXXNN, (SEQ ID NO: 80) SSGXXGXXN and (SEQ ID NO: 81) SGGXGGXHX, where X is any amino acid.

In a further embodiment, the plant, part thereof or plant cell comprises the nucleic acid construct of the invention. Preferably, the nucleic acid construct is stably incorporated into the plant genome.

In a further aspect of the invention, there is provided a genetically altered plant, plant part thereof, or plant cell, wherein said plant is characterised by one or more mutation in the plant genome, where the mutation is the insertion of at least one additional copy of a nucleic sequence encoding a tissue regeneration factor precursor and/or at least one additional copy of a nucleic acid sequence encoding a tissue regeneration factor receptor, such that said nucleic acid sequence(s) is operably linked to a regulatory sequence, and wherein preferably the mutation is introduced using targeted genome editing;
wherein the tissue regeneration factor precursor is selected from SEQ ID NO: 22 or a functional variant or homologue thereof, wherein the homologue is selected from SEQ ID NO: 24, 26, 28, 30, 32, 34, 36, 38, 91 or 40 or a functional variant or homologue thereof; and
wherein the tissue regeneration factor receptor is selected from SEQ ID NO: 42 or a functional variant or homologue thereof, wherein the homologue is selected from SEQ ID NO: 44, 46, 48, 50, 52, 54, 56, 58, 60, 62, 64, 66, 68, 70, 72, 74, 76, 93 or 78 or a functional variant or homologue thereof.

In another aspect of the invention, there is provided the use of the tissue regeneration factor of the invention, the plant tissue culture medium of the invention or the nucleic acid construct of the invention to increase the regenerative capacity of a plant or part thereof.

In one embodiment, the plant, part thereof or plant cell is a monocot or dicot.

In another aspect of the invention, there is provided a method of increasing the regenerative efficiency of a plant, plant part thereof or one or more plant cells, the method comprising
i. administering the tissue regeneration factor of the invention to the plant or part thereof or one or more plant cells; or
ii. administering the plant tissue culture medium of the invention to the plant or part thereof or one or more plant cells; and/or
iii. introducing and expressing in the plant, part thereof or one or plant cells the nucleic acid construct of the invention.

In another aspect of the invention, there is provided a method of producing a plant, plant part thereof or one or more plant cells with increased regeneration efficiency, the method comprising
iv. administering the tissue regeneration factor of the invention to the plant or part thereof or one or more plant cells; or
v. administering the plant tissue culture medium of any of the invention to the plant or part thereof or one or more plant cells; and/or
vi. introducing and expressing in the plant, part thereof or one or plant cells the nucleic acid construct of the invention.

In one embodiment, the method further comprising producing a plant from the one or more plant cells.

In another embodiment, 0.01-500 nmol/L of the tissue regeneration factor is administered exogenously to the plant, part thereof or plant cell.

In another embodiment, 0.1 to 0.5 mL of the plant tissue culture medium is administered exogenously to the plant, part thereof or plant cell.

In another embodiment, the method increases one or more of callus formation, shoot regeneration and root regeneration.

In another embodiment, the method increases transformation efficiency of a plant, part thereof or plant cell.

In a further embodiment, the plant part is an explant, wherein preferably the explant is selected from, but not limited to, a nodal segment, apical meristem, root, cotyledon, embryo, leaf disc, leaf blade, pedicle, petiole, anther, pollen, microspore ovary, hypocotyl.

In any of the aspects described herein, the plant may be a monocot or a dicot.

### DESCRIPTION OF THE FIGURES

The invention is further described in the following non-limiting figures.

FIG. 1—A schematic of signalling in plants following wounding, (a) shows root meristem formation, (b) shows de novo root formation, (c) shows shoot regeneration and (d) shows stem cell formation in moss.

FIG. 2—Identification of SIPep (REF1) as a Pep homolog in tomato.

FIG. 3—(a) A schematic showing SIPEP (REF1), a 23-amino-acid peptide cleaved from a precursor protein called PROSIPEP (also referred to as PROREF1 and PRR). (b) sequence alignments of PROSIPEP (PRR) and PROSIPEP knock out mutants (prr).

FIG. 4—Expression of PI-II locally (red) and systemically (blue) in wounded wild type tomato plants, PROSIPEP (PRR) knock out mutants (prr-1 and prr-2) and PROSYSTEM/N knock out mutants (prs-1 and prs-2) and a PROSIPEP PROSYSTEMIN double mutant (prr1 prs1).

FIG. 5—(a) images of callus formation in the tomato cultivar Ailsa Craig (AC) where the cultivar is wild-type (WT), a PROSIPEP knockout (prr-1 and prr-2), or a PROSYSTEM/N knockout (prs-1). (b) Relative callus area in WT, prr-1, prr-2 and prs-2 Aisla Craig tomato cultivar.

FIG. 6—(a) Hypocotyl explants of WT (Ailsa Craig, AC) and prr mutants were cultured on callus-inducing medium (CIM) in the absence of SIPEP (REF1) and CIM REF1 at a concentration of 1 nM for 14 days, photographs of the callus formation of these plants are shown. (b) The distribution of projected callus area of WT and prr mutants grown in the absence of REF1 in a callus inducing medium (control) and in a callus inducing medium in the presence of 1 nM SIPEP (REF1). The projected area of callus was quantified by Image J. Individual values (black dots) are shown and error bars represent SD from three independent experiments.

FIG. 7—Hypocotyl explants of wild-type (WT) Ailsa Craig and prr mutants. (a) shows photographs of said explants. (b) shows the relative regeneration frequency, (c) shows the number of hypocotyl for said explants, data are mean±standard deviation of three independent experiments. ***P<0.001, **P<0.01, *P<0.05 (Student's t-test).

FIG. 8—Hypocotyl explants of wild-type (WT) Ailsa Craig and prr mutants cultured in the presence or absence of SIPEP (REF1) at 1 nM in a shoot inducing medium for 21 days. (a) shows photographs of said explants. (b) shows the relative regeneration frequency, (c) shows the number of hypocotyl for said explants, data are mean±standard deviation of three independent experiments. ***P<0.001, **P<0.01, *P<0.05 (Student's t-test).

FIG. 9—Hypocotyl explants of WT (Ailsa Craig, AC) were cultured on callus-inducing medium (CIM) containing indicated concentrations of REF1, and callus phenotype was scored 14 days after incubation. (a) shows photographs of said explants. (b) shows box plots representing the distribution of projected callus area. The projected area of callus was quantified by Image J. Individual values (black dots) are shown and error bars represent SD from three independent experiments. ***P<0.001, **P<0.01, *P<0.05 (Student's t-test).

FIG. 10—Callus formation in WT (Ailsa Craig, AC), PROSIPEP knockouts (prr-1 and prr-2), and PROSIPEP (PRR) overexpressing tomato plants (PRR-OE-1 and PRR-OE-2). (a) photographs of callus formation in said plants. (b) relative callus area in said plants.

FIG. 11—Shoot regeneration in WT (Ailsa Craig, AC), PROSIPEP knockouts (prr-1 and prr-2), and PROSIPEP (PRR) overexpressing tomato plants (PRR-OE-1 and PRR-OE-2). (a) photographs of shoot regeneration in said plants. (b) relative regeneration frequency in said plants. (c) the number of hypocotyl in said plants.

FIG. 12—SIPEPR1 (RER) is the tomato orthologue of Arabidopsis thaliana PEP receptors.

FIG. 13—SIPEP (REF1), but not systemin triggers auto-phosphorylation of the REF1 receptor (RER). (a) shows a pull-down assay between HA-Flag-tagged ectodomains of RER (purified from tobacco leaves), biotinylated systemin, biotinylated REF1 and REF1. (b) shows an autophosphorylation assay of tobacco leaves expressing the construct in (c) said tobacco leaves were treated with 100 nM REF1, system or a control for 20 min before autophosphorylation assays.

FIG. 14—(a) REF1 or systemin-induced pFRK1::LUC expression in A. thaliana protoplasts. Protoplasts were transfected with pFRK1::LUC along with indicated constructs and induced with 100 nM of REF1 or systemin for 3 hours. Data are mean±SD, n=3 repeats. Experiments were repeated three times with similar results. ***P<0.001 (Student's t test); ns, not significant. (b) REF1 or systemin-induced pFRK1::LUC expression in A. thaliana protoplasts. Protoplasts were transfected with pFRK1::LUC along with indicated constructs and induced with indicated concentrations of REF1 or systemin for 3 h. Data are mean±SD, n=3 repeats. Experiments were repeated three times with similar results. ***P<0.001 (Student's t test); ns, not significant.

FIG. 15—(a) sequence alignment of SIPEPR1 (RER) and RER knock out mutants (rer-1 and rer-3). (b) Western blot of RER overexpressing plants (RER-OE-3 and RER-OE-5) compared to a wild type (WT) plant.

FIG. 16—SIPEPR1 (RER) knock out mutants are defective in SIPEP (REF1)-induced callus formation. (a) photos of wild type (WT) and RER knockout mutants (rer-1 and rer-3) grown in the presence and absence of REF1. (b) relative callus areas of wild type (WT) and RER knockout mutants (rer-1 and rer-3) grown in the presence and absence of REF1.

FIG. 17—SIPEPR1 (RER) knock out mutants are defective in SIPEP (REF1) induced shoot regeneration. (a) photos of shoot regeneration in wild type (WT) and RER knockout mutants (rer-1 and rer-3) grown in the presence and absence of REF1. (b) relative regeneration frequency in wild type (WT) and RER knockout mutants (rer-1 and rer-3) grown in the presence and absence of REF1. (c) number of hypocotyl in wild type (WT) and RER knockout mutants (rer-1 and rer-3) grown in the presence and absence of REF1.

FIG. 18—SIPEPR1 (RER) overexpressing plants have improved callus formation capacity. (a) photos of callus formation in wild type (WT), RER knockout mutants (rer-1 and rer-3) and RER overexpressing plants (RER-OE-3 and RER-OE-5). (b) relative callus area in wild type (WT), RER knockout mutants (rer-1 and rer-3) and RER overexpressing plants (RER-OE-3 and RER-OE-5).

FIG. 19—SIPEPR1 (RER) overexpressing plants have improved shoot regeneration capacity. (a) photos of shoot regeneration in wild type (WT), RER knockout mutants (rer-1 and rer1-3) and RER overexpressing plants (RER-OE-3 and RER-OE-5). (b) relative regeneration frequency in wild type (WT), RER knockout mutants (rer-1 and rer-3) and RER overexpressing plants (RER-OE-3 and RER-OE-5). (c) number of hypocotyl in wild type (WT), RER knockout mutants (rer-1 and rer-3) and RER overexpressing plants (RER-OE-3 and RER-OE-5).

FIG. 20—SIPEP (REF1) promotes shoot regeneration in S. habrochaites (LA1777). (a) photos of LA1777 grown in the presence and absence of 1 nM REF1. (b) relative regeneration frequency of LA1777 grown in the presence and absence of 1 nM REF1. (c) number of hypocotyl of LA1777 grown in the presence and absence of 1 nM REF1.

FIG. 21—SIPEP (REF1) promotes shoot regeneration in S. peruvianum (PI126944). (a) photos of PI126944 grown in the presence and absence of 1 nM REF1. (b) relative regeneration frequency of PI126944 grown in the presence and absence of 1 nM REF1. (c) number of hypocotyl of PI126944 grown in the presence and absence of 1 nM REF1.

FIG. 22—Constructs for plant transformation and genome editing to induce the expression of PROSIPEP (PRR) and the SIPEP/REF1 receptor (RER). (a) Overexpression vector for plant transformation (PRR+RER-OE). (b) CRISPR-Cas9 vector design (PRR+RER).

FIG. 23—The PRR+RER-OE construct improves the regenerative capacity of recalcitrant tomatoes. (a) Photos of P126944 tomatoes (WT, PRR transformed, RER transformed, and PRR+RER transformed, grown in presence or absence of 1 nM REF1 for 19 days. (b) relative regeneration frequency of P126944 tomatoes (WT, PRR transformed, RER transformed, and PRR+RER transformed, grown in presence or absence of 1 nM REF1 for 19 days.

FIG. 24—The pepper orthologue of SIPEP (CaREF1) promotes root hair formation, photos of pepper roots grown with 0 nM, 10 nM and 100 nM CaREF1.

FIG. 25—The pepper orthologue of SIPEP (CaREF1) promotes callus formation. Cotyledon explants were cultured on callus-inducing medium (CIM) containing indicated concentrations of CaREF1. (a) photos of cotelydon explants grown with 0 nM, 1 nM and 10 nM CaREF1. (b) relative callus area of cotelydon explants grown with 0 nM, 1 nM and 10 nM CaREF1. Box plots represent the distribution of projected callus area. The projected area of callus was quantified by Image J. Individual values (black dots) are shown and error bars represent SD from three independent experiments. **P<0.01, *P<0.05 (Student's t-test).

FIG. 26—The soybean orthologue of SIPEP (GmREF1) promotes shoot regeneration. (a) shows photos of soybean explants grown in the presence (10 nM, 20 nM 50 nM and 100 nM) and absence of GmREF1 (mock). (b) shows relative regeneration frequency of soybean shoots grown in the presence (10 nM, 20 nM 50 nM and 100 nM) and absence of GmREF1 for 28 days. The relative regeneration capacity was represented by the relative frequency of explants with the regenerated shoots. Data are mean±SD of three independent experiments. Bars show mean±SD. ***P<0.001, **P<0.01, *P<0.05 (Student's t-test).

FIG. 27—The rice orthologue of SIPEP (OsREF1) promotes callus formation in Japonica rice. Mature seeds of ZH11 were cultured on CIM containing indicated concentrations of OsREF1. (a) photos of ZH11 grown in presence of 1 nM and 10 nM OsREF1 or in the absence of OsREF1 (mock). (b) callus phenotype was scored 20 days after incubation on CIM. Callus formation capacity was represented by the percentage of mature seed explants with regenerated embryonic calli. Data are mean±SD of three independent experiments.

FIG. 28—The rice orthologue of SIPEP (OsREF1) promotes shoot regeneration in Japonica rice. ZH11 was cultured on SIM containing indicated concentrations of OsREF1. (a) photos of ZH11 grown in presence of 0 nM and 10 nM OsREF1. (b) shoot regeneration capacity of ZH11 grown in with either 0 nM or 10 nM OsREF1.

FIG. 29—The rice orthologue of SIPEP (OsREF1) promotes shoot regeneration in Japonica rice. Nipponbare was cultured on SIM containing indicated concentrations of OsREF1. (a) photos of nipponbare grown in presence of 0 nM and 10 nM OsREF1. (b) shoot regeneration capacity of nipponbare grown in with either 0 nM or 10 nM OsREF1.

FIG. 30—The rice orthologue of SIPEP (OsREF1) promotes shoot regeneration in indica rice. 9311 rice was cultured on SIM containing indicated concentrations of OsREF1. (a) photos of 9311 rice grown in presence of 0 nM and 10 nM OsREF1. (b) shoot regeneration capacity of 9311 rice grown in with either 0 nM or 10 nM OsREF1.

FIG. 31—The rice orthologue of SIPEP (OsREF1) promotes growth of wild allotetraploid rice. L-04 rice was grown in the presence of 10 nM OsREF1 or in the absence of OsREF1 (mock). (a) photos of L-04 rice grown in presence the of 10 nM OsREF1 or absence of OsREF1 (mock). (b) primary root length of L-04 rice grown in the presence of 10 nM OsREF1 or absence of OsREF1 (mock).

FIG. 32—The rice orthologue of SIPEP (OsREF1) promotes callus formation in wild allotetraploid rice. L-04 rice was grown in the presence of 10 nM or 1 nM OsREF1 or in the absence of OsREF1 (mock). (a) photos of L-04 rice grown in presence the of 10 nM, 1 nM OsREF1 or absence of OsREF1 (mock). (b) callus formation capacity of L-04 rice grown in the presence of 10 nM, 1 nM OsREF1 or absence of OsREF1 (mock).

FIG. 33—The wheat orthologue of SIPEP/REF1 (TaREF1) promotes root and shoot growth in wheat. (a) photos of wheat grown in the presence of 100 nm TaREF1, or in the absence of TaREF1 (mock). (b) primary root length in wheat grown in the presence of 100 nm TaREF1, or in the absence of TaREF1 (mock). (c) shoot length of wheat grown in the presence of 100 nm TaREF1, or in the absence of TaREF1 (mock).

FIG. 34—The peach orthologue of SIPEP/REF1 (PpREF1) promotes callus formation. (a) photos of peach cultivar GF677 grown on a CIM in the presence of 100 nM PpREF1 and in the absence of PpREF1 (mock). (b) callus formation capacity of peach cultivar PpREF1 grown on a CIM in the presence 100 nM PpREF1 and in the absence of PpREF1 (mock) after 24 days. Callus formation capacity was represented by the percentage of leaves explants with regenerated embryonic calli. Data are mean±SD of three independent experiments.

FIG. 35—Example vector constructions for transformation and genome editing to increase expression of the PROSIPEP/PROREF1 (PRR) gene. (a) Example vector construction (pCeGFP:PRR) for plant transformation to induce overexpression of the PRR gene. (b) Example vector construction (pTX041:PRR) for genome editing using CRISPR to induce expression of the PRR gene.

FIG. 36—Example vector constructions for transformation and genome editing to increase expression of the SIPEPR1 (RER) gene. (a) Example vector construction (pCeGFP:RER) for plant transformation to induce overexpression of the RER gene. (b) Example vector construction (pTX041:RER) for genome editing using CRISPR to induce expression of the RER gene.

FIG. 37—Example vector constructions for transformation and genome editing to increase expression of the SIPEPR1 (RER) gene and the PROSIPEP/PROREF1 (PRR) gene. (a) Example vector construction (pCeGFP:PRR+RER) for plant transformation to induce overexpression of the PRR gene and the RER gene. (b) Example vector construction (pTX041: PRR+RER) for genome editing using CRISPR to induce expression of the PRR gene and the RER gene.

FIG. 38—The “PRR+RER-OE” construct greatly improves the regenerative capacity of recalcitrant tomatoes. (a) photos of control LA1777 tomatoes, PRR expressing LA1777 tomatoes, RER expressing LA1777 tomatoes, and PRR and RER expressing LA1777 tomatoes exposed to 1 nM REF1 for 25 days, or absence of REF1 (mock). (b) relative regeneration frequency of control LA1777 tomatoes, PRR expressing LA1777 tomatoes, RER expressing LA1777 tomatoes, and PRR and RER expressing LA1777 tomatoes exposed to 1 nM REF1 for 25 days, or absence of REF1 (mock).

FIG. 39—The tomato “PRR+RER-OE” construct greatly improves the transformation efficiency of recalcitrant soybean. (a) photos of control recalcitrant soybean and PRR+RER overexpressing (OE) recalcitrant soybean grown in the presence of 50 nM gmREF1, 100 nM GmREF1 or a vehicle only control (mock). (b) relative regeneration frequency of control recalcitrant soybean and PRR+RER overexpressing (OE) recalcitrant soybean grown in the presence of 50 nM GmREF1, 100 nM GmREF1 or a absence of REF1 (mock).

FIG. 40—HaPEP1 promotes shoot regeneration in sunflower. (A) The shoot regenerative capacity was represented by the relative frequency of explants with the regenerated shoots. (B) The shoot regenerative capacity was represented by the number of explants with different number of regenerated shoots. Bars show mean±SD. ***P<0.001, **P<0.01, *P<0.05 (Student's t-test). (C) photos of sunflower plants grown in the presence of 1 nM HaREF1, 10 nM HaREF1 or absence of HaREF1 (mock).

FIG. 41—GaREF1 promotes callus formation. (a) photos of cotton grown in the presence of 0.1 nM, 1 nM 10 nM, 100 nM 500 nM GaREF1 or absence of GaREF1 (mock), (b) Hypocotyl explants of cotton were cultured on callus-inducing medium (CIM) containing indicated concentrations of GaREF1, and callus phenotype was scored at 14 days after incubation. Box plots represent the distribution of projected callus area. The projected area of callus was quantified by Image J. Individual values (black dots) are shown and error bars represent SD from three independent experiments. ***P<0.001, **P<0.01, *P<0.05 (Student's t-test).

FIG. 42—OsREF1 promotes shoot regeneration of wild allotetraploid rice. (a) images of L-04 rice grown in the presence of 10 nM OsREF1 or absence of OsREF1 (mock). (b) regeneration frequency of L-04 rice grown in the presence of 10 nM OsREF1 or absence of OsREF1.

FIG. 43—TaREF1 promotes shoot regeneration of wheat. (a) photos of Chinese Spring wheat grown in the presence or 0 nM, 0.01 nM or 0.1 nM TaREF1. (b) immature embryos of Chinese Spring were cultured on CIM containing indicated concentrations of TaREF1, and callus phenotype was scored at 14 days after incubation on CIM. Callus formation capacity was represented by the percentage of immature embryo explants with regenerated embryonic calli. Data are mean±SD of three independent experiments.

FIG. 44—TaREF1 promotes shoot regeneration of wheat. (a) photos of Kn199 wheat grown in the presence of 0.01 nM or 1 nM TaREF1 or absence of TaREF1 (mock). (b) photos of Kn199 wheat grown in the presence of 0.01 nM or 1 nM TaREF1 or absence of TaREF1.

FIG. 45—Leaf explants were cultured on callus-inducing medium (CIM) containing indicated concentrations of MsREF1, and callus phenotype was scored at 14 d after incubation. Callus formation capacity was represented by the percentage of leaves explants with regenerated embryonic calli. Data are mean±SD of three independent experiments. Bars show mean±SD. ***P<0.001, **P<0.01, *P<0.05 (Student's t-test).

FIG. 46—Leaf explants were cultured on medium containing indicated concentrations of CsREF1. Shoot regeneration was scored at 21 d after incubation. The shoot regenerative capacity was represented by the number of regenerated shoots per explant. Data are mean±SD of three independent experiments. Bars show mean±SD. ***P<0.001, **P<0.01, *P<0.05 (Student's t-test).

FIG. 47—GmREF1 improves transformation efficiency of soybean.

FIG. 48—The tomato “PRP+RER-OE” construct improves the shoot regenerative capacity of recalcitrant rice.

FIG. 49—REF1-RER1 regulates regeneration through SERK co-receptors. (a) RER1 complexes with SERK1, SERK3a and SERK3b in a REF1-dependent manner. Tobacco leaves transfected with SERKs-myc and RER1-HA-Flag were treated with 100 nM REF1 before IP with anti-myc agarose. RER1 and SERKs were detected by immunoblotting with anti-Flag or anti-myc antibodies, respectively. (b and c) REF1-induced callus formation of indicated genotypes. Hypocotyl explants were cultured on CIM with or without 1 nM REF1 for 14 d before callus phenotype was scored. Shown are representative callus images (b) and quantification of the callus projection area (c). Bars, 1 mm. Data are mean±SE. n=30 hypocotyl explants. (d and e) REF1-induced shoot organogenesis of indicated genotypes. Hypocotyl explants were cultured on CIM for 14 d and then transferred to SIM with or without 1 nM REF1 for 21d before shoot organogenesis phenotype was scored. Shown are representative images of hypocotyl explants (d) and the number of regenerated shoots per explant (e). Bars, 0.5 cm. Data are mean±SE. n=50 hypocotyl explants. In (c), the projected area of callus was quantified by Image J. Box plots represented the distribution of projected callus area. Callus area of each genotypes are normalized against WT. Individual values (black dots) were shown and error bars represent SD from three independent experiments. In (c) and (e), data points from three biological replicates were analysed with a one-sided Kruskal-Wallis test with Holm correction, followed by post hoc Dunn's test. Data points with different letters indicate significant differences of p<0.05.

FIG. 50—REF1 regulates regeneration through promoting SIWIND1 expression. (a) Induction of the SIWIND1 promoter activity at the wound sites of hypocotyl explants. Hypocotyl explants of 7-d-old pSIWIND1::GUS plants were cultured on MS medium with or without 1 nM REF1. Representative images of hypocotyls at indicated time after wounding are shown. Bars, 1 mm. (b) Wound-induced SIWIND1 expression revealed by RT-qPCR. Hypocotyls of 7-d-old seedlings of the indicated genotypes were cut and hypocotyl explants were cultured on MS medium for 9 h before gene expression analysis. Hypocotyls of undamaged seedlings were harvested as control. Data are mean±SD, n=3 biological repeats. (c and d) REF1-induced callus formation in WT and sIwind1 mutants. Hypocotyl explants were cultured on CIM with or without 1 nM REF1 for 14 d before callus phenotype was scored. Shown are representative callus images (c) and quantification of the callus projection area (d). Bars, 1 mm. Data are mean±SE. n=30 hypocotyl explants. (e and f) REF1-induced shoot organogenesis in WT and sIwind1 mutants. Hypocotyl explants were cultured on CIM for 14 d and then transferred to SIM with or without 1 nM REF1 for 21 d before shoot organogenesis phenotype was scored. Shown are representative images of hypocotyl explants (e) and the number of regenerated shoots per explant (f). Bars, 0.5 cm. Data are mean±SE. n=50 hypocotyl explants. (g and h) Callus formation in WT and SIWIND1-OE plants. Hypocotyl explants were cultured on CIM for 14 d before callus phenotype was scored. Shown are representative callus images (g) and quantification of the callus projection area (h). Bars, 1 mm. Data are mean±SE. n=30 hypocotyl explants. (i and j) Shoot organogenesis in WT and SIWIND1-OE plants. Hypocotyl explants were cultured on CIM for 14 d and then transferred to SIM for 21 d before shoot organogenesis phenotype was scored. Shown are representative images of hypocotyl explants (i) and the number of regenerated shoots per explant (j). Bars, 0.5 cm. Data are mean±SE. n=50 hypocotyl explants. In (d) and (h), the projected area of callus was quantified by Image J. Box plots represented the distribution of projected callus area. Callus area of each genotypes are normalized against WT. Individual values (black dots) were shown and error bars represent SD from three independent experiments. In (b), (d) and (f), data points from three biological replicates were analysed with a one-sided Kruskal-Wallis test with Holm correction, followed by post hoc Dunn's test. In (h) and (j), statistical significance was determined by a student's t test (***p<0.001, **p<0.01, *p<0.05).

FIG. 51—Positive feedback regulation between REF1 & SIWIND1 in response to wounding. (a) Induction of the PRP promoter activity at the wound sites of hypocotyl explants. Hypocotyl explants of 7-d-old pPRP::GUS plants were cultured on MS medium. Representative images of hypocotyls at indicated time after wounding are shown. Bars, 1 mm. (b) Wound-induced PRP expression in the indicated genotypes. Hypocotyls of 7-d-old seedlings were cut and cultured on phytohormone-free MS medium for 48 h before RNA extraction and gene expression analysis. Data are mean±SD, n=3 repeats. (c) REF1-induced PRP expression in the indicated genotypes. Eighteen-day-old seedlings were treated with or without 100 nM REF1. SIWIND1 expression was measured 24 h after REF1 treatment. Data are mean±SD, n=3 repeats. (d) Schematic representation of PRP promoter showing primers and probes used for ChIP-qPCR and EMSA. Blue boxes represent VWRE, red lines represent primers used for ChIP-qPCR, green triangles represent probe. (e) EMSA showing that SIWIND1 binds the VWRE motif of PRP promoter. Biotin-labeled probes were incubated with SIWIND1-His protein, and the free and bound DNAs (arrows) were separated on an acrylamide gel. As indicated, 10-, 20- and 50-fold excesses of unlabelled and 50-fold mutated probes were used for competition. Mu, mutated probe in which the core sequence (AAATTT) of VWRE was replaced by 5′-GGGGGG-3′. (f) ChIP-qPCR showing wound-induced SIWIND1 enrichment on the chromatin of PRP. The chromatin of damaged or undamaged SIWIND1-GFP seedlings was immunoprecipitated using anti-GFP antibody, and the immunoprecipitated DNA was quantified by qPCR. The enrichment of target gene promoters was displayed as a percentage of input DNA. Primers used for PCR amplicons are indicated in (d). Data are mean±SD, n=3 repeats. (g) Proposed model through which the immunomodulatory phytocytokine REF1 promotes wounding-triggered regeneration. Upon cellular damage, REF1 binds and activates the RER1-SERKs receptor complex to initiate both MYC2-dependent defence responses and SIWIND1-dependent regeneration responses. In addition to orchestrating the cellular reprogramming of regeneration, activated SIWIND1 also upregulates the expression of the REF1 precursor gene through promoter binding, thereby amplifying the primary wound signal for tissue regeneration. In (b-c) and (f), data points from three biological replicates were analysed with a one-sided Kruskal-Wallis test with Holm correction, followed by post hoc Dunn's test. Data points with different letters indicate significant differences of p<0.05.

FIG. 52—ZmREF1 promotes shoot regeneration.

FIG. 53—TaREF1 promotes shoot regeneration of Chinese Spring wheat.

FIG. 54—TaREF1 promotes callus formation in JM22 wheat.

FIG. 55—TaREF1 promotes shoot regeneration of JM22 wheat.

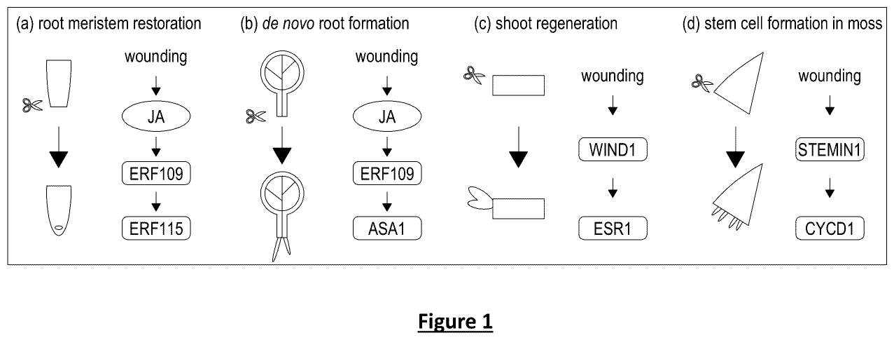

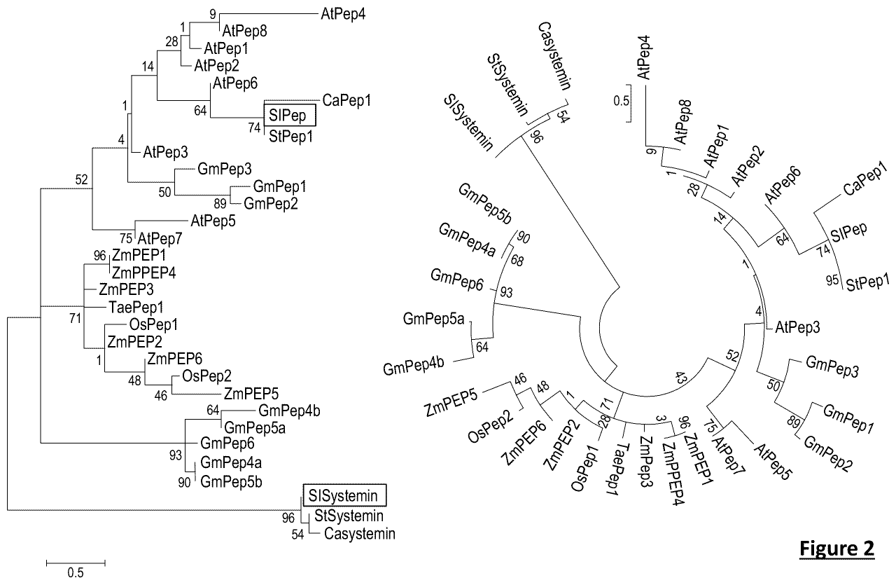

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of plant biology, microbiology, tissue culture, molecular biology, chemistry, biochemistry and recombinant DNA technology, bioinformatics, which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “peptide” “polypeptide” and “protein” are used interchangeably herein and refer to amino acids in a polymeric form of any length, linked together by peptide bonds.

For the purposes of the invention, a “genetically altered plant” is a plant that has been genetically altered compared to the naturally occurring wild type (WT) plant.

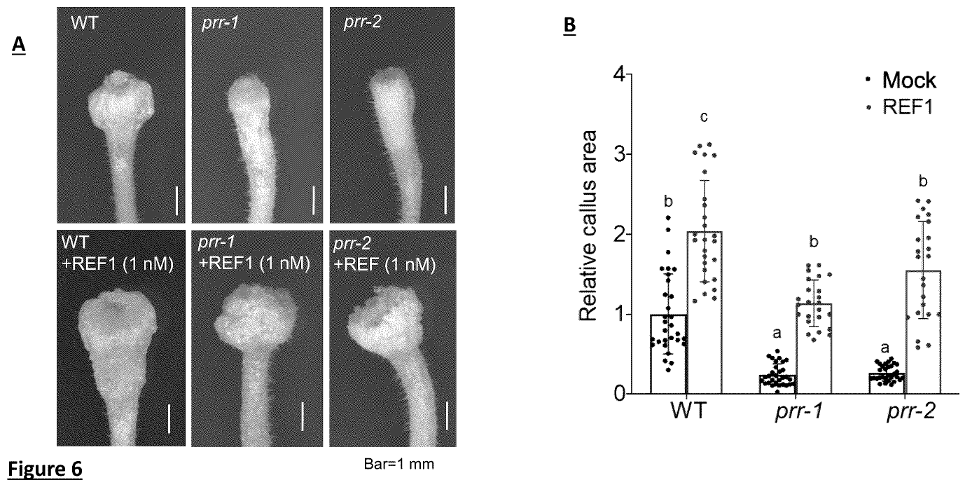

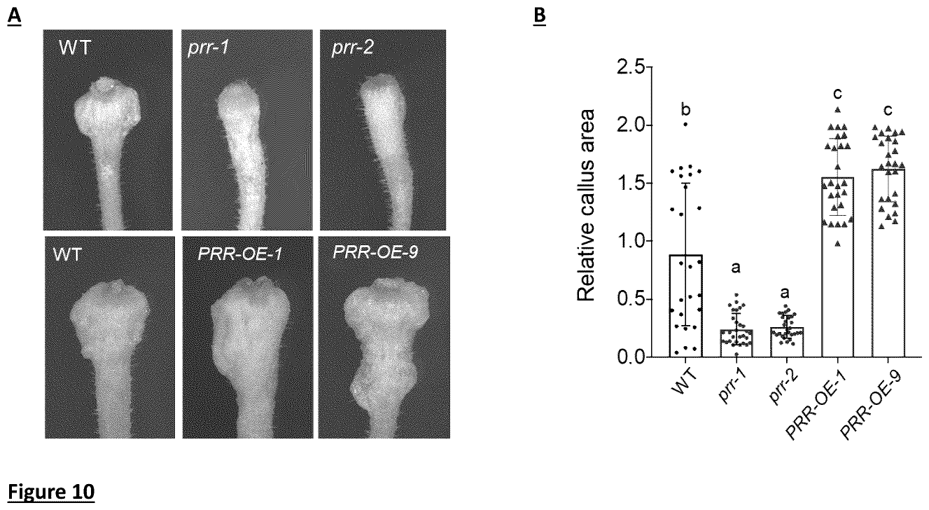

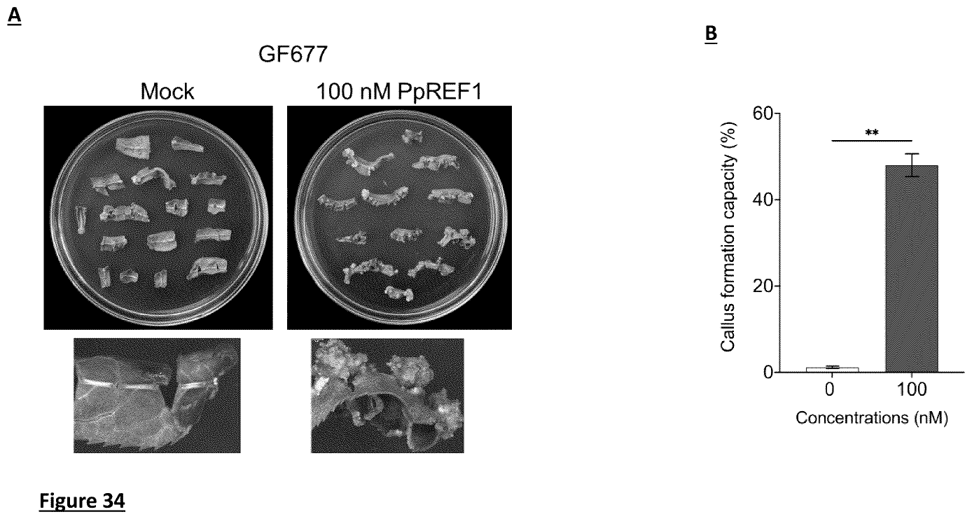

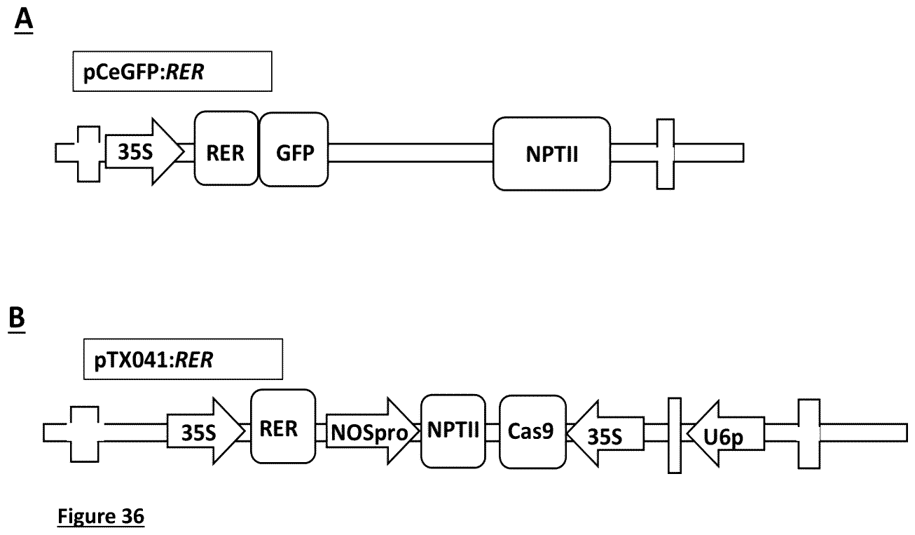

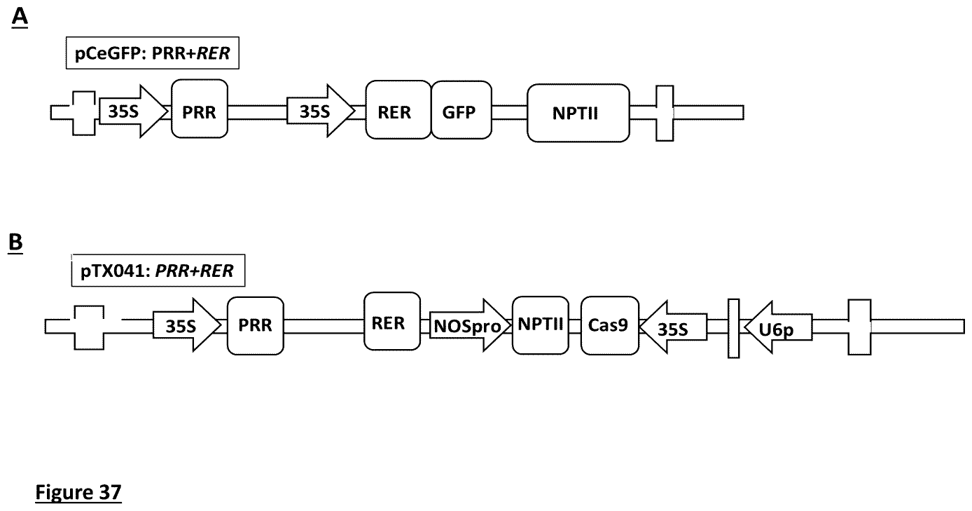

In vivo SIPEP (REF1) is a 23 amino acid peptide cleaved from its precursor PROSIPEP, also referred to as PROREF1, PRP and PRR (these terms are used interchangeably herein). We have shown in FIGS. 6, 8 and 18 to 44 that exogenous application of REF1 increases the regeneration capacity or efficiency (also referred to herein as regeneration frequency) of a plant, part thereof or at least one plant cell. Regeneration frequency can be considered to be the number of regenerated part of the plant, e.g. shoots per the total number of inoculated explants. Consistent with these findings, we have also shown in FIGS. 10, 11, 18 and 19 that increasing the expression levels of PRR and/or RER in the plant also increases the regeneration capacity or efficiency of a plant, part thereof or at least one plant cell. In a particular embodiment, we have shown that expression of a PRR/RER chimera is particularly effective at increasing the regeneration capacity of a plant, part thereof or at least one plant cell.

Accordingly, in a first aspect of the invention, there is provided a tissue regeneration factor comprising or consisting of an amino acid sequence as defined in SEQ ID NO: 2 or a functional variant or homologue thereof. This is REF1.

In one embodiment, the functional variant or homologue is defined by a conserved motif. This conserved motif allows binding of REF1 to RER. In one embodiment, the conserved motif is (SEQ ID NO: 79) GXPPXXNN, where X is any amino acid. In another embodiment, the conserved motif is (SEQ ID NO: 80) SSGXXGXXN, where X is any amino acid. In another embodiment, the conserved motif is (SEQ ID NO: 81) SGGXGGXHX, where X is any amino acid. Accordingly, in one embodiment, the functional variant or homologue may comprise one of these conserved motifs.

In a further embodiment, the homologue comprises or consists of a sequence as defined in SEQ ID NO: 4, 6, 8, 10, 12, 14, 16, 18, 89 or 20. Also included in the scope of the invention are functional variants and homologues of these sequences—e.g. of SEQ ID NO: 4, 6, 8, 10, 12, 14, 16, 18, 89 and 20.

In an embodiment binding of REF1 to RER regulates regeneration through kinase receptors, preferably somatic embryogenesis receptor kinases (SERKs), more preferably, SERK3a and SERK3b. In an embodiment REF1 is a SERK agonist.

In an embodiment REF1 promotes wound-induced regeneration through activating WOUND-INDUCED DEDIFFERENTIATION 1 (WIND1) expression.

In an embodiment WIND1 activates expression of PROREF1.

The tissue regeneration factor may be exogenously administered to a plant, part thereof or one or more plant cells alone, or as part of a plant tissue culture medium or plant growth medium (such terms may be used interchangeably herein). In another aspect of the invention there is provided a plant tissue culture medium comprising the tissue regeneration factor of the invention.

The composition of a plant growth or plant tissue culture medium is well known in the art.

As an example, the medium may comprise micro- and macronutrients, vitamins, organic supplements, and plant growth regulators that are necessary for the growth and multiplication of plant cells, tissues, and organs in vitro. A variety of plant tissue culture mediums are available commercially for different applications with altered compositions according to the user's needs.

In one embodiment, the plant tissue culture medium is a callus inducing medium (CIM).

In another embodiment, the plant tissue culture medium is a shoot inducing medium (SIM).

In another embodiment, the plant tissue culture medium is a root inducing medium (RIM).

Preferably, the plant tissue culture medium comprises auxin and/or cytokinin. In a preferred embodiment, the plant tissue culture medium is an auxin-rich medium. Of note, the ratio of auxin to cytokinin in a plant tissue culture medium determines the fate of regenerating organs. In one embodiment a lower auxin/cytokinin ratio promotes shoot regeneration. In another embodiment a higher ratio of auxin to cytokinin promotes root regeneration.

Typically, a plant tissue culture medium comprises a basal medium, such as Murashige & Skoog Basal Medium with one or more vitamins, glucose, phytol agar and auxin and cytokinin at varying concentrations or ratios depending on the intended purpose of the medium, as described above.

Accordingly, in one aspect of the invention, the plant tissue culture medium comprises Murashige & Skoog Basal Medium with one or more vitamins, glucose, phytol agar, REF1, auxin and cytokinin. In a preferred aspect of the invention, the plant tissue culture medium comprises Murashige & Skoog Basal Medium with one or more vitamins, 3% glucose, 0.8% phytol agar, REF1, auxin and cytokinin. The skilled person would be able to modify the concentration of REF1, auxin and cytokinin (and other medium components) depending on the intended use (e.g. callus-inducing, shoot-inducing or root-inducing).

In one embodiment a callus inducing medium (CIM) comprises, Murashige & Skoog Basal Medium, phytol agar, sucrose, zeatin riboside (ZR), Indole-3-acetic acid (IAA) and REF1.

In a preferred embodiment a callus inducing medium (CIM) may comprise 4.43 g/L Murashige & Skoog Basal Medium, 8 g/L phytol agar, 30 g/L sucrose, 0.1 mg/L zeatin riboside (ZR), 0.4 mg/L Indole-3-acetic acid (IAA) and 1 nmol/L REF1. In one embodiment a CIM with this composition may be used to culture tomato plants. In another embodiment the skilled person may alter the composition of the CIM to be optimised for inducing callus formation in a chosen plant, wherein said chosen plant is a monocot or a dicot.

In one embodiment a shoot inducing medium (SIM) comprises, Murashige & Skoog Basal Medium, sucrose, phytol agar, Indole-3-acetic acid (IAA), zeatin riboside (ZR), and REF1.

In a preferred embodiment a shoot inducing medium (SIM) comprises, 4.43 g/L Murashige & Skoog Basal Medium, 30 g/L sucrose, 8 g/L phytol agar, 0.1 mg/L Indole-3-acetic acid (IAA), 0.4 mg/L zeatin riboside (ZR), and 1 nmol/L REF1. In one embodiment a SIM with this composition may be used to culture tomato plants. In another embodiment the skilled person may alter the composition of the SIM to be optimised for inducing shoot formation in a chosen plant, wherein said chosen plant is a monocot or a dicot.

In one embodiment a root inducing medium (RIM) comprises, Murashige & Skoog Basal Medium, sucrose, phytol agar and REF1.

In a preferred embodiment a root inducing medium (RIM) comprises, 4.43 g/L Murashige & Skoog Basal Medium, 30 g/L sucrose, 8 g/L phytol agar and 1 nmol/L REF1. In one embodiment a RIM with this composition may be used to culture tomato plants. In another embodiment the skilled person may alter the composition of the RIM to be optimised for inducing root formation in a chosen plant, wherein said chosen plant is a monocot or a dicot, Plant growth medium may also refer to a material in which plants are grown. In one embodiment the plant growth medium optionally comprises at least one of the following components, soil, peat moss, perlite, vermiculite, zeolite, compost, water, wooden bark or sand.

In one embodiment, the tissue regeneration factor is incorporated in a plant tissue culture medium or plant growth medium at a concentration between 0.00001 and 1 mM. More preferably, between 0.001 and 800 nM, even more preferably at around 0.1 nM, 1 nM, 10 nM, 20 nM, 30 nM, 40 nM, 50 nM, 60 nM, 70 nM, 80 nM, 90 nM, 100 nM, 110 nM, 120 nM, 130 nM, 140 nM or 150 nM, 200 nM, 250 nM, 300 nM, 350 nM, 400 nM, 450 nM, 500 nM, 550 nM, 600 nM, 650 nM, 700 nM, 750 nM and 800 nM. Even more preferably, the plant tissue culture medium comprises the tissue regeneration factor at a concentration of 0.1 nM, 1 nM, 10 nM, 50 nM, 100 nM or 500 nM.

In another embodiment, the tissue regeneration factor or plant tissue culture medium may be directly applied to the plant, part thereof or one or more plant cells. Preferably, the plant part includes, but is not limited to, organs, tissues, seeds and cells of a plant. Preferably, the plant part is an explant. An explant is a plant part that is cut away from the whole plant and used to initiate culture in a plant tissue culture of medium. Examples of explants that are particularly suitable for plant regeneration include but are not limited to shoots, stems and leaves.

In another aspect of the invention, there is provided a nucleic acid construct comprising at least one nucleic acid sequence that encodes at least one of a tissue regeneration factor (REF1), a tissue regeneration factor precursor (PRR) and a tissue regeneration factor receptor (RER).

In one embodiment, there is provided a nucleic acid construct comprising at least one nucleic acid sequence that encodes a tissue regeneration factor (REF1), where the tissue regeneration factor is operably linked to a regulatory sequence.

In another embodiment, there is provided a nucleic acid construct comprising at least one nucleic acid sequence that encodes a tissue regeneration factor precursor (PRR), where the tissue regeneration factor precursor is operably linked to a regulatory sequence.

In another embodiment, there is provided a nucleic acid construct comprising at least one nucleic acid sequence that encodes a tissue regeneration factor receptor (RER), where the tissue regeneration factor precursor is operably linked to a regulatory sequence.

In one embodiment, there is provided a nucleic acid construct comprising at least one nucleic acid sequence that encodes a tissue regeneration factor (REF1) and a nucleic acid sequence that encodes a tissue regeneration factor precursor (PRR) or a tissue regeneration factor receptor (RER), where the nucleic acid sequences are operably linked to one or more regulatory sequence(s) where REF1 and PRR or RER are operably linked to the same or different regulatory sequence.

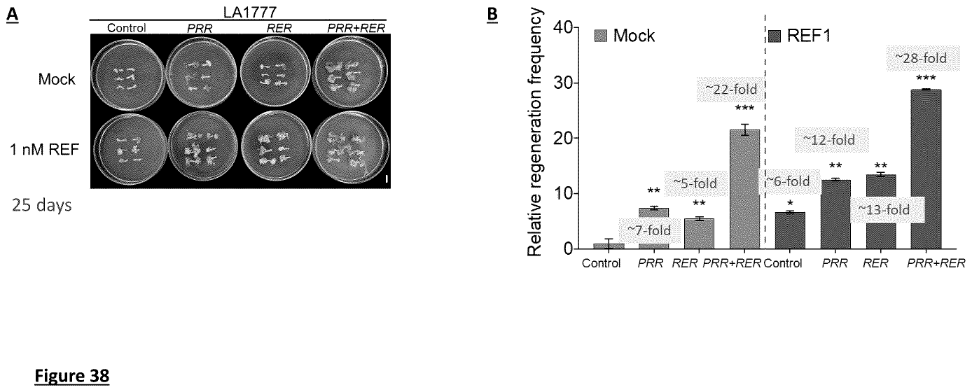

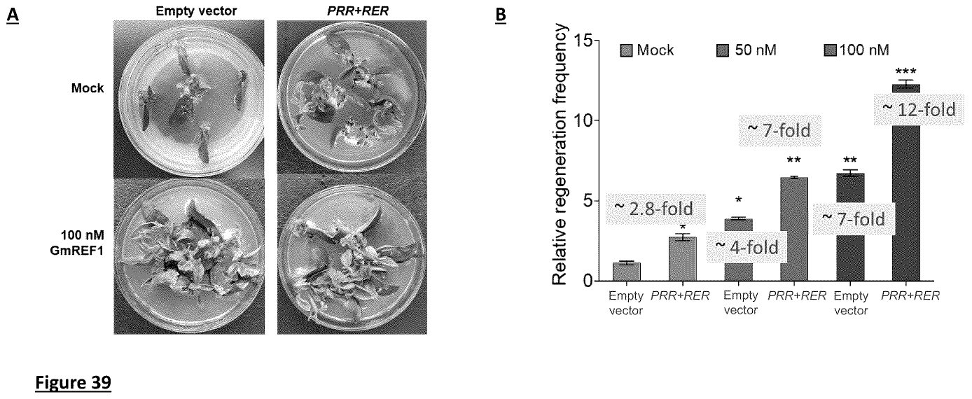

In one embodiment, there is provided a nucleic acid construct comprising at least one nucleic acid sequence that encodes a tissue regeneration factor precursor (PRR) and a nucleic acid sequence that encodes a tissue regeneration factor receptor (RER), where the nucleic acid sequences are operably linked to one or more regulatory sequence(s) where PRR and RER are operably linked to the same or different regulatory sequence. An example of such a nucleic acid construct is shown in FIG. 38. As further shown in FIG. 38, use of this construct (the “PRR+RER-OE”) construct significantly increases regeneration efficiency in tomatoes, buy up to 28-fold, compared to the control. FIG. 39 also shows that this construct significantly improves the regeneration frequency of recalcitrant soybean by up to 12-fold compared to the control.

The nucleic acid sequence may encode a tissue regeneration factor as defined in SEQ ID NO: 2 or a functional variant or homologue thereof. In one embodiment, the homologue encodes a tissue regeneration factor as defined in SEQ ID NO: 4, 6, 8, 10, 12, 14, 16, 18, 89 or 20 or a functional variant or homologue thereof. In one embodiment, the nucleic acid sequence comprises or consists of a nucleic acid sequence as defined in SEQ ID NO: 1 or a functional variant or homologue thereof. In one embodiment, the homologue comprises or consists of a nucleic acid sequence as defined in SEQ ID NO: 3, 5, 7, 9, 11, 13, 15, 17, 90 or 19 or a functional variant or homologue thereof.

The nucleic acid sequence may encode a tissue regeneration factor precursor as defined in SEQ ID NO: 22 or a functional variant or homologue thereof. In one embodiment, the homologue encodes a tissue regeneration factor precursor as defined in SEQ ID NO: 24, 26, 28, 30, 32, 34, 36, 38, 91 or 40 or a functional variant or homologue thereof. In one embodiment, the nucleic acid sequence comprises or consists of a nucleic acid sequence as defined in SEQ ID NO: 21 or a functional variant or homologue thereof. In one embodiment, the homologue comprises or consists of a nucleic acid sequence as defined in SEQ ID NO: 23, 25, 27, 29, 31, 33, 35, 37, 92 or 39 or a functional variant or homologue thereof.

The nucleic acid sequence may encode a tissue regeneration factor receptor as defined in SEQ ID NO: 42 or a functional variant or homologue thereof. In one embodiment, the homologue encodes a tissue regeneration factor receptor as defined in SEQ ID NO: 44, 46, 48, 50, 52, 54, 56, 58, 60, 62, 64, 66, 68, 70, 72, 74, 76, 93 or 78 or a functional variant or homologue thereof. In one embodiment, the nucleic acid sequence comprises or consists of a nucleic acid sequence as defined in SEQ ID NO: 41 or a functional variant or homologue thereof. In one embodiment, the homologue comprises a nucleic acid sequence as defined in SEQ ID NO: 43, 45, 47, 49, 51, 53, 55, 57, 59, 61, 63, 65, 67, 69, 71, 73, 75, 94 or 77 or a functional variant or homologue thereof.

The term “variant” or “functional variant” as used throughout with reference to any of the sequences described herein refers to a variant gene sequence or part of the gene sequence (such as a fragment) which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described throughout a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection.

When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identification of conserved domains. There are predictors in the art that can be used to identify such sequences. The function of the homologue can be identified as described herein and a skilled person would thus be able to confirm the function, for example when overexpressed in a plant.

In an embodiment REF1, PROREF1 and REF1 receptor orthologues can be identified in a given plant using a sequence analysis and alignment tool such as hmmsearch search (HMMER v1.9; (Finn et al., 2011)) against a sequence database known in the art such as NR, RefSeq, UniProtKB or any other known database by using the SIPROPEP/PROREF1, or any known PROPEP sequences as the input sequence. The inventors have additionally found that the PROPEP C-terminal end containing the Pep sequence (e.g. SEQ ID NO: 2) is strictly conserved. Thus, SIPROPEP/PROREF1 orthologues of any plant species can be identified using this sequence. Similarly, novel RER sequences were identified using NCBI BLASTP/BLASTN using the RER sequence in tomato.

Thus, the nucleotide sequences of the invention and described herein can also be used to isolate corresponding sequences from other organisms, particularly other plants. In this manner, methods such as PCR, hybridization, and the like can be used to identify such sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

In a further embodiment, a variant as used herein can comprise a nucleic acid sequence encoding a polypeptide as defined herein that is capable of hybridising under stringent conditions as defined herein to any one of the SEQ ID NOs: defined herein.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

As used herein, a regulatory sequence is a segment of a nucleic acid molecule capable of increasing or decreasing the expression of specific genes within an organism. An example of a regulatory sequence is a “promoter” which refers to a nucleotide sequence, usually upstream (5) to its coding sequence, which controls the expression of the coding sequence by providing the recognition for RNA polymerase and other factors required for proper transcription. In one embodiment, the promoter may be a constitutive promoter, an inducible promoter or a tissue-specific promoter.

“Constitutive promoter” refers to promoters that direct gene expression in nearly all tissues and at all times. Examples of constitutive promoters include but are not limited to the CaMV 35S promoter or StUbi promoter.

“Inducible promoter” refers to promoters that can be turned on in one or more cell types by an external stimulus (such as a chemical, light, hormone, stress, or pathogen). Examples of inducible promoters include, but are not limited to, the nopaline synthase promoter (NOSpro) which is wound inducible and the p0p6/LhGR promoter which is dexamethasone inducible.

“Tissue-specific promoter” refers to a promoter that has activity in only certain cell types. Use of a tissue-specific promoter in the nucleic acid construct can restrict unwanted transgene expression as well as facilitate persistent transgene expression.

The term “operably linked” as used herein refers to a functional linkage between the promoter sequence and the gene of interest, such that the promoter sequence is able to initiate transcription of the gene of interest.

The nucleic acid construct of the invention may further comprise one or more reporter genes. “Reporter genes” are genes whose products can be readily assayed subsequent to transfection, and can be used as markers for screening successfully transfected cells, for studying regulation of gene expression, or serve as controls for standardizing transfection efficiencies. Examples of reporter genes include but are not limited to, GFP, luciferase and lacZ.

In another embodiment, the nucleic acid construct of the invention may additionally comprise one or more selectable marker genes. “Selectable marker genes” are genes introduced into a cell that confer a trait suitable for artificial selection and are used to indicate the success of transformation of cells transformed with a vector containing the marker gene. Examples of selectable marker genes include but are not limited to NPTII, HPT, aadA, and DHPS.

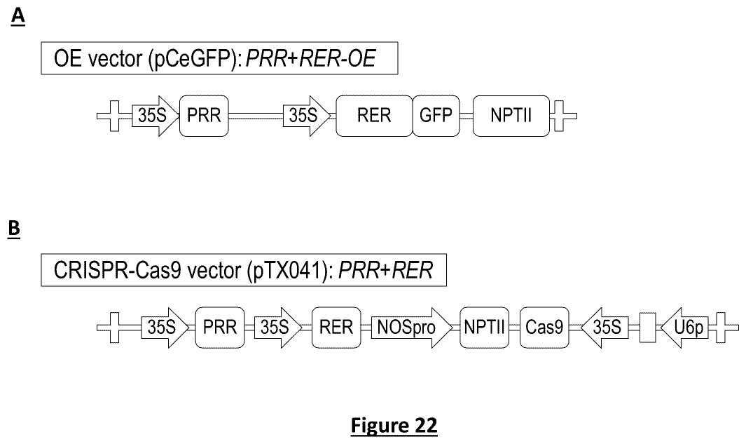

Examples of nucleic acid constructs of the invention are described in FIGS. 22 and 35 to 37

In another aspect of the invention there is provided a host cell comprising and preferably expressing the nucleic acid construct of the invention. Preferably, the host cell is a plant cell.

In another aspect of the invention, there is provided a genetically altered plant, part thereof or one or more plant cell, wherein the plant, part thereof or one or more plant cell is characterised by increased expression or protein levels of one or more of
(i) A tissue regeneration factor (REF1) as described herein; and/or
(ii) A tissue regeneration factor precursor (PRR) as described herein; and/or
(iii) A tissue regeneration factor receptor (RER) as described herein.

In one embodiment an increase in the expression and/or protein levels of (i) to (iii) above is achieved by introducing and expressing one of the nucleic acid constructs of the invention in a plant, part thereof or one or more plant cell. Accordingly, in another aspect of the invention, there is provided a genetically altered plant, part thereof or plant cell comprising and expressing one or more nucleic acid constructs of the invention.

In another embodiment, an increase in the expression and/or protein levels of (i) to (iii) above achieved using targeted genome editing, such as CRISPR, to introduce one or more additional copies of a gene encoding a tissue regeneration factor and/or a tissue regeneration factor precursor and/or a tissue regeneration factor receptor into the plant genome. Preferably, genome editing techniques are used to introduce one or more additional copies of the gene at a position in the genome, such that the gene or genes are under control of a suitable regulatory sequence. Preferably said regulatory sequence is the 35S promoter.

The gene encoding a tissue regeneration factor may comprise or consist of SEQ ID NO: 1, 3, 5, 7, 9, 11, 13, 15, 17, 19, 90 or a functional variant or homologue thereof.

The gene encoding a tissue regeneration factor precursor may comprise or consist of SEQ ID NO: 21, 23, 25, 27, 29, 31, 33, 35, 37, 39, 92 or 40 a functional variant or homologue thereof.

The gene encoding a tissue regeneration factor receptor may comprise or consist of SEQ ID NO: 41, 43, 45, 47, 49, 51, 53, 55, 57, 59, 61, 63, 65, 67, 69, 71, 73, 75, 77, 94 or a functional variant or homologue thereof.

In another aspect of the invention there is provided the use of the tissue regeneration factor of the invention to increase or improve regeneration efficiency of a plant, part thereof or one or more plant cells.

In another aspect of the invention there is provided the use of the plant tissue culture medium of the invention to increase or improve regeneration efficiency of a plant, part thereof or one or more plant cells.

In another aspect of the invention there is provided the use of one or more of the nucleic acid construct of the invention to increase or improve regeneration efficiency of a plant, part thereof or one or more plant cells.

In another aspect of the invention, there is provided a method of increasing the regenerative efficiency of a plant, plant part thereof or one or more plant cells, the method comprising
i. administering the tissue regeneration factor of the invention to the plant or part thereof or one or more plant cells; or
ii. administering the plant tissue culture medium of the invention to the plant or part thereof or one or more plant cells; and/or
iii. introducing and expressing in the plant, part thereof or one or plant cells the nucleic acid construct of the invention.

By “regeneration efficiency” is meant the capacity of a plant or plant part to regenerate after injury or exposure to a regenerative stimulus. In one example, an increased regenerative capacity can be measured by at least one of increased hypocotyl numbers, stem cell formation, shoot regeneration capacity, callus formation capacity or callus area, primary root length, root meristem formation and/or root hair formation.

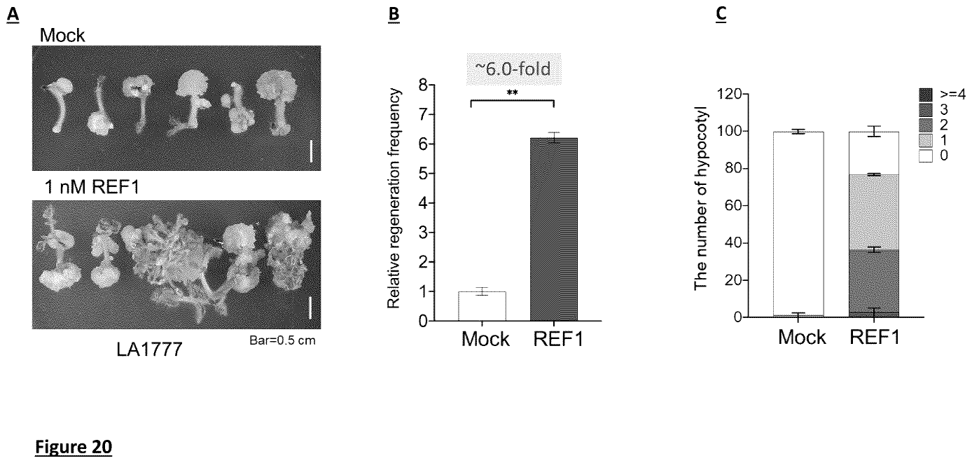

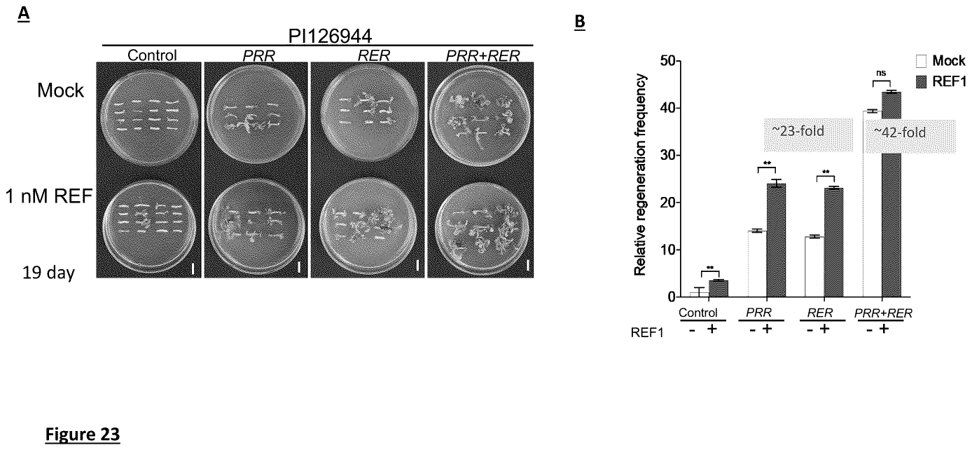

By an increase is meant an increase in regeneration capacity or efficiency compared to the amount of regeneration in a control or wild-type plant—that is, a plant where the tissue regeneration factor or plant tissue culture medium of the invention is not applied and/or a plant that does not express one or more of the nucleic acid constructs of the invention. The increase may be between 2 and 50-fold compared to the control or wild-type plant. Preferably, the increase may be at least 2-fold, at least 3-fold, at least 5-fold, at least 7-fold, at least 8-fold, at least 10-fold, at least 20-fold, at least 30-fold, at least 40-fold or at least 50-fold compared to the level of regeneration in the control or wild-type plant. For example, FIG. 20 shows that 1 nM REF1 is able to increase the regeneration frequency of wild-type tomatoes by ~6 fold, and FIG. 23 shows that tomatoes overexpressing the REF1 receptor (RER) and the REF1 precursor (PRR) grown in the presence of 1 nM REF1 have a regeneration frequency ~42 fold higher than than wild-type tomato grown in the absence of REF1.

In one embodiment, the tissue regeneration factor or the plant tissue culture medium is exogenously applied to the plant. In a further embodiment, the tissue regeneration factor or the plant tissue culture medium is applied to the genetically altered plant of the invention to increase the regenerative efficiency of the plant even further, as shown in FIG. 23.

In one embodiment REF1 is applied exogenously to a plant in combination with at least one phytohormone wherein preferably said phytohormone(s) is an auxin and/or a cytokinin. In another embodiment, REF1 is applied as part of a plant tissue culture medium, as described above. As also described above, the balance between auxin and cytokinin determines the fate of the regenerating organ. For example, an auxin-rich or high auxin:cytokinin ratio medium generates calluses that are competent for organ regeneration. The skilled person would be able to modify the concentration or ratio of phytohormones depending on the intended use.

In one embodiment where REF1 is applied exogenously to a plant in the matrix of a plant tissue culture medium the amount of plant tissue culture medium applied to the plant is adjusted according to the quantity and size of the explants to be cultured. For example, 1 nmole/L REF1 is equal to 2.5×10−3 mg/L and 100 tomato hypocotyl are preferably cultured in 250 ml of medium. In another non-limiting example 12-36 explants are preferably cultured in 100 ml of the plant tissue culture medium of the invention.

In another embodiment, the time taken between application of REF1 or the plant tissue culture medium of the invention and when regeneration is observed differs according to the properties of the plant—such as, but not limited to the type of plant the explant is derived from and the part of the plant the explant is taken from. In one embodiment, the time taken between application and observation of regeneration is up to 10 days, up to 20 days, up to 30 days, up to 40 days, up to 50 days, up to 60 days or up to 70 days facilitates plant regeneration. In a preferred embodiment, the time taken between application and observation of regeneration is around 40 days.

In another embodiment REF1 or the tissue culture medium of the invention is applied to the plant, preferably at a wound site. In a preferred embodiment, REF1 or the tissue culture medium of the invention is applied once. Alternatively, REF1 or the tissue culture medium of the invention is applied twice or more, with intervals between applications that differ according to the type of plant and desired regeneration. Where the plant is soybean, maize, sunflower, tomato or pepper only one application is needed. These plants can directly regenerate shoots from a wound site. Other plants require one or more applications. These plants, which include many monocotyledons, first form a callus before regenerating a shoot.

In one embodiment the concentration of REF1 or the plant tissue culture medium of the invention applied to the plant differs according to the properties of the plant—such as, but not limited to, the type of plant the explant is derived from and the part of the plant the explant is taken from. In an embodiment a mature explant requires a higher concentration of REF1 to be applied exogenously relative to an immature explant. In one embodiment REF1 is applied exogenously to a mature explant at a concentration in the range of 1-500 nmol/L and REF1 is applied exogenously to an immature explant at a concentration in the range of 0.01-1 nmol/L.

In a preferred embodiment, the method comprises administering a tissue regeneration factor or plant tissue culture medium, where said tissue regeneration factor is from the same plant that the factor is applied to—e.g. tomato REF1 is applied to tomatoes. In other words, heterologous administration is not a preferred embodiment—that is, application of rice REF1 to tomatoes. The same is equally applicable to the introduction of the nucleic acid constructs of the invention.

The nucleic acid constructs may be introduced into a plant cell using any suitable method known to the skilled person (the term “introduced” can be used interchangeably with “transformation”, which is described below).

Any of the nucleic acid constructs described herein, may be introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, microspores, anthers, ovules cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation. According to the invention, the nucleic acid is preferably stably integrated in the transgenic plants genome and the progeny of said plant therefore also comprises the transgene.

To select transformed plants, the plant material obtained in the transformation is, in certain embodiments, subjected to selective conditions so that transformed plants can be distinguished from untransformed plants. For example, the seeds obtained in the above-described manner can be planted and, after an initial growing period, subjected to a suitable selection. A further possibility is growing the seeds, if appropriate after sterilization, on agar plates using a suitable selection agent so that only the transformed seeds can grow into plants. Alternatively, the transformed plants are screened for the presence of a selectable marker.

Following DNA or nucleic acid transfer and regeneration, putatively transformed plants may also be evaluated, for instance using Southern Blot analysis.

The generated transformed plants may be propagated by a variety of means, such as by clonal propagation or classical breeding techniques. For example, a first generation (or T₁) transformed plant may be selfed and homozygous second-generation (or T₂) transformants selected, and the T₂ plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further embodiment of any of the methods described herein, the method may further comprise at least one or more of the steps of assessing the phenotype of the genetically altered plant, assessing the regenerative capacity of a plant by measuring for example at least one of shoot regeneration capacity (e.g. the frequency of explants with regenerated shoots) and/or callus formation capacity (e.g. percentage of explants with regenerated embryonic calli) and/or primary root length and comparing said phenotype to a wild-type or control plant to determine the increase in regenerative capacity compared to a wild-type or control plant. In other words, the method may involve the step of screening the plants for the desired phenotype.

In a further aspect of the invention, there is provided a method of producing a plant, plant part thereof or one or more plant cells with increased regeneration efficiency as described above, the method comprising
i. administering the tissue regeneration factor of the invention to the plant or part thereof or one or more plant cells; or
ii. administering the plant tissue culture medium of the invention to the plant or part thereof or one or more plant cells; and/or
iii. introducing and expressing in the plant, part thereof or one or plant cells the nucleic acid construct of the invention as described above.

In a further aspect of the invention there is provided a plant or plant part thereof or one or more plant cells obtained or obtainable by the above described methods. Preferably said plant is characterised by an increased regenerative capacity compared to a wild type or control plant. Preferably an increased regenerative capacity can be measured by at least one of, shoot regeneration capacity, callus formation capacity and/or primary root length.

Also included within the scope of the invention is progeny of the plants of the invention, where the progeny comprise the nucleic acid construct of the invention. Another embodiment of the invention is a genetically modified plant obtained or obtainable by the above method of modifying the genome of a plant cell and regenerating a plant from said cell as well as progeny or parts thereof, wherein the progeny or the part comprises the modification in the genome introduced by the above method of modification.

In an embodiment of the invention, there is provided a method of increasing the transformation efficiency of a plant, plant part thereof or one or more plant cells, the method comprising
i. administering the tissue regeneration factor of the invention to the plant or part thereof or one or more plant cells; or
ii. administering the plant tissue culture medium of the invention to the plant or part thereof or one or more plant cells; and/or
iii. introducing and expressing in the plant, part thereof or one or plant cells the nucleic acid construct of the invention.

Also within the scope of the invention is a plant, part thereof or plant cell obtained or obtainable from the above described method wherein said plant, part thereof or plant cell is characterised by an increased transformation efficiency.

By “transformation efficiency” is meant the capacity of an organism to take up and incorporate exogenous DNA such as plasmids during transformation. Transformation efficiency is calculated by dividing the number of transformants by the total number of organisms subjected to the transformation process and multiplying this by 100 to obtain a percentage.

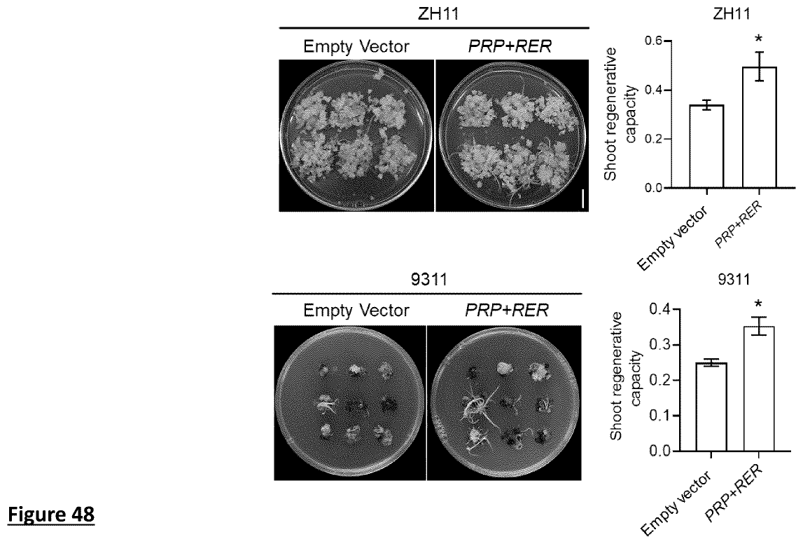

In the context of the present invention an increase is transformation efficiency is compared to the transformation efficiency in a control or wild-type plant—that is, a plant where the tissue regeneration factor or plant tissue culture medium of the invention is not applied and/or a plant that does not express one or more of the nucleic acid constructs of the invention. The increase may refer to an increase of at least 1%, 2%, 3%, 4%, 5%, 6%, 7%, 8%, 9%, 10%, 20%, 30%, 40%, 50%, 60%, 70%, 80%, 90, 95% or more compared to the control or wild-type plant. Alternatively, the increase may be at least a one-fold, two-fold, three-fold, four-fold, five-fold, ten-fold, twenty-fold or more compared to the transformation efficiency in the control or wild-type plant. For example, FIG. 47 shows that GmREF1 improves transformation efficiency in soybean.

In another aspect of the invention, there is provided a recombinant expression vector (also referred to as a “nucleic acid construct”), an expression cassette, a transgenic cell line or recombinant strain comprising the nucleic acid construct of the invention.

The recombinant expression vector comprising a protein, nucleic acid or DNA molecule described herein, preferably SIPEP (REF1) and/or PROSIPEP (PRR) and/or SIPEPR1 (RER) may be constructed by using an existing expression vector. Preferably the expression vector comprises binary Agrobacterium tumefaciens vector and vectors for microprojectile bombardment. When a recombinant expression vector is constructed with the comprising a protein, nucleic acid or DNA molecule described herein, any of an enhanced, constitutive, tissue-specific, or inducible promoter may be linked before the transcription initiation nucleotide, which may be used alone or in combination with other plant promoters. Moreover, when a recombinant expression vector is constructed comprising a protein, nucleic acid or DNA molecule described herein, an enhancer may be included, including a translational enhancer or a transcriptional enhancer. These enhancer regions may be the ATG initiation codon or an initiation codon of an adjacent region, which however needs to be co-framed with the coding sequence, to ensure the proper translation of the whole sequence. The translation control signal and the initiation codon are widely available, and may be natural, or synthesized. The translation initiation region may be from a transcription initiation region or a structural gene. To facilitate the identification and screening of transgenic plants or transgenic microorganisms, the expression vectors used may be processed, for example, by adding a gene expressing enzymes or luminescent compounds that produce colour changes in plants or microorganisms, resistant antibiotic markers, or chemical resistant marker genes. Considering the safety of the transgene, the plants or microorganisms may be directly transformed by phenotypic selection without adding a selective marker gene.

The term androgenesis refers to plant regeneration directly from microspore culture under in vitro conditions. The underlying principle of androgenesis is to stop the development of pollen cells, which normally become sexual cells, and to force their development directly into a complete plant. This process inhibits typical gametophytic differentiation and instead allows cell division and regeneration to occur.

As gametogenesis (microspore development) takes place, mature pollen grains are formed via mitosis. Since the developmental route is not yet determined during micro-gametogenesis, there is a chance to interrupt the normal gametophytic pathway and to induce sporophytic development. As the result of sporophytic divisions, multicellular microspores develop within the anthers. Differentiation of these multicellular units may result in pollen embryos, which then develop into haploid plants.

In some conditions, microspores may undergo sporophytic development instead of entering the gamete-producing pathway. Many of the microspores arrest and/or die, some develop pollen-like structures prior to death or arrest, and others develop a multinucleate, haploid callus-like structure. Meanwhile, other microspores are directly committed to embryogenesis and undergo numerous changes at different levels to become microspore-derived embryos.

From this, the microspores enlarge significantly, the nucleus repositions to the cell centre, the cytoplasm clears, and the large vacuole breaks apart into smaller fragments.

Included within the scope of the present invention is a method of inducing androgenesis mediated by exogenous application of REF1 and/or overexpression of PRR and/or RER.

Also included within the scope of this invention is a method of inducing organogenesis by exogenous application of REF1 and/or overexpression of PRR and/or RER. Organogenesis means formation of organs from the cultured explants, and consequently plant regeneration. In direct organogenesis, in vitro organs are directly induced from explant tissues; in indirect organogenesis, a de novo organ is typically formed from an intermediate tissue, i.e. a callus.

Also within the scope of this invention is a method of inducing somatic embryogenesis by exogenous application of REF1 and/or overexpression of PRR and/or RER. In somatic embryogenesis, the totipotent cells may undergo embryogenic pathway to form somatic embryos, which are grown to regenerate whole plants. Somatic embryogenesis occurs directly when an embryo develops from somatic cells and indirectly when the embryogenic structures are preceded by non-embryonic cell division.

Also within the scope of the invention is a method of enhancing the embryogenic response from the primary callus induced by REF1. Plant regeneration is the process by which plants regrow and develop after severe damage. The inventors have proved that REF1 is originated from wounding to promote regeneration as well as promoting root hair formation which has parallels with the embryogenic response.

A plant according to all aspects of the invention described herein may be a monocot or a dicot plant.

A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from tomato, soybean, lettuce, sunflower, Arabidopsis, broccoli, spinach, water melon, squash, cabbage, potato, yam, Capsicum, tobacco, cotton, okra, apple, rose, strawberry, alfalfa, bean, field (fava) bean, pea, lentil, peanut, chickpea, apricots, pears, peach, grape vine or citrus species.

A monocot plant may, for example, be selected from the families Arecaceae, Amaryllidaceae or Poaceae. For example, the plant may be a cereal crop. Examples of a crop plant include wheat, rice, barley, maize, oat, sorghum, rye, millet, buckwheat, turf grass, Italian rye grass, sugarcane or Festuca species, or a crop such as onion, leek, yam or banana.

Preferably, the plant is selected from tomato, pepper, rice, wheat, barley, soybean, maize, sunflower, potato, cotton, cucumber or peach.

In an embodiment, when the plant is rice the variety is selected from Japonica rice, indica rice, or wild allotetraploid rice, more preferably, ZH11 rice, 9311 rice or L-04 rice.

In an embodiment, when the plant is wheat, the variety is selected from Chinese Spring, JM22 or KN199.

In an embodiment, when the plant is maize, the variety is selected from B-104 or Chan 7-2 In one embodiment, the plant may be a variety or cultivar that is, or was previously recalcitrant to regeneration.

The term “plant” as used herein encompasses whole plants, ancestors and progeny of the plants and plant parts, including seeds, fruit, shoots, stems, pollen, microspores, anthers, ovules, leaves, roots (including tubers), flowers, tissues and organs, wherein each of the aforementioned comprise a nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct or mutations as described herein.

The invention also extends to harvestable parts of a plant of the invention as described herein, but not limited to seeds, leaves, fruits, flowers, stems, roots, rhizomes, tubers and bulbs.

In a most preferred embodiment, the plant part or harvestable part is a seed or the fruit. Therefore, in a further aspect of the invention, there is provided a seed or fruit produced from a genetically altered plant as described herein.

A control plant as used herein according to all of the aspects of the invention is a plant which has not been modified according to the methods of the invention. Accordingly, in one embodiment, the control plant does not have one or more of the above-described constructs and/or has not been exogenously exposed to SIPEP (REF1). In one embodiment, the control plant is a wild type plant. The control plant is typically of the same plant species, preferably having the same genetic background as the modified plant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

In the context of the present examples measurements were made as follows:

Relative callus area was determined by quantifying the area of each callus using ImageJ, the average of each group was calculated, for each group this was then divided by the average of the control group.

Relative regeneration frequency was determined by counting the number of regenerated shoots and calculating the average for each group, the average of each group was then divided by the average of the control group.

The number of hypocotyls means explants with varying numbers of regenerated shoots.

Shoot regeneration frequency was established by calculating the average number of shoots, in the context of the present invention shoot regenerative capacity refers to the number of shoots per explant in each treatment.

### EXAMPLES

### Example 1—SIPEP (REF1) as a Local Wound Signal in Tomato

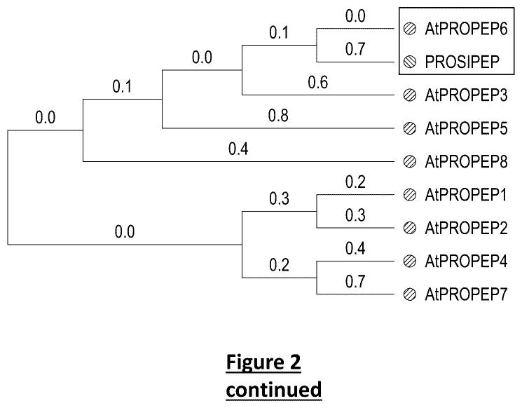

Green and Ryan (1972) found that PI-II (proteinase inhibitor II) is a marker of defence gene expression in plants, as shown in FIG. 2 PI-II expression increases locally at the site of injury i.e. from insect attack but also increases systemically in sites of the plant that were not subjected to injury.

Systemin is known to regulate systemic wound response in plants. A systemic wound response does not occur in wounded PROSYSTEMIN knock-out plants (prs), this is indicated by lack of systemic PI-II expression in prs plants, whereas wounded wild-type plants exhibit high expression of PI-II both systemically and locally compared to control unwounded tomato plants. PROSYSTEMIN (prr) knock outs expressed PI-II locally at intermediate levels but not systemically, indicating the existence of systemin-independent signals that regulate local wound response in plants.

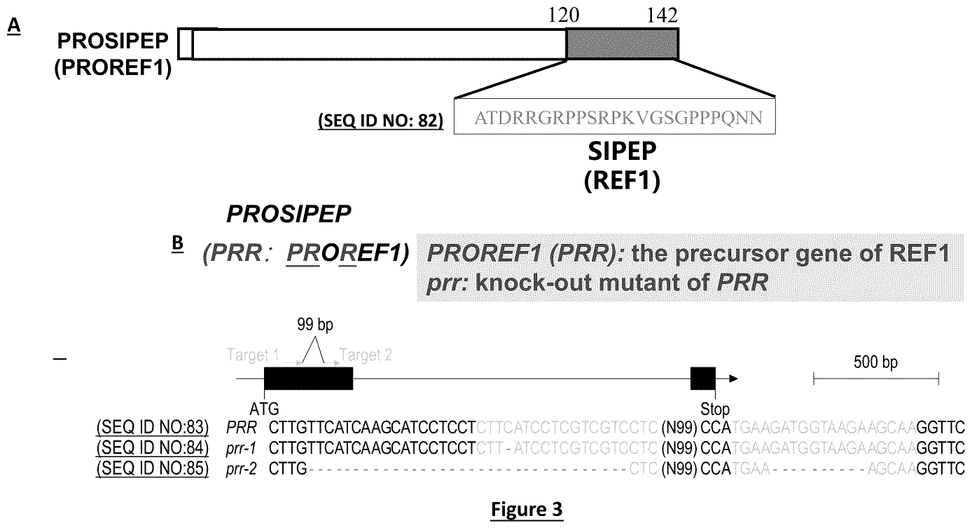

Plant elicitor peptides (PEPs) are peptide signals known to regulate innate immunity in Arabidopsis. A PEP homologue was identified in tomato, namely SIPep also referred to as REF1, shown in FIG. 2. SIPEP is a 23 amino acid peptide cleaved from its precursor PROSIPEP, also referred to as PROREF1 and PRR (these terms are used interchangeably) shown in FIG. 3.

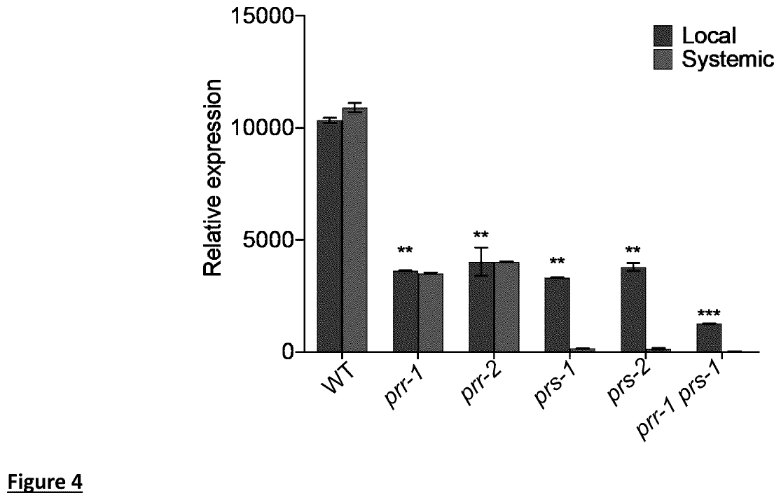

PROSIPEP knockout were generated as shown in FIG. 3B (prr-1 and prr-2) and PROSYSTEMIN knockouts were generated (prs-1 and prs-2). The effect of SIPEP (REF1) and systemin was then investigated by measuring the local (red) and systemic (blue) expression of PI-II in wounded tomato plants that were either wild-type, PROSIPEP knockouts (prr-1 and prr-2), PROSYSTEMIN knockouts (prs-1 and prs-2), or a PROSIPEP PROSYSTEMIN double knockout (prr-1 prs-1). FIG. 4 shows that PI-II expression was high both locally and systemically in wounded wild-type tomato plants showing that when both PROSIPEP and PROSYSTEMIN are expressed and consequently both SIPEP and systemin are produced both a local and systemic wound response occurs. In PROSIPEP knock outs (prr-1 and prr-2) PI-II was expressed both locally at the wound site and systemically albeit at a lower level than in wild-type plants. PROSYSTEM/N knockouts (prs-1 and prs-2) expressed PI-II locally at similar levels to those seen in PROSIPEP knockouts, however, they should very little systemic PI-II expression, indicating that systemin is a key regulator of systemic wound response. Critically, the PROSIPEP PROSYSTEM/N double mutant showed a local wound response (local PI-II expression) much lower than was observed for its parent single mutants, this suggests that SIPEP (REF1) is a local wound signal that is regulator of local wound response in tomato.

### Example 2—SIPEP (REF1) Rescues Defective Callus Formation in PROSIPEP (Prr) Mutants

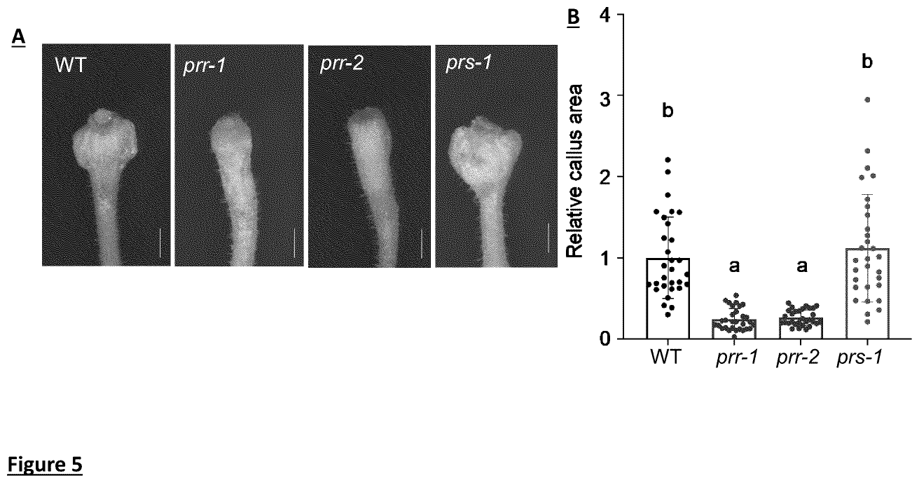

To further investigate the role of SIPEP as a local wound signal in tomato the tomato cultivar Aisla Craig (AC) was used. AC was analysed in its wild-type form, with PROSIPEP knocked out (prr-1 and prr2), and with PROSYSTEM/N knocked out (prs-1). It is clearly shown in FIG. 5A that calluses formed in both the wild-type and prs-1 plants, this shows that a callus can form in the absence of systemin and therefore systemin is not critical to local wound signalling in tomato. Conversely, FIG. 5A shows that PROSIPEP knockouts (prr-1 and prr-2) only formed extremely small calluses, this further exemplified by the relative callus area shown in FIG. 5B. From this data it is evident that depletion of the precursor gene of SIPEP (REF1) (PRR), but not that of systemin (PRS), abolishes callus formation during tomato tissue culture, supporting that SIPEP (REF1) is a wound signal promoting regeneration.

To further test whether SIPEP (REF1) is a wound signal promoting regeneration in tomato, wild type, prr-1 and prr-2 Aisla Craig hypocotyl explants were cultured on a callus inducing medium in the presence and absence of SIPEP (REF1) at 1 nM. The callus phenotype was then scored at 14 days after incubation (FIG. 6), here it can be seen that presence of 1 nM REF1 in the medium increased callus area in all explants tested, crucially, the relative callus area of prr-1 and prr-2 was restored when REF1 was present in the medium. Further to this, REF1 also induced larger callus areas in wild type plants when it was included in the callus inducing medium. This data provided compelling evidence that REF1 is a wound signal that promotes regeneration in plants.

### Example 3—SIPEP (REF1) Rescues Defective Shoot Regeneration in PROSIPEP (Prr) Mutants

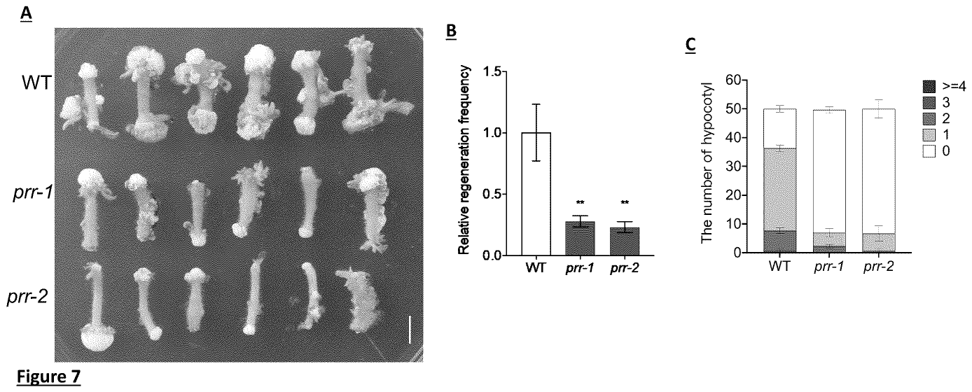

To further investigate the role of SIPEP as in regeneration in the tomato cultivar Aisla Craig (AC) was used. AC was analysed in its wild-type form, and with PROSIPEP knocked out (prr-1 and prr2). FIG. 7A shows visually that shoot regeneration capacity was abolished in PROSIPEP mutants (prr-1 and prr-2). This is quantified in FIG. 7B where it can be seen that the relative regeneration frequencies and the number of hypocotyl is greatly reduced in prr-1 and prr-2 compared to wild type (WT).

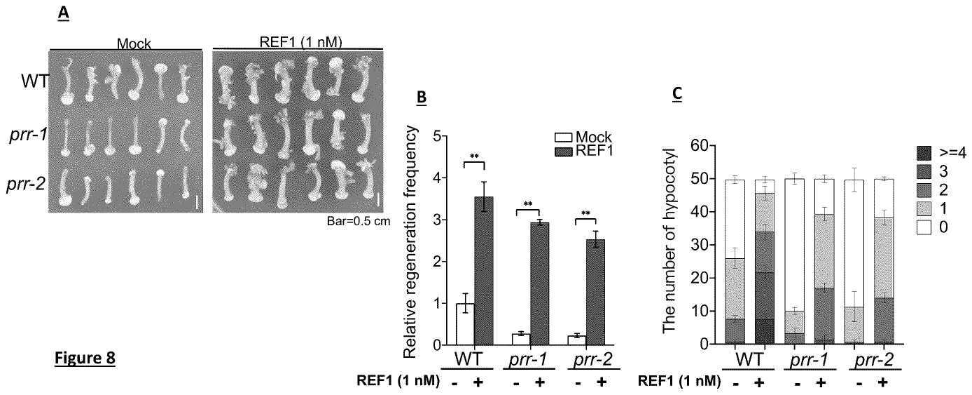

To test whether SIPEP (REF1) could rescue shoot regeneration defects in prr mutants were cultured in shoot inducing medium (SIM) in the presence and absence of 1 nM REF1. Shoot regeneration was then scored after 21 days. FIG. 8 clearly shows that when cultured with 1 nM REF1 shoot regeneration was rescued in prr-1 and prr-2 mutants, and was increased in wild-type plants. FIG. 8B shows that REF-1 increased relative regeneration frequency was increased in WT, prr-1 and prr-2 mutants, whilst FIG. 8C shows that REF-1 increased the number of hypocotyl in WT, prr-1 and prr-2 mutants. This data shows that REF1 plays a role in plant regeneration.

Relative regeneration frequency was determined by counting the number of regenerated shoots and calculating the average for each group, the average of each group was then divided by the average of the WT-Mock group.

‘The number of hypocotyls’ means explants with different number of regenerated shoots (0, 1, 2, 3 and ≥4) in each of the genotypes (WT, prr-1, prr-2).

### Example 4—SIPEP (REF1) Dose Dependently Promotes Callus Formation

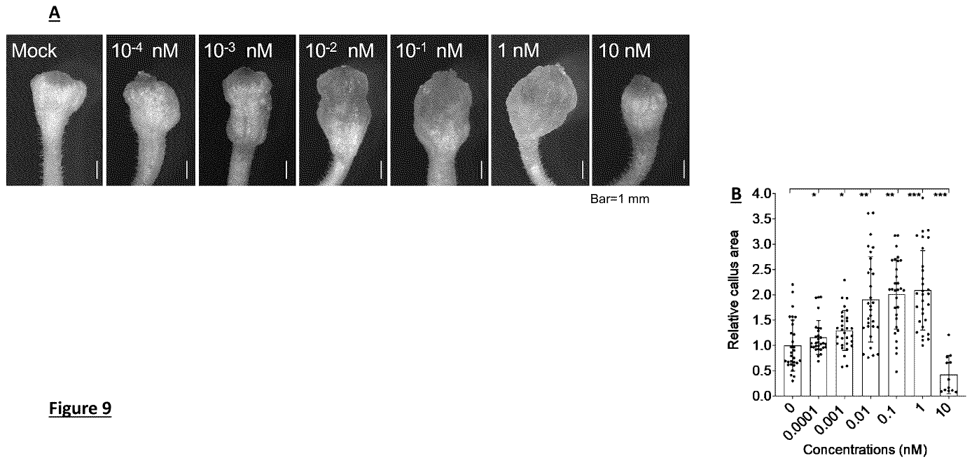

To test the effect of the dose of SIPEP (REF1) on callus formation hypocotyl explants of WT (Ailsa Craig) were cultured on callus-inducing medium (CIM) containing indicated concentrations of REF1 in FIG. 9 (mock with 0, 10⁻⁴, 10⁻³, 10², 10¹, 1, and 10 nM) and callus phenotype was scored 14 days after incubation. Box plots represent the distribution of projected callus area. Here it is shown that relative callus area increased in a dose dependent manner, with the highest mean being seen at 1 nM and then a sharp decrease at 10 nM. This suggests that for explants of WT Ailsa Craig 1 nM of REF1 is the optimum concentration to apply in a medium to maximize callus formation.

### Example 5—the Effect of Overexpression of PROSIPEP (PRR)

The effect of overexpression of SIPEP was tested on both callus formation and shoot regeneration.

FIG. 10 shows the effect of overexpression of SIPEP (PRR) on callus formation. FIG. 10A shows visually that callus formation is enhanced in SIPEP (PRR) overexpressing plants (PRR-OE-1 and PRR-OE-9) compared to wild-type and SIPEP (PRR) knockouts (prr-1 and prr-2). This is quantified in FIG. 10B which shows that relative callus area is increased in both PRR-OE-1 and PRR-OE-9 compared to WT and prr-1 and prr-2.

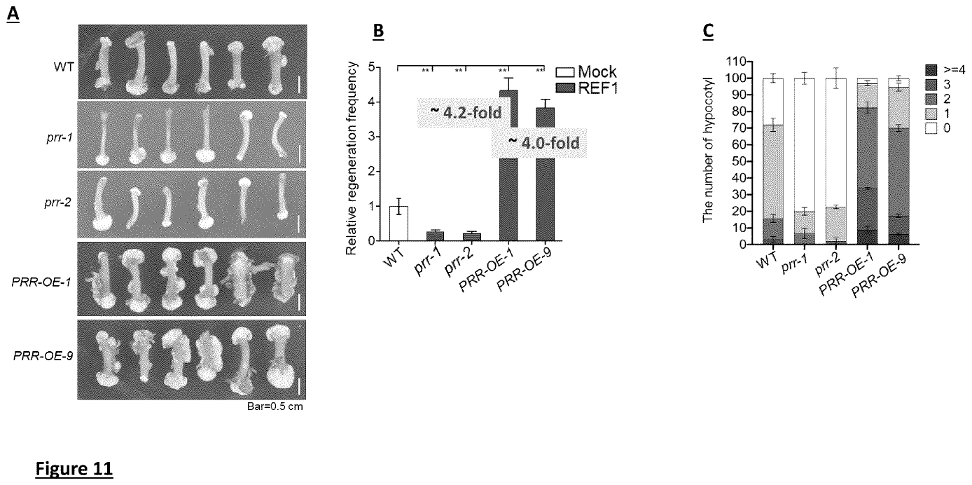

FIG. 11 shows the effect of overexpression of SIPEP (PRR) on shoot regeneration. FIG. 10A shows visually that shoot regeneration is enhanced in SIPEP (PRR) overexpressing plants (PRR-OE-1 and PRR-OE-9) compared to wild-type and SIPEP (PRR) knockouts (prr-1 and prr-2). This is quantified in FIG. 10B which shows that relative regeneration frequency is increased in both PRR-OE-1 and PRR-OE-9 compared to WT and prr-1 and prr-2, with the relative increase being ~4.2 fold and ~4 fold compared to WT for PRR-OE-1 and PRR-OE-9 respectively. Further to this, FIG. 10C shows that the number of hypocotyl is increased in both PRR-OE-1 and PRR-OE-9 compared to WT and prr-1 and prr-2.

This data shows that increasing the expression of PROSIPEP (PRR) increases plant regenerative capacity.

### Example 6—SIPEPR1 (RER) is the Receptor of SIPEP (REF1)

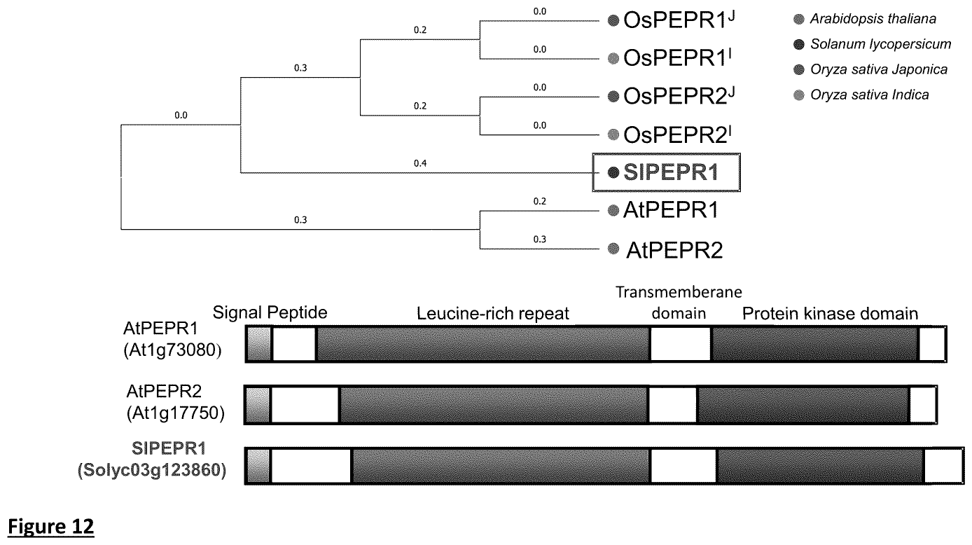

SIPEPR1 (RER) was identified as the tomato orthologue of Arabidopsis thaliana PEP receptors, as shown in FIG. 12. Therefore, RER was studied to establish if this is the receptor for SIPEP (REF1). RER consists of an extracellular LRR domain, a transmembrane domain and a cytoplasmic kinase domain.

An investigation was carried out to see if systemin was capable for triggering auto-phosphorylation of the SIPEP (REF1) receptor (RER).

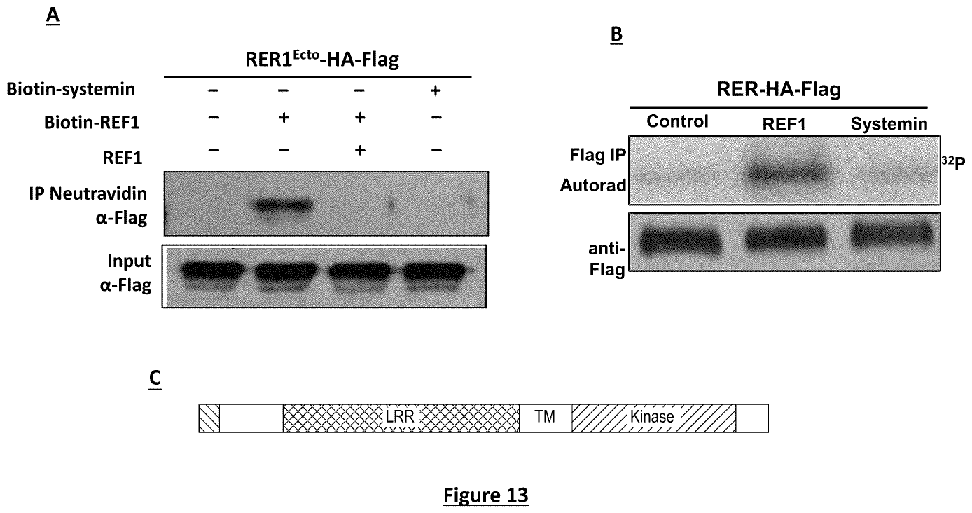

FIG. 13A shows results from a pull-down assay between HA-Flag-tagged ectodomains of RER (purified from tobacco leaves) and biotinylated REF1. Here it is shown that RER1 binds SIPEP (REF1), but systemin does not.

FIG. 13B shows an autophosphorylation assay of tobacco leaves expressing the construct illustrated in FIG. 13C said tobacco leaves were then treated with 100 nM REF1 for 20 min before autophosphorylation assays. Here it can be seen that SIPEP (REF1) triggers auto-phosphorylation of RER in vivo but systemin does not.

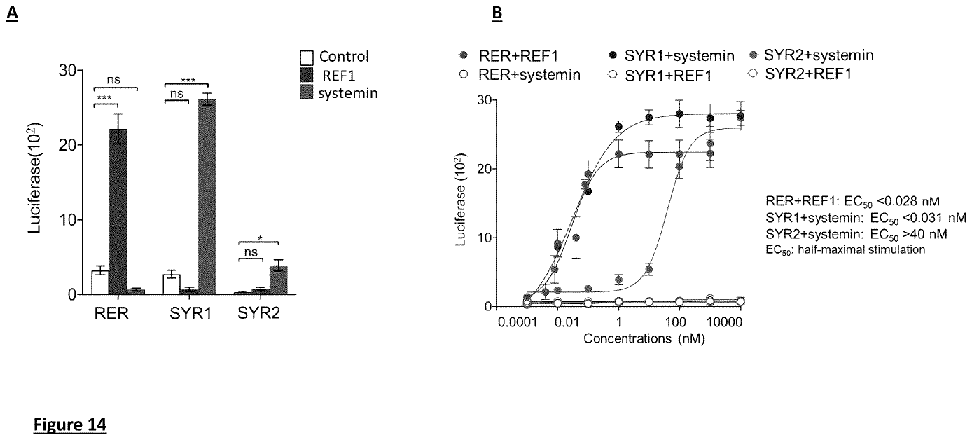

Receptor activation was tested by using FRK1 expression as a marker gene. Protoplasts were transfected with pFRK1::LUC along with indicated constructs and induced with 100 nM of REF1 or systemin for 3 h. Data are mean±SD, n=3 repeats. ***P<0.001 (Student's t test); ns, not significant FRK-Luciferase expression was used as a marker to test REF1 binding-mediated activation of its receptor. Based on these assay, the EC50 was calculated (half-maximal stimulation) of REF1-receptor binding as an assessment of the binding affinity of REF1 to its receptor RER. In these assays, systemin and its two receptors SYR1 and SYR2 were used as controls. FIG. 14a shows that REF1 bound to RER, indicating that RER is the receptor of REF1. FIG. 14b shows that RER binds REF1 with high affinity providing further confirmation that RER is the receptor of REF1.

### Example 7—SIPEPR1 (RER) is Involved in Determining Plant Regenerative Capacity

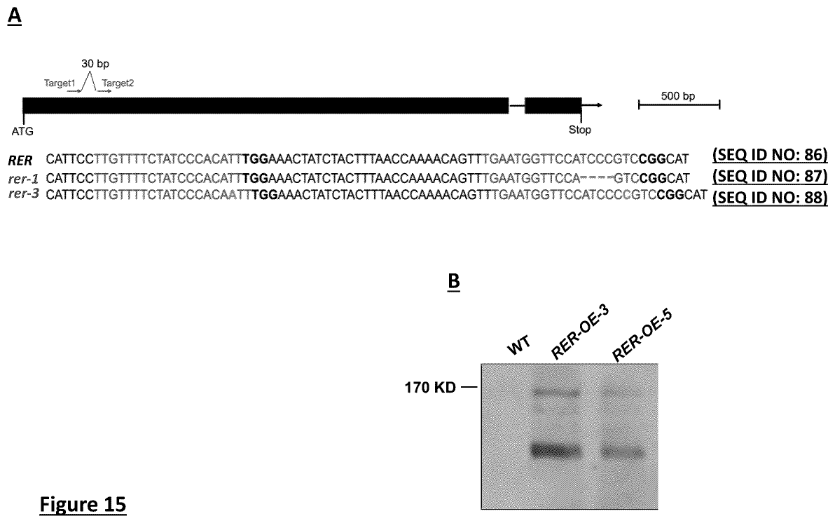

To determine whether SIPEPR1 (RER) is directly involved in determining plant regenerative capacity, RER knock out mutants (rer-1 and rer-3) were generated as shown in FIG. 15A. RER over expressing plants were also generated (RER-OE-3 and RER-OE-5), FIG. 15B shows a western blot and illustrates that both RER-OE-3 and RER-OE05 express RER at higher levels than the wild type (WT) plant.

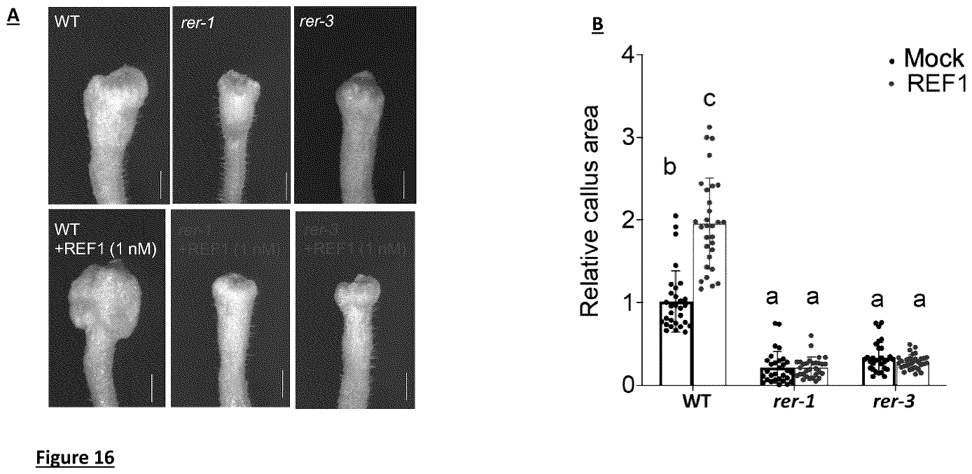

Callus formation was measured in RER knock out mutants (rer-1 and rer-3) compared to wild type (WT) tomato plants (Ailsa Craig) in the presence and absence of SIPEP (REF1). FIG. 16A visually shows that callus formation was incredibly small in rer-1 and rer-3 plants in the presence and absence of REF1 in comparison to WT. FIG. 16B shows the relative callus area of each plant. Here it can be seen that callus area was highest in WT plant grown with REF1, as REF1 induces callus formation, as shown in Examples 2 and 4. Callus formation in rer-1 and rer-3 plants was very small in both the presence and absence of REF1 with REF1 failing to lead to any increase in relative callus area. This shows that if RER is absent then REF1 induced callus formation does not occur, i.e. rer knockouts are defective in REF1 induced callus formation.

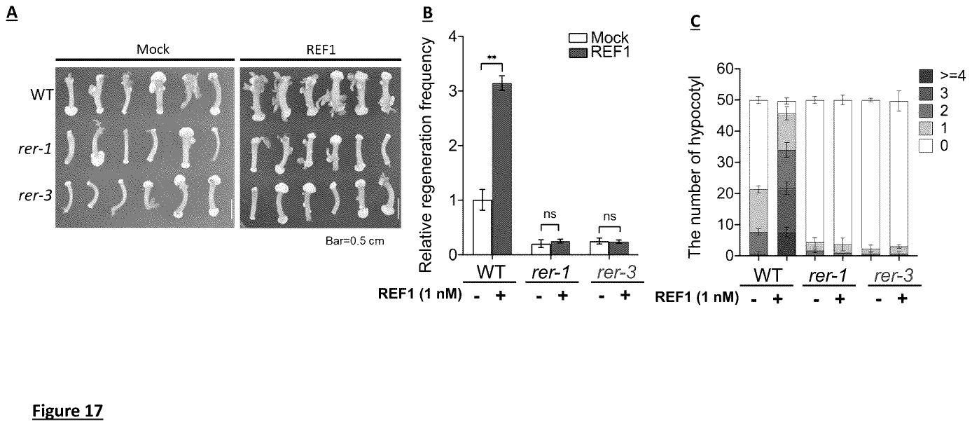

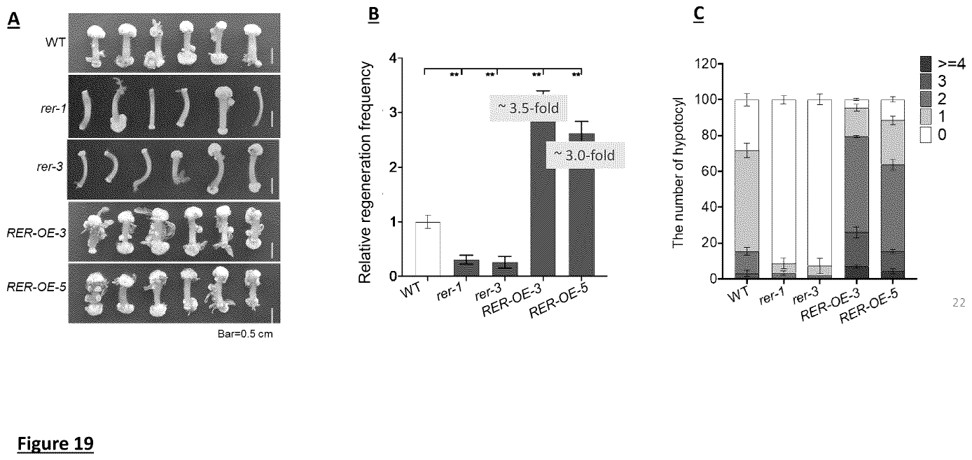

Shoot regeneration was also measured in RER knock out mutants (rer-1 and rer-3) compared to wild type (WT) tomato plants (Ailsa Craig) in the presence and absence of SIPEP (REF1). FIG. 19A visually shows that shoot regeneration was incredibly small in rer-1 and rer-3 plants in the presence and absence of REF1 in comparison to WT. FIGS. 17B and C shows that relative regeneration frequency and number of hypocotyl was highest in WT plants grown with REF1 as REF1 induces shoot regeneration as shown in Example 3. Shoot regeneration frequency and number of hypocotyl was very small in rer-1 and rer-3 knockouts, and growth in the presence of REF1 did not lead to any increase in either shoot regeneration frequency or number of hypocotyl. This shows that if RER is absent then REF1 induced shoot regeneration does not occur, i.e. rer knockouts are defective in REF1 induced shoot regeneration.

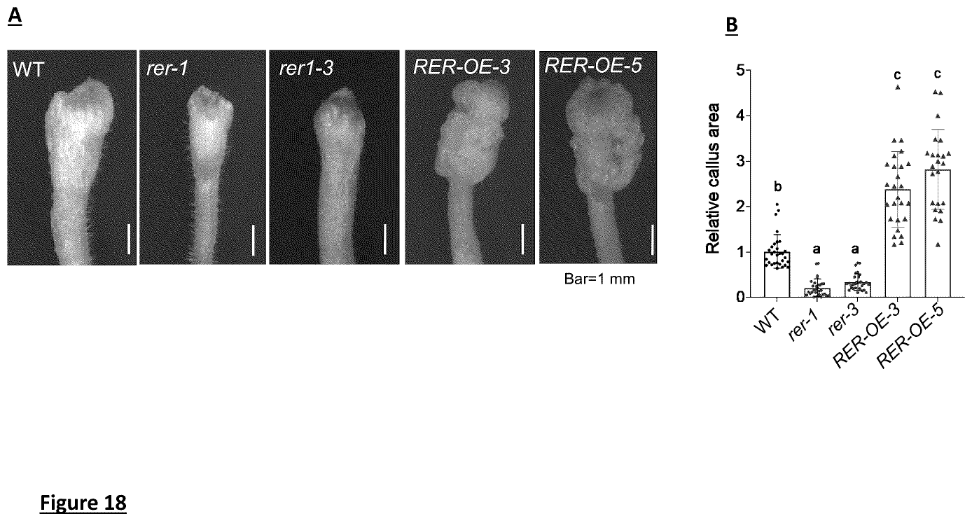

Overexpression of RER was also tested to see if this could increase plant regenerative capacity, with callus formation and shoot regeneration being measured. FIG. 18 shows that callus formation was much larger in RER-OE-3 and RER-OE-5 plants compared to WT and rer knockout plants, indicating that increased levels of RER increases plant regenerative capacity. Likewise, FIG. 19 shows that shoot regeneration capacity was much higher in RER-OE-3 and RER-OE-5 plants compared to WT, rer-1 and rer-3 plants, with RER-OE-3 and RER-OE-5 plants showing a ~3.5 fold and ~3 fold increase in relative regeneration frequency over WT plants.

Together this data shows that RER enhances the regenerative capacity of plants.

### Example 8—SIPEP (REF1) Enhances Regeneration Capacity in Recalcitrant Wild Tomatoes

Examples 1-7 were all performed with the tomato cultivar Ailsa Craig (AC), it is known in the art that AC has a high transformation efficiency, therefore the inventors investigated the effect of SIPEP (REF1) technology in recalcitrant tomatoes.

LA1777 is an accession of the wild tomato S. habrochaites that shows very low transformation efficiency. The effect of exogenously applied REF1 in a shoot inducing growth medium was investigated in LA1777 tomatoes by growing LA1777 tomatoes in the presence and absence of 1 nM REF1. Visually, FIG. 20A shows greater shoot regeneration in LA1777 when grown in the presence of 1 nM REF1 compared to when REF1 was absent in the growth medium. Likewise, FIG. 20B shoes that relative regeneration frequency of LA1777 was 6-fold higher when grown in the presence of REF1 compared to when REF1 was absent, similarly, the number of hypocotyl was also higher in the presence of REF1. This data demonstrates that REF1 increases the regenerative capacity of LA1777 tomatoes.

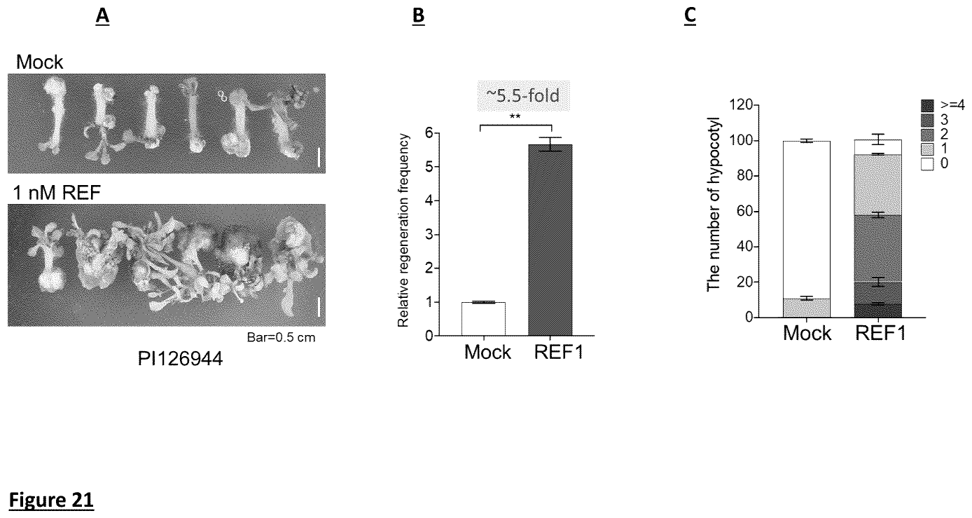

PI126944 is an accession of the wild tomato S. peruvianum that shows very low transformation efficiency. The effect of exogenously applied REF1 in a shoot inducing growth medium was investigated in PI126944 tomatoes by growing PI126944 tomatoes in the presence and absence of 1 nM REF1. Visually, FIG. 21A shows greater shoot regeneration in PI126944 when grown in the presence of 1 nM REF1 compared to when REF1 was absent in the growth medium. Likewise, FIG. 21B shows that relative regeneration frequency of PI126944 was 5.5-fold higher when grown in the presence of REF1 compared to when REF1 was absent, similarly, the number of hypocotyl was also higher in the presence of REF1. This data demonstrates that REF1 increases the regenerative capacity of PI126944 tomatoes.

So far this data had shown that exogenous application of REF1 is able to increase the regenerative capacity of recalcitrant tomatoes. Next, it was necessary to establish whether the regenerative capacity of recalcitrant tomatoes could also be increased by means of plant transformation and/or genome editing. Previously, it had been shown that expression of the PROSIPEP (PRR) gene and/or expression of the SIPEP receptor SIPEPR1 gene (RER) effectively increase the regenerative capacity of AC tomatoes. Therefore, constructs were designed to induce the expression of PRR and RER in tomatoes and other plants. The overexpression vector (pCeGFP) designed for plant transformation, referred to herein as the PRR+RER-OE construct is shown in FIG. 22A and the CRISPR-Cas9 vector (pTX041) designed for genome editing referred to herein as the PRR+RER construct is shown in FIG. 22B.

PI126944 tomatoes were transformed with the PRR+RER-OE construct as well as an equivalent construct only containing PRR and another construct only containing RER. Plants expressing these constructs along with a wild type control were grown in the presence or absence of REF1 for 19 days. FIG. 23 shows that shoots regeneration was increased in plants transformed with constructs containing PRR and RER separately, in each case relative regeneration frequency was increased ~25 fold compared to wild-type untransformed plants. The greatest increase in regenerative capacity was observed for PI126944 tomatoes transformed with the PRR+RER-OE construct, in this case a ~42 fold increase in relative regeneration frequency was observed compared to wild type plants grown in the absence of REF1. However, importantly, the PRR+RER-OE construct also led to a very large increase in regeneration frequency of PI126944 tomatoes in the absence of REF1. Overall this data shows that overexpression of PRR and/or RER increases the regenerative capacity of plants in the presence or absence of exogenous REF1, with exogenous exposure to REF1 enhancing plant regenerative capacity even further.

### Example 9—the Pepper Orthologue of SIPEP/REF1 (CaREF1) Promotes Regeneration in Pepper

SIPEP, PROSIPEP, and the SIPEP receptor/SIPEPR1 (REF1, PRR and RER) are conserved in the plant kingdom with orthologues in most species. Here, we demonstrate that the effect of SIPEP (REF1) is replicated across different plants.

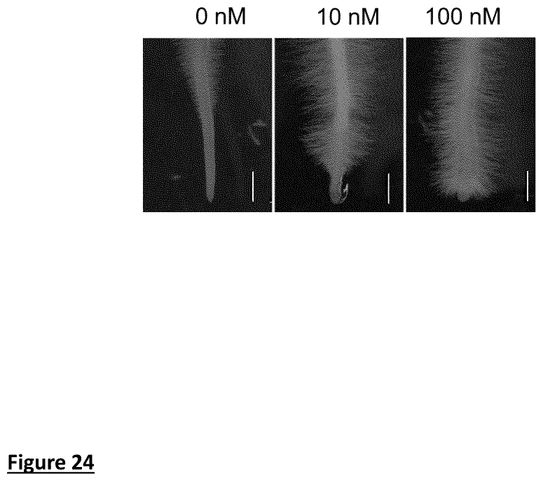

The pepper SIPEP (REF1) orthologue (CaREF1) was investigated to establish whether CaREF1 can increase the regenerative capacity of pepper (Capsicum annuum). It was established that CaREF1 promotes root hair formation, as is shown in FIG. 24.

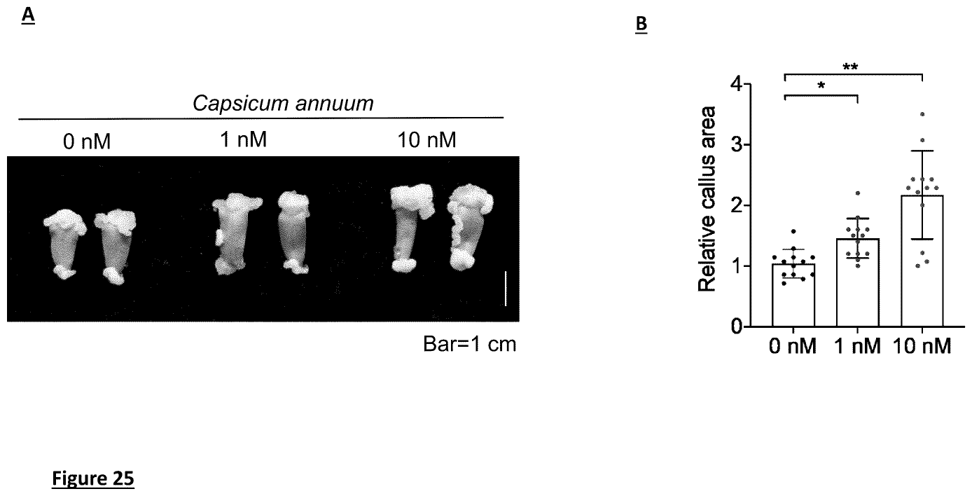

To further support a role of CaREF1 in increasing regenerative capacity in pepper, we show that callus formation was also increased in plants grown in the presence of CaREF1 (FIG. 25).

### Example 10—the Soybean Orthologue of SIPEP/REF1 (GmREF1) Promotes Regeneration in Soybean

The soybean SIPEP (REF1) orthologue (GmREF1) was investigated to establish that GmREF1 can increase the regenerative capacity of soybean (Glycine max).

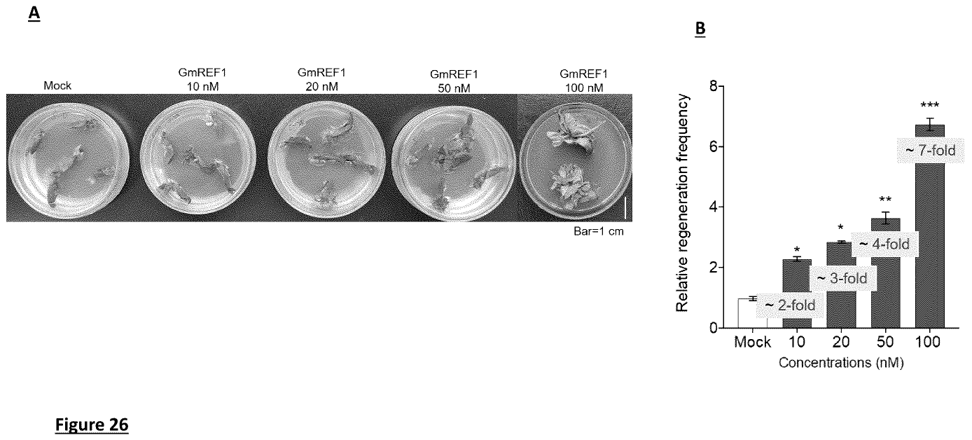

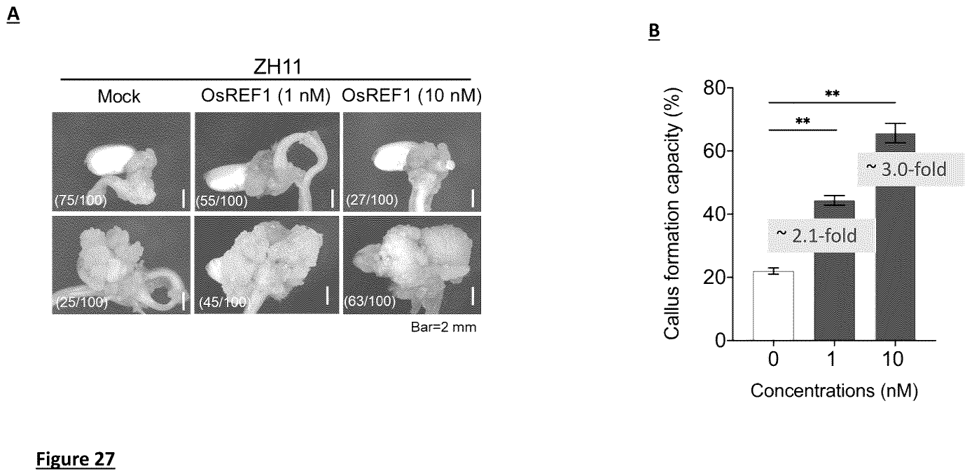

Soybean explants were cultured on medium containing indicated concentrations of REF1. Shoot regeneration was scored 21 days after incubation. As shown in FIG. 27, GmREF1 increases the regenerative capacity of soybean in plants grown in the presence of GmREF1. An increase of ~2.1, ~2.8 and ~3.8 fold was observed for plants grown in the presence of 10, 20 and 50 nM GmREF1 respectively compared to plants grown in the absence of GmREF1 (FIG. 26).

The tomato PRR+RER-OE construct was also found to greatly improve the transformation efficiency of recalcitrant soybean, and recalcitrant soybean exhibited a ~12-fold increase in regeneration frequency when grown in the presence of 100 nM GmREF1 compared to a control recalcitrant soybean grown in the absence of GmREF1.

### Example 11—the Rice Orthologue of SIPEP/REF1 (OsREF1) Promotes Regeneration in Rice

The rice SIPEP (REF1) orthologue (OsREF1) was investigated to establish whether OsREF1 can increase the regenerative capacity of rice (Oryza sativa).

Mature seeds of ZH11 were cultured on CIM containing indicated concentrations of OsREF1 (1 nM and 10 nM). As shown in FIG. 27A calluses formed that were larger in the presence of OsREF1 than in the absence of OsREF1. Further to this, FIG. 27B shows a ~2 and ~3 fold increase in callus formation capacity in ZH11 grown in the presence of 1 nM and 10 nM OsREF1 respectively compared to ZH11 grown in the absence of OsREF1. This data shows that OsREF1 promotes callus formation in Japonica rice.

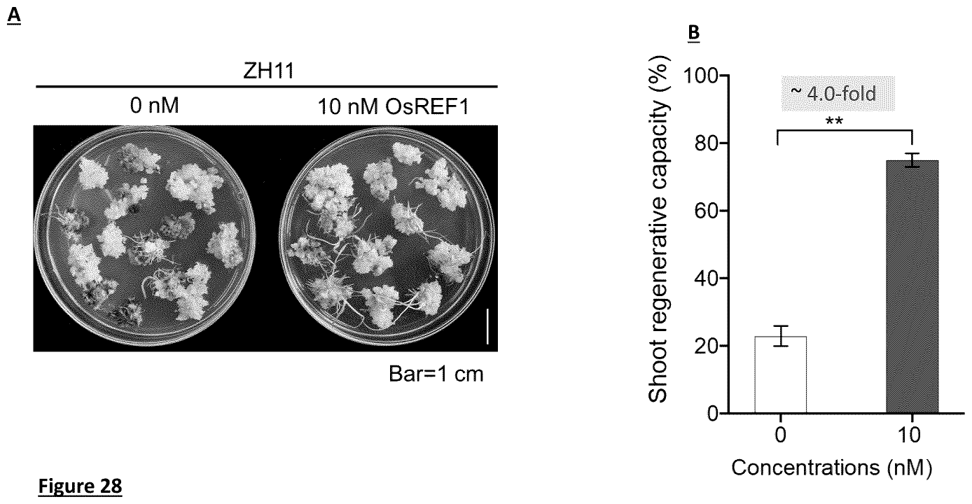

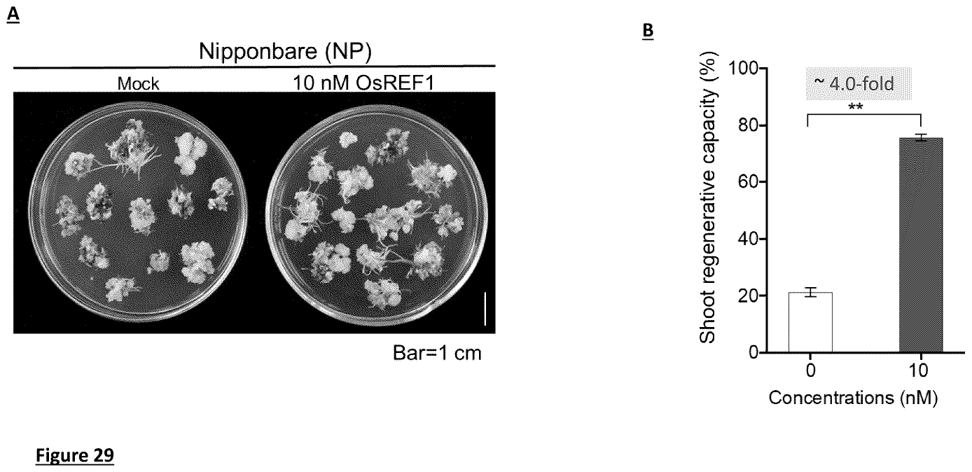

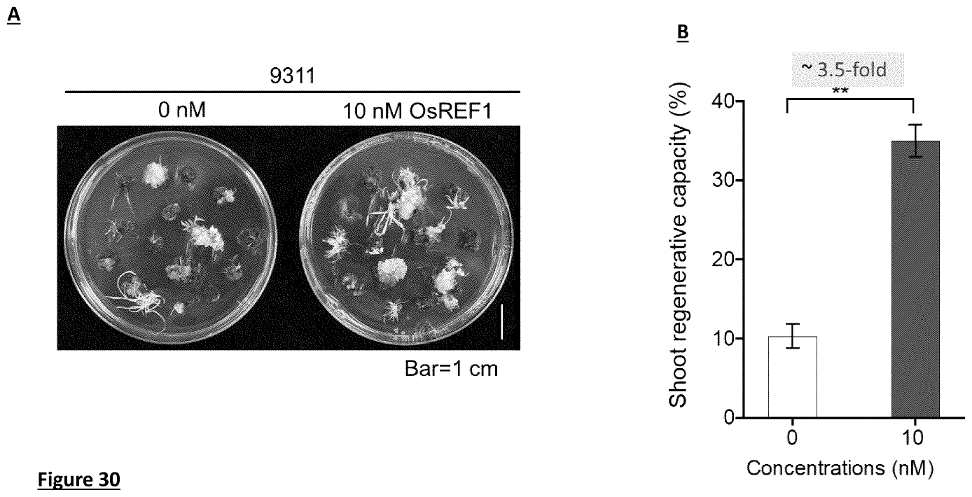

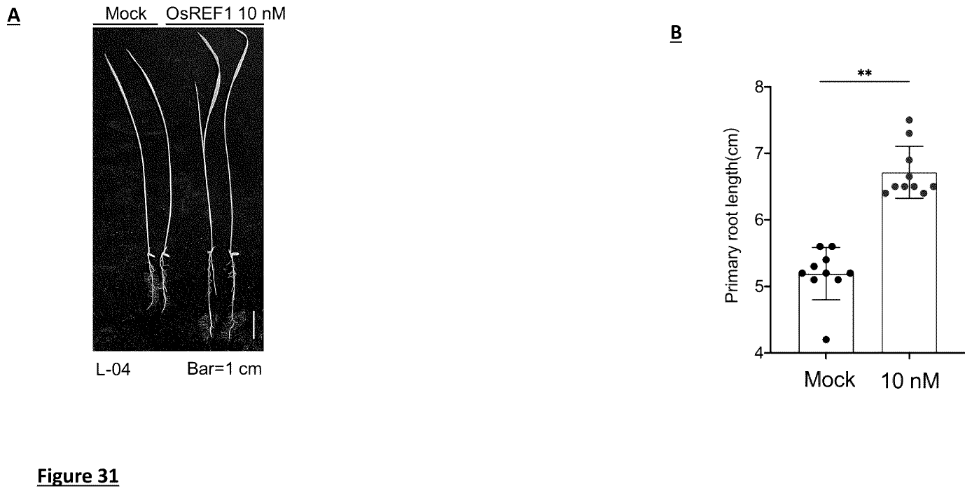

Shoot regeneration was investigated in ZH11 and Nipponbare Japonica rice as well as 9311 indica rice. OsREF1 promoted shoot generation in all three of these rice varieties. A ~4 fold increase was observed in shoot regeneration capacity for ZH11 rice and Nipponbare rice (FIG. 28 and FIG. 29), whilst a ~3.5 fold increase in shoot regeneration capacity was seen for 9311 rice (FIG. 30) grown in the presence of 10 nM OsREF1 compared to rice grown in the absence of OsREF1.

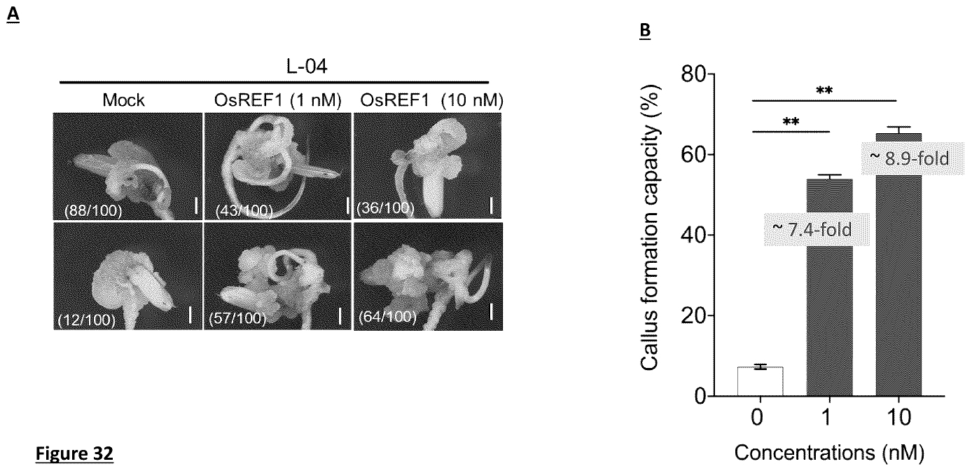

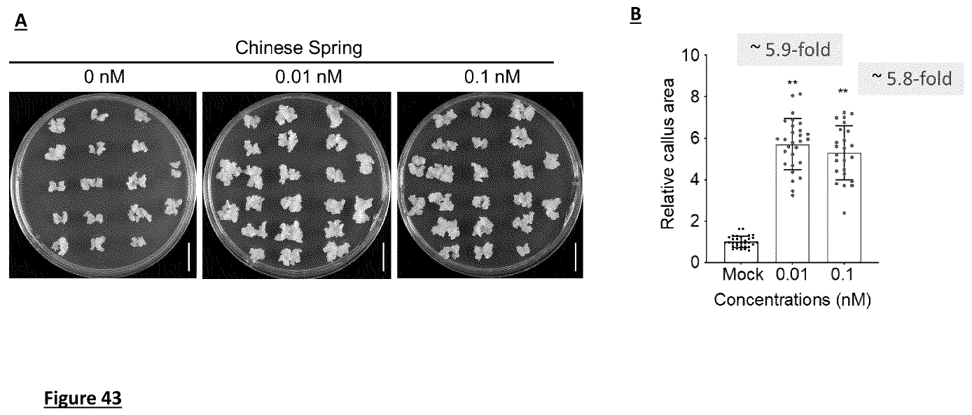

Wild allotetraploid rice (L-04) was also tested as this particular rice variety shows low transformation efficiency. We found that OsREF1 promotes the growth of wild allotetraploid rice (L-04). This is shown in FIG. 31 where the primary root length is significantly increased in wild allotetraploid rice grown in the presence of 10 nM OsREF1. OsREF1 was also shown to promote shoot regeneration in wild allotetraploid rice (L-04), with shoot regeneration frequency being ~12.2 fold increased in L-04 rice grown in the presence of 10 nM OsREF1 compared to L-04 rice grown in the absence of OsREF1 (FIG. 43).

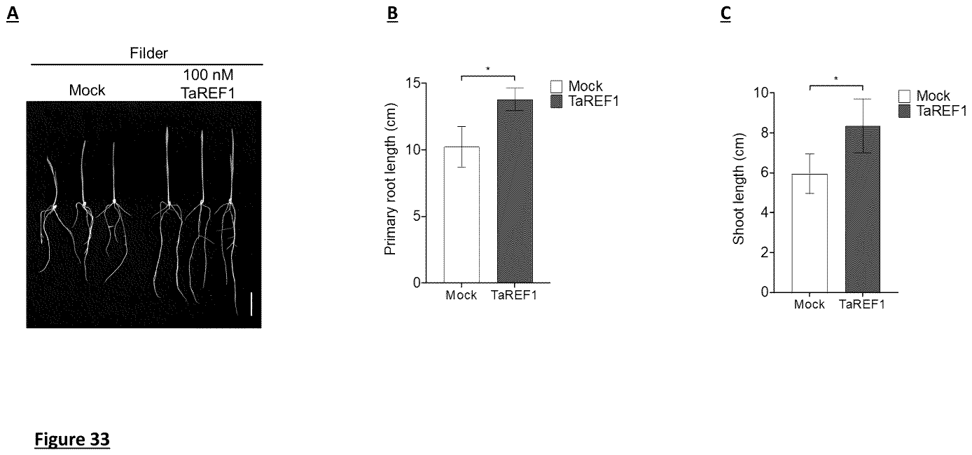

OsREF1 also promotes callus formation capacity in wild allotetraploid rice. Seeds of wild allotetraploid rice L-04 were cultured on CIM containing indicated concentrations of OsREF1 (1 nM and 10 nM) for 20 days. As shown in FIG. 32, growing L-04 wild allotetraploid rice showed a ~7.4 fold and ~8.9 fold increase in callus formation capacity when grown in the presence of 1 and 10 nM OsREF1 for 20 days respectively compared to wild allotetraploid rice (L-04) grown in the absence of OsREF1.

This data shows that the SIPEP (REF1) orthologue (OsREF1) is able to enhance the regenerative capacity of rice, including Japonica rice, indica rice and wild allotetraploid rice.

### Example 12—the Wheat Orthologue of SIPEP/REF1 (TaREF1) Promotes Root and Shoot Growth in Wheat

The wheat SIPEP (REF1) orthologue (TaREF1) was investigated to establish whether REF1 can increase the regenerative capacity in wheat. It was observed that wheat grown in the presence of 100 nM TaREF1 displayed increased root and shoot length compared to wheat grown in the absence of TaREF1.

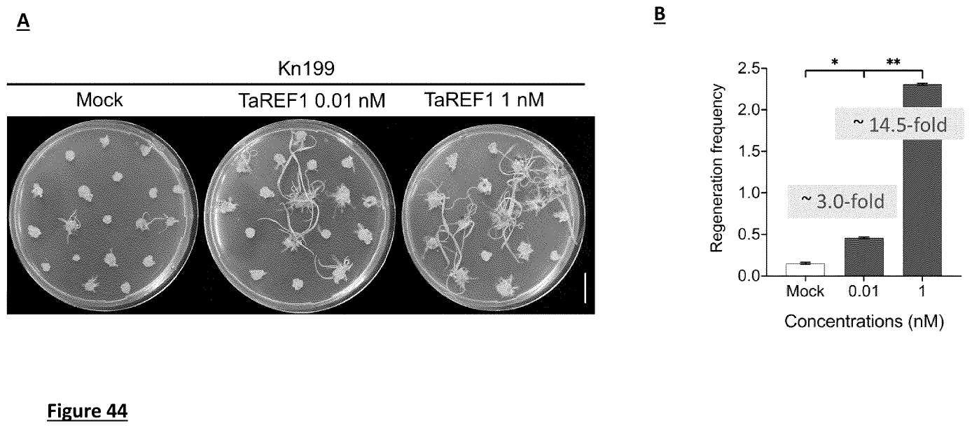

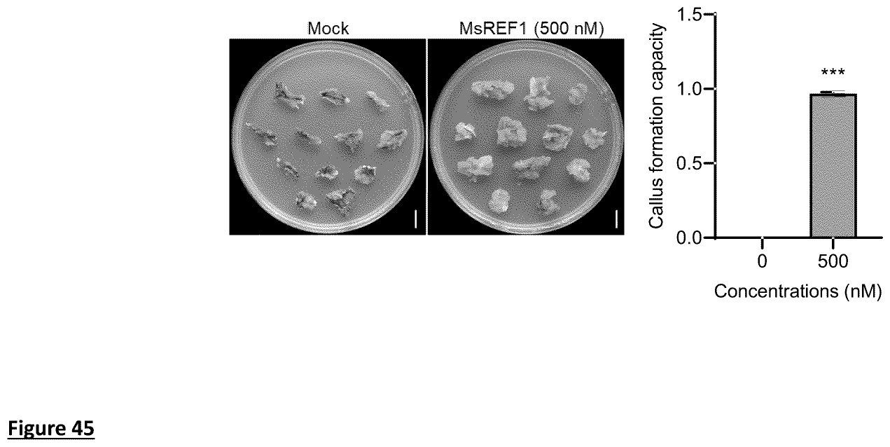

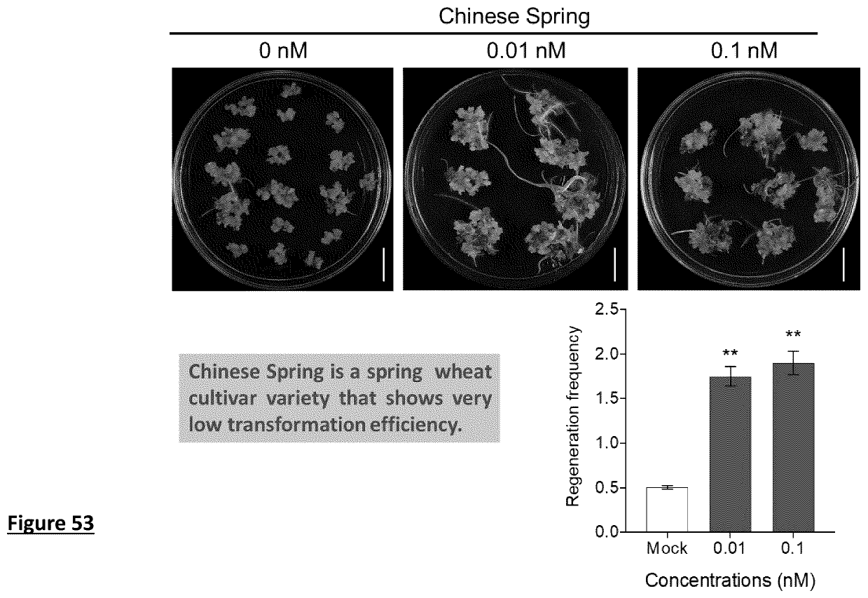

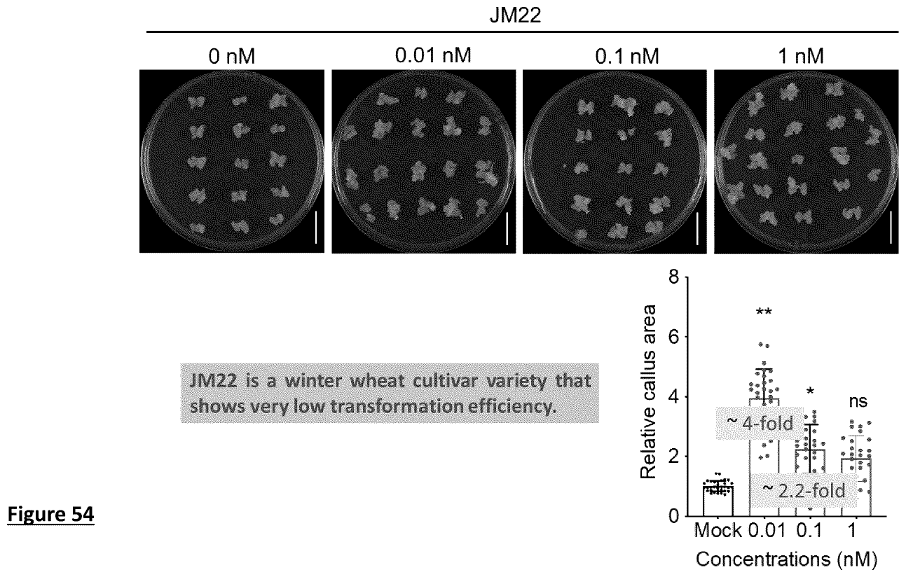

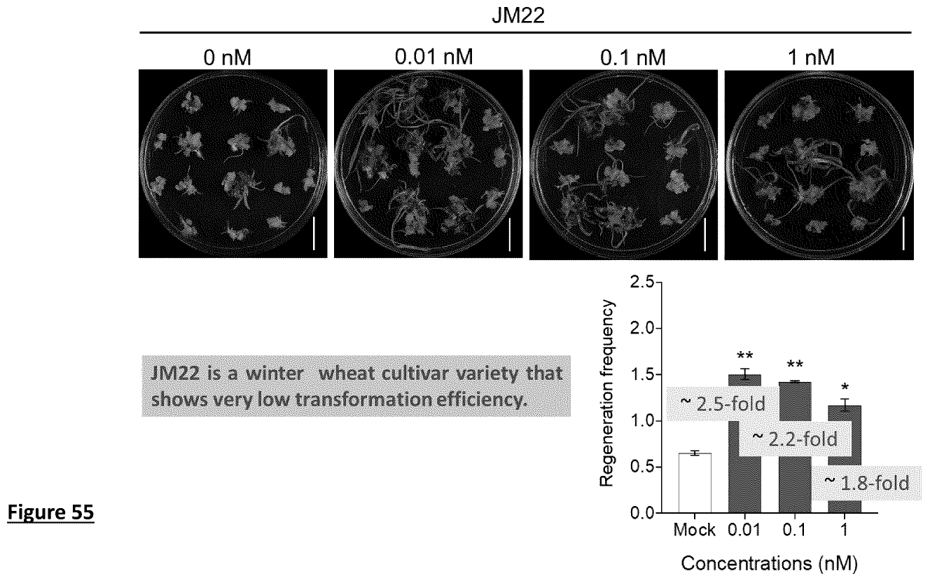

TaREF1 was also shown to increase the callus formation capacity of Chinese spring wheat when applied exogenously (FIG. 43) and JM22 wheat (FIG. 54). TaREF1 was also able to promote shoot regeneration in Chinese Spring wheat (FIG. 53), Kn199 wheat (FIG. 44), and JM22 wheat (FIG. 55) when applied exogenously.

This shows that REF1 is able to increase the regenerative capacity of wheat across a range of cultivars.

The area of each callus was quantified using ImageJ, then the average of each group was calculated. The average for each group was then divided by the average of WT-Mock.

Shoot regeneration frequency was established by calculating the average number of shoots, in the context of the present invention shoot regenerative capacity refers to the number of shoots per explant in each condition.

### Example 13—the Peach Orthologue of SIPEP/REF1 (PpREF1) Promotes Callus Formation in Peach

The peach SIPEP (REF1) orthologue (PpREF1) was investigated to establish its effect on peach regenerative capacity.

Leaves of peach cultivar GF677 were cultured on CIM containing indicated concentrations of PpREF1, and callus phenotype was scored at 24 days after incubation on CIM. Callus formation capacity was represented by the percentage of leaves explants with regenerated embryonic calli. Data are mean±SD of three independent experiments. Callus formation capacity was increased by 10-fold in peach cultivar GF677 leaves grown in the presence of 100 nM PpREF1 compared to leaves grown in the absence of PpREF1.

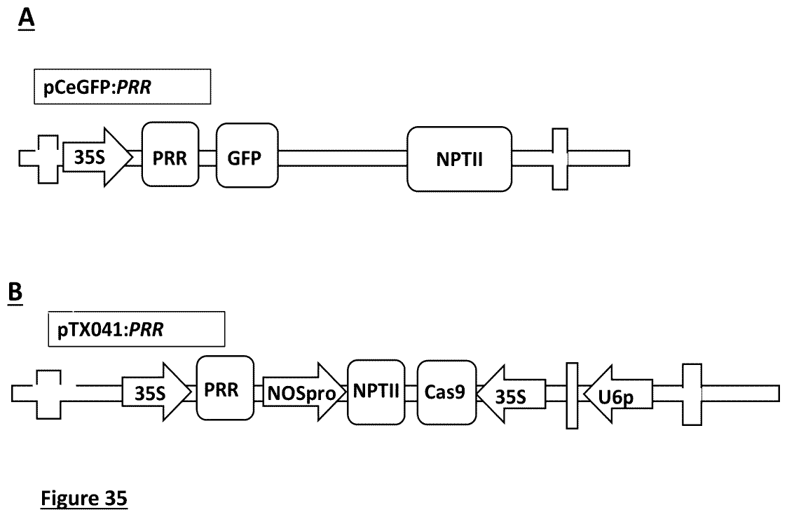

This data shows that PpREF1 increases regenerative capacity of peach (FIG. 34).

### Example 14—the Sunflower Orthologue of SIPEP/REF1 (HanPEP1) Promotes Shoot Regeneration in Sunflower

The sunflower SIPEP (REF1) orthologue (HanREF1) was investigated to establish its effect on sunflower regenerative capacity. Shoot regeneration was shown to be increased in sunflower explants grown in the presence of HanREF1 compared to its absence with an increase of ~2.1 fold in the presence of 1 nM HanREF1 and an increase of ~4.5-fold in the presence of 10 nM HanREF1.

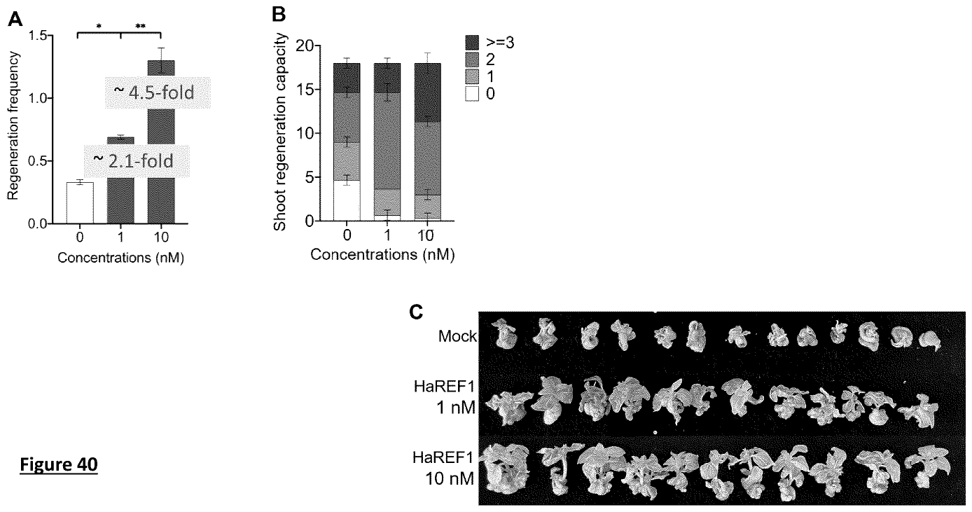

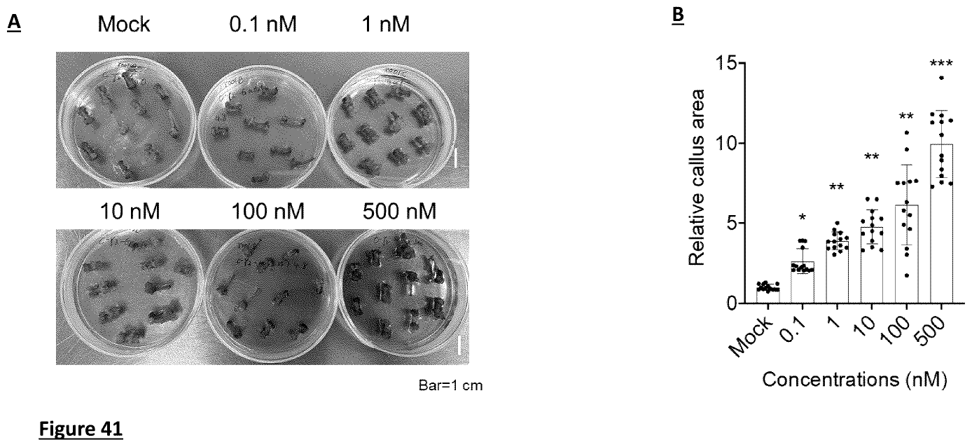

This data shows that HanREF1 increases the regenerative capacity of sunflower (FIG. 40).

For the regeneration protocol we referred to Radonic et al., 2015. The explant used in the sunflower regeneration assay was cotyledon.

Shoot regeneration frequency was established by calculating the average number of shoots, in the context of the present invention shoot regenerative capacity refers to the number of shoots per explant in each treatment.

### Example 15—the Cotton Orthologue of SIPEP/REF1 (GaPEP1) Promotes Callus Formation in Cotton

The cotton SIPEP (REF1) orthologue (GaREF1) was investigated to establish its effect on cotton regenerative capacity. Relative callus area was shown to be increased in hypocotyl explants of cotton grown in the presence of GaREF1 compared to its absence, with relative callus area increasing as concentration of GaREF1 is increased (0.1-500 nM).

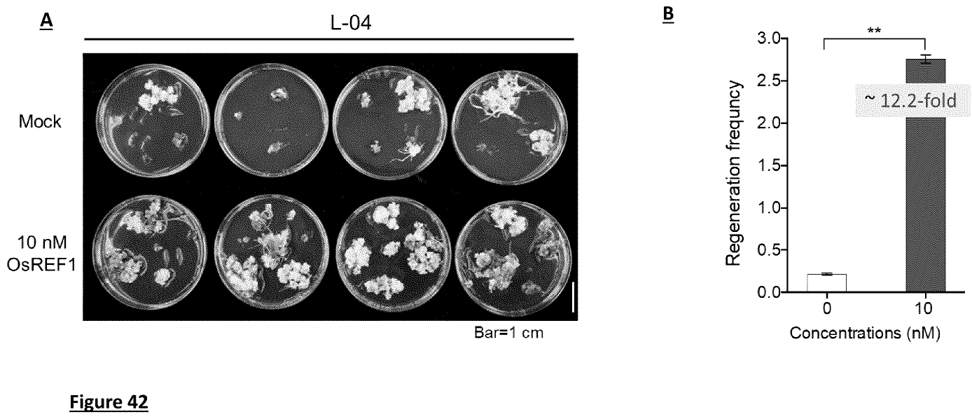

This data shows that GaREF1 increases regenerative capacity of cotton (FIG. 41).

### Example 16—MsREF1 Promotes Callus Formation in Medicago sativa

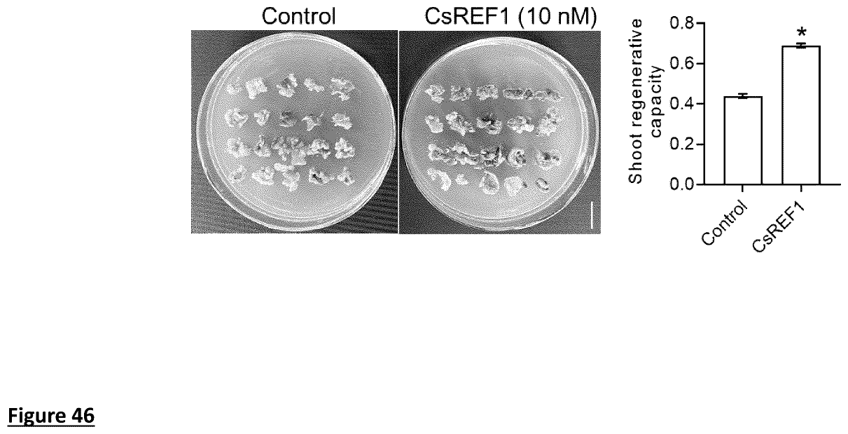

Leaf explants were cultured on callus-inducing medium (CIM) containing indicated concentrations of MsREF1, and callus phenotype was scored at 14 d after incubation. It can be seen in FIG. 45 that callus formation was significantly increased in Medicago sativa treated with 500 nM MsREF1.

### Example 17—CsREF1 Promotes Shoot Regeneration in Cucumber

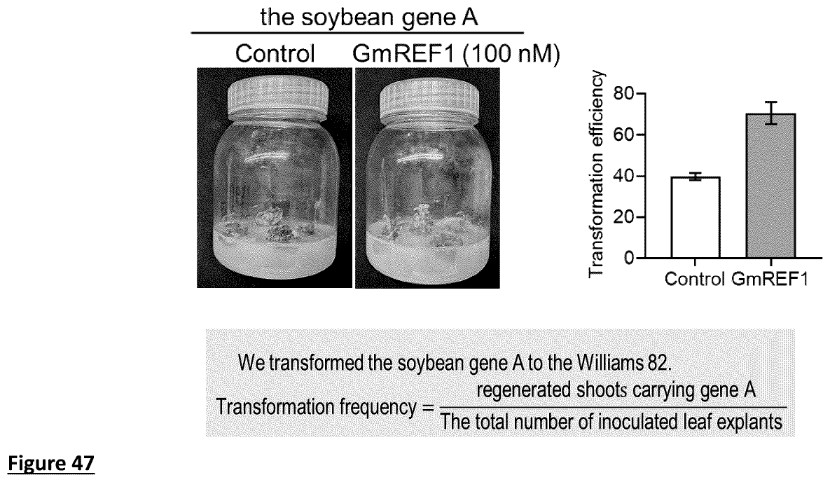

Leaf explants were cultured on a medium containing 10 nM CsREF1. Explants treated with CsREF1 exhibited a significantly increased shoot regenerative capacity compared to control explants that were not treated with CsREF1, this is shown in FIG. 46.

### Example 18—GmREF1 Improves the Transformation Efficiency of Soybean

The soybean gene A was transformed into the Williams 82 soybean cultivar and treated with 100 nM GmREF1 or a control. Gene A is a transcription factor gene which is used as a reporter for transformation efficiency. FIG. 47 shows that plants treated with GmREF1 appeared visually healthier (in a representative image of the experiments) and had a greater transformation efficiency. The data obtained in these experiments is given below:

| | Regenerated | | |
|---|---|---|---|
| | shoots | | |
| GmREF1 | carrying the | Total number | Transformation |
| concentrations | reporter | of inoculated | efficiency |
| (nM) | gene | leaves | (%) |
| 0 | 122 | 299 | 40.8 |
| | 107 | 284 | 37.6 |
| | 118 | 290 | 40.6 |
| 100 | 200 | 274 | 72.9 |
| | 177 | 276 | 64.1 |
| | 156 | 211 | 73.9 |

### Example 19—the Tomato PRP+RER-OE Construct Improves the Shoot Regenerative Capacity of Recalcitrant Rice

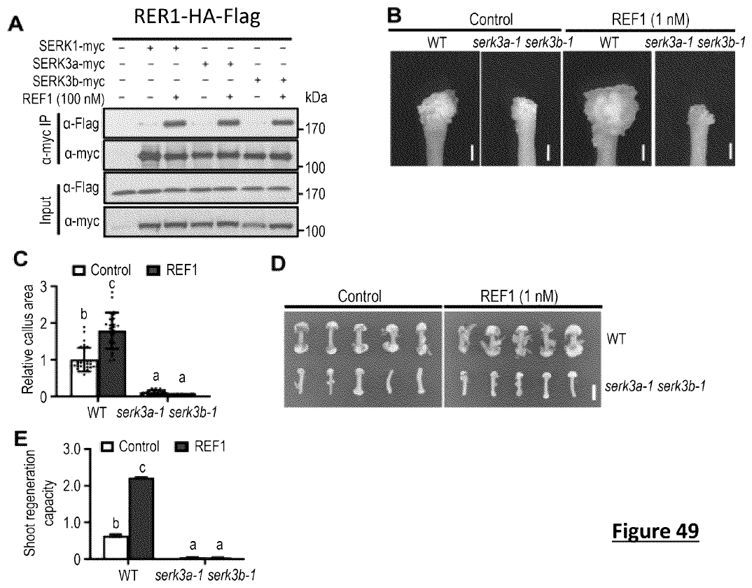

Recalcitrant rice were transformed either with an empty vector or with the PROSIPEP (PRP)+RER-OE construct. FIG. 48 shows that shoot regenerative capacity was increased in both ZH11 and 9311 rice transformed with the PROPEP (PRP)+RER-OE construct.

### Example 20—ZmREF1 Promotes Shoot Regeneration

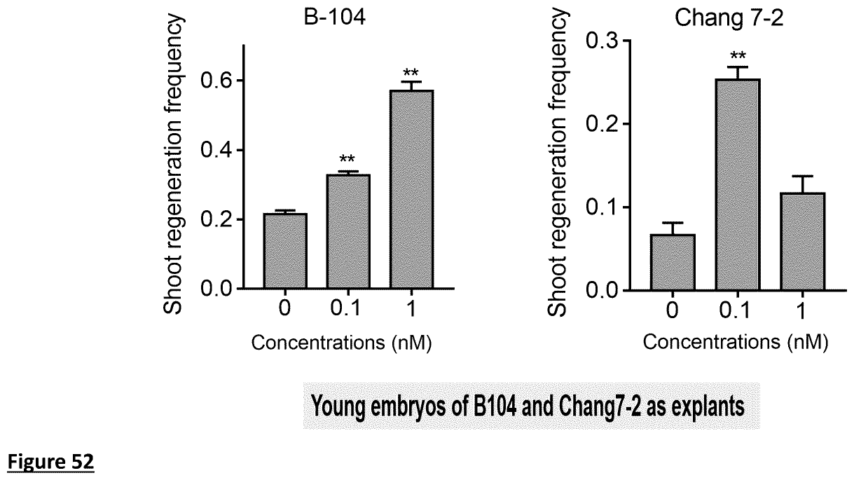

Young embryos of B104 and Chang 7-2 were treated with 0, 0.1 or 1 nM of ZmREF1. For both B-104 and Chan 7-2 shoot regeneration frequency was increased when ZmREF1 was applied, for B-104 this was greatest at 1 nM, however, for Chan 7-2 this was greatest at 0.1 nM, as shown in FIG. 52.

### Example 21—REF1-RER Regulates Regeneration Through SERK Co-Receptors

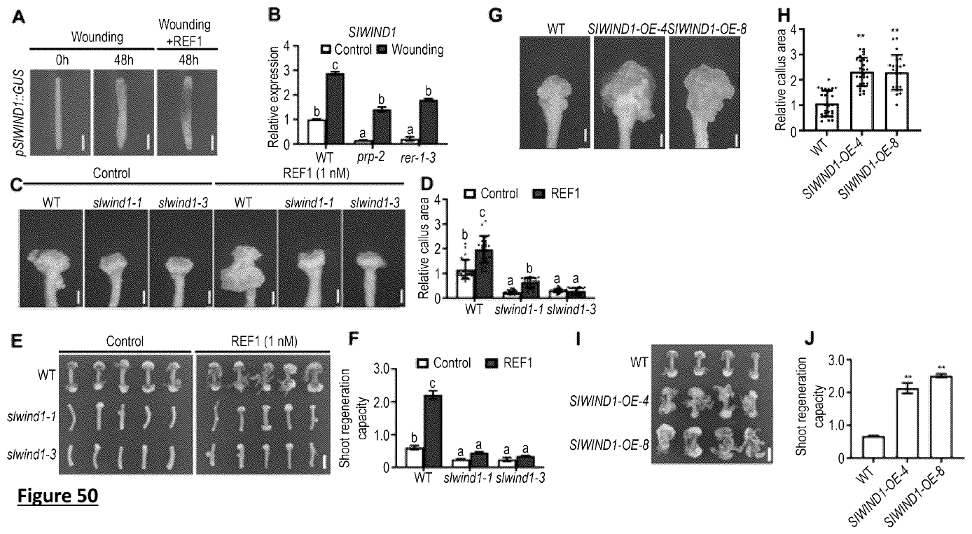

Somatic Embryogenesis Receptor Kinase (SERKs) are co-receptors of multiple LRR-RLKs (Ma et al., 2016; Peng and Kaloshian, 2014). Co-immunoprecipitation (co-IP) assays indicated that REF1 could simulate complex formation of RER with the 3 SERK members in tomato (FIG. 49A). Callus formation and shoot organogenesis capacity was slightly reduced in serk3a-1 or serk3b-1 single mutants but was largely compromised in the serk3a-1 serk3b-1 double mutant plants (FIGS. 49B-49E). Moreover, REF1 failed to rescue the regenerative defects of the above serk mutants (FIGS. 49B-49E). These results imply that REF1-RER regulates regeneration through the co-receptors SERK3a and SERK3b.

### Example 22—REF1 Regulates Regeneration Through Promoting SW/ND1 Expression

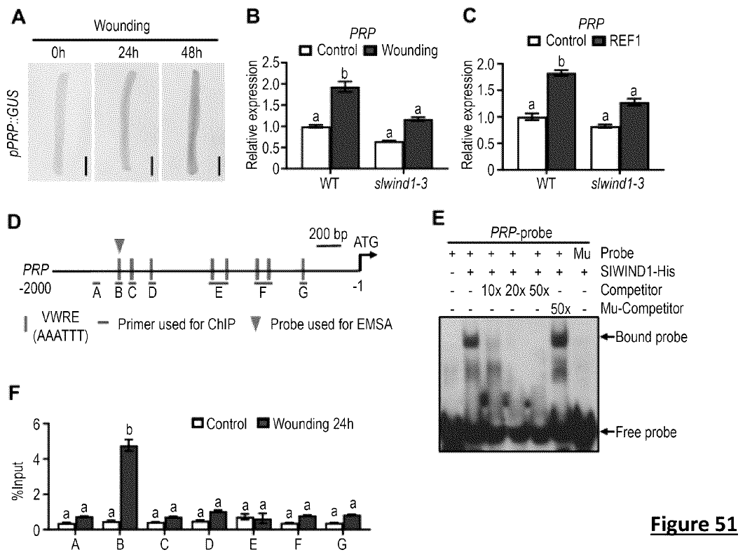

To understand the mechanisms by which REF1 regulates organ regeneration, the inventors examined whether REF1 induces the tomato homologue of WOUND-INDUCED DEDIFFERENTIATION 1 (WIND1), an AP2/ERF transcription factor in Arabidopsis that acts as a central regulator of wound-induced cellar reprogramming (Iwase et al., 2011). Using transgenic plants carrying a SIWIND1 promoter-driven β-glucuronidase (pSIWIND1::GUS) fusion, it was found that the SIWIND1 promoter activity was induced in cutting hypocotyls and that this induction was enhanced by REF1 treatment (FIG. 50A). Notably, quantitative PCR with reverse transcription (RT-qPCR) analysis revealed that the wound-induced SIWIND1 expression was compromised in prp-2 and rer1-3 mutants (FIG. 50B), indicating that the REF1-RER1 module is important for wounding-induced activation of SIWIND1 expression.

To determine the function of SIWIND1 in plant regeneration, we generated null mutants (sIwind1) and overexpression plants (SIWIND1-OE) of SIWIND1 and examined their regenerative phenotypes in conventional tissue culture. Results showed that while sIwind1 mutants displayed severely compromised capacity of callus formation and shoot organogenesis (FIGS. 50C-50F), SIWIND1-OE plants displayed enhanced capacity of callus formation and shoot organogenesis as compared to the WT (FIGS. 50G-50J), indicating that SIWIND1 plays a pivotal role for the acquisition of regeneration competency in tissue culture. Moreover, the compromised regenerative capacity of sIwind1 mutants could not be rescued by REF1 (FIGS. 50C-50F). Together, these results support that REF1 promotes wound-induced regeneration through activating SIWIND1 expression.

### Example 23—SIWIND1 Activates PRP Expression in Response to Wounding

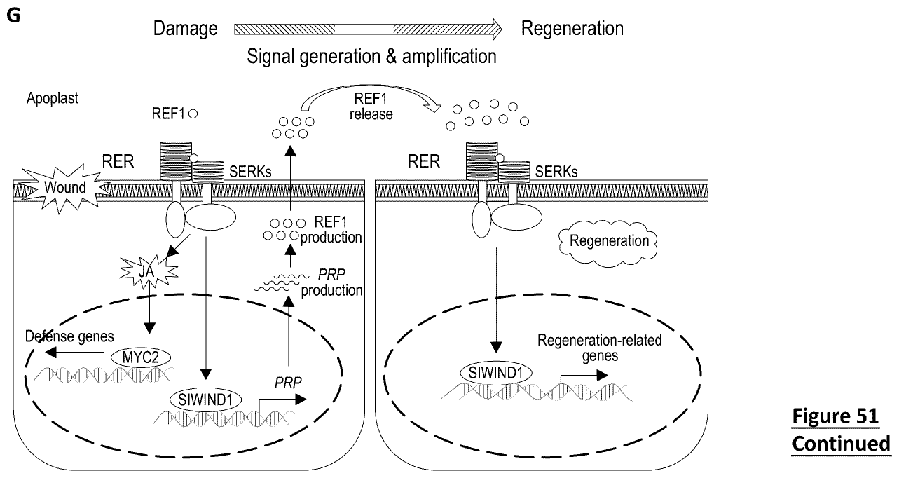

Having established that REF1 promotes regeneration through activating the master cellular reprogramming regulator SIWIND1, we asked whether and how activated SIWIND1 further amplifies the REF1 signal. Indeed, the promoter activity of the REF1 precursor gene was induced by wounding in cutting hypocotyls, as assessed by the pPRP::GUS reporter assays (FIG. 51A). Furthermore, wound- and REF1-mediated induction of PRP expression was compromised in sIwind1-3 mutants compared to the WT (FIGS. 51B and 51C), suggesting that SIWIND1 plays an important role in wound-triggered induction of PRP expression. To understand the transcriptional mechanism through which SIWIND1 regulates wound-induced expression of PRP, we examined the PRP promoter for putative SIWIND1 binding motifs, and identified 8 vascular system-specific and wound-responsive cis-element (VWRE)-like motifs (Iwase et al., 2017; Sasaki et al., 2002; Sasaki et al., 2006) within a 2,000-bp region of the PRP promoter (FIG. 51D). Of note, the wound-responsive VWRE was also present within the promoter of SIWIND1, RER1, PRS and the systemin receptor genes SYR1 and SYR2, reinforcing the notion that these genes are involved in plant wound responses. In line with the observations that WIND family proteins preferentially bind VWRE (Iwase et al., 2017; Sasaki et al., 2007), electrophoretic mobility shift assays (EMSAs) showed that SIWIND1-His recombinant protein bound a DNA probe containing the VWRE (5′-AAATTT-3′) motif but failed to bind a DNA probe in which the VWRE motif was mutated (FIG. 51E). Consistently, chromatin immunoprecipitation-qPCR (ChIP-qPCR) assays (Du et al., 2017) using SIWIND1-GFP plants and GFP antibody revealed that SIWIND1-GFP was enriched in the PRP promoter (FIGS. 51D and 51F). Collectively, these results indicate that SIWIND1 binds the VWRE within the PRP promoter and activates its expression. Thus, REF1-initiated activation of SIWIND1 positively feeds back on the REF1 precursor gene to amplify the primary wound signal (FIG. 51G).

### SEQUENCE LISTING

SEQ ID NO: 1-SIPEP (REF1) Tomato DNA Sequence (SiREF1)
GCAACAGATAGGAGGGGAAGACCACCATCAAGACCTAAAGTTGGTAGTGGACCTCCT
CCTCAGAACAAT
SEQ ID NO: 2-SIPEP (REF1) Tomato Amino Acid Sequence (SiREF1)
ATDRRGRPPSRPKVGSGPPPQNN
SEQ ID NO: 3-SIPEP (REF1) Pepper DNA Sequence (CaREF1)
ACAGGCCGGAGGAGGGGAAGACCACCATCAAGACCTGGAGTTGGAAGGGGACCTC
CTCCTGAGAACAAT
SEQ ID NO: 4-SIPEP (REF1) Pepper Amino Acid Sequence (CaREF1)
TGRRRGRPPSRPGVGRGPPPENN
SEQ ID NO: 5-SIPEP (REF1) Soybean DNA Sequence (GmREF1)
GGAAGTGTAGTAGTTCTACAAACGAAAAGAAAACATGATGGAGGAAAAGGACGTGAT
CCACAAACTAAT
SEQ ID NO: 6-SIPEP (REF1) Soybean Amino Acid Sequence (GmREF1)
GSVVVLQTKRKHDGGKGRDPQTN
SEQ ID NO: 7-SIPEP (REF1) Sunflower DNA Sequence (HanREF1)
AGGAGACTCACAAGAAGACCCCCTCCGCCAAGGGGGCCGATTAGCTCTGGAGGAGG
CGGTCAAACCAACT
SEQ ID NO: 8-SIPEP (REF1) Sunflower Amino Acid Sequence (HanREF1)
RRLTRRPPPPRGPISSGGGGQTN
SEQ ID NO: 9-SIPEP (REF1) Cotton DNA Sequence (GaREF1)
CTTCCCATGGTTTCTCTCTTTACTCCTAAAAGGCCAGGGACAAGCGCCGGCAGTGGA
CCTCAGATTAAT
SEQ ID NO: 10-SIPEP (REF1) Cotton Amino Acid Sequence (GaREF1)
LPMVSLFTPKRPGTSAGSGPQIN
SEQ ID NO: 11-SIPEP (REF1) Rice DNA Sequence (OsREF1)
GATGATTCCAAGCCAACGCGGCCTGGAGCGCCGGCGGAGGGCTCCGGCGGCAACG
GCGGAGCAATCCAC
SEQ ID NO: 12-SIPEP (REF1) Rice Amino Acid Sequence (OsREF1)
DDSKPTGPGAPAEGSGGDGGAIH
SEQ ID NO: 13-SIPEP (REF1) Maize DNA Sequence (ZmREF1)
AGGAGGCCGCGGCCGCGGCCGCCGGATCACGCGAGAGAAGGGAGCGGTGGCAAT
GGAGGCGTACACCAC
SEQ ID NO: 14-SIPEP (REF1) Maize Amino Acid Sequence (ZmREF1)
RRPRPRPPDHAREGSGGNGGVHH
SEQ ID NO: 15-SIPEP (REF1) Wheat DNA Sequence (TaeREF1)
GCGGTGAGGAGGCCGCGGCCGCCCGGCAACCCGAGGGAAGGGCGCGGCGGCGGT
GGAGGAAGCCACAAC
SEQ ID NO: 16-SIPEP (REF1) Wheat Amino Acid Sequence (TaeREF1)
AVRRPRPPGNPREGRGGGGGSHN
SEQ ID NO: 17-SIPEP (REF1) Cucumber DNA Sequence (CsaREF1)
TATAAATTGGAAGGAGAGCTACAAAAGAGATTCGAGGATTTATTAAAAACAACCACGG
ATAATGATATTCCTCGTGGTGGAAACTAT
SEQ ID NO: 18-SIPEP (REF1) Cucumber Amino Acid Sequence (CsaREF1)
YKLEGELQKRFEDLLKTTTDNDIPRGGNY
SEQ ID NO: 19-SIPEP (REF1) Peach DNA Sequence (PpREF1)
AAAAGACCGAGAATTACTCTTCGTGCGGCAAGGCCAGAAATAAGCACAGGGAGTGGT
GCTCAGACTAAT
SEQ ID NO: 20-SIPEP (REF1) Peach Amino Acid Sequence (PpREF1)
KRPRITLRAARPEISTGSGAQTN
SEQ ID NO: 21-PROSIPEP (PROREF1)-(PRR) Tomato DNA Sequence (SiPRR)
ATGGAAGAGAAAAAAGAGAGTAGATCATCAGTTTATGATGAAGTTATCACCAAGAATC
CATTTTTTTACTTACAAGAAGGCATTAAAGCTATTCTAAAATGCCTTGGTTTTGAATCTT
CAAAACTTGTTCATCAAGCATCCTCTTCATCCTCGTCGTCCTCATCATCATCGTCTTCT
TCTTCTTCGAGTATGTTAGGTACTAATAATAAAAAAGAAGAAGAAGAGAGTGAAAAAC
AAGAACAAGAGTGTGTTTTGTTCCATGAAGATGGTAAGAAGCAAGGTTCAGATTCAAC
AAATGATAATTACGAAAATGACCCTCCAGCTGAAACTAATGATGAAGATCCAACTCTTA
TATTAGCAACAGATAGGAGGGGAAGACCACCATCAAGACCTAAAGTTGGTAGTGGAC
CTCCTCCTCAGAACAATTAA
SEQ ID NO: 22-PROSIPEP (PROREF1)-(PRR) Tomato Amino Acid Sequence
(SIPRR)
MEEKKESRSSVYDEVITKNPFFYLQEGIKAILKCLGFESSKLVHQASSSSSSSSSSSSSSS
SSMLGTNNKKEEEESEKQEQECVLFHEDGKKQGSDSTNDNYENDPPAETNDEDPTLILA
TDRRGRPPSRPKVGSGPPPQNN
SEQ ID NO: 23-PROSIPEP (PROREF1)-(PRR) Pepper DNA Sequence (CaPRR)
ATGGAAGAGGGTGCAGATACCAGAGAGAAAAGAGAGAGTGGCATAACAACAGGTTAT
GATGTTACAAAGAATCCAATGTTTTGCTTACAAGAAGCCATAAGAACTATTCTCAAATG
TCTTGGTTTTGAATCTTCAAAACCTGATCACCCTTCTTCTTCTTCTTCTTCGTCAAGTAT
GTCAAATGATACTAAAAAAGAAGAGGAGAATAATATTATAGAAGAACAAAGTGAACAA
CAAAAGTGTGTGTTCCAACAAGATGATGGTCAAGGGAATCAGAAACAGGATGATGAC
CCTCCAGCTGAAGTAGATGATCCACCACCAGTTATAGAAGCAGCAACAGGCCGGAGG
AGGGGAAGACCACCATCAAGACCTGGAGTTGGAAGGGGACCTCCTCCTGAGAACAA
TTAA
SEQ ID NO: 24-PROSIPEP (PROREF1) Pepper Amino Acid Sequence (CaPRR)
MEEGADTREKRESGITTGYDVTKNPMFCLQEAIRTILKCLGFESSKPDHPSSSSSSSSMS
NDTKKEEENNIIEEQSEQQKCVFQQDDGQGNQKQDDDPPAEVDDPPPVIEAATGRRRG
RPPSRPGVGRGPPPENN
SEQ ID NO: 25-PROSIPEP (PROREF1)-(PRR) Soybean DNA Sequence (GmPRR)
ATGATGGCTCTAGCTACTCCAGGTAGCCCTAGCTCCATGCACATGCCACTGGTGCAA
ACGTCCAACCCTAGCTCACAAAATTCTATGACGACAATTACCGAAAGGAGAGGAAGT
GTAGTAGTTCTACAAACGAAAAGAAAACATGATGGAGGAAAAGGACGTGATCCACAA
ACTAATAGCATAGGGTCACTCAAAAGCACAACAACCAAAAGGAGAGGGAGTGTTCTA
CAAACGAAAAGAAGACATGACGGAGGGAAAGGACGTGATCCGCAAACGAATAGCGT
AGGGCTACTCAAAAGCACAGAAAATGGTCCAAAAACTAATAGTGTAGGGCTACTCGA
AACCACAACAAATAATCGATCTTCAGAAAATTGA
SEQ ID NO: 26-PROSIPEP (PROREF1)-(PRR) Soybean Amino Acid Sequence
(GmPRR)
MMALATPGSPSSMHMPLVQTSNPSSQNSMTTITERRGSVVVLQTKRKHDGGKGRDPQT
NSIGSLKSTTTKRRGSVLQTKRRHDGGKGRDPQTNSVGLLKSTENGPKTNSVGLLETTT
NNRSSEN
SEQ ID NO: 27-PROSIPEP (PROREF1) Sunflower DNA Sequence (HanPRR)
ATGGTGGAGGAAAGGGCTGAAGTTGTGTATGATATTGGATATGGTTATGGTAACCCTT
GTAGGTATTTACAAGAAGTTTTTAGAAGCTTTTTAAGGTGTTTGGGGTTAGAGAAACAA
GAAGGAAAAGAAAGAAACACTAGTGGTGGTGTTGGTGGCCGCGACACAGGCGGTGG
TGACGGTGACGGTGACGGTGACGGTGACGGAGGTGGAGGTGAGGTGGATCCACCT
ACAACTTCTCCTATTATGGATCCTACTGATGAACCTATGTCTACAAGGAGACTCACAA
GAAGACCCCCTCCGCCAAGGGGGCCGATTAGCTCTGGAGGAGGCGGTCAAACCAAC
TAA
SEQ ID NO: 28-PROSIPEP (PROREF1) Sunflower Amino Acid Sequence (HanPRR)
MVEERAEVVYDIGYGYGNPCRYLQEVFRSFLRCLGLEKQEGKERNTSGGVGGRDTGGG
DGDGDGDGDGGGGEVDPPTTSPIMDPTDEPMSTRRLTRRPPPPRGPISSGGGGQTN
SEQ ID NO: 29-PROSIPEP (PROREF1)-(PRR) Cotton DNA Sequence (GaPRR)
ATGGAAGATACTTCATCAGCAAAGGAAGTAACGTTGCAAGGAACTCCAGCTAATTACT
TCCCCAGGTCCTTCCATGAAGTTGTAGGTGCCATTCTCAGGTGTTTGGGACTTGAAAC
TGGATTCCAACAAAACCCTAATCCATGTCCAAAGAAAGAAGATGACAGTAAAGCCAAT
CATAATCAATCTGTTTCTCAGAAGGAAAGTCCAGATCCACCTTCATCAACAGACAATT
CAGATCCATCAACCACTGTGATCGACCCACCAGCTGATCCTCCTCCTTCCACCACTG
GAGACACGAACGATGGCGAACTTCCCATGGTTTCTCTCTTTACTCCTAAAAGGCCAG
GGACAAGCGCCGGCAGTGGACCTCAGATTAATTAA
SEQ ID NO: 30-PROSIPEP (PROREF1)-(PRR) Cotton Amino Acid Sequence
(GaPRR)
MEDTSSAKEVTLQGTPANYFPRSFHEVVGAILRCLGLETGFQQNPNPCPKKEDDSKANH
NQSVSQKESPDPPSSTDNSDPSTTVIDPPADPPPSTTGDTNDGELPMVSLFTPKRPGTS
AGSGPQIN
SEQ ID NO: 31-PROSIPEP (PROREF1)-(PRR) Rice DNA Sequence (OsPRR)
ATGGCGATGTCGTCGTCTCCCGCCTCGCCGCCGCCATCCTTCCTGATCGGCGGCGC
CCAGGCCCAGCTCCTGCGCCACCGGGAGGAAATGCTGCTTGTTCTTCCCTCCCCTCC
CTCCGGCCGGCAGCTGCCATCGGAGGAGGAAGAAGCAGCACCATGCGCCGTCAATG
GCCGGAGTACGATCCTTGCCGCCGCCGATGATTCCAAGCCAACGCGGCCTGGAGCG
CCGGCGGAGGGCTCCGGCGGCAACGGCGGAGCAATCCACACCGCTGCTTCTTCCTG
A
SEQ ID NO: 32-PROSIPEP (PROREF1)-(PRR) Rice Amino Acid Sequence (OsPRR)
MAMSSSPASPPPSFLIGGAQAQLLRHREEMLLVLPSPPSGRQLPSEEEEAAPCAVNGRS
TILAAADDSKPTRPGAPAEGSGGNGGAIHTAASS
SEQ ID NO: 33-PROSIPEP (PROREF1)-(PRR) Maize DNA Sequence (ZmPRR)
ATGGATGAGCACGGGGAAAAGGAAGAGAACAAGTCTCAAGATTCGGCTTTGGCGGC
GGAGCAGCGCGAGGAGACGGCGGCGGCGGAGGGAGAGGACACGTCTGAGGAATC
CACGGACCAGCGCGAGGACGGGCACGGGTATAAAGCGGACGAATCGGCGGGCCTG
CTGCCCGAGGACGACGTGGGCTCTGGAGAAGCCTCGGCGGCGCCACACTTCGGGC
ACCCGTGCTCGTTGTTGCGCGCCTGCGCCGGATTCCTGGGCCTGCACGGCTGCGGC
GGCGATCAGAAGCCGGCGGCGGCTGCCGTTGCTGCATCTGCAGCTGAAGCCGCCAC
GGCGGCGGCGGCGAGCTCGTCCCAGGATGAAGAAGACGGCGGCGTCGACATGGCG
AAGGCTAACGGTTTCTACATGCATGAGGTGATCACCCGCGTATGGGCGGTCGCGGT
GAGGAGGCCGCGGCCGCGGCCGCCGGATCACGCGAGAGAAGGGAGCGGTGGCAA
TGGAGGCGTACACCACTAG
SEQ ID NO: 34-PROSIPEP (PROREF1)-(PRR) Maize Amino Acid Sequence (ZmPRR)
MDEHGEKEENKSQDSALAAEQREETAAAEGEDTSEESTDQREDGHGYKADESAGLLPE
DDVGSGEASAAPHFGHPCSLLRACAGFLGLHGCGGDQKPAAAAVAASAAEAATAAAAS
SSQDEEDGGVDMAKANGFYMHEVITRVWAVAVRRPRPRPPDHAREGSGGNGGVHH
SEQ ID NO: 35-PROSIPEP (PROREF1)-(PRR) Wheat DNA Sequence (TaePRR)
ATGGACCAGCACCAGGAGCTGGAGGTGGATGTGGAGGGAGAGCCAACGGATCAGC
GCAAGGAGGAGGAGGCGGACGCGGACGCGGGCAAGCCTGAAGAGCCGACGGATCA
GCGCAAGGAGGAGGAGGAGGAGCGGGAGGAGGGAGACAACCTCAAAGAGCCAACG
GATCAGCGCAAGGAGGACAAGCCTGACGGGCTGACGGAGCAGCACAAGGCGGAGG
CGGTGGCGGTTGCGGTGGTGGTGGCGGAGGTGGACAAGTCTGAAGAGCTGACGGA
CCAGCGCAAGGCGGGGGCGGTGGTGGAGGTGGAGGTGGACAAGTTTGAAGAGCTC
ACCGACCAGCGCAAGGAGGAGGCGGCGGAGGACAAGGACAAGGAGACGGAGAGGA
GCTCCAGCGAAGAGTCGCCGTCGCTCTTCGTGCACCCGTGCTCGCTGCTGCTGCGG
TACCTCGTCCGTGCCTACGCCTGGTGCATGGGCATGGCCGACTGGTTCGGCGGCGG
CGGCACAAGGCCGAGCGCCGCTCCTGCCGCGTCCCTTAACTCGTCACGGGAAGAAG
CAGGCGAGGCGGCGGGCGTCAGGACCAGGGAGATCTCCAGCTCCAAGACCACTGA
CAGAGGGTTCTACGTGCGGGAGATGATCGTGAGCATGTGGGCGGTGAGGAGGCCG
CGGCCGCCCGGCAACCCGAGGGAAGGGCGCGGCGGCGGTGGAGGAAGCCACAAC
TAG
SEQ ID NO: 36-PROSIPEP (PROREF1)-(PRR) Wheat Amino Acid Sequence
(TaePRR)
MDQHQELEVDVEGEPTDQRKEEEADADAGKPEEPTDQRKEEEEREEGDNLKEPTDQRK
EDKPDGLTEQHKAEAVAVVVVAEVDKSEELTDQRKAGAVVEVEVDKFEELTDQRKEEAA
EDKDKETERSSSEESPSLFVHPCSLLLRYLVRAYAWCMGMADWFGGGGTRPSAAPAAS
LNSSREEAGEAAGVRTREISSSKTTDRGFYVREMIVSMWAVRRPRPPGNPREGRGGGG
GSHN
SEQ ID NO: 37-PROSIPEP (PROREF1)-(PRR) Cucumber DNA Sequence (CsaPRR)
ATGGCTTCCAACAGGGTTTCGATGATTTTGTATGTGCTGTTCTTCATTATTATGCTTTC
AATTTCATCTAATAATAATTATAATGCTTCTGCAAGAAAATTTCTTGACGTTAATTTCCC
TCTTCTTGACGATGCCTTCGACCTTCAAATGGGTGCATCTTCATTATTTTATAAATTGG
AAGGAGAGCTACAAAAGAGATTCGAGGATTTATTAAAAACAACCACGGATAATGATAT
TCCTCGTGGTGGAAACTATTAA
SEQ ID NO: 38-PROSIPEP (PROREF1)-(PRR) Cucumber Amino Acid Sequence
(CsaPRR)
MASNRVSMILYVLFFIIMLSISSNNNYNASARKFLDVNFPLLDDAFDLQMGASSLFYKLEGE
LQKRFEDLLKTTTDNDIPRGGNY
SEQ ID NO: 39-PROSIPEP (PROREF1)-(PRR) Peach DNA Sequence (PpPRR)
ATGGAGAAGGCATCATCAGGAAAGGAGGTCATCATCACTACTGAGAAGATGGTTGCT
GTTTCTTATGATCGTTACCAATTCCTTGGAGCAGCTGTTAGGTCTCTTTTCAAGTGTTT
GGGTGTTGGGGATTCTTCAACTTCAACCACACAACAAGTGCTTACAGATTTTTCGGTT
GAAGGGACACCAGCCATGTCAGCTACGGCAGCAGAGCAGGTTGTTCCACCGCCAGC
GGCAGTAGTCGCCCCTGAAGCTGACCCTGCTGCTGCTTCTAACGCCCCTAAAAGACC
GAGAATTACTCTTCGTGCGGCAAGGCCAGAAATAAGCACAGGGAGTGGTGCTCAGAC
TAATTGA
SEQ ID NO: 40-PROSIPEP (PROREF1)-(PRR) Peach Amino Acid Sequence (PpPRR)
MEKASSGKEVIITTEKMVAVSYDRYQFLGAAVRSLFKCLGVGDSSTSTTQQVLTDFSVEG
TPAMSATAAEQVVPPPAAVVAPEADPAAASNAPKRPRITLRAARPEISTGSGAQTN*
SEQ ID NO: 41-SIPEPR1 (RER) Tomato DNA Sequence (SiRER)
ATGAAGATAGCTGTTCATAATTTGATCTTTTTCTACTGCTGCTATTTCTCAGTGTCGGC
TTTTGCTGTATGTGGTCTCACTTCTGATGGCACCGCTTTAGTTTCCCTTTCTAGTGACT
GGATTGGTGTACCCTCTTCTTGGAATGCTTCAGATACCAATCCATGTTCTTGGGTTGG
CGTTGAGTGTGATGACAACCACTTTGTCACCTCTTTGAACCTCTCTGGCTATGATATC
TCTGGCCAATTGGGACCTGAAATTGCTTATTTGAAACACTTGCTTACCATGGACTTGA
GTTATAATGCTTTCTCTGCCTCCATTCCTTCTCAACTCACTAACTGCACTCTTCTTCGT
TACTTGGATCTATCCTACAACACCTTTACTGGAGAAATCCCTTCAAACATTGGAAATTT
ACACAAGTTAACCTACATAAGCCTCTTCTCTAATTCACTCACTGGTAATATTCCTCATT
CCTTGTTTTCTATCCCACATTTGGAAACTATCTACTTTAACCAAAACAGTTTGAATGGT
TCCATCCCGTCCGGCATTGCTAACTTGACTCACCTGTTGTCTCTCTATCTTTATCAGAA
TGACTTGTCCGGTCCAATTCCCTCTTCCATAGGAAACTGTACAAACTTGCAAGAATTG
TATTTGAATGATAATCATTTAGTTGGTAGTTTGCCTGAGAGTTTACAAAAGCTACAACA
CCTTGTTTATTTGGACTTAAGCAATAATAGTCTACAAGGAAGTATCCCTTTCAGTTTAG
GCAACTACAAACATCTCGACACTCTGGTCTTGTCATCCAATAGTTTCAATGGGGAACT
TCCACCAACTTTGATGAATTCTACGAACCTAAAAGTTCTTGCTGCTTTTTCTTCGGGTT
TAAGTGGCCCTATTCCTGCTACTCTAGGCCAGCTCACAAAGTTGGAGAAACTTTACCT
TACTGACAACAATTTCTCTGGAAAAATACCACCTGAGCTGGGAAAGTGCCAGGCCTT
GATGGAATTACATTTACCCGGAAACCAACTGGAAGGTGAAATTCCTAGTGAACTAGGA
TCACTCACCCAGTTGCAGTATCTCTCTCTATATTCCAACAAATTGAGTGGTGAGATTCC
CCCCACTATTTGGAAGATTCAAAGTCTTCAACATATTCTTGTCTATCGGAACAACCTGA
CTGGGGAGTTACCTCTTGAAATGACTGAGTTGAAACAATTGAAGAACATATCCTTATTT
GAGAACCAGTTTACCGGAGTTATACCTCAAGGTTTGGGGATTAACAGCAGTCTAACG
CTGCTGGATTTCACAAACAACACCTTCACAGGTCCTGTTCCACCTAATCTTTGTTTTGG
AAAGAAACTGGAGAAGCTTCTTTTAGGTTATAATCATCTTGAAGGTGGCATTCCTTCTC
AATTAGGACAATGTCATACTTTGACGAGAGTAATTCTCAAAAAAAATAATCTGTCAGGT
GCCATTCCAGATTTTGTTAAGAATATAAATCCTATTTTCTTGGATCTCAGTGAGAATGG
CTTCAGTGGGAAAATATCTCCAAGTTTGGCAAATCTTGAAAATGCTACATCAATTGACT
TATCAGTGAATAAGCTTTCAGGATTTATGCCACCAGAGCTGGCAAACCTTGCCAACCT
CCAGGGTTTAAATCTGTCGTACAATGGTTTGGAAGGTGTACTGCCATCTCAACTTTCA
AACTGGCAGAGACTGTTGAAGTTTGATGCAAGTCATAATTTGCTGAGTGGATCGATCC
CATCCGCGTTTGGAAGCTTGGAGGAGCTATCCATTTTGAGTCTGTGTGAGAATAATCT
TTCAGGAGGTATTCCCACCTCCTTGTTTGCACTCAAGAAGCTGTCCAAGCTGCAGCTT
GGTGGAAATGCACTTGGTGGGGAAATTCATTCAGCGATTGCGACTGCATCAAGAGAA
ACACTGAGGTGTTTAAATCTGAGTAGTAACAGGTTGACAGGTGAACTTCCTGCGGAG
CTAGGAAAATTTACTTTCCTGGAGGAGTTAGATATAGCTGGCAATAACATTTCTGGAA
CTTTAAGAGTTCTTGATGGAATGCATTCATTGTTATTCATCAATGTATCAGACAACCTC
TTTTCTGGTCCAGTACCTGCACATCTGATGAAGTTCCTCAACTCAACTCCTACCTCTTT
CTCGGGAAACTTGGGCCTTTGTGTGCACTGTGATCCAGAAGAAGGCTCAAATTGCCC
TGAGAATATCACGTTAAGGCCCTGTGATCTTCAATCAAACAATGGAAGGCACCTAAGT
GTAGCAGAAACGGCAATGATTGCACTTGGGGCACTGATATTTACCATTTCCTTACTCC
TAGTAATTGCTTACATGCTTCTGTGGCGCAAAAGTTCTGGGAAAGGAGTTGCAATCTC
TGCTCAAGAAGGTGCATCGTCCCTGCTTAATAAAGTATTGGAAGCTACTGGAAACCTA
AACGATAAGTATGTCATTGGGAGGGGAGCTCATGGAGTTGTATATAAGGCCATCTTG
GGTCCAGGGAAAGTGTATGCTGTGAAAAAGCTGGTGTTTGTTGGTATGAAGGATGGA
AGCAGAAGTATGGTTAGAGAAATTCAAACAATTGGAAAGGTTAGGCACCGCAATCTTG
TCAAATTAGAAGACTTCTGGCTGAGAAAGGATTATGGGTTGATTCTTTATAATTACATG
GAGAACGGGAGCCTTCATGATATTCTCCATGAGACTAAGCCACCTGTAACATTAGAAT
GGAGCGTTCGGTACCAAATTGCTATTGGAGTTGCTCAAGGGTTGTCATATCTCCATTT
CGACTGTGATCCTGCCATCGTGCATCGAGACATCAAGCCCATGAATATCTTGTTGGAC
TCTGATCTGGAGCCTCACATATCAGATTTTGGTATTGCCAAGCTTCTGGATCAGTCTG
CAGCAACTTCCGCCTCCAATGCCCTTCAGGGCACAGTTGGGTACATGGCTCCAGAAA
CTGCATTTGCAGCTACAAAGAGCAAGGAGTCGGATGTTTATAGTTATGGCATTGTCCT
ATTAGAACTTATAACTCGAAAGAAGGTGTTGGACCGTTCACTCTATGGGGAAACAGAT
ATTGTATGTTGGGTTAGGTCTGTTTGGACAGAAACTGAAGAAATTGAGAAGATTGTGG
ATCCAAGACTTTTGGACGAATTCATAGACTCAAGTGTCATGGAACAAGTAATTGAAGT
GCTTTCACTAGCTCTTAGATGTACAGAGAAGGAAGTCAGCAAAAGACCCTCAATGAAA
GAGGTAGTCAAGCTATTAACAAGGTCAAGTTCGAGTATACGAAGCAAGTACTAG
SEQ ID NO: 42-SIPEPR1 (RER) Tomato Amino Acid Sequence (SiRER)
MKIAVHNLIFFYCCYFSVSAFAVCGLTSDGTALVSLSSDWIGVPSSWNASDTNPCSWVGV
ECDDNHFVTSLNLSGYDISGQLGPEIAYLKHLLTMDLSYNAFSASIPSQLTNCTLLRYLDLS
YNTFTGEIPSNIGNLHKLTYISLFSNSLTGNIPHSLFSIPHLETIYFNQNSLNGSIPSGIANLT
HLLSLYLYQNDLSGPIPSSIGNCTNLQELYLNDNHLVGSLPESLQKLQHLVYLDLSNNSLQ
GSIPFSLGNYKHLDTLVLSSNSFNGELPPTLMNSTNLKVLAAFSSGLSGPIPATLGQLTKLE
KLYLTDNNFSGKIPPELGKCQALMELHLPGNQLEGEIPSELGSLTQLQYLSLYSNKLSGEI
PPTIWKIQSLQHILVYRNNLTGELPLEMTELKQLKNISLFENQFTGVIPQGLGINSSLTLLDF
TNNTFTGPVPPNLCFGKKLEKLLLGYNHLEGGIPSQLGQCHTLTRVILKKNNLSGAIPDFV
KNINPIFLDLSENGFSGKISPSLANLENATSIDLSVNKLSGFMPPELANLANLQGLNLSYNG
LEGVLPSQLSNWQRLLKFDASHNLLSGSIPSAFGSLEELSILSLCENNLSGGIPTSLFALKK
LSKLQLGGNALGGEIHSAIATASRETLRCLNLSSNRLTGELPAELGKFTFLEELDIAGNNIS
GTLRVLDGMHSLLFINVSDNLFSGPVPAHLMKFLNSTPTSFSGNLGLCVHCDPEEGSNCP
ENITLRPCDLQSNNGRHLSVAETAMIALGALIFTISLLLVIAYMLLWRKSSGKGVAISAQEG
ASSLLNKVLEATGNLNDKYVIGRGAHGVVYKAILGPGKVYAVKKLVFVGMKDGSRSMVR
EIQTIGKVRHRNLVKLEDFWLRKDYGLILYNYMENGSLHDILHETKPPVTLEWSVRYQIAIG
VAQGLSYLHFDCDPAIVHRDIKPMNILLDSDLEPHISDFGIAKLLDQSAATSASNALQGTVG
YMAPETAFAATKSKESDVYSYGIVLLELITRKKVLDRSLYGETDIVCWVRSVWTETEEIEKI
VDPRLLDEFIDSSVMEQVIEVLSLALRCTEKEVSKRPSMKEVVKLLTRSSSSIRSKY
SEQ ID NO: 43-SIPEPR1 (RER) Pepper DNA Sequence (CaRER)
ATGAAGATTGATGTCAAAAATGTCATCTTTTTCTACCTTTGCTGCTTCTCTGTGTCGGT
TTGTGCTCTCAATTCCCATGGCACAGTTCTACTTTCCCTTTTCAGGCACTGGACTGCT
GTGCCCTCCTCTGTTAAGTCTTCTTGGAATGCTTCAGATTCCACTCCCTGTTCTTGGG
TTGGTGTGGAATGTGATACCACTCACCTTGTCACTTCTTTGAACCTCTCTGGCTATGG
AATCTCTGGCCAATTGGGTCCTGAAATTGCTTATTTGGAGCACTTGCGTACCATTGAT
CTGAGCTATAATGCTTTCTTTGGCCCTATTCCTTCTCAACTCGTAAATTGCACTCTTCT
TGATTACTTGGACCTGTCCTACAACACCTTTACTGGAGAAATCCCTTCAAAGATTGGA
AATTTACACAAGTTGACCTACATAAGCCTCTACGCTAATTCATTGACTGGTAATATTCC
TGATTCCTTGTTTTCTATCCCACATTTGGACTCCATTTACCTTTTCCAAAACAGGTTAAA
TGGTTCCATCCCATCTCGCATTGGCAACTTGACTAAGCTTGTGTCTCTCTTTCTTTATG
ACAATGAGTTGTTCGGTCCAATTCCGTCTTCCATTAGTAACTGTACTAACTTGCAAGAA
CTGTATTTGAATGAAAACAATGTAGTTGGTAGTTTGCCTGAGAATTTAGATAAGCTAGA
ACACCTTGTTTATTTGGACTTAAGCAGTAATAGACTACAAGGAAGTATCCCTTTCAGTT
TAGGTGGGAACTGCAAAGATCTTGACACTTTGGTCTTGTCATCCAACAATTTAAATGG
AAATCTTCCACCAAGTTTGAGCAATTGTACTAACCTACGAGTTCTTGCTGCTTTCTCTT
CTAGTTTAAGTGGCCCTATACCTGCTTCTCTAGGCCAGCTCACAAAGTTAGAGAAGCT
TTACCTTGCTGACAACAATTTCTCTGGAAAAATACCACCTGAGCTGGGGAAGTGCCAG
TCCTTGCTGGAATTACTTTTACCAGAAAACCAACTGGAAGGTGAAATTCCTAGTGAAC
TAGGGTCACTCAGCCAGTTGCAGTATCTCGCTCTATATTCCAACAAATTGAGCGGCGA
GATTCCCCGTGCCATTTGGAAGATTCAAAGTCTTCAACATTTTCTTGTCTATCGGAACA
ACCTGACCGGGGAGGTACCTCTTGAAATGACCGAGTTGAAACAACTGAAGAACATAT
CTTTATTTGACAACCGGTTCACTGGAGTTATACCTCAAGGTTTGGGGATTAACAGCAG
TCTAACGCTGCTGGATTTCACGAACAACGCCTTCACAGGTCCTGTTCCACCTAATCTT
TGTTTTGGCAAGAAACTACAGAAACTTATGCTAGGTTATAATCATTTTGAAGGTGGCAT
TCCTTCTCAATTAGGTAAGTGTGCTACTTTGGCGAGAGTAATTCTCAAAAAGAATAAAC
TCTCAGGTGCCATTCCAGATTTTGGTAAGAATATAAATCCTATTTTCTTGGATCTCAGT
GAAAATGGCTTCAGTGGGAGAATACCCCCAAGTTTGGCAAATCTTGGAAATGTTACAT
TAATTGACTTATCAGTGAATAAGCTCTCGGGGTTTATACCACCAGAGCTGGCAAACCT
TGTCAACCTCCAGGTTCTTGATTTGTCGTACAATAGTTTGGAAGGTGTACTGCCATCT
CAACTTTCAAACTGGCAGAGACTGTTGCAGTTTGATGCAAGCCATAATTTGTTGAGTG
GATCGATCCCATCCACGTTTGGAAGCTTGGGAGAGCTATCCATTTTGAGTCTGAGTGA
GAATAATCTTTCAGGAGGTATTCCAACCTCCTTGTTTGAACTCAAGAAGCTGTCTGAG
CTGCAGCTTGGCGGAAATGCACTTGGTGGTGAAATTCATTCAGCAATTGCGACTTCAT
CAAGAGAAACACTGAGGTTGTTGAATCTGAGTAGTAACGGGTTGACAGGTGAAATTC
CTGCAGAGCTAGGGAAATTTACTTTCCTGGAGAAGTTAGATGTAGCTGGAAACAATAT
TACTGGAACTTTAAGAATTTTTGAGGGGATGCGTTCATTGATCTTCGTCAATGTGTCAC
ACAACCTCTTTTCTGGTCCAGTACCTGCAAATTTGATGAAGTTCCTAAACTCAACACCT
ACCTCTTTCTCGGGAAACTTTGGCCTTTGCGTGCACTGTGATCCAGAAAAAGGCTCAA
ATTGCCCTGAGAATGGCACTCTAAGGCCTTGTGATCTCCAATCAAAAAATGGAAGACA
CCTAAGTGGAGCAGAAACGACAATGATTGCACTTGGGGTACTGTTATTTACCATTTGC
TTACTCCTAGTAATTGCTTACATGCTTCTGTGGCGCAAAAATTCTGGGAAAGGAGTTG
CAATCTGTGCTCAAGAAGGTGCTTCATCCCTGCTTAATAAAGTATTGGAAGCTACAGA
GAACCTAAATGATAAGTATGTCATTGGGAGGGGAGCACATGGAATTGTATTTAAGGCC
ATCTTGGGTCCAGGGAAAGTGTATGCCGTGAAGAAGCTGGTGTTTGTTGGTATAAAG
GATGGAAGCACAAGTATGGTTAGGGAAATTCAGACAATTGGAAAGGTTAGACACCGC
AATCTTGTCAAATTAGAAGACTTCTGGCTGAGAAAGGATTATGGACTGATTCTTTATAA
CTACATGGAGAACGGGAGCCTTCATGATATTCTCCATGAGATTAATCCACCTGTAGCA
TTAGAATGGAGTGTTCGGTACCGAATTGCTATTGGAACTGCTCAAGGGTTGTCATATC
TCCACTTTGACTGTGATCCTGCAATCGTTCATCGAGATATCAAGCCCATGAATATCTT
GTTGGACTCAGATCTGGAGCCTCACATATCTGATTTTGGCATTGCCAAGCTTCTTGAT
CAGTCTGGAGCAACTTCCACCTCCAATACCCTCCAGGGTACAGTTGGATACATGGCT
CCAGAAACTGCATTTGCAGCTTCAAAGAGCAAGGAGTCAGATGTTTATAGTTATGGTG
TTGTCCTGTTAGAACTTATAACTCGAAAGAAGGCCTTGGATCCTTCATTGTATGGGGA
TACAGATATTGTCAGTTGGGTTAGGTCTATTTGGACAGAAATTGAAGAAATTGAGGAG
ATTGTGGATCCAAGCCTTTTGGACGAATTCATGGACTCAAGTGTCATGGAACAAGTAA
TTGAAGTGCTTTCACTAGCTCTTAGATGTACAGAGAAGGATGTAAGCAGAAGACCCTC
TATGAAAGAGGTGGTGAAGCTATTAACAAGGTCCAGTTCGAGTATAAGAATCAAATAC
TAG
SEQ ID NO: 44-SIPEPR1 (RER) Pepper Amino Acid Sequence (CaRER)
MKIDVKNVIFFYLCCFSVSVCALNSHGTVLLSLFRHWTAVPSSVKSSWNASDSTPCSWV
GVECDTTHLVTSLNLSGYGISGQLGPEIAYLEHLRTIDLSYNAFFGPIPSQLVNCTLLDYLD
LSYNTFTGEIPSKIGNLHKLTYISLYANSLTGNIPDSLFSIPHLDSIYLFQNRLNGSIPSRIGNL
TKLVSLFLYDNELFGPIPSSISNCTNLQELYLNENNVVGSLPENLDKLEHLVYLDLSSNRLQ
GSIPFSLGGNCKDLDTLVLSSNNLNGNLPPSLSNCTNLRVLAAFSSSLSGPIPASLGQLTK
LEKLYLADNNFSGKIPPELGKCQSLLELLLPENQLEGEIPSELGSLSQLQYLALYSNKLSGE
IPRAIWKIQSLQHFLVYRNNLTGEVPLEMTELKQLKNISLFDNRFTGVIPQGLGINSSLTLLD
FTNNAFTGPVPPNLCFGKKLQKLMLGYNHFEGGIPSQLGKCATLARVILKKNKLSGAIPDF
GKNINPIFLDLSENGFSGRIPPSLANLGNVTLIDLSVNKLSGFIPPELANLVNLQVLDLSYNS
LEGVLPSQLSNWQRLLQFDASHNLLSGSIPSTFGSLGELSILSLSENNLSGGIPTSLFELKK
LSELQLGGNALGGEIHSAIATSSRETLRLLNLSSNGLTGEIPAELGKFTFLEKLDVAGNNIT
GTLRIFEGMRSLIFVNVSHNLFSGPVPANLMKFLNSTPTSFSGNFGLCVHCDPEKGSNCP
ENGTLRPCDLQSKNGRHLSGAETTMIALGVLLFTICLLLVIAYMLLWRKNSGKGVAICAQE
GASSLLNKVLEATENLNDKYVIGRGAHGIVFKAILGPGKVYAVKKLVFVGIKDGSTSMVREI
QTIGKVRHRNLVKLEDFWLRKDYGLILYNYMENGSLHDILHEINPPVALEWSVRYRIAIGTA
QGLSYLHFDCDPAIVHRDIKPMNILLDSDLEPHISDFGIAKLLDQSGATSTSNTLQGTVGY
MAPETAFAASKSKESDVYSYGVVLLELITRKKALDPSLYGDTDIVSWVRSIWTEIEEIEEIV
DPSLLDEFMDSSVMEQVIEVLSLALRCTEKDVSRRPSMKEVVKLLTRSSSSIRIKY
SEQ ID NO: 45-SIPEPR1 (RER) Soybean DNA Sequence (GmRER)
ATGGGGTATCTGTATCTCTTGCTGCTTCTATGTTTTTCTTCCTTGTTATATGCTGCTTCT
GCATTGAACTCTGATGGTTTGGCTTTGTTGTCCCTCTTGAGGGATTGGACTACTGTGC
CTAGTGACATAAACTCCACATGGAGGTTGTCTGATTCCACTCCATGCTCATCTTGGGC
AGGAGTGCATTGTGATAATGCCAACAATGTGGTTTCTCTAAACCTCACTAGTTATTCG
ATTTTGGGTCAATTAGGACCTGATCTTGGACGTTTGGTTCACTTGCAAACCATAGACT
TATCATATAATGATTTCTTTGGGAAAATCCCCCCAGAATTAGAGAACTGTAGCATGCTT
GAGTACTTGAACCTTTCTGTAAACAACTTTAGCGGAGGAATACCTGAGAGCTTCAAAA
GCTTGCAAAATTTGAAGCATATATACCTTTTATCTAATCACCTTAATGGTGAAATTCCT
GAATCCTTGTTTGAAATTTCTCACCTGGAAGAAGTGGATCTTAGCAGAAACAGTTTAA
CTGGTTCAATCCCCTTAAGTGTTGGGAATATCACTAAGCTTGTCACTCTGGATCTTTCT
TATAATCAGTTGTCAGGGACAATTCCAATATCCATTGGAAATTGTAGTAACTTAGAGAA
TCTGTATTTGGAAAGGAATCAATTAGAGGGAGTTATTCCTGAGAGTCTAAATAATCTCA
AAAATCTTCAAGAGTTATATCTCAATTATAATAACCTTGGAGGTACTGTTCAATTGGGA
TCTGGATATTGCAAAAAGTTGTCTATTTTGAGTATTTCTTACAATAACTTTAGTGGGGG
TATACCATCAAGCTTGGGGAATTGTAGTGGTCTAATAGAGTTTTATGCTTCAGGGAAT
AACTTAGTTGGCACTATACCATCAACCTTCGGCCTCCTGCCCAACCTTTCTATGTTATT
CATTCCGGAGAACCTATTGTCAGGGAAAATACCTCCACAGATTGGTAATTGCAAATCA
CTGAAAGAGTTGAGTTTGAATTCCAATCAACTTGAAGGAGAAATTCCAAGCGAATTAG
GAAACTTGAGTAAATTGCGTGATCTTAGATTGTTTGAAAACCATTTGACAGGAGAAATT
CCACTTGGCATATGGAAAATTCAAAGCCTTGAGCAGATCCATATGTACATTAATAACC
TCTCGGGCGAGCTACCACTTGAGATGACAGAGCTTAAACATCTTAAGAATGTCTCCTT
GTTTAACAACCAGTTCTCCGGAGTCATACCTCAAAGCTTAGGAATCAATAGCAGTTTG
GTGGTGTTAGATTTTATGTATAATAATTTCACTGGTACCCTCCCACCAAATCTTTGTTTT
GGAAAGCACCTGGTCAGGCTGAATATGGGTGGCAATCAATTTATTGGCAGCATACCT
CCTGATGTAGGAAGGTGTACAACTCTTACAAGGTTGAGACTTGAAGATAATAATTTAA
CTGGGGCACTTCCTGATTTTGAAACTAATCCAAACCTCTCTTACATGAGCATCAACAA
CAACAATATCAGTGGAGCAATTCCATCAAGTTTGGGAAACTGCACAAATCTCTCTCTTT
TAGATTTGTCCATGAACAGCTTGACGGGTCTTGTACCTTCAGAGCTAGGAAACCTTGT
GAATCTTCAGACTTTGGATCTTTCTCACAATAACTTGCAAGGTCCTTTGCCACATCAGC
TGTCAAACTGTGCCAAAATGATCAAGTTTAATGTCGGATTCAATTCTTTGAATGGTTCG
GTTCCTTCAAGTTTTCAGAGCTGGACAACATTAACAACTTTAATTCTCTCAGAGAATCG
TTTTAATGGTGGTATTCCAGCTTTCTTGTCAGAATTTAAAAAGCTCAACGAGTTACGAC
TTGGTGGAAATACGTTTGGAGGAAACATTCCTAGATCAATTGGAGAATTGGTGAATTT
GATATATGAGCTAAATTTAAGTGCTAATGGGCTGATAGGTGAACTTCCTAGGGAGATT
GGAAACCTGAAGAATCTGCTAAGCCTGGATCTATCTTGGAACAATTTGACAGGAAGTA
TACAAGTTCTTGATGAGCTCAGTTCATTATCTGAATTCAACATCTCATTTAATTCTTTTG
AAGGTCCTGTGCCACAACAGCTAACAACATTACCAAATTCTTCTTTATCATTTTTGGGC
AATCCTGGTCTGTGTGACTCGAATTTCACTGTGAGCAGCTATTTACAGCCATGTAGCA
CAAATTCAAAAAAGTCAAAAAAGCTGAGTAAAGTTGAAGCTGTGATGATAGCACTTGG
ATCCTTAGTATTTGTTGTTCTGCTGCTGGGGTTAATCTGTATATTCTTTATCAGAAAAAT
TAAGCAGGAAGCCATAATCATTGAGGAGGATGATTTTCCAACACTTCTTAATGAAGTA
ATGGAAGCGACAGAAAATCTAAATGATCAATATATTATTGGCAGAGGAGCTCAAGGAG
TGGTTTACAAAGCAGCAATAGGTCCAGACAAAATTTTGGCTATAAAGAAATTTGTATTT
GCTCATGATGAAGGGAAAAGCTCAAGCATGACCAGAGAAATTCAAACCATTGGAAAG
ATTAGGCATCGAAATTTGGTCAAATTGGAAGGGTGCTGGTTGAGAGAAAACTATGGTC
TAATTGCATACAAATACATGCCAAATGGAAGCCTTCATGGTGCTTTGCATGAGAGGAA
TCCACCATACTCCTTAGAATGGAATGTCCGGAATAGGATAGCTCTTGGAATTGCTCAT
GGATTGGCTTATCTCCATTATGACTGTGATCCTGTCATAGTGCACAGAGACATCAAAA
CCAGCAATATACTTCTAGATTCTGACATGGAGCCTCATATTGCAGATTTTGGTATTTCC
AAGCTTTTAGATCAGCCTTCTACCTCAACACAGTCGTCATCTGTTACTGGTACACTTG
GATATATAGCACCAGAGAAATCCTATACAACAACAAAGGGTAAGGAATCTGATGTATA
CAGTTATGGGGTAGTTTTGCTGGAACTGATATCCAGAAAGAAGCCATTGGATGCATCA
TTTATGGAAGGGACGGATATAGTTAATTGGGCTAGATCTGTCTGGGAGGAAACAGGA
GTTATTGACGAAATTGTTGATCCAGAGATGGCTGATGAAATTTCAAATTCTGATGTGAT
GAAACAAGTTGCCAAGGTGCTTTTGGTGGCTTTGAGATGCACATTAAAGGATCCACG
CAAGAGACCTACGATGAGGGATGTTATCAAGCATTTGTAG
SEQ ID NO: 46-SIPEPR1 (RER) Soybean Amino Acid Sequence (GmRER)
MGYLYLLLLLCFSSLLYAASALNSDGLALLSLLRDWTTVPSDINSTWRLSDSTPCSSWAG
VHCDNANNVVSLNLTSYSILGQLGPDLGRLVHLQTIDLSYNDFFGKIPPELENCSMLEYLN
LSVNNFSGGIPESFKSLQNLKHIYLLSNHLNGEIPESLFEISHLEEVDLSRNSLTGSIPLSVG
NITKLVTLDLSYNQLSGTIPISIGNCSNLENLYLERNQLEGVIPESLNNLKNLQELYLNYNNL
GGTVQLGSGYCKKLSILSISYNNFSGGIPSSLGNCSGLIEFYASGNNLVGTIPSTFGLLPNL
SMLFIPENLLSGKIPPQIGNCKSLKELSLNSNQLEGEIPSELGNLSKLRDLRLFENHLTGEIP
LGIWKIQSLEQIHMYINNLSGELPLEMTELKHLKNVSLFNNQFSGVIPQSLGINSSLVVLDF
MYNNFTGTLPPNLCFGKHLVRLNMGGNQFIGSIPPDVGRCTTLTRLRLEDNNLTGALPDF
ETNPNLSYMSINNNNISGAIPSSLGNCTNLSLLDLSMNSLTGLVPSELGNLVNLQTLDLSH
NNLQGPLPHQLSNCAKMIKFNVGFNSLNGSVPSSFQSWTTLTTLILSENRFNGGIPAFLSE
FKKLNELRLGGNTFGGNIPRSIGELVNLIYELNLSANGLIGELPREIGNLKNLLSLDLSWNN
LTGSIQVLDELSSLSEFNISFNSFEGPVPQQLTTLPNSSLSFLGNPGLCDSNFTVSSYLQP
CSTNSKKSKKLSKVEAVMIALGSLVFVVLLLGLICIFFIRKIKQEAIIIEEDDFPTLLNEVMEAT
ENLNDQYIIGRGAQGVVYKAAIGPDKILAIKKFVFAHDEGKSSSMTREIQTIGKIRHRNLVKL
EGCWLRENYGLIAYKYMPNGSLHGALHERNPPYSLEWNVRNRIALGIAHGLAYLHYDCD
PVIVHRDIKTSNILLDSDMEPHIADFGISKLLDQPSTSTQSSSVTGTLGYIAPEKSYTTTKGK
ESDVYSYGVVLLELISRKKPLDASFMEGTDIVNWARSVWEETGVIDEIVDPEMADEISNSD
VMKQVAKVLLVALRCTLKDPRKRPTMRDVIKHL
SEQ ID NO: 47-SIPEPR1 LIKE1 (RER-LIKE1) Soybean DNA Sequence (GmRER-
LIKE1)
ATGTCCATGATTTGGATTGTTTTCTTTTCCTTGTCTTGCATGTCTTGTGCTGTTGTTTCT
TCACTCACCTCCGATGGGGTGACTCTCTTGTCACTCTTGAGGCACTGGACATCCGTG
CCTCCTTCCATAAACGCCACCTGGCTTGCCTCCGATACCACTCCATGCTCCTCCTGG
GTAGGAGTACAATGTGACCATTCCCACCATGTCGTCAACCTTACCCTCCCAGATTATG
GTATTGCTGGTCAATTAGGACCTGAAATTGGAAATTTAAGTCGCCTAGAGTACTTAGA
ACTTGCTAGCAACAACCTTACTGGTCAAATACCTGACGCCTTCAAAAACATGCACAAC
CTCAATTTACTCAGCCTTCCATATAATCAACTGTCTGGTGAAATTCCAGATTCCTTGAC
TCATGCTCCCCAACTAAATCTTGTTGATCTTTCTCATAACACTTTAAGTGGATCCATCC
CCACAAGTATTGGGAACATGACTCAGCTCTTGCAGTTGTATCTTCAGAGTAACCAGTT
GTCTGGGACAATTCCCTCATCCATTGGGAACTGCAGCAAATTACAAGAATTGTTTTTG
GATAAAAATCACTTGGAGGGTATCCTGCCTCAGAGTCTTAACAATCTCAATGATCTTG
CTTATTTTGATGTTGCCAGCAATAGACTTAAGGGTACCATTCCTTTTGGTTCTGCTGCC
AGTTGTAAAAATTTGAAGAATTTGGATCTCTCATTCAATGACTTTAGTGGAGGCCTTCC
CTCAAGCTTGGGGAACTGTAGCGCTTTATCTGAATTTTCTGCCGTGAACTGCAATTTG
GATGGCAATATTCCTCCCTCCTTTGGTCTACTGACCAAGCTTTCTATTCTATACCTTCC
CGAGAACCACTTATCTGGAAAAGTACCTCCAGAAATTGGCAACTGCATGTCTTTGACA
GAGTTACATCTGTATTCCAATCAACTTGAGGGAAACATTCCAAGTGAACTGGGAAAAC
TAAGAAAACTAGTGGATCTTGAATTGTTTTCAAATCAATTGACGGGTGAAATTCCACTC
AGCATCTGGAAGATTAAATCTCTGAAGCATCTCCTTGTGTATAATAACAGTCTTTCTGG
GGAACTTCCTTTGGAGATGACAGAGCTCAAGCAACTGAAAAACATCTCATTGTTTAGC
AACCAGTTCTCCGGAGTCATACCGCAAAGCTTGGGAATTAACAGCAGTTTAGTTCTTT
TGGATTTTACAAATAATAAATTCACTGGCAACATCCCACCAAATCTGTGTTTTGGCAAG
AAATTAAACATCCTGAATTTGGGCATCAACCAACTTCAAGGCAGCATTCCTCCTGATG
TTGGGAGATGTACAACCCTTAGAAGGTTAATTCTTCAACAAAATAACTTTACTGGGCCT
CTTCCTGATTTCAAAAGCAATCCAAATCTCGAACATATGGATATCAGCAGCAACAAAAT
CCATGGTGAAATTCCATCAAGTTTGCGAAATTGCAGACATATCACTCACCTAATTTTGT
CCATGAACAAATTTAATGGGCCTATACCCTCAGAGCTAGGGAACATTGTCAATCTTCA
AACTTTGAATCTCGCTCACAACAACTTAGAAGGTCCTTTGCCCTCTCAGCTGTCAAAG
TGTACCAAAATGGACAGGTTTGATGTTGGATTTAATTTCCTAAATGGTTCATTGCCATC
AGGTCTGCAGAGCTGGACAAGGCTAACCACATTAATTTTGAGTGAGAATCACTTTAGT
GGGGGCCTCCCAGCTTTCTTGTCGGAATATAAAATGCTTTCTGAACTACAACTTGGTG
GCAATATGTTTGGTGGCAGAATTCCTAGATCGGTTGGAGCATTGCAGAGTTTGAGGTA
TGGTATGAATCTGAGTTCAAATGGGCTGATAGGAGACATTCCTGTTGAGATTGGAAAC
TTGAACTTTTTGGAAAGACTGGATCTGTCTCAGAACAATTTGACCGGAAGCATAGAAG
TTCTTGGTGAACTCCTCTCCTTAGTTGAAGTCAATATTTCATACAATTCTTTTCATGGTC
GTGTACCAAAGAAGCTAATGAAATTGCTCAAGTCTCCCTTGTCATCATTTTTGGGCAAT
CCTGGCCTATGTACCACCACCAGGTGTTCAGCATCTGATGGCTTGGCTTGCACTGCA
AGAAGCTCTATAAAACCATGTGATGACAAATCTACTAAACAGAAAGGCCTCAGTAAAG
TTGAAATTGTGATGATAGCTCTCGGGTCCTCAATACTTGTTGTTTTGCTGTTGCTGGG
ATTAGTTTATATTTTTTATTTTGGAAGAAAAGCTTACCAGGAAGTCCATATCTTTGCTGA
AGGGGGTTCTTCTTCCCTTCTTAACGAAGTCATGGAGGCTACAGCAAACCTAAATGAT
CGGTATATTATTGGCAGAGGAGCCTATGGAGTTGTTTATAAAGCCCTGGTGGGTCCA
GACAAAGCCTTTGCTGCGAAGAAGATAGGATTTGCTGCGAGCAAAGGTAAGAACTTG
AGCATGGCCAGAGAAATTGAAACCCTTGGGAAAATTCGGCATCGAAATCTGGTCAAA
TTGGAAGACTTTTGGTTGAGAGAAGATTATGGTATAATTTTGTACAGCTACATGGCAAA
TGGAAGTCTTCATGATGTTTTGCACGAAAAGACACCACCACTAACCTTAGAGTGGAAT
GTCCGGAATAAGATAGCTGTTGGAATTGCTCATGGATTGGCTTATCTCCATTATGACT
GTGATCCTCCCATAGTGCACCGAGACATCAAGCCAAGCAATATACTTCTAGACTCTGA
TATGGAGCCTCACATTGCTGACTTTGGTATTGCCAAACTTCTGGATCAGTCTTCTGCT
TCAAATCCTTCCATTTCTGTTCCGGGTACAATTGGTTATATTGCACCAGAGAATGCTTA
TACAACAACAAATAGTAGGGAGTCTGATGTATACAGTTACGGGGTAGTTTTGCTTGAG
CTGATAACCAGAAAGAAGGCAGCAGAATCAGATCCTTCCTTCATGGAGGGTACTATA
GTAGTGGATTGGGTTAGGTCTGTGTGGAGGGAAACAGGAGACATTAATCAAATTGTT
GATTCAAGCCTTGCAGAGGAATTTCTAGATATCCATATAATGGAAAATATTACCAAAGT
GCTTATGGTGGCTCTGAGATGTACTGAGAAGGATCCACACAAGAGACCCACAATGAG
AGATGTTACCAAGCAGTTAGCAGATGCTAATCCACGGGCAAGAAGTACAAAGGGCTA
G
SEQ ID NO: 48-SIPEPR1 LIKE1 (RER-LIKE1) Soybean Amino Acid Sequence
(GmRER-LIKE1)
MSMIWIVFFSLSCMSCAVVSSLTSDGVTLLSLLRHWTSVPPSINATWLASDTTPCSSWVG
VQCDHSHHVVNLTLPDYGIAGQLGPEIGNLSRLEYLELASNNLTGQIPDAFKNMHNLNLLS
LPYNQLSGEIPDSLTHAPQLNLVDLSHNTLSGSIPTSIGNMTQLLQLYLQSNQLSGTIPSSI
GNCSKLQELFLDKNHLEGILPQSLNNLNDLAYFDVASNRLKGTIPFGSAASCKNLKNLDLS
FNDFSGGLPSSLGNCSALSEFSAVNCNLDGNIPPSFGLLTKLSILYLPENHLSGKVPPEIG
NCMSLTELHLYSNQLEGNIPSELGKLRKLVDLELFSNQLTGEIPLSIWKIKSLKHLLVYNNS
LSGELPLEMTELKQLKNISLFSNQFSGVIPQSLGINSSLVLLDFTNNKFTGNIPPNLCFGKK
LNILNLGINQLQGSIPPDVGRCTTLRRLILQQNNFTGPLPDFKSNPNLEHMDISSNKIHGEIP
SSLRNCRHITHLILSMNKFNGPIPSELGNIVNLQTLNLAHNNLEGPLPSQLSKCTKMDRFD
VGFNFLNGSLPSGLQSWTRLTTLILSENHFSGGLPAFLSEYKMLSELQLGGNMFGGRIPR
SVGALQSLRYGMNLSSNGLIGDIPVEIGNLNFLERLDLSQNNLTGSIEVLGELLSLVEVNIS
YNSFHGRVPKKLMKLLKSPLSSFLGNPGLCTTTRCSASDGLACTARSSIKPCDDKSTKQK
GLSKVEIVMIALGSSILVVLLLLGLVYIFYFGRKAYQEVHIFAEGGSSSLLNEVMEATANLND
RYIIGRGAYGVVYKALVGPDKAFAAKKIGFAASKGKNLSMAREIETLGKIRHRNLVKLEDF
WLREDYGIILYSYMANGSLHDVLHEKTPPLTLEWNVRNKIAVGIAHGLAYLHYDCDPPIVH
RDIKPSNILLDSDMEPHIADFGIAKLLDQSSASNPSISVPGTIGYIAPENAYTTTNSRESDVY
SYGVVLLELITRKKAAESDPSFMEGTIVVDWVRSVWRETGDINQIVDSSLAEEFLDIHIMEN
ITKVLMVALRCTEKDPHKRPTMRDVTKQLADANPRARSTKG
SEQ ID NO: 49-SIPEPR1 LIKE 2 (RER-LIKE1) Soybean DNA Sequence (GmRER-
LIKE2)
ATGGGGTATTTGTATCTCTTGCTGATTTCATATTTGTCTGCCTTGTTGTATGCTGCTTC
TGCATTGAACTCTGATGGGTTGGCTTTGTTGTCCCTCTTGAGGGACTGGACTATTGTG
CCTAGTGACATAAACTCCACATGGAAGTIGTCTGATTCCACTCCGTGCTCATCTTGGG
CAGGAGTGCATTGTGATAATGCCAATAATGTGGTTTCTCTAAACCTCACTAGTTATTCT
ATTTTTGGTCAATTAGGACCTGATCTTGGACGTATGGTTCACTTGCAAACCATAGACTT
ATCATATAATGATCTATTTGGAAAAATTCCCCCAGAATTAGACAACTGTACCATGCTTG
AGTACTTGGACCTTTCTGTAAACAACTTTAGTGGAGGAATACCTCAGAGCTTCAAAAA
CTTGCAAAATTTGAAGCATATAGACCTTTCATCTAATCCGCTGAATGGTGAAATTCCTG
AACCCTTGTTTGACATTTATCACCTGGAAGAAGTGTATCTTAGCAACAACAGTTTGACT
GGTTCAATTTCCTCAAGTGTTGGGAATATCACTAAGCTTGTCACACTGGATCTTTCTTA
TAATCAGCTGTCAGGGACAATTCCCATGTCCATTGGAAATTGTAGTAACTTAGAGAAT
CTATATTTGGAAAGGAATCAATTAGAGGGAGTTATTCCTGAGAGTCTAAATAATCTCAA
AAATCTTCAGGAGTTATTTCTCAATTATAATAACCTTGGAGGCACTGTTCAATTGGGAA
CTGGAAATTGCAAAAAGTTGTCTAGTTTAAGTCTTTCTTACAATAACTTCAGTGGGGGT
ATACCATCAAGCTTGGGGAATTGTAGCGGTCTAATGGAGTTTTATGCTGCACGGAGTA
ACTTAGTTGGCAGTATACCATCAACCTTGGGCCTCATGCCCAACCTTTCTCTTCTAAT
CATTCCAGAGAACCTATTGTCTGGGAAAATACCTCCACAGATTGGTAATTGCAAAGCA
CTGGAAGAGTTGCGTTTGAATTCCAATGAACTTGAGGGAGAAATTCCCAGTGAATTGG
GAAACTTGAGTAAATTACGCGACCTTAGATTGTATGAAAACCTTTTGACAGGAGAAAT
TCCACTTGGCATATGGAAAATTCAAAGCCTTGAGCAGATCTATCTGTACATTAATAACC
TTTCGGGGGAGCTACCTTTTGAGATGACAGAGCTCAAACATCTTAAGAATATCTCCTT
GTTTAACAACCAGTTCTCCGGAGTCATACCTCAAAGTTTAGGAATCAATAGCAGTTTG
GTGGTGTTAGACTTCATGTATAATAATTTCACTGGTACCCTTCCACCAAATCTTTGTTT
TGGAAAGCAACTGGTGAAGCTGAATATGGGTGTCAATCAATTTTATGGTAACATACCT
CCAGATGTGGGAAGGTGTACAACTCTTACAAGGGTGAGACTTGAAGAAAATCATTTCA
CTGGGTCTCTTCCTGATTTTTATATTAATCCAAATCTCTCTTACATGAGCATCAACAAC
AACAATATCAGTGGAGCAATTCCATCAAGTTTGGGAAAATGCACAAATCTCTCTCTTTT
AAATTTGTCCATGAACAGCTTGACGGGTCTTGTACCTTCAGAGCTTGGAAACCTTGAG
AATCTCCAGACTTTGGATCTTTCTCACAATAACTTGGAAGGTCCTTTGCCACATCAGCT
GTCAAACTGTGCCAAAATGATCAAGTTTGATGTCAGATTCAATTCCTTGAATGGTTCG
GTTCCATCAAGTTTTCGGAGCTGGACAACATTAACAGCTTTAATTCTCTCAGAGAATCA
TTTTAATGGTGGTATCCCAGCTTTCTTGTCAGAATTTAAAAAGCTCAACGAGTTACAAC
TTGGTGGAAACATGTTTGGAGGAAACATTCCTAGATCAATCGGAGAGCTGGTGAATTT
GATATATGAACTAAATCTAAGTGCTACTGGGCTGATAGGTGAGCTTCCTAGGGAGATT
GGAAACCTGAAGAGTCTGCTAAGCCTGGATCTATCTTGGAACAATTTGACAGGAAGTA
TACAAGTTCTTGATGGGCTCAGTTCATTATCTGAATTCAACATCTCATATAATTCTTTTG
AAGGTCCTGTGCCACAACAGCTAACAACATTACCAAACTCTTCTTTATCATTTTTGGGC
AATCCTGGCCTGTGTGGCTCGAATTTCACTGAGAGCAGCTATTTAAAGCCTTGTGACA
CAAATTCAAAAAAGTCAAAAAAGCTCAGTAAAGTTGCAACTGTGATGATAGCACTTGG
ATCTGCAATATTTGTTGTTCTGCTGCTGTGGTTAGTATATATATTCTTTATCAGAAAAAT
TAAGCAAGAAGCCATAATCATTAAGGAAGATGATTCTCCAACCCTTCTTAACGAAGTG
ATGGAAGCTACAGAAAATCTAAATGATGAGTATATTATTGGCAGAGGAGCTCAAGGAG
TTGTTTATAAAGCAGCAATAGGTCCAGACAAAACATTGGCTATAAAGAAGTTTGTATTT
TCTCATGAAGGGAAAAGCTCAAGCATGACCAGAGAAATTCAAACCCTTGGAAAGATTA
GGCATCGAAATTTAGTCAAATTGGAAGGGTGCTGGTTGAGAGAAAACTATGGTCTAAT
TGCATACAAATACATGCCAAATGGAAGCCTACATGATGCTTTGCATGAGAAGAATCCA
CCATACTCCTTAGAATGGATTGTTCGGAATAACATAGCACTTGGAATTGCTCACGGAT
TGACTTATCTCCATTATGACTGTGATCCTGTCATAGTGCACAGAGATATCAAAACAAG
CAACATACTTCTAGATTCAGAAATGGAGCCTCATATTGCAGATTTTGGTATTGCTAAAC
TTATAGATCAGCCTTCTACCTCAACACAGTTATCATCTGTTGCTGGTACACTTGGTTAT
ATAGCACCAGAGAATGCTTATACAACAACAAAGGGTAAGGAATCTGATGTATACAGTT
ATGGGGTAGTTTTGCTGGAGCTGATATCCAGAAAGAAGCCATTGGATGCATCATTTAT
GGAAGGAACGGATATAGTTAATTGGGCAAGATCTGTCTGGGAGGAAACGGGAGTTGT
TGATGAAATTGTTGATCCAGAGCTGGCTGATGAAATTTCAAATTCTGAAGTGATGAAA
CAAGTTACCAAGGTGCTTTTGGTGGCTTTGAGATGCACTGAAAAGGATCCACGTAAG
AGACCTACGATGAGGGATGTTATCAGGCATTTGTAG
SEQ ID NO: 50-SIPEPR1 LIKE2 (RER-LIKE1) Soybean Amino Acid Sequence
(GmRER-LIKE2)
MGYLYLLLISYLSALLYAASALNSDGLALLSLLRDWTIVPSDINSTWKLSDSTPCSSWAGV
HCDNANNVVSLNLTNLSYNDLFGKIPPELDNCTMLEYLDLSVNNFSGGIPQSFKNLQNLK
HIDLSSNPLNGEIPEPLFDIYHLEEVYLSNNSLTGSISSSVGNITKLVTLDLSYNQLSGTIPM
SIGNCSNLENLYLERNQLEGVIPESLNNLKNLQELFLNYNNLGGTVQLGTGNCKKLSSLSL
SYNNFSGGIPSSLGNCSGLMEFYAARSNLVGSIPSTLGLMPNLSLLIIPENLLSGKIPPQIG
NCKALEELRLNSNELEGEIPSELGNLSKLRDLRLYENLLTGEIPLGIWKIQSLEQIYLYINNL
SGELPFEMTELKHLKNISLFNNQFSGVIPQSLGINSSLVVLDFMYNNFTGTLPPNLCFGKQ
LVKLNMGVNQFYGNIPPDVGRCTTLTRVRLEENHFTGSLPDFYINPNLSYMSINNNNISGA
IPSSLGKCTNLSLLNLSMNSLTGLVPSELGNLENLQTLDLSHNNLEGPLPHQLSNCAKMIK
FDVRFNSLNGSVPSSFRSWTTLTALILSENHFNGGIPAFLSEFKKLNELQLGGNMFGGNIP
RSIGELVNLIYELNLSATGLIGELPREIGNLKSLLSLDLSWNNLTGSIQVLDGLSSLSEFNISY
NSFEGPVPQQLTTLPNSSLSFLGNPGLCGSNFTESSYLKPCDTNSKKSKKLSKVATVMIA
LGSAIFVVLLLWLVYIFFIRKIKQEAIIIKEDDSPTLLNEVMEATENLNDEYIIGRGAQGVVYK
AAIGPDKTLAIKKFVFSHEGKSSSMTREIQTLGKIRHRNLVKLEGCWLRENYGLIAYKYMP
NGSLHDALHEKNPPYSLEWIVRNNIALGIAHGLTYLHYDCDPVIVHRDIKTSNILLDSEMEP
HIADFGIAKLIDQPSTSTQLSSVAGTLGYIAPENAYTTTKGKESDVYSYGVVLLELISRKKPL
DASFMEGTDIVNWARSVWEETGVVDEIVDPELADEISNSEVMKQVTKVLLVALRCTEKDP
RKRPTMRDVIRHL
SEQ ID NO: 51-SIPEPR1 (RER) Sunflower DNA Sequence (HanRER)
ATGGAAATGGAAACAACAAGAAGAAGAAGAAGTCTTTTGTTTTTGTTTTCTCTTCTAGT
TCTACCATTCTATCTTGTCTCTGGGTTGAGCTCTGATGGGTTAAACCTATTATCATTGA
TGACCCACTGGACTTCAGTACCTCCTTCCATCTATTCTACCTGGAATGCTTCACACCC
AACACCATGCTCATGGGTTGGAGTTCACTGTAACCCTAACAACCATCATGTCCACTCT
CTCAACCTCTCCTCTTACCTCATTTCCGGTCATATAGGAGGCCCAGAGCTTGCAAACC
TCACCCATCTCACATCCCTTGACTTGAGCTTCAATAACCTCTCTGGACCCTTACCTCC
ACAACTTGGTAACTGTACCCGTCTTCGTTATCTGGACCTTTCCTACAACATCCTCTCTG
GACCCATTCCACACACTCTAGGGAACCTCATCCACTTAACGCAGATCATTCTTTACAG
TAACAAACTTACCGGTTCTATCCCTTACCAGATTGGCAACTTGAGTAATCTTGAGGAA
CTTGATTTCTCTTTAAACCACCTCACAGGAACAATACCAGAAAGCCTATGGAAGTTGC
AAAATCTTAGGGTCTTAAGTCTTTATGGCAACTCTCTTCTAGGTTCTGTACCCTTCTTA
GTTAACGCTCCCTATTTGGAAGCTGTTTATCTCACTTCTAATCAGCTTAGTGGTCCCAT
TTCATCGACCATAGGTAACCTTAGCAACCTAGTTGACTTGCAGTTAGACAGCAATCAG
TTCTGGGGTCCTATTCCCTCATCCATTGGGAAGTGCAGCAGGCTGCAGACCCTCATG
CTTGGTAACAATCGCTTCACTGGTGAATTGCCTAATACCCTCAATCATCTTTCTCAGCT
TACTAAATTAGACGTCCATAACAACAGTTTAAAAGGTAGCATTCGCTTTGGCGGTGGT
AACTGTTTGAATTTGGTCTACTTGGATATTTCATTTAACCAGTTTCATGGCCTCCTGCC
TCATGAACTGGGAAACTGCAGCAGCTTAAACCAAGTTGCTGCTGTTAACTCTGGGTTA
GGTGGCTCTATTCCTTCTTCTATTGGTAAACTTACTGCACTCACCACTCTTTACCTTTC
TATTAATCATTTTTCTGGAAATATACCACCTGAGCTTGGCGGTTGTAAGTCCTTGGTTG
ATCTACAACTCCATGCTAACCAACTGGAGGGCCTTATCCCGAACGAACTCGGGATGT
TAAGTCAACTGCAAGTACTTGAAGCGTTTGATAACCGTCTGACAGGAGAGGTGCCTCT
GGGAATCTGGAAGATTAAGGGCCTCAAGAAGCTCAACATATACAACAATAATCTTTCA
GGGGAACTGCCCGATGAGGTTGCTGGAATGAAGCAACTAAGGGAGATTACGTTGTAC
AACAATCGCTTCACTGGAGCTATACCTCAGGGGTTGGGTATCAATAGCAGTTTGACTG
TGATCGATTTCACCAATAACTCATTCACCGGGAAAATTCCACCAAGCCTCTGCTTTCG
AAAGCAGCTGCAAAGGCTACTTTTGGGTTATAATTATTTACAAGGCAGTGTGCCTCAT
GATGTTGGAAGCTGTTCTAGTCTTTCAAGATTAATTCTGCAACATAACAACCTCACAG
GAGTCCTTCCAGAGTTTATGGAGAACCGCAACATGCTATACATGAACCTTAGGGGCA
ACTTTTTTAGCGGAAAAATTCCAGCCAGCTTCGGAAAGCTTACCAATATCACAGAAAT
TGACCTCTCAATGAATAAACTCTCTGGTAACATACCTCTAGAGTTGGGAAATCTTGTG
CAGATTCAAGCTCTGAATATTTCCTACAATGAACTCCAAGGTCCTTTGCCATCCCAGC
TAGGAAATTGTAGCAGGTTGTTGGAATTCGATGCAAGTCATAATTCGGTTAGTGGTTC
CATCCCAGCCGCATTTGGGAAGTTGACTCAGCTTTCAACTTTAAGTCTAAGCGATAAT
CATTTCTCTGGGGAAATTCCGCATTTTATATCTCAACTCCAAGATTTAATAGATCTCCA
ATTAGGTGGGAATTCATTCAGTGGTCATATCCCGTCATCGATAGTAGAGCTTCATAGT
CTAAACAGGTTGAATCTCAGCAGCAACAAACTGATTGGTGGCATTCCATCAAATTTTA
GGAAGATGGTAATGCTAGAGCATTTGGATGTTTCACACAACAATCTAACTGGGGATTT
AGCATCACTTGGCGACACCGTCGGGCTGGTCACCTTAGATGTTTCTTTTAATCATTTC
ACAGGCCCGATACCAGCAACTTTATTGAAGTCTATCAGCTCGTCGTCCTTCGTGGGG
AACACAGGTCTTTGTGTAGATTGTTGCATTGAAAACAGCAATATCAAACCATGTGTTGT
TCGATCACGGGGCCTGAAGAAACGACATTATATAATGATAGCAGTTGGAATGTGTCTG
TTTGTGATGGTTGTCTATCTAGTACTCTATCTGGCGGTTATCTGTCGCAGAAAACAAAA
ACAGGAAATGGACATGCTTGCTGAAGGGTGCTGTTCATCTTTGGTCATAAAGGCGCT
GGAAGCCACTGAGAATTTGAATGATAAGTATATTATTGGGAGGGGGGCCCATGGTAC
AGTGTACAAGGCTTGCTTGGGTCATGGTAGGGTCTATGCTGTGAAGAGGCTTGGTTT
TGGAGGGGGGCAAACAAGTATGAAAAGAGAAGTTGAGACGGTTGGGAAGGTGAGAC
ACAGGAATCTGGTGACGATACAAGATGTGTTGATAAAGAAGGATTATAGTTTGATTTT
GTACAAGTATATGGAAAACGGTAGCCTATATGATGTTTTGTATGAAAAAGAACCAACA
TTAGTCTTAAGTTGGAGCGTTCGGTACAGGATAGCTTTAGGAACTGCCCAGGGTTTG
GCATATCTCCATTTTGACTGTGATCCTGTTATCATACACCGGGATATAAAACCCATGAA
CATTCTTTTGGATGGTGATATGGAAGCTCATATCTCGGACTTTGGCATTGCCAAGCTT
CTAGATCAGTCTGCTACTGGTGCACTAGCAACCAGCACACTTATGGGCACAATAGGTT
ACATCGCTCCAGAAAACGCGTTCACGAGGTCAGCAAGCAGGGAGTTGGACGTATATA
GCTATGGAGTTGTGTTGCTGGAGCTTATAAGTAGGCAGAAGCCTGTGTTGATAGAAAA
TGTGGACATGGTGTCGTGGGTAAGGTCGAGATCGGAAGAAATCGAGAAGATTGTAGA
CAGAGGGCTGTTAGAGGAAATTGAAGAAGATAAGAGGGTGAGGGAACAAGTGAGAA
AAGTGGTGTTGGTGGCCTTGAGATGTACAGAGTGGGAACCAACCAGAAGACCAACAA
TGAGGGAGGTGGTCAAGTGTCTTCAAGATTCAGTTTAG
SEQ ID NO: 52-SIPEPR1 (RER) Sunflower Amino Acid Sequence (HanRER)
MEMETTRRRRSLLFLFSLLVLPFYLVSGLSSDGLNLLSLMTHWTSVPPSIYSTWNASHPT
PCSWVGVHCNPNNHHVHSLNLSSYLISGHIGGPELANLTHLTSLDLSFNNLSGPLPPQLG
NCTRLRYLDLSYNILSGPIPHTLGNLIHLTQIILYSNKLTGSIPYQIGNLSNLEELDFSLNHLT
GTIPESLWKLQNLRVLSLYGNSLLGSVPFLVNAPYLEAVYLTSNQLSGPISSTIGNLSNLVD
LQLDSNQFWGPIPSSIGKCSRLQTLMLGNNRFTGELPNTLNHLSQLTKLDVHNNSLKGSI
RFGGGNCLNLVYLDISFNQFHGLLPHELGNCSSLNQVAAVNSGLGGSIPSSIGKLTALTTL
YLSINHFSGNIPPELGGCKSLVDLQLHANQLEGLIPNELGMLSQLQVLEAFDNRLTGEVPL
GIWKIKGLKKLNIYNNNLSGELPDEVAGMKQLREITLYNNRFTGAIPQGLGINSSLTVIDFT
NNSFTGKIPPSLCFRKQLQRLLLGYNYLQGSVPHDVGSCSSLSRLILQHNNLTGVLPEFM
ENRNMLYMNLRGNFFSGKIPASFGKLTNITEIDLSMNKLSGNIPLELGNLVQIQALNISYNE
LQGPLPSQLGNCSRLLEFDASHNSVSGSIPAAFGKLTQLSTLSLSDNHFSGEIPHFISQLQ
DLIDLQLGGNSFSGHIPSSIVELHSLNRLNLSSNKLIGGIPSNFRKMVMLEHLDVSHNNLTG
DLASLGDTVGLVTLDVSFNHFTGPIPATLLKSISSSSFVGNTGLCVDCCIENSNIKPCVVRS
RGLKKRHYIMIAVGMCLFVMVVYLVLYLAVICRRKQKQEMDMLAEGCCSSLVIKALEATEN
LNDKYIIGRGAHGTVYKACLGHGRVYAVKRLGFGGGQTSMKREVETVGKVRHRNLVTIQ
DVLIKKDYSLILYKYMENGSLYDVLYEKEPTLVLSWSVRYRIALGTAQGLAYLHFDCDPVII
HRDIKPMNILLDGDMEAHISDFGIAKLLDQSATGALATSTLMGTIGYIAPENAFTRSASREL
DVYSYGVVLLELISRQKPVLIENVDMVSWVRSRSEEIEKIVDRGLLEEIEEDKRVREQVRK
VVLVALRCTEWEPTRRPTMREVVKCLQDSV*
SEQ ID NO: 53-SIPEPR1 (RER) Cotton DNA Sequence (GaRER)
ATGAAAGGTAACTTTCTCTTTCCATTAACCTTGCTCTTGTGTTTCTTGTTTTTAGAACAA
AACACTGGTCTTTTTGTTTATGCTTTGAATTCTGATGGGGAAACCCTGTTTTCTTTGTT
ACCTCACTGGTCATCCTTACCTTCTTCTTTAACCTCTTCATGGAATGCTTCTGATCCAA
ATCCATGCAAATGGGTTGGTGTTGAATGTGATTCTGAAACCCATAATGTCCTTACTCTT
AACCTTACAAACTTAGCCATTTCAGGCCAATTGCCACCTCAAATTGGTGCTTTACACC
ATTTGAACACCCTTGATTTGAGCAACAATAGGTTCTTTGGTCCAATACCCTCAGCTTTG
GCCAATTGTAGCTCCCTTCAACACTTGGATTTGTCTAACAATGAACTCATTGGACCAAT
GCCTAATACTTTCAATTCCTTGCAAAAGCTGAATTACTTGAACTTGTTTTCCAATTCCTT
GAGTGGTGAAATACCAGAAACATTGTTTCATGTTACTGGTTTGGTGTCCGTGTATTTG
AATAATAACAACTTGAGTGGTTCAATACCTATGAATGTTGGTAACTTGAGCGAGCTTGT
AGCTTTAAATTTGTATGATAATGGGTTGATAGGTACCATACCAGAGTCAATAGGGAAT
TGTAGTAAACTACAAGAACTTTTTTTAGATGGGAACTATTTTGTTGGGGGTTTGCCTTC
CAGTGTTATGAATCTCCAAAACTTAATGTATTTGTATTTGCGTCACAATAGTTTCAATG
GTGAAATCCCAAGTCCTACCAAGTGTAAAAATTTGAGTGTTTTGGATTTATCATTCAAT
AGTTTCAATGGGGGGATACCACCAGGGTTAGCCAATTGTAGCACCTTGACTGAGCTG
GTTATTGTACATAGTAACTTAACAGGTTATATTCCTTCTTCATTAGGCTTATTAGATCAG
CTTTCAAAGCTTGATCTTTCTGAAAACCATTTATATGGTGAAATCCCATTTCAGCTTGG
GAAATGTAAGTCTTTGAAACAGCTATTATTGTATGATAATCAACTCAAAGGTGAAATCC
CTAATGAATTAGGGATGTTAAGTGAGTTGAATGATCTTGAGTTGTTTATGAACCATTTG
AGTGGTGAGATTCCTATTAGTATTTGGAGGATTCCAAGCTTAGAGTATTTACTTGTTTA
TAAGAACAACCTAACAGGGGAATTGCCTTTAGAGATCACTGAGTTCAAGCAATTGAAA
AACATTTCGTTGTACGATAATGGTTTCTTCGGGGTCATACCTCGGAATTTGGGGATTA
ATGCTAGTTTACAGCAGTTGGATTTCACAAACAATATGTTCAGTGGTACAATCCCTCCA
TTTCTTTGCTTTGGAAAGAAGTTAAGGGTGTTGAATTTGGGTCAAAATCAGCTTACCG
GCAGTGTAACCGATGATATCGGAGGCTGTAAAACTCTGTGGAGATTGATCCTTAAGC
AGAATAACCTCAACGGTGTGCTGCCCGAGTTTGCAGAGAATAAAAATCTTGTACACAT
GGATATTAGTGAAAATGACATCTCCGGTCCAATTCCATCCAGCTTGGGGAACTGTAGA
AACCTTACTTCTATCAATTTGTCCATGAATCGCTTCACGGGATTTATACCTTCGGAGTT
AAGCAACCTAGCAGATCTTCAGACTTTGAATGTTTCTCACAACCTTTTACAAGGTTCTT
TGCCATCTCAGTTGTCAAATTGTAGCAGATTGGTAGAGTTCGACGTGGGGTTTAATTC
GCTGAATGGTTTGATTCCGAATGCTTTGACGAGCTGGAAACAGCTGTCAACTCTTATT
CTGAGTGAAAACCGATTTACTGGTGGCATTCCTTCTTTCTTGTTGGAACTCGAAATGC
TTTCGGACCTGCAGCTCGGGGGAAATCCATTTGGTGGTAGTATTCCTTCGTCGATTG
GAGCTATGAAGAATCTGATTTATGGCTTGAATTTGAGCAGCAATAGTTTGACTGGAGA
GATTCCCTCGGAGCTAAGAAATCTGTTCAAGCTGGTTAGATTGGATTTATCTAATAACA
ATCTAACAGGGACTTTAACAGTTCTTGATGGAATGGATTCCTTGGTTGAGGTCAATGT
TTCCTACAATCACTTCACTGGTCCGATACCGACAACAATGATGCGTTTCATGAACTTG
TCCTCATCATCTTTCCTCGGCAATCCCGGGTTATGCATTGATTGTCTTTCATCAGGTG
AGGAAACTTGTCCTACAAGAAATTACCTTAATCCTTGTAATGAAATGAAAAATCAAAGG
GGCCTTACCAAATTGGAAGTTGCAATGATAGCCCTCGGTTCATCTCTACTTGTTGTGG
CACTCCTAGTTGTGGCTTCGATATTCATTTTCTGCAGGATAAAAAAGCAAGAACATGA
GGTCTGTGTCGAAGAAGGCGCATCGACCTTATTGAACAAAGTGATGGAAGCTACCGA
GAATTTAAATGAGAGGTATATTATCGGCAGAGGAGCACATGGAGTCGTTTATAGAGCA
TCATTGAGTCCAGGGAGTGATTTTGCTGTAAAAAGGATCAATGTAGCAAAGCATAAAG
GAGGAAACCAAAGCATGGTTCGAGAGATCGAAACTATTGGGAAAGTTAAGCATCGAA
ACCTGGTGAGATTAGAAGATTTTTGGTTGAGAAAAGATTATGGATTACTCTTATACAGG
TACATGCAAAATGGGAGCCTACATGATGTTTTACATAATAATAATGATCATACAACAAA
TGAGGTACAAATCCTGAAATGGAGTGCTCGGTATAGAATAGCACTAGGAACGGCTCA
TGGGTTGGAATATCTCCATTATGATTGTGATCCCGGTATAGTTCATCGAGACATCAAG
CCAGAAAACATCCTCCTTGACTCCGATATGGAACCACACATCTCCGATTTTGGAATTG
CTAAGCTCCTTGATGAGTCTGTGGCTTCCGAACCGTCTATGGTAGTTGCGGGCACGG
TTGGATATATAGCTCCAGAAAATGCATTCAGAACAACATGGAGCAAAGAAGCGGATGT
GTATAGCTATGGGGTGGTTTTGCTCGAGCTGATAACCGGGAAGAGGGCATTGGATCC
ATCATTTATGGGGGAGACTGATATCGTGGGGTGGGTCAGATCAGTTTTGAGCGGCGA
AATCGAAACCGAAATCGAGAGGATCGTGGATTCGAGGATTGTGGATGAGTTGATGGA
ATGGGAAGTGAGGGAGCAGGTTATTAATGTGGTTTTGGTAGCTTTGAGATGTACTGAA
AAGGAACCAAGCAGAAGACCTGCAATGAGAGATGTTGTGAGGCAGCTCTTAAACACC
AAACTCCCTAGAAAATCTAAACATCGGAGCTAA
SEQ ID NO: 54-SIPEPR1 (RER) Cotton Amino Acid Sequence (GaRER)
MEDTSSAKEVTLQGTPANYFPRSFHEVVGAILRCLGLETGFQQNPNPCPKKEDDSKANH
NQSVSQKESPDPPSSTDNSDPSTTVIDPPADPPPSTTGDTNDGELPMVSLFTPKRPGTS
AGSGPQIN
SEQ ID NO: 55-SIPEPR1 (RER) Rice DNA Sequence (OsRER)
ATGGGACTGCACATATGGTGTTGGTTGGTTGTCTTGTTCAGCTTGGCCCCATTGTGTT
GTAGTTTGAGCGCAGATGGCCTGGCTCTTCTGGATCTAGCCAAGACTCTGATACTGC
CCAGCTCCATAAGCTCGAATTGGAGTGCTGATGATGCAACTCCGTGTACATGGAAAG
GAGTTGATTGTGATGAAATGAGCAATGTGGTTTCTCTTAACTTATCATATTCTGGATTG
TCTGGTTCTCTAGGTCCTCAGATAGGACTCATGAAGCACCTGAAAGTCATTGATTTAT
CAGGTAATGGTATATCAGGACCAATGCCCAGTTCCATTGGCAACTGCACCAAACTGG
AGGTGCTCCATCTACTACGTAATCGATTGAGTGGGATCCTTCCAGATACATTGAGCAA
TATTGAAGCATTAAGGGTTTTTGATCTCTCCCGCAATAGCTTCACAGGCAAGGTCAAT
TTCAGATTTGAGAACTGCAAGCTTGAGGAGTTCATCTTGTCATTCAATTATCTCAGAG
GCGAAATCCCGGTGTGGATAGGGAATTGCAGCAGCTTGACACAGCTTGCATTTGTCA
ACAATAGTATCACCGGTCAAATACCAAGTTCAATCGGTTTATTGAGAAACCTTTCTTAC
CTTGTACTTTCCCAGAACTCCTTGTCTGGCACAATCCCTCCTGAGATTGGTAACTGCC
AATTGCTGATATGGCTGCATCTAGATGCAAACCAGCTCGAGGGCACTATACCAAAAG
AACTAGCAAACCTGAGGAACTTGCAGAAGCTCTATCTTTTTGAGAATTGCCTCACTGG
GGAGTTTCCTGAAGATATATGGGGAATCCAAAGCCTACTATCTGTCGACATCTATAAA
AACAATTTCACTGGGCAGCTGCCTATAGTGTTGGCTGAGATGAAGCAGCTCCAGCAA
ATTACGCTATTCAATAATTCATTCACTGGTGTCATACCACAGGGGTTGGGTGTAAATA
GCAGTTTGTCCGTAATTGATTTCATAAACAATAGTTTTGTTGGCACAATCCCTCCAAAA
ATTTGTTCAGGGGGAAGATTGGAAGTTTTGAACTTGGGTTCAAATCTTCTCAATGGTA
GCATCCCCTCTGGTATCGCTGACTGCCCAACTTTGAGACGAGTAATTCTCAACCAAAA
TAATCTCATTGGATCAATTCCACAATTTGTAAATTGTAGCAGTCTTAATTATATTGATCT
CAGCTATAATTTATTAAGTGGGGACATTCCTGCTAGCTTGAGCAAATGTATCAATGTTA
CATTTGTGAACTGGTCATGGAACAAGCTTGCTGGTCTAATACCATCAGAAATTGGGAA
CTTAGGGAACTTAAGTAGTCTTAACCTCTCAGGAAACAGACTATATGGTGAACTCCCT
GTGGAAATTTCTGGATGCTCCAAGTTATATAAGCTTGATTTGAGCTACAACTCTTTGAA
CGGTTCGGCACTCACAACTGTAAGTAGCCTTAAATTTCTGTCACAGCTACGGTTGCAG
GAGAATAAATTCAGTGGAGGTATACCTGATTCTTTATCTCAGTTGGATATGCTTATTGA
ACTGCAACTTGGTGGCAACATTCTTGGGGGTAGTATCCCTTCATCGTTAGGAAAGTTA
GTTAAACTGGGCATTGCATTAAACCTCAGTAGAAATGGACTAGTTGGTGACATTCCAC
CACTAGGCAATTTGGTGGAGCTGCAGAGTTTAGATTTGTCATTTAATAACCTCACCGG
AGGTCTTGCTTCATTAGGAAACCTACAGTTTTTGTATTTCTTGAATGTTTCCTACAACA
TGTTTAGTGGACCAGTACCAAAAAATCTTGTGAGGTTTCTGAATTCCACTCCAAGTTCA
TTTAGTGGAAATGCAGATCTATGTATCTCTTGCCATGAAAATGATTCATCTTGCACAGG
TTCTAATGTTTTGAGACCTTGTGGTTCAATGAGTAAAAAAAGTGCACTCACACCACTCA
AGGTTGCTATGATAGTTCTTGGTTCGGTTTTTGCTGGTGCATTTCTGATACTCTGTGTC
CTTCTAAAATATAATTTCAAGCCTAAGATTAACAGTGATTTAGGTATATTATTTCAAGGA
TCTTCTTCTAAATTAAATGAGGCTGTAGAAGTGACTGAAAACTTCAATAACAAGTACAT
TATCGGTTCCGGGGCCCATGGAATTGTCTACAAGGCAGTACTGAGGTCAGGAGAAGT
ATATGCTGTAAAGAAGCTTGTACATGCTGCTCACAAGGGCTCAAATGCAAGCATGATC
CGCGAGCTGCAGACGCTTGGTCAAATTAGGCACAGGAACCTGATAAGACTTAATGAA
TTCTTGTTTAAGCATGAGTATGGTTTGATCCTATATGATTTTATGGAGAATGGTAGCCT
GTATGATGTGTTGCATGGGACTGAGCCCACTCCAACTTTGGACTGGAGCATCCGCTA
CAGCATAGCTCTTGGAACAGCCCATGGTCTAGCATATCTCCATAATGACTGTCACCCT
GCTATCATACATCGAGATATTAAACCAAAAAATATATTGCTGGACAACGACATGGTAC
CGCATATCTCAGATTTTGGCATTGCAAAGCTCATGGATCAATATCCTGCTGCTTTACA
GACCACAGGAATCGTTGGTACTATTGGATATATGGCCCCAGAAATGGCCTTTTCAACC
AAGGCTACCACAGAATTCGATGTGTACAGTTACGGTGTGGTATTACTTGAGTTGATCA
CCAGAAAGATGGCTGTGGATTCCTCATTCCCTGGCAACATGGACATAGTTAGCTGGG
TATCCTCCAAGTTGAATGAGACTAATCAGATCGAAACTATTTGCGACCCAGCTCTCAT
TACTGAAGTATATGGAACACATGAAATGGAAGAAGTGCGCAAGCTGTTGTCATTAGCT
CTTAGATGCACAGCAAAGGAGGCAAGCCAAAGGCCTTCCATGGCCGTTGTTGTCAAA
GAGCTGACAGATGCAAGACATGTTGCTGGCTCATACTCGAAGCAGAATTCAGGCCCC
AGCAATTCTTGA
SEQ ID NO: 56-SIPEPR1 (RER) Rice Amino Acid Sequence (OsRER)
MGLHIWCWLVVLFSLAPLCCSLSADGLALLDLAKTLILPSSISSNWSADDATPCTWKGVD
CDEMSNVVSLNLSYSGLSGSLGPQIGLMKHLKVIDLSGNGISGPMPSSIGNCTKLEVLHLL
RNRLSGILPDTLSNIEALRVFDLSRNSFTGKVNFRFENCKLEEFILSFNYLRGEIPVWIGNC
SSLTQLAFVNNSITGQIPSSIGLLRNLSYLVLSQNSLSGTIPPEIGNCQLLIWLHLDANQLEG
TIPKELANLRNLQKLYLFENCLTGEFPEDIWGIQSLLSVDIYKNNFTGQLPIVLAEMKQLQQI
TLFNNSFTGVIPQGLGVNSSLSVIDFINNSFVGTIPPKICSGGRLEVLNLGSNLLNGSIPSGI
ADCPTLRRVILNQNNLIGSIPQFVNCSSLNYIDLSYNLLSGDIPASLSKCINVTFVNWSWNK
LAGLIPSEIGNLGNLSSLNLSGNRLYGELPVEISGCSKLYKLDLSYNSLNGSALTTVSSLKF
LSQLRLQENKFSGGIPDSLSQLDMLIELQLGGNILGGSIPSSLGKLVKLGIALNLSRNGLVG
DIPPLGNLVELQSLDLSFNNLTGGLASLGNLQFLYFLNVSYNMFSGPVPKNLVRFLNSTPS
SFSGNADLCISCHENDSSCTGSNVLRPCGSMSKKSALTPLKVAMIVLGSVFAGAFLILCVL
LKYNFKPKINSDLGILFQGSSSKLNEAVEVTENFNNKYIIGSGAHGIVYKAVLRSGEVYAVK
KLVHAAHKGSNASMIRELQTLGQIRHRNLIRLNEFLFKHEYGLILYDFMENGSLYDVLHGT
EPTPTLDWSIRYSIALGTAHGLAYLHNDCHPAIIHRDIKPKNILLDNDMVPHISDFGIAKLMD
QYPAALQTTGIVGTIGYMAPEMAFSTKATTEFDVYSYGVVLLELITRKMAVDSSFPGNMDI
VSWVSSKLNETNQIETICDPALITEVYGTHEMEEVRKLLSLALRCTAKEASQRPSMAVVVK
ELTDARHVAGSYSKQNSGPSNS
SEQ ID NO: 57-SIPEPR1-LIKE1 (RER-LIKE1) Rice DNA Sequence (OsRER-LIKE1)
ATGAGGCTGGTTGTGTGGCACTGGTTTTTCTTCTTCTTCTTCACTTCTGTTTCATCGTC
TTGGAGTTTGACTTCAGATGGTCTAGCCCTTCTTTCTCTGTCTAGGGATCTCATATTAC
CTCATTCCATAAGCTCCACTTGGAAAGCTTCTGATACAACTCCTTGTAATTGGGATGG
GGTTTCCTGCAACAAAAAGAATAGTGTGGTTTCTCTTGACCTGTCATCTTCTGGAGTTT
CTGGTTCTCTTGGACCCCAAATAGGACTTATGAAGAGCCTACAAGTACTCAGTTTGTC
AAATAACAGCATATCTGGTTCAATCCCTCAAGAATTGGGCAATTGTAGCATGCTTGAT
CAATTGGATTTGTCCAGTAACAGTTTTTCTGGTGAGATACCAGCATCCCTTGGTGACA
TCAAAAAGCTTTCGTCTCTCTCTTTGTACAGTAACTCCCTCACTGGTGAAATACCAGA
GGGGTTGTTCAAGAATCAGTTTCTGGAGCAAGTGTACCTCCATTACAATAAACTCAGT
GGTTCTATCCCCTTGACAGTTGGAGAAATGACTAGCCTTAGGTACCTGTGGCTGCAT
GGCAATAAATTATCTGGAGTTCTACCAGATTCAATTGGCAACTGCACCAAGTTGGAGG
AGCTCTATCTACTAGATAATCAATTGAGTGGGAGTCTTCCGAAAACCTTGAGCTATAT
CAAAGGACTGAAGATTTTCGATATTACCGCAAATAGTTTCACAGGTGAGATCACATTT
AGTTTTGAGGATTGCAAGCTTGAGGTATTCATATTGTCATTCAATCAGATTAGCAACGA
AATTCCATCATGGCTAGGGAATTGTAGTAGCTTGACACAGCTTGCATTTGTCAACAAT
AATATATCTGGCCAGATTCCATCGTCTCTGGGTTTATTGAGAAACCTCTCTCAACTTTT
ACTTTCTGAGAACTCACTTTCTGGGCCAATTCCTCCTGAGATAGGTAACTGCCAGTTG
CTGGTGTGGCTGGAGTTGGATGCAAACCAGCTCAATGGCACTGTTCCTAAAGAGCTG
GCAAATCTGAGAAAATTGGAGAAACTCTTTCTGTTTGAAAACCGCCTCATTGGGGAGT
TCCCTGAGGATATTTGGAGCATCAAGAGCCTGCAAAGTGTCCTTATCTATGAAAACAG
TTTTACTGGGAGGCTACCTCCAGTGCTAGCTGAGCTGAAGTTCCTGAAGAACATTACA
CTTTTCAACAATTTCTTCACTGGAGTTATACCACCAGATTTGGGTGTTAATAGTCGTTT
AACCCAAATTGATTTCACAAACAACAGTTTTGTTGGTGGAATCCCGCCTAACATTTGTT
CAGGGAAAAGATTGAGAATTTTGGACTTGGGGCTTAATCTTCTCAATGGTAGCATCCC
ATCCAATGTTATGGACTGCCCAAGTTTGGAACGATTTATTCTCCAAAACAACAATCTAA
GTGGGCCCATTCCACAATTTAGGAACTGTGCAAATCTGAGCTATATAGATCTGAGTCA
TAATTCCTTAAGTGGCAACATTCCAGCAAGCTTGGGGAGATGTGTAAATATTACGATG
ATAAAATGGTCAGAAAACAAGTTGGTTGGTCCAATACCATCTGAAATTAGAGACTTGG
TGAATTTGAGAGTGCTAAACCTCTCGCAAAACAGCCTGCAAGGTGTTCTTCCAGTGCA
GATTTCTAGTTGCTCCAAGCTGTACTTGCTTGACTTGAGTTTCAACTCTTTGAATGGTT
CGGCACTCACAACCGTAAGCAACCTTAAGTTTCTGTCACAACTACGGTTACAAGAGAA
TAAATTCAGTGGAGGCATACCTGATTCCCTCTCGCAGTTGGATATGCTTATTGAGCTG
CAACTTGGTGGCAACGTTCTTGGGGGCAGTATCCCTTCATCGTTAGGAAGGTTGGTA
AAACTGGGCATTGCATTGAATATTTGTAGCAATGGACTCGTTGGTGGCATTCCGCCAT
TATTGAGCAATTTGGTGGAGCTGCAAAGTTTAGATTTGTCACTTAATGGCCTCACTGG
AGACCTAGACATGTTAGGAAACTTACAATTACTGCATGTATTGAATGTTTCCTACAATA
GATTCAGTGGTCCGGTCCCAGAAAATCTTCTGAATTTTCTGGTTTCCTCACCGAGCTC
CTTTAATGGCAATCCAGACCTCTGTATCTCTTGCCATACCAATGGTTCTTATTGCAAGG
GGTCTAATGTTTTGAAACCTTGTGGAGAGACCAAAAAACTACACAAACACGTCAAGAT
TGCTGTTATAGTTATTGGTTCATTGTTCGTTGGAGCAGTTTCCATACTTATACTGAGTT
GCATCCTTTTAAAGTTTTATCATCCAAAGACAAAAAATTTAGAATCAGTCAGTACTCTG
TTTGAAGGTTCTTCTTCTAAATTAAATGAGGTTATAGAGGCTACTGAAAACTTTGATGA
CAAGTATATCATCGGTACTGGTGCTCATGGAACTGTTTACAAGGCAACACTGAGGTCA
GGAGAAGTATATGCTGTAAAGAAGCTTGCAATATCTGCACAGAAAGGTTCGTACAAAA
GCATGATCAGAGAACTGAAGACATTAGGCAAAATCAAGCATCGGAACTTGATAAAGCT
GAAAGAGTTTTGGTTAAGAAGTGAGTATGGGTTCATGCTTTATGTTTATATGGAGCAA
GGTAGCCTTCAAGATGTTCTGCATGGGATCCAACCTCCTCCAAGTTTGGACTGGAGT
GTGCGCTATACCATAGCTCTTGGTACTGCCCATGGGCTAGCGTATCTTCATGATGACT
GTCAACCTGCAATTATTCACCGAGATATTAAGCCCAGTAATATACTTCTGAATGGGGA
CATGGTTCCACATATAGCAGATTTTGGCATTGCAAAGCTCATGGACCAGTCTTCTTCT
GCTCCACAGACTACTGGAGTTATTGGCACCTTTGGATATATGGCCCCAGAGTTGGCA
TTTTCCACCAGGAGTAGTATCGAGTCCGATGTATACAGCTACGGCGTCATACTCCTTG
AGCTGCTAACAAAAAAACAGGTGGTGGATCCCTCGTTCCCCGACAACATGGACATTG
TCGGTTGGGTGACCGCAACGCTCAACGGCACCGACCAAATCGAACTCGTCTGCGACT
CGACGCTGATGGAGGAAGTCTATGGCACGGTGGAAATAGAGGAAGTCAGCAAGGTC
CTGTCCTTGGCTCTTAGGTGCGCAGCGAAGGAAGCGAGCCGAAGGCCGCCCATGGC
CGATGTTGTGAAGGAGCTGACTGATGTCAGGAAATCCGCCGGGAAGTTGTCCAAGCC
GGAGAAGACGGCCTCCCGGAGCTCGTCCTGA
SEQ ID NO: 58-SIPEPR1-LIKE1 (RER-LIKE1) Rice Amino Acid Sequence (OsRER-
LIKE1)
MRLVVWHWFFFFFFTSVSSSWSLTSDGLALLSLSRDLILPHSISSTWKASDTTPCNWDGV
SCNKKNSVVSLDLSSSGVSGSLGPQIGLMKSLQVLSLSNNSISGSIPQELGNCSMLDQLD
LSSNSFSGEIPASLGDIKKLSSLSLYSNSLTGEIPEGLFKNQFLEQVYLHYNKLSGSIPLTV
GEMTSLRYLWLHGNKLSGVLPDSIGNCTKLEELYLLDNQLSGSLPKTLSYIKGLKIFDITAN
SFTGEITFSFEDCKLEVFILSFNQISNEIPSWLGNCSSLTQLAFVNNNISGQIPSSLGLLRNL
SQLLLSENSLSGPIPPEIGNCQLLVWLELDANQLNGTVPKELANLRKLEKLFLFENRLIGEF
PEDIWSIKSLQSVLIYENSFTGRLPPVLAELKFLKNITLFNNFFTGVIPPDLGVNSRLTQIDFT
NNSFVGGIPPNICSGKRLRILDLGLNLLNGSIPSNVMDCPSLERFILQNNNLSGPIPQFRNC
ANLSYIDLSHNSLSGNIPASLGRCVNITMIKWSENKLVGPIPSEIRDLVNLRVLNLSQNSLQ
GVLPVQISSCSKLYLLDLSFNSLNGSALTTVSNLKFLSQLRLQENKFSGGIPDSLSQLDMLI
ELQLGGNVLGGSIPSSLGRLVKLGIALNICSNGLVGGIPPLLSNLVELQSLDLSLNGLTGDL
DMLGNLQLLHVLNVSYNRFSGPVPENLLNFLVSSPSSFNGNPDLCISCHTNGSYCKGSN
VLKPCGETKKLHKHVKIAVIVIGSLFVGAVSILILSCILLKFYHPKTKNLESVSTLFEGSSSKL
NEVIEATENFDDKYIIGTGAHGTVYKATLRSGEVYAVKKLAISAQKGSYKSMIRELKTLGKI
KHRNLIKLKEFWLRSEYGFMLYVYMEQGSLQDVLHGIQPPPSLDWSVRYTIALGTAHGLA
YLHDDCQPAIIHRDIKPSNILLNGDMVPHIADFGIAKLMDQSSSAPQTTGVIGTFGYMAPEL
AFSTRSSIESDVYSYGVILLELLTKKQVVDPSFPDNMDIVGWVTATLNGTDQIELVCDSTL
MEEVYGTVEIEEVSKVLSLALRCAAKEASRRPPMADVVKELTDVRKSAGKLSKPEKTASR
SSS
SEQ ID NO: 59-SIPEPR1 (RER) Maize DNA Sequence (ZmRER)
ATGAAGCTGGTTTTCTGGCATTGGATTTTTCTATTCTTCGTGTTGCTTTCAACATCACA
GGGTATGAGTTCAGATGGCCTAGCTCTTCTTGCTCTGTCCAAAACCCTAATACTACCA
AGTTTCATAAGGACCAACTGGAGTGCTTCTGATGCAACTCCTTGTACATGGAACGGTG
TTGGCTGCAATGGAAGGAACAGAGTGATTTCTCTCGACCTATCGTCATCAGAGGTCTC
AGGTTTTATAGGACCTGAAATAGGGCGTCTGAAATACCTGCAGGTTCTCATTTTATCT
GCTAACAACATATCTGGTTTGATCCCTCTAGAATTGGGCAACTGCAGTATGCTTGAAC
AATTGGATCTGTCCCAAAACTTGCTTTCTGGCAATATACCGGCATCAATGGGCAGCCT
CAAGAAATTGTCATCACTGTCGCTGTACTACAACTCTTTCCATGGAACAATACCAGAG
GAGTTGTTCAAGAACCAGTTTCTGGAGCAAGTGTACCTACATGGAAATCAGCTCAGTG
GTTGGATACCCTTCTCGGTTGGTGAAATGACAAGCCTTAAGTCATTGTGGTTGCACGA
AAATATGTTGTCCGGAGTTTTGCCCAGTTCAATTGGCAACTGCACCAAGTTGGAGGAG
CTGTATCTACTCCATAATCAACTGAGTGGCAGTATTCCAGAAACCTTGAGTAAGATCG
AAGGCCTCAAGGTTTTTGATGCCACTGCCAATAGTTTCACGGGCGAGATCTCTTTCAG
TTTTGAGAACTGCAAGCTGGAAATATTCATCTTGTCATTCAATAATATAAAGGGTGAAA
TTCCGTCATGGCTAGGGAATTGCAGGAGCTTGCAACAACTTGGATTTGTCAATAATAG
TCTGTCTGGCAAAATTCCAAATTTTATAGGCTTATTCAGCAACCTCACGTATCTTTTAC
TTTCACAGAACTCCCTGACTGGGCTGATCCCACCTGAGATTGGTAACTGTCGGTTGCT
GCAGTGGCTAGAGCTAGATGCAAATCAGCTGGAGGGCACTGTTCCTGAAGAATTTGC
AAATTTAAGGTATTTGTCAAAGCTCTTTCTTTTCGAGAATCACCTCATGGGAGACTTCC
CTGAGAGTATTTGGAGTATCCAAACCCTCGAGAGTGTCCTTCTTTATAGCAACAAATT
CACAGGGAGGCTACCTTCAGTGTTAGCTGAGCTGAAGTCCCTAAAGAACATCACACT
GTTTGATAATTTCTTCACTGGAGTCATACCACAGGAGCTGGGTGTTAATAGCCCCTTG
GTCCAGATAGATTTCACAAATAACAGTTTTGTTGGTGGTATCCCACCAAACATTTGTTC
AGGAAAAGCATTGAGAATTTTGGACTTGGGGTTTAATCATCTCAACGGTAGCATCCCA
TCCAGTGTTCTGGACTGCCCAAGTCTGGAGCGAGTCATTGTCGAAAACAATAACCTTG
TTGGGTCTATTCCGCAATTTATAAACTGTGCAAATCTAAGTTATATGGATCTGAGCCAC
AATTCCTTGAGTGGTAACATACCATCAAGTTTCAGCAGGTGTGTAAAAATTGCTGAGA
TAAACTGGTCAGAGAACAATATTTTTGGGGCAATACCACCAGAAATTGGAAAGTTGGT
GAATCTGAAAAGGCTTGACCTCTCACACAATCTATTGCATGGTTCGATCCCTGTGCAA
ATTTCTAGTTGCTCCAAGTTGTATTCACTTGATTTGGGTTTTAACTCTTTGAATGGTTC
GGCCCTCAGCACAGTAAGCAGCCTGAAGTTTCTGACACAGCTACGATTGCAAGAGAA
TAGATTCAGCGGAGGTTTGCCTGATCCTTTCTCACAATTGGAAATGCTTATTGAGCTG
CAACTTGGTGGAAATATTCTTGGGGGCAGTATCCCTTCATCATTAGGACAGCTGGTGA
AACTGGGTACAACCTTGAACCTTAGTAGCAATGGTCTAGTGGGTGACATTCCATCACA
ATTCGGTAATTTGGTGGAGTTGCAAAACTTAGATTTGTCATTTAATAATCTCACAGGAG
GCCTTGCTACATTGCGAAGTCTACGCTTTTTGCAGGCCTTGAATGTTTCTTACAACCA
ATTTAGTGGACCAGTTCCAGATAATCTTGTGAAGTTTCTGAGTTCCACAACAAATTCTT
TTGATGGAAACCCAGGCCTCTGTATCTCTTGCAGCACCAGTGATTCTTCTTGCATGGG
AGCTAATGTTCTGAAACCTTGTGGCGGGTCAAAGAAAAGAGCAGTGCATGGCCGATT
CAAAATTGTTCTCATAGTTCTTGGCTCATTATTTGTGGGAGCAGTTCTGGTACTCATAC
TCTGGTGCATCCTTCTGAAATCTCGAGATCAGAAGAAGAATAGTGAGGAAGCAGTCA
GTCATATGTTTGAAGGTTCCTCATCTAAATTAAATGAGGTTATAGAGGCAACTGAATGT
TTTGATGACAAGTATATCATTGGTAAAGGTGGTCACGGAACCGTTTACAAGGCAACAC
TGAGGTCAGGGGATGTTTATGCTATAAAGAAACTTGTGATTTCTGCACACAAAGGTTC
ATACAAAAGCATGGTTGGAGAACTGAAGACACTAGGTAAAATCAAGCACAGGAACTT
GATTAAGCTGAAAGAATCTTGGTTGAGAAATGACAATGGATTCATACTGTATGATTTTA
TGGAAAAAGGTAGCCTTCATGATGTTCTACATGTAGTTCAGCCAGCACCAGCCTTAGA
CTGGTGTGTGCGGTATGACATAGCCCTCGGCACTGCCCATGGGTTAGCATATCTACA
TGATGACTGCCGCCCTGCGATCATTCATCGCGACATCAAGCCAAGTAATATACTGCTG
GACAAGGACATGGTGCCACATATTTCAGATTTTGGCATTGCAAAGCTCTTGGAGCAGC
CTTCTACTGCTCCTCAGACCACTGGTGTTGTTGGCACCATTGGATATATGGCCCCAGA
GTTAGCGTTCTCCACCAAGAGCAGCATGGAGTCCGACGTGTACAGCTACGGCGTGGT
GCTGCTGGAGCTGCTCACGAGGAGGGGGGCGGTGGATCCCTCGTTTCCCGACGGCA
CGGACATAGTCAGCTGGGCGTCGTCCGCCCTGAACGGCACTGACAAAATCGAGGCC
GTCTGCGACCCGGCCCTCATGGAGGAAGTCTTCGGCACGGTGGAGATGGAGGAGGT
GAGTAAGGTCCTGTCAGTGGCGCTGCGGTGCGCGGCCAGGGAGGCGAGCCAAAGG
CCCTCCATGACCGCGGTCGTGAAGGAGCTGACGGATGCACGGCCTGCCACTGGCGG
CGGCCGGTCGTTGTCCAAGTCGAAGCAGGGGAAACCAGGATCGCAATCCAACAGCA
GCGCCTACCGGCAGTAG
SEQ ID NO: 60-SIPEPR1 (RER) Maize Amino Acid Sequence (ZmRER)
MKLVFWHWIFLFFVLLSTSQGMSSDGLALLALSKTLILPSFIRTNWSASDATPCTWNGVG
CNGRNRVISLDLSSSEVSGFIGPEIGRLKYLQVLILSANNISGLIPLELGNCSMLEQLDLSQ
NLLSGNIPASMGSLKKLSSLSLYYNSFHGTIPEELFKNQFLEQVYLHGNQLSGWIPFSVGE
MTSLKSLWLHENMLSGVLPSSIGNCTKLEELYLLHNQLSGSIPETLSKIEGLKVFDATANSF
TGEISFSFENCKLEIFILSFNNIKGEIPSWLGNCRSLQQLGFVNNSLSGKIPNFIGLFSNLTY
LLLSQNSLTGLIPPEIGNCRLLQWLELDANQLEGTVPEEFANLRYLSKLFLFENHLMGDFP
ESIWSIQTLESVLLYSNKFTGRLPSVLAELKSLKNITLFDNFFTGVIPQELGVNSPLVQIDFT
NNSFVGGIPPNICSGKALRILDLGFNHLNGSIPSSVLDCPSLERVIVENNNLVGSIPQFINCA
NLSYMDLSHNSLSGNIPSSFSRCVKIAEINWSENNIFGAIPPEIGKLVNLKRLDLSHNLLHG
SIPVQISSCSKLYSLDLGFNSLNGSALSTVSSLKFLTQLRLQENRFSGGLPDPFSQLEMLIE
LQLGGNILGGSIPSSLGQLVKLGTTLNLSSNGLVGDIPSQFGNLVELQNLDLSFNNLTGGL
ATLRSLRFLQALNVSYNQFSGPVPDNLVKFLSSTTNSFDGNPGLCISCSTSDSSCMGANV
LKPCGGSKKRAVHGRFKIVLIVLGSLFVGAVLVLILWCILLKSRDQKKNSEEAVSHMFEGS
SSKLNEVIEATECFDDKYIIGKGGHGTVYKATLRSGDVYAIKKLVISAHKGSYKSMVGELKT
LGKIKHRNLIKLKESWLRNDNGFILYDFMEKGSLHDVLHVVQPAPALDWCVRYDIALGTAH
GLAYLHDDCRPAIIHRDIKPSNILLDKDMVPHISDFGIAKLLEQPSTAPQTTGVVGTIGYMA
PELAFSTKSSMESDVYSYGVVLLELLTRRAAVDPSFPDGTDIVSWASSALNGTDKIEAVC
DPALMEEVFGTVEMEEVSKVLSVALRCAAREASQRPSMTAVVKELTDARPATGGGRSLS
KSKQGKPGSQSNSSAYRQ
SEQ ID NO: 61-SIPEPR1-LIKE1 (RER-LIKE1) Maize DNA Sequence (ZmRER-LIKE1)
ATGAAGCTGGTTTTATGGCATCAGTTTTTTCTCTTCTTCGTGTTAGTTTCAACATCACA
GGGTATGAGTTCAGATGGCCTAGCTCTTCTTGCTCTGTCCAAAAGCCTCATACTACCA
AGTCCCATAAGAACCAACTGGAGTGATTCTGATGCAACTCCCTGTACATGGAGCGGT
GTTGGTTGCAATGGAAGGAACAGAGTCATCTCTCTCGACCTATCATCGTCAGGTGTTT
CGGGTTCTATAGGACCTGCAATAGGGCGTCTGAAATACCTGCGGATTCTCATCTTATC
AGCTAACAACATATCTGGTTTGATCCCTCTAGAATTGGGAGACTGCAATATGCTTGAA
GAACTGGATTTGTCCCAAAACCTGTTTTCTGGCAATATACCAGCATCATTGGGCAACC
TCAAGAAATTGTCATCACTGTCACTGTACCGCAACTCCTTCAATGGAACAATACCAGA
GGAGTTGTTCAAGAACCAGTTTCTGGAGCAAGTGTACCTACATGACAATCAGCTCAGT
GGTTCGGTGCCCTTATCGGTTGGTGAAATGACAAGCCTTAAGTCACTGTGGTTACAG
GAAAATATGTTGTCTGGAGTTTTGCCCAGTTCAATTGGAAACTGCACCAAGTTGGAGG
ATCTGTATCTACTCGATAATCAACTGAGTGGCAGTATTCCTGAAACCTTGGGTATGAT
CAAAGGTCTCAAGGTTTTTGATGCTACTACCAATAGTTTCACAGGTGAGATCTCTTTCA
GTTTTGAGGACTGCAAGCTAGAAATATTCATCTTGTCTTTCAATAATATAAAAGGTGAA
ATTCCATCATGGCTGGGGAACTGCATGAGCTTGCAACAACTTGGATTTGTCAATAATA
GTTTGTATGGCAAAATTCCAAATTCTCTTGGCTTATTGAGCAACCTCACATATCTTTTA
CTTTCACAGAACTCCCTTTCTGGGCCGATCCCACCTGAGATTGGTAACTGTCAGTCGC
TGCAGTGGCTAGAGTTAGATGCAAACCAGCTGGATGGCACTGTTCCTGAAGAATTTG
CAAATTTACGGAGTTTGTCAAAGCTCTTTCTTTTTGAGAATCGCCTCATGGGAGACTTC
CCTGAGAATATTTGGAGTATCCAAACCCTCGAGAGTGTCCTACTTTATAGCAACAGAT
TCACAGGGAAGCTACCTTCAGTGTTAGCTGAGCTGAAGTTCCTAAAGAACATCACACT
GTTTGATAATTTCTTCACTGGAGTCATACCACAGGAGCTAGGTGTTAATAGCCCCTTG
GTCCAGATAGATTTCACAAACAACAGTTTTGTTGGTAGTATCCCACCAAACATCTGTTC
AAGAAAAGCATTGAGAATTTTGGACTTAGGGTTTAATCATCTCAACGGTAGCATCCCA
TCCAGTGTTGTGGACTGCCCAAGTTTGAAGCGAGTCATTTTACAAAACAATAACCTTA
ACGGGTCTATTCCACAATTTGTAAACTGTGCAAATCTAAGTTATATGGACCTAAGCCA
CAATTCCTTGAGTGGTAACATTCCAGCAAGCTTCAGCAGATGTGTAAACATTACTGAG
ATAAACTGGTCAGAGAACAAGCTTTTTGGAGCAATACCACCTGAAATTGGAAACTTAG
TGAATCTGAAAAGACTTGACCTCTCACACAACATATTGCATGGTTCGATCCCTGTGCA
AATTTCCAGTTGCTCCAAGTTGTACTCACTTGATTTGAGTTTTAACTCATTGAATGGTT
CGGCCCTCCGCACCGTAAGCAACTTGAAGTTTCTGACACAGCTACGATTGCAAGAGA
ATAGATTCAGCGGAGGCTTGCCTGATTCTCTCTCACAATTGGAAATGCTTATTGAGCT
GCAACTTGGTGGAAATATTCTTGGGGGTAGTATCCCTTCGTCATTAGGACAGCTGGT
GAAACTGGGCACAGCCTTGAACCTTAGTAGCAATGGCCTAATGGGTGACATTCCAAC
ACAATTGGGTAATTTGGTGGAGTTGCAAAACTTAGATTTTTCATTTAATAATCTCACAG
GAGGCCTTGCTACATTGAGAAGTCTAGGCTTTTTGCAGGCCTTGAATGTTTCTTACAA
CCAATTTAGTGGACCAGTCCCAGATAATCTTCTGAAGTTTCTGAGTTCCACACCATATT
CTTTTGATGGAAACCCAGGCCTCTGTATCTCCTGCAGCACCAGTGGCTCTTCTTGCAT
GGGAGCTAATGTTTTGAAACCTTGTGGTGGGTCGAAGAAAAGAGGAGTACATGGCCA
ATTGAAAATTGTTCTCATAGTTCTCGGTTCATTATTTGTGGGAGGAGTTCTTGTACTTG
TACTGTGTTGCATCCTTCTGAAATCTCGAGATTGGAAGAAAAATAAAGTCAGTAACAT
GTTTGAAGGTTCCTCATCTAAATTAAATGAGGTTACAGAGGCCACTGAAAATTTCGAT
GACAAGTATATCATCGGTACAGGTGCTCACGGAACTGTTTACAAGGCAACACTGAGG
TCAGGGGATGTTTATGCTATAAAGAAGCTTGCGATTTCTGCACACAAAGGTTCATACA
AAAGCATGGTTAGAGAACTGAAGACACTGGGTGAAATTAAGCACAGAAACTTGATAAA
GCTGAAAGAATTTTGGTTGAGAAGTGATAATGGATTCATACTGTATGATTTTATGGAAA
AGGGCAGCCTCCATGATATTCTGCATGTAATTCAGCCAGCACCAGCTTTGGACTGGT
GTGTGAGGTATGACATAGCTCTTGGCACCGCCCATGGGTTAGCATATCTTCATGATGA
CTGCCGCCCTGCGATCATTCACCGTGATATTAAACCAAGAAATATACTGCTCGACAAG
GACATGGTGCCACATATTTCAGATTTTGGCATTGCAAAGCACATGGACCAGTCTTCTA
CTACTGCTCCACAGACCACTGGAATCGTTGGCACTATTGGATATATGGCCCCAGAATT
GGCGTTTTCCACCAAGAGCAGCATGGAGTCTGACGTGTACAGCTACGGTGTGGTGCT
ACTGGAGCTGTTGACCAGGAGGACGGCGGTGGATCCTTTGTTCCCCGACAGCGCGG
ACATAGTCGGCTGGGTGTCGTCCGTGCTGGACGGCACCGACAAAATCGAGGCCGTC
TGTGACCCGGCCCTCATGGAGGAAGTCTTCGGCACGGTGGAGATGGAGGAGGTGCG
TAAGGTCCTGTCGGTGGCGCTCCGGTGCGCGGCCAGGGAGGTGAGCCAAAGGCCC
TCCATGACTGCCGTCGTGAAGGAGCTGACGGATGCGCGGCCAGCCTCTGCCAGCAG
CGGCAGCCGGTCGTTGTCCAAGTCGAGGGAAGGGAAACCGGGATTGCAATCCAGCA
GCAGCGCGTACTGGCAGTAG
SEQ ID NO: 62-SIPEPR1-LIKE1 (RER-LIKE1) Maize Amino Acid Sequence (ZmRER-
LIKE1)
MKLVLWHQFFLFFVLVSTSQGMSSDGLALLALSKSLILPSPIRTNWSDSDATPCTWSGVG
CNGRNRVISLDLSSSGVSGSIGPAIGRLKYLRILILSANNISGLIPLELGDCNMLEELDLSQN
LFSGNIPASLGNLKKLSSLSLYRNSFNGTIPEELFKNQFLEQVYLHDNQLSGSVPLSVGEM
TSLKSLWLQENMLSGVLPSSIGNCTKLEDLYLLDNQLSGSIPETLGMIKGLKVFDATTNSF
TGEISFSFEDCKLEIFILSFNNIKGEIPSWLGNCMSLQQLGFVNNSLYGKIPNSLGLLSNLTY
LLLSQNSLSGPIPPEIGNCQSLQWLELDANQLDGTVPEEFANLRSLSKLFLFENRLMGDFP
ENIWSIQTLESVLLYSNRFTGKLPSVLAELKFLKNITLFDNFFTGVIPQELGVNSPLVQIDFT
NNSFVGSIPPNICSRKALRILDLGFNHLNGSIPSSVVDCPSLKRVILQNNNLNGSIPQFVNC
ANLSYMDLSHNSLSGNIPASFSRCVNITEINWSENKLFGAIPPEIGNLVNLKRLDLSHNILH
GSIPVQISSCSKLYSLDLSFNSLNGSALRTVSNLKFLTQLRLQENRFSGGLPDSLSQLEMLI
ELQLGGNILGGSIPSSLGQLVKLGTALNLSSNGLMGDIPTQLGNLVELQNLDFSFNNLTGG
LATLRSLGFLQALNVSYNQFSGPVPDNLLKFLSSTPYSFDGNPGLCISCSTSGSSCMGAN
VLKPCGGSKKRGVHGQLKIVLIVLGSLFVGGVLVLVLCCILLKSRDWKKNKVSNMFEGSS
SKLNEVTEATENFDDKYIIGTGAHGTVYKATLRSGDVYAIKKLAISAHKGSYKSMVRELKTL
GEIKHRNLIKLKEFWLRSDNGFILYDFMEKGSLHDILHVIQPAPALDWCVRYDIALGTAHGL
AYLHDDCRPAIIHRDIKPRNILLDKDMVPHISDFGIAKHMDQSSTTAPQTTGIVGTIGYMAP
ELAFSTKSSMESDVYSYGVVLLELLTRRTAVDPLFPDSADIVGWVSSVLDGTDKIEAVCDP
ALMEEVFGTVEMEEVRKVLSVALRCAAREVSQRPSMTAVVKELTDARPASASSGSRSLS
KSREGKPGLQSSSSAYWQ
SEQ ID NO: 63-SIPEPR1 (RER) Wheat DNA Sequence (TaeRER)
ATGGGGCTTGTATTGTGGCATTCATTGTTTCTCTTCTTAAGTTTGGTTTCCACGTCATG
GAGTTTGAATTCAGATGGTCGCGCCCTTCTTGCTCTGTCCAAAAATCTCATATTGCCT
AGTTCCATAAAGTCAAGTTGGAATGCTTCTGATACAACCCCATGTAACTGGACTGGAA
TTAGTTGTGATGAAAGGAACAATGTGGTTTCTCTTGACCTAACATCGTCTGGAGTTTCT
GGTTCATTAGGAGTTCAAATAGGTCTTCTAAAGTACATACAAGTCATCATTTTGCTAAA
CAATAGCATATCTGGTCCAATCCCTCAAGAACTGGGCAATTGTAGCATGCTTGAACAG
TTGGATCTTTCCATGAACTTCCTTTCTGGTGAAATACCAGAATCACTGTCCAACCTAAA
AAAACTATCATCATTCTCGTTGTACACTAACTCCCTCAGTGGGGAAATACCAGAGGGG
TTGTTCAAGAACCAGTTTCTGCAGGACGTGTACCTCCATGGGAATAATCTCAGTGGTT
CTATCCCTTCATCAGTTGGTGAAATGACAAGCCT
TAGATCATTTTGGTTGACGAAGAATGCATTATCTGGAGGTCTGCCAGATTCAATTGCA
ACTGCACCAAGTTGGAGGAGCTCTATCTACTGGATAATCAGTTGAGCGGGAGCCTTC
CAAAAACCTTGAGCTATGTCAAAGGACTGAAAGTTTTAGATGCCACTGGAAATAGCTT
TACCGGAGAGATTGATTTCAGTTTTGAGAACTGCAAGTTGGAGATATTCATATTGTCG
TTCAATCATATGAGTGGCGGTATTCCATCATGGCTAGGGAATTGCACTAGCTTGACAC
AACTTGCACTTGTCGACAACCGTTTCACTGGCCAAATTCCGGCTTCTCTTGGCCTATT
GAGCAACCTCACCTTGCTTATGCTTTCTCAAAACTCCTTGTCTGGCTCAATCCCTCCT
GAGATTGGTAACTGTTGGTTGCTGGAGTGGCTAGAGTTGGATCATAACATGCTCGAG
GGCACTGTTCCTAAAGAGCTGGCTAATCTGAGACACTTGCAGAAGCTCTTTCTTTTCG
AGAATCGCCTGACCGGGGAGTTTCCTGAGGGTATTTGGAGCATCCGGTACCTTCAAA
GTGTTCTTATTTACAGCAATGGTTTTACTGGGAAGCTACCTCTTAAGTTAGCTGAATTG
AAGTTATTGGAGAACATCACATTGTTCGATAATTTCTTCACCGGAGTCATACCCCCGG
GTTTGGGTGTTAATAGCCCTTTACAGCAAATTGATTTCACCAACAACAGTTTTACCGGT
GGAATACCTCCGTACATTTGTTCAAGGAAAAGACTCAGGGTGTTGGTTTTGGGTCTCA
ATCATCTGAATGGTAGCATCCCATCCAATGTTGCAGATTGCCCAGGTTTGGAACGAAT
CATTCTCAAAAACAATGATCTTACTGGGCCCATTCCACATTTTAGAAACTGTGCAGCG
CTGGGCTATATGGATTTCAGTCATAATTCTTTAAGTGGAGATATTCCAGCAAGCTTGG
GAAAATGCTTAAATACTACAATGATAAACTGGTCAGCAAACAAACTTGTTGGTCCAATA
CCACCTGAAGTTAGAAACTTGGTGAATTTGGAAGTTCTTAACCTCTCACAAAACAGTC
TACAGGGTGCACTTCCAGCTCAGGTTTCTAGTTGCTCCAAGCTGCTTATTTTGGATTT
GAGTTTTAACTCTCTGCATGGTCCGGCACTCACGACATTGGTTATGCTTCTTGAGCTG
CAACTTGGTGGCAACATTCTTGGAGGCAGAATCCCTTCGTCGTTAGGAAAGTTGATCA
AACTCATTGCATTCAATCTCAGCAGCAACGGACTGGTGGGTGATATTCCAACACCATT
GGGCATTTGGTGGAACTGCAAAGTTTAGATTTGTCAGTTAATAACCTCACTGGAGCTC
TCGGCGCATTAGGGAGTCTACATTCATTGCACGCCTTGAATCTTTCCTATAATAGGTT
CAGTGGACCAGTGCCAGAATATCTTCTGAAATTTCTGAACTCCACACCAAGCTCTTTT
AATGGAAATTCGGGTCTCTGTATCTCTTGCCGTGACAATGATTCTTCTTGCAAGAGAT
CTAATGTGCTGAAACCTTGTGGAGGATCAGGGAAAAAAGGAATAAAGCGTCGATTCA
AGGTTGCTCTTATTATTCTTGGTTCATTGTTCATTGGAGCAGTAGCGGTACTTAT
CCTCTGCTGTATCCTTCTAAAGAATCGAGATTCGAAGACAAAAAGTGAGGAGACAATC
AGTAATTTGCTTGAAGGCTCTTCTTCTAAATTAAATGAGATCATAGAAAAGACAGAAAA
CTTTGATGACAAGTATATCATAGGCGCAGGTGCGCATGGGACTGTGTACAAGGCAAT
ATTGAATTCAGGGGAGGTCTTTGCCATAAAGAAGCTTGCGATTTCAGCGCGCAGCAG
CTCCTACAAAAGCATGATCAGAGAGCTGAAGACACTTGGTAAAGTTCGGCACAGGAA
CTTGATAAAGCTGAAGGAATTTTGGGTGAGAGGCGACTCTGGGTTCATACTGTATGAC
TTCATGGAGCATGGTAGCCTCTATGATGTCCTGCACAGGATCCGGACGCCGAGTCTG
GACTGGAGCATGCGCTATAACATAGCTCTTGGAACTGCCCATGGTCTGGCATATCTTC
ACCATGACTCTGTCCCTGCGATCATTCATCGAGATATTAAGCCAAGCAATATATTGTTA
AACAAGGACATGGTGCCGCGCATCGCGGATTTCGGCATAGCAAAGATCATGGATCAG
TGTTCTGCTGCTCCACAGTCCACCGGGGTCGTCGGCACCACTGGATATATGGCACCA
GAGCTGGCATTTTCCACCAGAAACAGTATCAAGACCGACGTGTACAGCTACGGCGTC
GTCCTGCTTGAGCTTATAACAGGAAAGACGGCGGTGGATCCCTCATTCCCGGAGAAC
ATGGACATTGTCGCCTGGGTGCCCCACGCCCTGAACGGCGCTGAGCAGATCGGGCC
CGTCTGCGACCCGGCCCTCCTGGACGAAGTGTACAGCACTGTCGAAATGGAGGAGG
TGCGCAAGGTCCTGCGCTTGGCCCTTAGGTGCACAGCGAAGGAGCCGAGTCAACGA
CCGTCCATGGTCGACGTCGTGAAGGAGCTGACTGACGCGAGGTTCGCGGGCATCCC
TTCGTCGTCGAAGCAGGGGAAGCCAGGCTCCTCCTCCGGCGGCGGCTCTTCTTGA
SEQ ID NO: 64-SIPEPR1 (RER) Wheat Amino Acid Sequence (TaeRER)
MGLVLWHSLFLFLSLVSTSWSLNSDGRALLALSKNLILPSSIKSSWNASDTTPCNWTGISC
DERNNVVSLDLTSSGVSGSLGVQIGLLKYIQVIILLNNSISGPIPQELGNCSMLEQLDLSMN
FLSGEIPESLSNLKKLSSFSLYTNSLSGEIPEGLFKNQFLQDVYLHGNNLSGSIPSSVGEM
TSLRSFWLTKNALSGGLPDSIGNCTKLEELYLLDNQLSGSLPKTLSYVKGLKVLDATGNSF
TGEIDFSFENCKLEIFILSFNHMSGGIPSWLGNCTSLTQLALVDNRFTGQIPASLGLLSNLT
LLMLSQNSLSGSIPPEIGNCWLLEWLELDHNMLEGTVPKELANLRHLQKLFLFENRLTGEF
PEGIWSIRYLQSVLIYSNGFTGKLPLKLAELKLLENITLFDNFFTGVIPPGLGVNSPLQQIDF
TNNSFTGGIPPYICSRKRLRVLVLGLNHLNGSIPSNVADCPGLERIILKNNDLTGPIPHFRN
CAALGYMDFSHNSLSGDIPASLGKCINTTMINWSANKLVGPIPPEVRNLVNLEVLNLSQNS
LQGALPAQVSSCSKLLILDLSFNSLHGPALTTLVMLLELQLGGNILGGRIPSSLGKLIKLIAF
NLSSNGLVGDIPTPLGNLVELQSLDLSVNNLTGALGALGSLHSLHALNLSYNRFSGPVPEY
LLKFLNSTPSSFNGNSGLCISCRDNDSSCKRSNVLKPCGGSGKKGIKRRFKVALIILGSLFI
GAVAVLILCCILLKNRDSKTKSEETISNLLEGSSSKLNEIIEKTENFDDKYIIGAGAHGTVYKA
ILNSGEVFAIKKLAISARSSSYKSMIRELKTLGKVRHRNLIKLKEFWVRGDSGFILYDFMEH
GSLYDVLHRIRTPSLDWSMRYNIALGTAHGLAYLHHDSVPAIIHRDIKPSNILLNKDMVPRI
ADFGIAKIMDQCSAAPQSTGVVGTTGYMAPELAFSTRNSIKTDVYSYGVVLLELITGKTAV
DPSFPENMDIVAWVPHALNGAEQIGPVCDPALLDEVYSTVEMEEVRKVLRLALRCTAKEP
SQRPSMVDVVKELTDARFAGIPSSSKQGKPGSSSGGGSS
SEQ ID NO: 65-SIPEPR1-LIKE1 (RER-LIKE1) Wheat DNA Sequence (TaeRER-LIKE1)
ATGGGGCTAATTTTATGGCATCATTTGCTTCTCTTCTCCAACCTAGTCTCGCTCTGCTG
CGGTTTGAGTTCAGATGGCCATGCCCTTCTGGCTCTCTCCAGGCGGCTCATACTGCC
TGATATCATAAGCTCGAACTGGAGTTCTTCTGATACAACTCCCTGTGGATGGAAGGGA
GTTCAGTGTGAGATGAACATTGTGGTTCACCTCAATCTATCATACTCCAAAGTTTCTG
GTTCAATTGGTCCTGAGGTAGGGCGTATGAAGTACCTGCGGCAACTCGATCTGTCAA
GCAACAACATCTCCGGTCCGATCCCTTATGAATTGGGAAATTGTCTTCTGCTTGACCT
GCTGGATCTGTCAGGGAACAGCCTTTCCGGTGGAATTCCGACATCCCTCATGAACCT
AAAGAAGCTATCACAGCTCGGCTTGTACAGCAACTCCTTCAGTGGAGAGATACCTGA
GGGGCTGTTCAAGAACCGGTTTCTGGAGCGGGTGTATCTCCAAGACAATAAACTCAG
TGGCTCCATCCCTCCATCGGTTGGTGAAATGAAGAGTCTGAAATACTTTAGGTTGGAT
GGGAACATGTTGTCCGGAGCTCTGCCGGACTCAATTGGCAACTGCACCAAGTTGGAG
AATCTATATCTATATGATAATAAACTGAATGGGAGTCTTCCGAGATCATTGAGCAACAT
CAAAGGGCTCGTGCTCTTTGAGGCCTACAACAACAGTTTTACAGGCGACATCTCTTTC
AGATTCAAGAGCTGCAAGCTTGAAGTGTTTGTGCTATCTTGGAATCAGATCAGTGGGG
AAATCCCGGGGTGGCTGGGAAATTGTAGCAGCTTAATCAGACTTGCATTTCTCCACAA
CCGTCTCTCTGGCCAGATACCAACTTCACTCGGTTTATTGAAAAAGCTATCAATCCTTA
TACTTACTCAGAATTCTTTGTCTGGGCTGATCCCTCCTGAGATTGGTAGCTGTCGGTC
GCTGGTGTGGCTGCAGCTGGACGCAAACCAGCTTGAGGGCACCGTTCCGAAACAGC
TGGCTAATCTGCGTAACTTGCAGCAGCTATTTCTGTTTGAGAATCGCCTCAGTGGGGA
GTTCCCTCAGGATATTTGGGGCATCCAAGGCCTCGAATATGTCCTTCTGTACAACAAC
AGCTTATCTGGGGGCCTACCTCCAATGTCGGCCGAGTTGAAGCACCTTAAGTTCGTC
AAACTACAGGATAATCTGTTCACTGGAGTCATACCACCAGGATTTGGGATTAACAGCC
CTTTAATAGAGATCGACTTCACAAATAATAGGTTTGTTGGTGGAATCCCACCGAACATT
TGTTCGGGTAAAAGATTAACAGTTTGGAATTTGGGGCATAATTTTCTCAATGGTACCAT
CCCGTTCACTGTTGCTAACTGCCCAAGTTTAGAGCGAGTTCGACTCCATAACAACAAT
CTCAGTGGGCAAGTTCCGCAATTCCGAGACTGTGCAAATCTGCGGTACATAGATTTG
AGTCACAATTCCTTAAGTGGTCATATTCCTGCAAGCTTGGGCAGGTGTGCTAACATTA
CAGCGATAAACTGGTCTGCAAACAAGCTTGGTGGTCCAATACCACCCGAACTCGGAC
AATTAGTGAAGTTGGAAAGTCTTGACCTCTCCCACAACAGCCTAGAGGGGGCAATTC
CTGCACAGATTTCCAGTTGCTCGAAGTTGCACTTGTTTGATTTGAGTTTCAACTCTTTG
AATGGTTCTGCGCTCACAACAGTATGCAAGCTGGAGTTTATGTTAAATCTGCGGCTAC
AGGGGAATAGATTAAGTGGAGGCATTCCAGATTGTATCTCGCAGTTACATGGGCTAG
TCGAGCTACAACTTGGCGGCAATGCTCTTGGTGGTCATCTCCCTTCATCATTAGGAAT
TTTGAAAAGATTGAGTACTGCACTAAACCTTAGCAGCAATGGGCTGGAGGGCAGCAT
TCCATCTCAATTACGCTACTTGGTGGATCTTGCCAGCTTAGATTTGTCTGGTAATAATC
TCAGTGGGGATCTTGCTCCGTTAGGAAGTCTACGTGCATTGTATACATTGAATCTTTC
CAATAACAGATTCAGCGGGCCAGTGCCAGAGAATCTTTTACGGTTTATGAATTCTACA
CCAAGCCCATTTAGTGGAAATTCAGATCTCTGTGTGTCTTGTCATGATGGTGATTCTT
CTTGCAAGGGAGCTAATGTTTTGGAACCTTGTAGTTCATTGAGGAGAAGAGGAGTAC
ATGGCCGAGTCAAGATAGCTATGATATGTCTTGGTTCAGTTTTTGTTGGTGTGTTTCT
GATACTCTGTATCTTTCTGAAATACAGAGGTTCCAAGACTAAACCTGAGGGAGAGTTA
AATCCATTTTTTGGGGAGTCATCTTCTAAATTAAATGAGGTTTTGGAATCAACTGAAAA
CTTTGATGACAAGTACATCATCGGCACAGGTGGCCAAGGAACTGTATACAAGGCAAC
ATTGAGGTCAGGAGAAGTATATGCCGTGAAGAAGCTTGTGGGTCATGCACACAAGAT
TTTGCATGGAAGCATGATAAGGGAGATGAATACGCTTGGTCAAATTAGGCATAGGAA
CTTAGTAAAGCTAAAGGATGTTTTGTTCAGGCGTGAGTATGGGTTGATCCTCTATGAA
TTTATGGACAATGGTAGCCTTTATGATGTTCTCCATGGGGCTGAGCCAGCTCCAATTC
TGGAGTGGAGGATACGGTATGACATAGCTCTTGGTACAGCACATGGTTTAGCTTATCT
CCACAACGACTGCCACCCAGCCATTATTCACCGCGACATTAAACTGAAAAATATACTG
TTGGACAAGGACATGGTGCCACATATTTCAGACTTTGGCATTGCAAAGCTTATCGACC
TGTCTCCTGCTGCTTCAGAAACTACCGGAATCATTGGCACTGTTGGATATATGGCCCC
AGAGATGGCATTTTCAACCAGGAGTACCATTGAGTTCGATGTGTACAGCTACGGAGT
GGTATTACTTGAACTGATTACCAGAAAGATGGCCCTGGATCCCTCGTTCCCTCACGAT
GTGGACCTAGTCAGCTGGGTATCCTCCACCCTGAATGAGGGCAATGTGATCGAGTCC
GTGTGCGACCCTGCCCTAATGCGTGAGGTATGTGGCACTGCTGAACTGGAGGAAGTA
TGCAGTGTGCTGTCGATAGCCCTTAGATGCACCACGGAGGACGCAAGACAGAGGCC
TTCCATGATGGATGTTGTGAAAGAGCTGACACGTGCTAGGCGTGATGCTGTATCGCT
ACCGAAGCAGGCCATATCTGGTTCCAGCAGTTCCTGTCTAAACCTGGCAACCTGA
SEQ ID NO: 66-SIPEPR1-LIKE1 (RER-LIKE1) Wheat Amino Acid Sequence (TaeRER-
LIKE1)
MGLILWHHLLLFSNLVSLCCGLSSDGHALLALSRRLILPDIISSNWSSSDTTPCGWKGVQC
EMNIVVHLNLSYSKVSGSIGPEVGRMKYLRQLDLSSNNISGPIPYELGNCLLLDLLDLSGN
SLSGGIPTSLMNLKKLSQLGLYSNSFSGEIPEGLFKNRFLERVYLQDNKLSGSIPPSVGEM
KSLKYFRLDGNMLSGALPDSIGNCTKLENLYLYDNKLNGSLPRSLSNIKGLVLFEAYNNSF
TGDISFRFKSCKLEVFVLSWNQISGEIPGWLGNCSSLIRLAFLHNRLSGQIPTSLGLLKKLSI
LILTQNSLSGLIPPEIGSCRSLVWLQLDANQLEGTVPKQLANLRNLQQLFLFENRLSGEFP
QDIWGIQGLEYVLLYNNSLSGGLPPMSAELKHLKFVKLQDNLFTGVIPPGFGINSPLIEIDF
TNNRFVGGIPPNICSGKRLTVWNLGHNFLNGTIPFTVANCPSLERVRLHNNNLSGQVPQF
RDCANLRYIDLSHNSLSGHIPASLGRCANITAINWSANKLGGPIPPELGQLVKLESLDLSHN
SLEGAIPAQISSCSKLHLFDLSFNSLNGSALTTVCKLEFMLNLRLQGNRLSGGIPDCISQLH
GLVELQLGGNALGGHLPSSLGILKRLSTALNLSSNGLEGSIPSQLRYLVDLASLDLSGNNL
SGDLAPLGSLRALYTLNLSNNRFSGPVPENLLRFMNSTPSPFSGNSDLCVSCHDGDSSC
KGANVLEPCSSLRRRGVHGRVKIAMICLGSVFVGVFLILCIFLKYRGSKTKPEGELNPFFG
ESSSKLNEVLESTENFDDKYIIGTGGQGTVYKATLRSGEVYAVKKLVGHAHKILHGSMIRE
MNTLGQIRHRNLVKLKDVLFRREYGLILYEFMDNGSLYDVLHGAEPAPILEWRIRYDIALG
TAHGLAYLHNDCHPAIIHRDIKLKNILLDKDMVPHISDFGIAKLIDLSPAASETTGIIGTVGYM
APEMAFSTRSTIEFDVYSYGVVLLELITRKMALDPSFPHDVDLVSWVSSTLNEGNVIESVC
DPALMREVCGTAELEEVCSVLSIALRCTTEDARQRPSMMDVVKELTRARRDAVSLPKQAI
SGSSSSCLNLAT
SEQ ID NO: 67-SIPEPR1-LIKE2 (RER-LIKE2) Wheat DNA Sequence (TaeRER-LIKE2)
ATGGGGCTTGTATTGTGGCATTCATTGTTTCTCTTCTTATGTTTGGTTTCCTCGTCATG
GAGTTTGAATTCAGATGGTCGTGCCCTTCTTGCTCTGTCCAAAAATCTCATATTGCCTA
GTTCCATAAAGTCAAGTTGGAATGCTTCTGATACAACCCCATGTAACTGGACTGGAAT
TAGTTGTGATAAAAGGAACAATGTGGTTTCTCTTGACCTAACATTGTCTGGAGTTTCTG
GTTCACTAGGAGTTCATATAGGGCTTCTAAAGTACATAAAAGTCATCAATTTGCCGAG
CAATAACATATGTGGTCCAATCCCCCAAGAATTGGGCAATTGTAGCATGCTTGAACAG
TTGGATGTTTCCGGGAACTTCCTTTCTGGTGAAATACCAGAATCGCTTGGCAACCTCA
AAAAACTATCATACCTCTCGTTGTACAATAACTCCCTCAGTGGGGAAATACCAGAGGG
GTTGTTCAAGAACCAGTTTCTGCAGGACGTGTTCCTCAATGAGAATAAACTCAGTGGT
TCTATCCCTTCATCAGTTGGTGAAATGACAAGCCT
TAGATCATTTTGGTTGACGCAGAATGCATTATCTGGAGGTCTGCCAGATTCAATTGGC
AACTGCACCAAGTTGGAGGAGCTCTATCTGCTGGATAATCGGTTGAGCGGGAGCCTT
CCGAAAACCTTGAGCTATGTCAAAGGACTGAAAGTTTTAGATGCCACTGGAAATAGCT
TCACCGGAGAGATTGATTTCAGTTTTGAGAACTGCAAGTTGGAGAAATTCATATTCTC
GTTCAATCAGATGAGGGGCGGTATTCCAGCGTGGCTAGGTAATTGCAGTAGCTTGAC
AGAACTTGCACTTGTCAACAACAGTTTCTCTGGCCAAATTCCGCCTTCTCTTGGCCTA
TTGAGCAACCTCACCTTGCTTATGCTTTCTCAAAACTCCTTGTCTGGCCCAATCCCTC
CTGAGATTGGTAATTGTCGGTTGCTGGAGTGGCTAGAGTTGGATCATAACATGCTGG
AGGGCACTGTTCCTAAAGAGCTGGCTAATCTGAGACACTTGCAGAAGCTCTTTCTTTT
CGAGAATCGCCTGACCGGGGAGTTTCCTGAGGGTATTTGGAGCATCCGGTACCTTCG
AAGTGTTCTTATTTACAGCAATGGTTTTACTGGGAAGCTACCTCTTAAGTTAGCTGAAT
TGAAGTTATTGGAGAACATCACATTGTTCGATAATTTCTTCACTGGAGTCATACCCCTG
GGTTTGGGTGTTAATAGCCCTTTACAGCAAATTGATTTCACTAACAACAGTTTTACCGG
TGGAATACCTCCGTACATTTGTTCAAGGAAAAGACTCAGGGTGTTGGTTTTGGGTTTC
AATCTTCTGAATGGTAGCATCCCATCCAATGTTGCAGACTGCCCAGGCTTGCAACGG
ATCATTCTCAGAAACAATGATCTTACTGGGCCCATTCCACATTTTAGAAACTGTGCAG
CTCTGGGCTATACAGATTTCAGTCATAATTCTTTAAGTGGAGATATTCCAGCAAGCTTG
GGCAAATGCATAAATACTACAATGATAAACTGGTCAGCAAACAAACTTGTTGGTCCAA
TACCACCTGAAATTGGAAACTTGGTGAATTTGGGAGTTCTTAACCTCTCACAAAACAG
TCTCCAGGGTGCACTTCCAGCGCAGGTTTCTAGTTGCTCCAAGCTGTATATTCTGGAT
TTGAGTTTCAACTCTCTGCATGGTTCGGCACTCACGACAGTAAGCAGCCTGAAGCTTC
TCGCACAACTACGGTTGCAGGAGAATAAATTCAGTGGGGGCTTACCTGATTCTCTTTC
GCAGT
TGGTTATGCTTCTTGAGCTGCAACTTGGTGGCAACATTCTTGGAGGCAGCATCCCTTC
GTCGTTAGGAAAGTTGATCAAACTCATTGCATTGAATCTCAGCAGCAACGGACTGGTG
GGTGATCTTCGAACACCATTGGGCAATTTGGTGGAACTGCAAAGTTTAGATTTGTCAG
TTAATAACCTCACTGGAGGTCTTGGCGCATTAGGAAGTCTACATTCATTGCATGCCTT
GAATCTTTCCTATAATAGGTTCAGTGGACCAGTGCCAGAATATCTTCTGAAATTTCTGA
ACTCCGCACCAAGCTCTTTTAATGGAAATTCGGGTCTCTGTATCTCTTGCCGTGACAG
TGATTCTTCTTGCAAGAGATCTAATGTGCTGAAACCTTGTGGAGGATCAGGGAAAAAA
GGAATAAAGCGTCGATTCAAGGTTGCTCTTATTATTCTTGGTTCATTGTTCATTGGAGC
AGTAGCGGTACTTATCCTCTGCTGTATCCTTCTAAAGAATCGAGATTCGAAGACAAAA
AGTGAGGAGACAATCAGTAATTTGCTTGAAGGCTCTTCTTCTAAATTGAATGAGATCAT
AGAAAAGACAGAAAACTTTGATGACAAGTATATCATAGGCGCAGGTGCTCATGGGAC
TGTGTACAAGGCAATATTGAATTCAGGGGAGGTCTTTGCTATAAAGAAGCTTGCGATT
TCCGCGCGCAGCAGTTCCTACAAAAGCATGGTCAGAGAACTGAAGACGCTTGGTAAA
GTTCGGCACAGGAACTTGATAAAGCTGAAAGAATTTTGGGTGAGAGGCGACTCTGGG
TTCATACTGTATGACTTTATGGAGCATGGTAGCCTCTATGATGTCCTGCACAGGATCC
GGACGCCGAGTCTGGACTGGAGCATGCGCTATAACATAGCTCTTGGAACTGCCCATG
GTCTGGCATATCTTCACCATGACTCTGTCCCTGCGATCATTCATCGAGATATTAAGCC
GAGCAACATATTGCTGAACAAGGACTTGGTGCCGCGCATCGCAGATTTCGGCATAGC
GAAGATCATGGATCAGTGTTCAGCTGCTCCACAGACCACCGGGGTCGTCGGCACAA
CTGGATATATGGCACCAGAGCTGGCATTTTCCACCAGAAACAGTATCAAGACCGACG
TGTACAGCTACGGCGTTGTCCTGCTCGAGCTGATAACGGGAAAGACGGCAGTGGATC
CCTCGTTCCCGGAGAACATGGACATTGTCGGCTGGGTGCCCCACGCCCTGAACGGC
GCTGAGCAGATCGGGCCCGTCTGCGACCCGGCCCTCCTGGACGAAGTGTACAGCAC
TGTCGAAATGGAGGAGGTGCGCAAGGTCCTGCGCCTGGCCCTCAGGTGCACGGCGA
AGGAGCCGAGTCAACGACCGTCCATGGTCGATGTCGTGAAGGAGTTGACTGACGCG
AGGTTTGCGGGCATCCCTTCATCGTCGAAGCAGGGGAAGCCTGGCTCCTCCTCCGG
CGGCGGCTCTTCTTGA
SEQ ID NO: 68-SIPEPR1-LIKE2 (RER-LIKE2) Wheat Amino Acid Sequence (TaeRER-
LIKE2)
MGLVLWHSLFLFLSLVSSSWSLNSDGRALLALSKNLILPSSIKSSWNASDTTPCNWTGISC
GKRNNVVSLDLTSSGVSGSLGVQIGLLKYIQVIILLNNSISGPIPQELGNCSMLEQLDLSEN
FLSGEIPESLSNLKKLSSLSLYTNSLSGEIPEGLFKNQFLQDVYLNENKLSGSIPLSVGETT
SLRSFWLMHNALSGGLSDSIGNCTKLEELYLLDNRLSGSLPKTLSYVKGLKVLDATGNSF
TGEIDFSFENCKLEKFILSFNQMRGGIPARLGNCSSLTELALVNNSFSGQIPASLGLLSNLT
LLMLSQNSLSGSIPPEIGNCRLLEWLELDHNMLEGTVPKELANLRHLQKLFLFENRLTGEF
PEDIWSIRYLRSVLIYSNGFTGKLPLKLAELKLLENITLFDNFFTGVIPPGLGVNSPLQQIDF
TNNSFTGGIPPYICSRKRLRVLILGFNLLNGSIPSNVADCPGLERIILKNNDLTGPIPHFRNC
ARLGYMDFSHNSLSRDIPASLGKCINTTMINWSGNKLVGPIPPEIGNLVNLGVLNLSQNSL
HGALPAQVSSCSKLYILDLSFNSLHGSALMTVSSLKLLAQLRLQENKFSGGLPDSLSQLV
MLLELQLGGNILGGSIPSSLGKLIKLIALNLSSNGLVGDLPTPLGNLVELQSLDLSVNNLTG
GLGALGTLHSLHALNLSYNRFSGPVPEYLLKFLNSTPSSFNGNSGLCVSCHDSDSSCKRS
DVLKPCGGSGKKGIKRRFKVALIILGSLFIGAVAVLILCCILLQNRDSKTKSEETISNLLEGSS
SKLNEIIEKTENFDDKYVIGTGAHGTVYKATLNSGEVFAIKKLAISARSSSYKSMIRELKTLG
KVRHRNLIKLKEFWVRGDSGFILYDFMEHGSLYDVLHRIRTPSLDWSMRYNIALGTAHGL
AYLHHDSVPAIIHRDIKPSNILLNKDMVPRIADFGIAKIMDQCSAAPQSTGVVGTTGYMAPE
LAFSTRNSIKTDVYSYGVVLLELITGKTAVDPSFPENMDIVGWVPHALNGTEQIGPVCDPA
LLDEVYSTVEMEEVRKVLRLALRCTANEPSRRPSMVDVVKELTDARFAGIPSSSKQGKPG
SSSGGGSS
SEQ ID NO: 69-SIPEPR1-LIKE3 (RER-LIKE3) Wheat DNA Sequence (TaeRER-LIKE3)
ATGGGGCTTGTATTGTGGCATTCATTGTTTCTCTTCTTATGTTTGGTTTCCTCGTCATG
GAGTTTGAATTCAGATGGTCGTGCCCTTCTTGCTCTGTCCAAAAATCTCATATTGCCTA
GTTCCATAAAGTCAAGTTGGAATGCTTCTGATACAACCCCATGTAACTGGACTGGAAT
TAGTTGTGATAAAAGGAACAATGTGGTTTCTCTTGACCTAACATTGTCTGGAGTTTCTG
GTTCACTAGGAGTTCATATAGGGCTTCTAAAGTACATAAAAGTCATCAATTTGCCGAG
CAATAACATATGTGGTCCAATCCCCCAAGAATTGGGCAATTGTAGCATGCTTGAACAG
TTGGATGTTTCCGGGAACTTCCTTTCTGGTGAAATACCAGAATCGCTTGGCAACCTCA
AAAAACTATCATACCTCTCGTTGTACAATAACTCCCTCAGTGGGGAAATACCAGAGGG
GTTGTTCAAGAACCAGTTTCTGCAGGACGTGTTCCTCAATGAGAATAAACTCAGTGGT
TCTATCCCTTCATCAGTTGGTGAAATGACAAGCCT
TAGATCATTTTGGTTGACGCAGAATGCATTATCTGGAGGTCTGCCAGATTCAATTGGC
AACTGCACCAAGTTGGAGGAGCTCTATCTGCTGGATAATCGGTTGAGCGGGAGCCTT
CCGAAAACCTTGAGCTATGTCAAAGGACTGAAAGTTTTAGATGCCACTGGAAATAGCT
TCACCGGAGAGATTGATTTCAGTTTTGAGAACTGCAAGTTGGAGAAATTCATATTCTC
GTTCAATCAGATGAGGGGCGGTATTCCAGCGTGGCTAGGTAATTGCAGTAGCTTGAC
AGAACTTGCACTTGTCAACAACAGTTTCTCTGGCCAAATTCCGCCTTCTCTTGGCCTA
TTGAGCAACCTCACCTTGCTTATGCTTTCTCAAAACTCCTTGTCTGGCCCAATCCCTC
CTGAGATTGGTAATTGTCGGTTGCTGGAGTGGCTAGAGTTGGATCATAACATGCTGG
AGGGCACTGTTCCTAAAGAGCTGGCTAATCTGAGACACTTGCAGAAGCTCTTTCTTTT
CGAGAATCGCCTGACCGGGGAGTTTCCTGAGGGTATTTGGAGCATCCGGTACCTTCG
AAGTGTTCTTATTTACAGCAATGGTTTTACTGGGAAGCTACCTCTTAAGTTAGCTGAAT
TGAAGTTATTGGAGAACATCACATTGTTCGATAATTTCTTCACTGGAGTCATACCCCTG
GGTTTGGGTGTTAATAGCCCTTTACAGCAAATTGATTTCACTAACAACAGTTTTACCGG
TGGAATACCTCCGTACATTTGTTCAAGGAAAAGACTCAGGGTGTTGGTTTTGGGTTTC
AATCTTCTGAATGGTAGCATCCCATCCAATGTTGCAGACTGCCCAGGCTTGCAACGG
ATCATTCTCAGAAACAATGATCTTACTGGGCCCATTCCACATTTTAGAAACTGTGCAG
CTCTGGGCTATACAGATTTCAGTCATAATTCTTTAAGTGGAGATATTCCAGCAAGCTTG
GGCAAATGCATAAATACTACAATGATAAACTGGTCAGCAAACAAACTTGTTGGTCCAA
TACCACCTGAAATTGGAAACTTGGTGAATTTGGGAGTTCTTAACCTCTCACAAAACAG
TCTCCAGGGTGCACTTCCAGCGCAGGTTTCTAGTTGCTCCAAGCTGTATATTCTGGAT
TTGAGTTTCAACTCTCTGCATGGTTCGGCACTCACGACAGTAAGCAGCCTGAAGCTTC
TCGCACAACTACGGTTGCAGGAGAATAAATTCAGTGGGGGCTTACCTGATTCTCTTTC
GCAGT
TGGTTATGCTTCTTGAGCTGCAACTTGGTGGCAACATTCTTGGAGGCAGCATCCCTTC
GTCGTTAGGAAAGTTGATCAAACTCATTGCATTGAATCTCAGCAGCAACGGACTGGTG
GGTGATCTTCGAACACCATTGGGCAATTTGGTGGAACTGCAAAGTTTAGATTTGTCAG
TTAATAACCTCACTGGAGGTCTTGGCGCATTAGGAAGTCTACATTCATTGCATGCCTT
GAATCTTTCCTATAATAGGTTCAGTGGACCAGTGCCAGAATATCTTCTGAAATTTCTGA
ACTCCGCACCAAGCTCTTTTAATGGAAATTCGGGTCTCTGTATCTCTTGCCGTGACAG
TGATTCTTCTTGCAAGAGATCTAATGTGCTGAAACCTTGTGGAGGATCAGGGAAAAAA
GGAATAAAGCGTCGATTCAAGGTTGCTCTTATTATTCTTGGTTCATTGTTCATTGGAGC
AGTAGCGGTACTTATCCTCTGCTGTATCCTTCTAAAGAATCGAGATTCGAAGACAAAA
AGTGAGGAGACAATCAGTAATTTGCTTGAAGGCTCTTCTTCTAAATTGAATGAGATCAT
AGAAAAGACAGAAAACTTTGATGACAAGTATATCATAGGCGCAGGTGCTCATGGGAC
TGTGTACAAGGCAATATTGAATTCAGGGGAGGTCTTTGCTATAAAGAAGCTTGCGATT
TCCGCGCGCAGCAGTTCCTACAAAAGCATGGTCAGAGAACTGAAGACGCTTGGTAAA
GTTCGGCACAGGAACTTGATAAAGCTGAAAGAATTTTGGGTGAGAGGCGACTCTGGG
TTCATACTGTATGACTTTATGGAGCATGGTAGCCTCTATGATGTCCTGCACAGGATCC
GGACGCCGAGTCTGGACTGGAGCATGCGCTATAACATAGCTCTTGGAACTGCCCATG
GTCTGGCATATCTTCACCATGACTCTGTCCCTGCGATCATTCATCGAGATATTAAGCC
GAGCAACATATTGCTGAACAAGGACTTGGTGCCGCGCATCGCAGATTTCGGCATAGC
GAAGATCATGGATCAGTGTTCAGCTGCTCCACAGACCACCGGGGTCGTCGGCACAA
CTGGATATATGGCACCAGAGCTGGCATTTTCCACCAGAAACAGTATCAAGACCGACG
TGTACAGCTACGGCGTTGTCCTGCTCGAGCTGATAACGGGAAAGACGGCAGTGGATC
CCTCGTTCCCGGAGAACATGGACATTGTCGGCTGGGTGCCCCACGCCCTGAACGGC
GCTGAGCAGATCGGGCCCGTCTGCGACCCGGCCCTCCTGGACGAAGTGTACAGCAC
TGTCGAAATGGAGGAGGTGCGCAAGGTCCTGCGCCTGGCCCTCAGGTGCACGGCGA
AGGAGCCGAGTCAACGACCGTCCATGGTCGATGTCGTGAAGGAGTTGACTGACGCG
AGGTTTGCGGGCATCCCTTCATCGTCGAAGCAGGGGAAGCCTGGCTCCTCCTCCGG
CGGCGGCTCTTCTTGA
SEQ ID NO: 70-SIPEPR1-LIKE3 (RER-LIKE3) Wheat Amino Acid Sequence (TaeRER-
LIKE3)
MGLVLWHSLFLFLCLVSSSWSLNSDGRALLALSKNLILPSSIKSSWNASDTTPCNWTGISC
DKRNNVVSLDLTLSGVSGSLGVHIGLLKYIKVINLPSNNICGPIPQELGNCSMLEQLDVSG
NFLSGEIPESLGNLKKLSYLSLYNNSLSGEIPEGLFKNQFLQDVFLNENKLSGSIPSSVGE
MTSLRSFWLTQNALSGGLPDSIGNCTKLEELYLLDNRLSGSLPKTLSYVKGLKVLDATGN
SFTGEIDFSFENCKLEKFIFSFNQMRGGIPAWLGNCSSLTELALVNNSFSGQIPPSLGLLS
NLTLLMLSQNSLSGPIPPEIGNCRLLEWLELDHNMLEGTVPKELANLRHLQKLFLFENRLT
GEFPEGIWSIRYLRSVLIYSNGFTGKLPLKLAELKLLENITLFDNFFTGVIPLGLGVNSPLQQ
IDFTNNSFTGGIPPYICSRKRLRVLVLGFNLLNGSIPSNVADCPGLQRIILRNNDLTGPIPHF
RNCAALGYTDFSHNSLSGDIPASLGKCINTTMINWSANKLVGPIPPEIGNLVNLGVLNLSQ
NSLQGALPAQVSSCSKLYILDLSFNSLHGSALTTVSSLKLLAQLRLQENKFSGGLPDSLSQ
LVMLLELQLGGNILGGSIPSSLGKLIKLIALNLSSNGLVGDLRTPLGNLVELQSLDLSVNNLT
GGLGALGSLHSLHALNLSYNRFSGPVPEYLLKFLNSAPSSFNGNSGLCISCRDSDSSCKR
SNVLKPCGGSGKKGIKRRFKNRDSKTKSEETISNLLEGSSSKLNEIIEKTENFDDKYIIGAG
AHGTVYKAILNSGEVFAIKKLAISARSSSYKSMVRELKTLGKVRHRNLIKLKEFWVRGDSG
FILYDFMEHGSLYDVLHRIRTPSLDWSMRYNIALGTAHGLAYLHHDSVPAIIHRDIKPSNILL
NKDLVPRIADFGIAKIMDQCSAAPQTTGVVGTTGYMAPELAFSTRNSIKTDVYSYGVVLLE
LITGKTAVDPSFPENMDIVGWVPHALNGAEQIGPVCDPALLDEVYSTVEMEEVRKVLRLA
LRCTAKEPSQRPSMVDVVKELTDARFAGIPSSSKQGKPGSSSGGGSS
SEQ ID NO: 71-SIPEPR1-LIKE4 (RER-LIKE4) Wheat DNA Sequence (TaeRER-LIKE4)
ATGGGGCTGATTTCATGGCATCGTTTGCTTCTCTTCTCCAACCTAGTCTCGCTCTGTT
GCGGTTTGAGTTCAGATGGCCATGCCCTTCTGGCTCTCTCCAGGAGGCTCATACTAC
CTGATACCATAAGCTCGAACTGGAGTTCTTCTGATACAACTCCCTGTGGATGGAAGG
GAGTTCAGTGTGAGATGAACAATGTGGTTCACCTCAATCTATCATACTACAAAGTTTCT
GGTTCAATTGGTCCCGAGGTAGGGCGTATGAAGTACCTGCGGCAACTCGATCTGTCA
AGCAACAACATCTCCGGTCCGATCCCTCATGAATTAGGCAATTGTGTTCTGCTTGATC
TGCTTGATCTGTCAGGTAACAGCCTTTCCGGTGGAATTCCGACATCCCTCATGAACCT
AAAGAAGCTTTCACAGCTCGGTTTGTACAGCAACTCCCTCAGTGGAGAGATACCCGA
GGGGCTGTTCAAGAACCGGTTTCTGGAGCGGGTGTATCTCCAAGACAATAAACTCAG
TGGCTCTATCCCTTCATCAGTTGGTGAAATGAAGAGTCTGAAGTACTTTAGGTTGGAT
GGGAACATGTTGTCCGGAGCTCTGCCGGACTCAATTGGCAACTGCACCAAGTTGGAG
AATCTATATCTATACGGTAATAAACTGAATGGGAGTCTTCCGAGATCATTGAGCAACA
TCAAAGGGCTCGTGCTCTTTGAAGCCAAC
AACAACAGTTTTACAGGTGACATCTCTTTCAGATTCAAGAGCTGCAAGCTTGAAGTGT
TTGTGCTATCTTGGAATCAGATCAGTGGGGAAATCCCGGGGGGCTGGGAAATTGTA
GCAGCTTAATCAGACTTGCATTTCTCCACAACCGTCTCTCTGGCCAGATACCGACTTC
ACTCGGTTTATTGAAAAAACTATCAATCCTTATACTTACCCAGAATTCTTTGTCTGGGC
TGATCCCTCCTGAGATTGGTAGCTGTCGGTCGCTGGTGTGGCTGGAGCTGGACGCA
AACCAGCTCGAGGGCACCGTCCCGAAACAGCTGGCTAATCTGCGTAACTTGCAGCA
GCTATTTCTGTTTGAGAATCGCCTCAGTGGGGAGTTCCCTCAGGATATTTGGGGCATC
CAAGGCCTCGAATCCGTCCTTCTGTACAACAACAGCTTATCTGGGGGCCTACCTCCA
ATGTCGGCCGAGTTGAAGCACCTAAAGTTCGTCAAACTACAGGATAATTTGTTCACTG
GAGTCATACCACCAGGATTTGGGATTAACAGCCCTTTAGTAGAGATTGACTTCACAAA
TAATAGGTTTGTTGGTGGAATCCCGCCGAACATTTGTTCGGGTAAAAGATTGACAGCT
TGGAATTTGGGGCATAATTTTCTCAATGGTACCATACCGTTCACTGTTGCTAGCTGCC
CAAGTTTAGAACGAGTTCGACTCCATAAC
AACAATCTCAGTGGGCAAGTTCCGCAATTCCGAGACTGTGCAAATCTGCGGTACATA
GATTTGAGTCACAATTCCTTAAGTGGTCATATTCCTGCAAGCTTGGGCAGGTGTGCTA
ACATTACAGCGATAAACTGGTCTCAAAACAAGCTTGGTGGTCCAATACCACCTGAACT
CGGACAATTAGTGAAGTTGGAAAGTCTTGACCTCTCCCACAACAGCCTAGAGGGGGC
AATTCCTGCACAGATTTCCAGTTGCTCGAAGTTGCACTTGTTTGATTTGAGTTTCAACT
CTTTGAATGGTTCTGCACTCACAACAGTATGCAAGCTGGAGTTTATGTTAAATCTGCG
GCTACAGAGGAATAGATTAAGTGGAGGCATTCCAGATTGTATCTCGCAGTTACATGG
GCTAGTCGAGCTACAACTTGGCGGCAATGTTCTTGGGGGTCATCTCCCTTCAGCATT
AGGAACTTTGAAAAGATTGAGTACTGCACTAAACCTTAGCAGCAATGGGCTGGAGGG
CAGCATTCCATCTCAATTACGCTTCTTGGTGGATCTTGCAAGCTTAGATTTGTCTGGTA
ATAATCTAAGTGGGGATCTTGCTCCGTTAGGAAGTCTACATGCATTGTATACCTTGAA
TCTTTCGAATAACAGATTCAGCGGGCCAGTGCCAGAGAATCTTGTACAATTTATTAATT
CCACACCAAGTCCATTCAGTGGAAATTCAGATCTCTGTGTGTCTTGCCATGATGATGA
TTCTTCTTGCAAGGGAGCTAATGTTTTGGAACCTTGTAGTTCATTGAGGAGAAGAGGA
GTACATGGCCGCGTCAAGATAGCTATGATATGTCTTGGTTCAGTTTTTGTTGGTGCGT
TTCTGATACTCTGTATCTTTCTGAAATACAGAGGTTCCAAGACTAAACCTGAGGGAGA
GTTAAATCCATTTTTTGGGGAGTCATCTTCTAAATTAAATGAGGTTTTGGAATCAACTG
AAAACTTTGATGACAAGTACATCATCGGCACAGGTGGCCAAGGAACTGTATACAAGG
CAACATTGAGGTCAGGAGAAGTATATGCCGTGAAGAAGCTTGTGGGTCATGCACACA
AGATTTTGCATGGAAGCATGATAAGGGAGATGAATACGCTTGGTCAAATTAGGCATAG
GAACTTAGTAAAACTAAAGGATGTTTTGTTCAGGCGTGAGTATGGGTTGATCCTCTAT
GAATTTATGGACAATGGTAGCCTTTATGATGTTCTGCATGGGACTGAGGCAGCTCCAG
TTCTAGAGTGGAGGACACGGTATGACATAGCTCTTGGTACAGCACATGGTTTAGCTTA
TCTCCACAATGACTGCCACCCAGCCATTATTCACCGTGACATTAAACCGAAAAATATA
CTGTTGGATAAGGACATGGTGCCACATATTTCAGACTTTGGCATTGCAAAGCTTATCG
ACCTGTCTCCTGCTGCTTCAGAAACTACTGGAATCGTTGGCACTGTTGGGTATATGGC
CCCAGAGATGGCATTTTCAACCAGGAGTACCATTGAGTTCGACGTGTACAGCTACGG
AGTCGTATTACTTGAATTGATTACCAGAAAGATGGCCCTGGATCCCTCGTTCCCTCAC
GATGTGGACCTAGTCAGCTGGGTATCCTCCACCCTGAATGAGGGCAACGTGATCGAG
TCCGTGTGCGACCCTGCCCTAATGCGTGAGGTATGTGGCACTGCTGAACTGGAGGAA
GTATGCAGTGTGCTGTCGATAGCCCTTAGATGCACCGCGGAGGACGCAAGACAGAG
GCCTTCCATGATGGATGTTGTGAAAGAGCTGACACGTGCTAGGCATGATGTTGTATC
GCTACCGAAGCAGGCGGTGTCTGGTTCCAGCAGTTCCTGTCAAAACCTGGCAACCTG
A
SEQ ID NO: 72-SIPEPR1-LIKE4 (RER-LIKE4) Wheat Amino Acid Sequence (TaeRER-
LIKE4)
MGLISWHRLLLFSNLVSLCCGLSSDGHALLALSRRLILPDTISSNWSSSDTTPCGWKGVQ
CEMNNVVHLNLSYYKVSGSIGPEVGRMKYLRQLDLSSNNISGPIPHELGNCVLLDLLDLS
GNSLSGGIPTSLMNLKKLSQLGLYSNSLSGEIPEGLFKNRFLERVYLQDNKLSGSIPSSVG
EMKSLKYFRLDGNMLSGALPDSIGNCTKLENLYLYGNKLNGSLPRSLSNIKGLVLFEANN
NSFTGDISFRFKSCKLEVFVLSWNQISGEIPGWLGNCSSLIRLAFLHNRLSGQIPTSLGLLK
KLSILILTQNSLSGLIPPEIGSCRSLVWLELDANQLEGTVPKQLANLRNLQQLFLFENRLSG
EFPQDIWGIQGLESVLLYNNSLSGGLPPMSAELKHLKFVKLQDNLFTGVIPPGFGINSPLV
EIDFTNNRFVGGIPPNICSGKRLTAWNLGHNFLNGTIPFTVASCPSLERVRLHNNNLSGQV
PQFRDCANLRYIDLSHNSLSGHIPASLGRCANITAINWSQNKLGGPIPPELGQLVKLESLD
LSHNSLEGAIPAQISSCSKLHLFDLSFNSLNGSALTTVCKLEFMLNLRLQRNRLSGGIPDCI
SQLHGLVELQLGGNVLGGHLPSALGTLKRLSTALNLSSNGLEGSIPSQLRFLVDLASLDLS
GNNLSGDLAPLGSLHALYTLNLSNNRFSGPVPENLVQFINSTPSPFSGNSDLCVSCHDDD
SSCKGANVLEPCSSLRRRGVHGRVKIAMICLGSVFVGAFLILCIFLKYRGSKTKPEGELNP
FFGESSSKLNEVLESTENFDDKYIIGTGGQGTVYKATLRSGEVYAVKKLVGHAHKILHGS
MIREMNTLGQIRHRNLVKLKDVLFRREYGLILYEFMDNGSLYDVLHGTEAAPVLEWRTRY
DIALGTAHGLAYLHNDCHPAIIHRDIKPKNILLDKDMVPHISDFGIAKLIDLSPAASETTGIVG
TVGYMAPEMAFSTRSTIEFDVYSYGVVLLELITRKMALDPSFPHDVDLVSWVSSTLNEGN
VIESVCDPALMREVCGTAELEEVCSVLSIALRCTAEDARQRPSMMDVVKELTRARHDVVS
LPKQAVSGSSSSCQNLAT
SEQ ID NO: 73-SIPEPR1 (RER) Cucumber DNA Sequence (CsaRER)
ATGCAGCTTCTCACCCGCCATTTCTTCTTACTGGTATGCTTCTCTTTCCATTTCGTTGT
TGTTGTTTTTGGTTTAACTTCAGATGGGTTGGCCTTGTTATCACTCCAAAGCCGCTGG
ACTACTCATACCTCCTTTGTCCCTGTTTGGAATGCCTCTCATTCCACTCCCTGTTCTTG
GGCTGGGATTGAATGTGATCAAAACCTCCGTGTCGTCACCTTCAATCTCTCTTTCTAT
GGGGTTTCGGGTCACCTTGGACCTGAAATTTCAAGTTTGACTCAGTTGCGTACTATTG
ATTTGACCACCAACGATTTCTCTGGTGAAATTCCTTATGGGATTGGTAACTGTAGCCA
TTTAGAGTACTTGGATCTCTCCTTCAACCAATTTAGTGGACAAATTCCTCAGTCATTGA
CCCTCCTTACGAACTTGACGTTTTTGAACTTCCATGAAAATGTTTTAACTGGTCCAATA
CCCGACTCCTTATTTCAGAATCTTAATTTCCAGTATGTGTATCTTAGTGAAAACAATCT
CAACGGTTCTATCCCTTCAAATGTGGGGAATTCGAATCAGCTGTTGCATTTGTATCTG
TATGGAAATGAGTTTTCTGGTTCCATACCTTCTTCCATAGGGAACTGTAGTCAATTGGA
GGATCTTTATTTGGATGGGAACCAGTTAGTGGGAACATTGCCCGATAGTCTTAACAAT
CTTGATAATCTTGTTAATCTGGGTGTAAGCCGAAACAATCTCCAGGGTCCAATTCCTTT
GGGTTCAGGAGTTTGTCAGAGTTTAGAATATATAGATTTGTCATTCAATGGTTATACAG
GAGGTATACCTGCCGGGTTGGGCAACTGTAGTGCCCTAAAAACCTTACTTATTGTAAA
TTCCAGTTTAACAGGTCATATTCCTTCCTCCTTTGGCCGCCTAAGGAAGCTTTCACATA
TTGATCTCTCTAGAAATCAACTGTCTGGGAATATACCTCCTGAATTTGGGGCTTGCAA
ATCCTTGAAAGAATTGGATTTGTACGATAACCAACTCGAGGGACGTATCCCTAGTGAA
CTGGGCTTGCTAAGTAGATTAGAGGTCCTTCAATTGTTTTCAAACCGGTTGACTGGTG
AAATTCCAATTAGCATCTGGAAGATTGCAAGTCTCCAACAGATTCTTGTGTATGACAA
CAACCTTTTTGGGGAATTACCCTTGATAATAACAGAACTCAGGCACCTCAAAATCATTT
CTGTGTTCAACAATCATTTTTCTGGAGTCATACCTCAAAGTTTGGGACTCAACAGTAG
CTTAGTGCAGGTGGAGTTCACCAATAATCAGTTCACTGGTCAAATTCCACCTAATTTAT
GCTCTGGAAAGACATTAAGGGTGCTAAATTTAGGTTTGAATCAATTTCAAGGAAATGT
ACCTCTTGATATTGGAACTTGTTTGACTCTTCAGAGATTAATTTTAAGAAGGAATAATC
TAGCAGGGGTTCTGCCAGAATTTACGATAAATCACGGTCTTCGATTCATGGATGCCAG
TGAAAATAACCTCAATGGAACAATTCCCTCAAGTTTGGGAAATTGCATCAATCTTACCT
CAATTAATCTTCAAAGCAACAGGCTTTCAGGCCTTATACCTAATGGATTGAGAAATCTT
GAGAATCTTCAAAGTTTGATTTTGTCTCATAACTTCTTGGAAGGACCTTTGCCATCTTC
CCTCTCAAATTGCACGAAGCTGGATAAATTTGACGTAGGATTTAATTTATTGAATGGTT
CTATACCTCGTAGTTTAGCCAGCTGGAAAGTTATATCCACGTTTATAATTAAAGAGAAT
CGATTTGCTGGAGGTATCCCGAATGTATTATCAGAACTTGAAAGCCTTTCACTACTAG
ATCTTGGTGGCAATCTGTTTGGAGGTGAAATCCCTTCATCTATTGGAAATTTGAAGAG
TCTGTTTTACTCCCTGAATCTTAGTAACAATGGGTTAAGTGGCACACTACCTTCTGAG
CTGGCGAATCTTGTCAAGTTACAGGAATTAGATATATCTCACAATAATTTGACTGGAA
GTTTAACTGTCCTTGGCGAACTAAGTTCGACATTAGTTGAGCTTAACATTTCATATAAT
TTTTTCACCGGTCCAGTGCCACAAACATTAATGAAGTTATTGAATTCTGATCCCTCATC
GTTCTTAGGTAACCCTGGGTTGTGCATTAGTTGTGATGTACCAGATGGTCTAAGTTGC
AATAGAAATATCAGTATTAGTCCTTGTGCTGTTCATTCAAGCGCCCGTGGTAGCTCTC
GACTTGGAAATGTACAGATTGCAATGATAGCTCTGGGGTCTTCACTTTTTGTTATTCTT
TTGCTTCTTGGGTTGGTTTATAAGTTTGTTTATAACAGAAGAAACAAGCAAAACATTGA
GACTGCTGCTCAAGTCGGAACAACTTCCTTGCTCAACAAGGTAATGGAGGCCACCGA
TAATTTAGATGAACGTTTCGTCATTGGAAGAGGAGCACATGGAGTTGTTTATAAGGTC
TCCCTGGATTCAAATAAAGTTTTTGCTGTAAAGAAGCTTACATTTTTAGGACATAAAAG
GGGAAGCCGGGATATGGTTAAAGAAATTAGAACTGTCAGCAACATCAAGCACCGGAA
CTTGATCTCTTTGGAAAGTTTTTGGTTGGGAAAAGATTATGGTCTATTGCTTTACAAAT
ACTATCCAAATGGGAGCCTTTATGATGTGTTGCACGAGATGAATACAACTCCATCTCT
CACATGGAAAGCCCGCTATAATATAGCGATCGGTATTGCTCATGCATTGGCATATCTC
CATTACGATTGTGATCCTCCCATTATACACCGAGACATCAAACCACAGAATATACTTCT
AGATTCGGAGATGGAACCTCATATCGCCGACTTTGGTCTTGCAAAGCTATTGGATCAA
ACTTTCGAACCCGCGACTTCATCTTCTTTTGCGGGTACAATTGGCTACATTGCACCAG
AGAATGCATTTTCAGCAGCAAAGACCAAAGCCTCAGATGTGTACAGTTATGGGGTGG
TTTTACTGGAGCTGGTGACGGGAAAGAAGCCATCAGATCCATCATTTATAGAAGTCG
GGAATATGACGGCTTGGATTCGGTCGGTTTGGAAGGAGAGAGATGAAATAGATAGAA
TTGTTGATCCAAGGCTTGAGGAAGAACTTGCTAATTTGGATCATAGGGAGCAGATGAA
TCAGGTGGTTCTTGTGGCTTTGAGATGCACAGAAAACGAGGCTAACAAAAGACCTATA
ATGAGAGAGATTGTGGATCACTTGATTGATTTAAAGATCAGTCGTTAG
SEQ ID NO: 74-SIPEPR1 (RER) Cucumber Amino Acid Sequence (CsaRER)
MGLISWHRLLLFSNLVSLCCGLSSDGHALLALSRRLILPDTISSNWSSSDTTPCGWKGVQ
CEMNNVVHLNLSYYKVSGSIGPEVGRMKYLRQLDLSSNNISGPIPHELGNCVLLDLLDLS
GNSLSGGIPTSLMNLKKLSQLGLYSNSLSGEIPEGLFKNRFLERVYLQDNKLSGSIPSSVG
EMKSLKYFRLDGNMLSGALPDSIGNCTKLENLYLYGNKLNGSLPRSLSNIKGLVLFEANN
NSFTGDISFRFKSCKLEVFVLSWNQISGEIPGWLGNCSSLIRLAFLHNRLSGQIPTSLGLLK
KLSILILTQNSLSGLIPPEIGSCRSLVWLELDANQLEGTVPKQLANLRNLQQLFLFENRLSG
EFPQDIWGIQGLESVLLYNNSLSGGLPPMSAELKHLKFVKLQDNLFTGVIPPGFGINSPLV
EIDFTNNRFVGGIPPNICSGKRLTAWNLGHNFLNGTIPFTVASCPSLERVRLHNNNLSGQV
PQFRDCANLRYIDLSHNSLSGHIPASLGRCANITAINWSQNKLGGPIPPELGQLVKLESLD
LSHNSLEGAIPAQISSCSKLHLFDLSFNSLNGSALTTVCKLEFMLNLRLQRNRLSGGIPDCI
SQLHGLVELQLGGNVLGGHLPSALGTLKRLSTALNLSSNGLEGSIPSQLRFLVDLASLDLS
GNNLSGDLAPLGSLHALYTLNLSNNRFSGPVPENLVQFINSTPSPFSGNSDLCVSCHDDD
SSCKGANVLEPCSSLRRRGVHGRVKIAMICLGSVFVGAFLILCIFLKYRGSKTKPEGELNP
FFGESSSKLNEVLESTENFDDKYIIGTGGQGTVYKATLRSGEVYAVKKLVGHAHKILHGS
MIREMNTLGQIRHRNLVKLKDVLFRREYGLILYEFMDNGSLYDVLHGTEAAPVLEWRTRY
DIALGTAHGLAYLHNDCHPAIIHRDIKPKNILLDKDMVPHISDFGIAKLIDLSPAASETTGIVG
TVGYMAPEMAFSTRSTIEFDVYSYGVVLLELITRKMALDPSFPHDVDLVSWVSSTLNEGN
VIESVCDPALMREVCGTAELEEVCSVLSIALRCTAEDARQRPSMMDVVKELTRARHDVVS
LPKQAVSGSSSSCQNLAT
SEQ ID NO: 75-SIPEPR1 (RER) Peach DNA Sequence (PpRER)
ATGCAGCTTTATCTGTTCAATTTCTTCTTGTTGTTGCTGTTGCTGTGCTTCTCTGTGTC
CATATCTACTGTATCTAGTTTGAGCTCTGATGGGTTGGCTTTATTGTCACTCTCCAAGC
ATTGGACGTCTGTCCCTGCTTCCATATCCTCAAGCTGGAGTGCTTCTGATGCCACTCC
ATGCCAATGGGTTGGAATAGAATGTGACAATGCCCACAATGTGGTTACCTTGAACCTC
ACTGGTTATGGAATTTCTGGTCAATTGGGACCTGAAGTTGGCAGCTTTAGGCACTTGC
AGACTCTTGATTTGAGTGTCAACAATTTTTCTGGCAAAATCCCAAAAGAATTGGCCAAT
TGTAGTCTTCTTGAAAACTTGGACTTGTATAAAAATGGCTTTTCTGGTGCAATACCTGA
ATCCTTGTTTGCAATTCCTGCCTTAGCTTATGTACATCTGTATACTAACAATTTGAATG
GTTCCATCCCTGGAAATGTTGGGAATCTGAGTGAGCTTGTGCATCTGTATTTGTATGA
GAATCAGTTTTCAGGAGTGATTCCTTCATCCATTGGAAACTGTAGTAAATTGCAGGAG
TTGTTTTTGGGTCGGAACCAGTTGACAGGAGAGCTTCCTATGAGCCTGAACAACCTTC
AGAACCTGGTATATCTAGATGTAGCTATAAATAGTCTTGAGGGTAGCATTCCTTTGGG
TTCAGGCACTTGCAAGAATTTAATTTACTTGGATTTGTCATACAATAAGTTTAGTGGAG
GTATTCCACCAGGACTTGGAAATTGTAGTAATTTAACACAATTTTCTGCTGTGGGTAGT
AATTTAGAGGGGACTATTCCATCTTCCTTTGGCCAACTCAAGTACCTTTCAACCCTGTA
CCTTCCTCTAAATCATTTGTCTGGTAAAATACCTCCTGAACTTGGCAAGTGTGAATCCT
TGAAGATACTACGCTTGTATAAGAACCAACTAGTGGGAGAAATTCCTAGTGAATTGGG
AATGCTGACTCAATTAGAGGACCTTGAATTATTTGAGAACCGGTTAACTGGTGAAATT
CCAGTTAGCATATGGAAGATTCAAAGTCTTCAGCATATCCTTGTGTACAATAATAGCCT
CACTGGGGAGCTGCCTGAAGTGATGACTGAGCTGAAGCAACTAAAAAATATATCATT
GTACAACAACCTATTTTTTGGAGTTATACCTCAAAGTTTGGGGATTAACAGCAGTTTGT
GGCAGTTAGATTTTATCAATAACAAGTTCACTGGTAAAATCCCTCCAAATCTTTGCCAT
GGAAAGCAGTTAAGGGTGTTGAATCTGGGTTTCAATCGATTTCAAGGCACCATACCTT
CTGATGTTGGAAATTGCTCTACTCTTTGGAGGTTGAAACTGGAACAGAACAGACTCAT
TGGGGCTCTTCCACAGTTTGCAAAAAATTCAAGCCTTTCATATATGGACATCAGCAAC
AATGAGATTAGTGGAGAAATTCCATCAAGCTTGGGAAACTGTAGCAATCTCACAGCCA
TCAATTTGTCCATGAACAATTTAACAGGAGTTATACCCCAGGAACTAGGGAGTCTTGC
AGAACTTGGTTCTTTGATTCTTTTCAAAAACAATTTGGTTGGTCCTCTGCCTCCTCACC
TATCAAATTGTACCAAAATGTATAAGTTTGATGTGGGATCAAATTTGTTGAATGGCTCC
ATTCCATCGAGTTTGAGAAGTTGGACAGGTTTATCAACGCTGATTTTAAGTGACAACA
GTTTTACTGGCGGTGTTCCACCTTTCTTGTCGGAGTTTGAAAAGCTTTCAGAGCTACA
ACTTGGTGGAAATTTTTTGGGAGGTGCCATTCCCTCATCAATTGGAGCATTGGTTAGT
ATGTTTTATGCATTGAATCTTAGCAATAATGCATTGACAGGTCCGATTCCTTCAGAGCT
GGGGAAGTTGGCAAGACTACAACGACTAGATCTATCTCATAACAATTTGACAGGGACT
TTAAAAGCTCTTGATTATATCAATTCATTGATTGAGGTTGATGTTTCAGACAATAACTTC
ACAGGTGCAGTACCAGAGACATTGATGAACCTTTTGAACTCATCTCCATTATCATTTTT
GGGCAATCCCTACCTATGTGTCGATTACCTTCCATCATGTGGCTCAACTTGTGCAAGA
AGAAACAACAGTTTTAAGCCATGCAACAGTCAATCAAGCAAGCATAGAGGCCTTAGTA
AAGTGGCAATTGCATTTATATCCCTAGGTTCTTCATTATTTGTTGTTTTTGTGCTTCATG
TACTGGTTTATATGTTTCTCTTGCGCAAAAAGACAAAGCAGGAACTTGAGATCTCTGC
TCAAGAGGGGCCATCTGGCTTACTCAACAAAGTATTGGAGGCTACAGCGAACCTTAA
TGGTCAGTATATCATTGGGAAAGGAGCCCATGGAACTGTCTATAAGGCCTCTTTGGCT
CCAGACAAAGATTATGCTGTCAAGAAGCTTCTGTTTGCGGGGCATGAAGGAACGCGT
TTGAGCATGGTTAGAGAAATTCAAACCCTCGGGACTATTAGGCATCGGAATCTGGTTA
AATTGGAAGACTTCTGGTTAAGAAAGGACCACGGTTTAATCTTGTATAGGTACATGCA
AAATGGGAGCCTTAATGATGTTTTGCATGAAATTAAACCCCCACCAACTCTTGAGTGG
AGTGTCCGCTATAGGATAGCACTTGGAACTGCATACGGGTTGGAGTATCTCCATTATG
ATTGTGACCCCCCTATAGTTCATCGAGATGTCAAACCAATGAACATCCTCTTAGACGC
TGATATGGAGCCTCATATTGCTGATTTTGGTATTGCTAAACTTCTGGATCAGTCTTCTG
CATCAACGACGTCCATTGCAGTTGTGGGAACAACTGGATATATTGCACCAGAAAATGC
ATTTAGACCAGCAAAGAGCGTGGAATCTGATGTGTACAGTTATGGGGTTGTTCTACTT
GAGCTGATAACTAGAAAGAAGGCATTGGATCCATCATTTGTGGAGCAAACTGACATTG
TAGGATGGGTTAGGTCAGTGTGGAGCAACACAGAAGAAATTCATCAAATTGTTGATTC
AAGCCTTAAGGAGGAATTTCTGGATTCATGTATTATGGACCAAGTTGTCGATGTGCTT
ATGGTGGCATTCAGATGTACTGATAAAGATCCTAGAAAGAGGCCGACGATGAGAGAT
GTTGTGAAGCAATTATTAGATGCAAATCCCCAAGTGAGAAGCATAAAAGGCTAG
SEQ ID NO: 76-SIPEPR1 (RER) Peach Amino Acid Sequence (PpRER)
MQLYLFNFFLLLLLLCFSVSISTVSSLSSDGLALLSLSKHWTSVPASISSSWSASDATPCQ
WVGIECDNAHNVVTLNLTGYGISGQLGPEVGSFRHLQTLDLSVNNFSGKIPKELANCSLL
ENLDLYKNGFSGAIPESLFAIPALAYVHLYTNNLNGSIPGNVGNLSELVHLYLYENQFSGVI
PSSIGNCSKLQELFLGRNQLTGELPMSLNNLQNLVYLDVAINSLEGSIPLGSGTCKNLIYLD
LSYNKFSGGIPPGLGNCSNLTQFSAVGSNLEGTIPSSFGQLKYLSTLYLPLNHLSGKIPPEL
GKCESLKILRLYKNQLVGEIPSELGMLTQLEDLELFENRLTGEIPVSIWKIQSLQHILVYNNS
LTGELPEVMTELKQLKNISLYNNLFFGVIPQSLGINSSLWQLDFINNKFTGKIPPNLCHGKQ
LRVLNLGFNRFQGTIPSDVGNCSTLWRLKLEQNRLIGALPQFAKNSSLSYMDISNNEISGE
IPSSLGNCSNLTAINLSMNNLTGVIPQELGSLAELGSLILFKNNLVGPLPPHLSNCTKMYKF
DVGSNLLNGSIPSSLRSWTGLSTLILSDNSFTGGVPPFLSEFEKLSELQLGGNFLGGAIPS
SIGALVSMFYALNLSNNALTGPIPSELGKLARLQRLDLSHNNLTGTLKALDYINSLIEVDVS
DNNFTGAVPETLMNLLNSSPLSFLGNPYLCVDYLPSCGSTCARRNNSFKPCNSQSSKHR
GLSKVAIAFISLGSSLFVVFVLHVLVYMFLLRKKTKQELEISAQEGPSGLLNKVLEATANLN
GQYIIGKGAHGTVYKASLAPDKDYAVKKLLFAGHEGTRLSMVREIQTLGTIRHRNLVKLED
FWLRKDHGLILYRYMQNGSLNDVLHEIKPPPTLEWSVRYRIALGTAYGLEYLHYDCDPPIV
HRDVKPMNILLDADMEPHIADFGIAKLLDQSSASTTSIAVVGTTGYIAPENAFRPAKSVESD
VYSYGVVLLELITRKKALDPSFVEQTDIVGWVRSVWSNTEEIHQIVDSSLKEEFLDSCIMD
QVVDVLMVAFRCTDKDPRKRPTMRDVVKQLLDANPQVRSIKG
SEQ ID NO: 77-SIPEPR1-LIKE1 (RER-LIKE1) Peach DNA Sequence (PpRER-LIKE1)
ATGCAGCTTTATCTGTTCAATTTTTTGTTGTTGTTGTGCTTGTCTGTGTCCATATCCACT
GTATCTAGTTTGAACTCTGATGGGGTGGCTTTGTTGTCACTCTCCAAGCATTGGACCT
CTGTCCCTGCTTCCATATCCTCAAGCTGGAATGCTTCTGATTCCACTCCATGCCAATG
GGTTGGGATAGAATGTGACAATGACCACAATGTGGTTTCCCTGAAGCTCACTGGTTAT
GGAATTTCTGGTCAATTGGGACCTGAAATTAGCAGATTTAGGTACTTGAAGATTCTTG
ATTTGAGTGTTAACAAATTTTCTGGCAAAATTCCAACAGAATTGGCCAATTGTAGTCTT
CTTGAAAACTTGGACTTGTATGAAAATGGCTTTTCTGGTGAAATTCCTGAATCCTTCTT
TGCAATTCCTGCCTTAGCTTATGTGCATCTGTATAGTAACCGTTTGAATGGTTCCATCC
CTGGAAATGTTGGGAATCTGAGTGAGCTTGTGCATCTGGATTTATATGAGAATCAGTT
TTCAGGAGTCATTCCTTCATCCGTTGGAAACTGTAGTAAATTGGAGGACTTGTATTTG
GCTGAGAACCAGTTGATAGGAGAACTTCCTAAGAGTCTGAACAAGCTTGAGAACCTG
GTATATCTAGATGTGGCTAACAATAGTCTTGAGGGTAGCATTCCCTTGGGTTCAGGTA
CTTGCAAGAATTTAATTTACTTGGATTTTTCATACAATAAGTTTAGTGGAGGTATTCCA
CCAGGACTTGGAAATTGTAGTAATTTAACACAATTTTCTGCTGTGGGTAGCAATTTAGA
GGGGACTATTCCATCTTCCTTTGGCCAACTCAAGTACCTTTCCATCCTGTACCTTCCT
CTAAATCATTTGTCTGGTAAAATACCTCCTGAACTTGGCAAGTGTGAATCCTTGAAGG
AACTACACTTGTATACAAACCAACTAGTGGGAGAAATTCCTGGTGAATTGGGGATGCT
GACTCAATTACAGGACCTTAAATTATTTGAGAATCGGTTAACTGGTGAAATTCCAGTTA
GCATATGGAAGATTCAAAGTCTTCAGCATATCCTTGTGTACAATAATAGCCTCACCGG
GGAACTGCCTGTAGTGATGACTGAGTTGAAGCAGCTAAAAAATATATCATTGTTCAAC
AACCTGTTTTTTGGAGTTATACCTCAAACTTTGGGGATTAACAGCAGTTTGTGGCTGTT
AGATTTTACCAATAATAAGTTCACTGGTAAAATCCCTCCAAGTCTTTGCCGTGGAAAG
CAGTTATGGAAGTTGAATATGGGTTTCAATCGAATTCAAGGTACCATTCCTTCTGATGT
TGGAAATTGCTCTAGTCTAAGCAGGTTGAAACTGGGACACAATAACCTCACTGGGGTT
CTACCACAGTTTGCAAAAAATTCAAGACTTTTATATATGGACATCAGCAACAATGAGAT
TAGTGGAGAAATCCCATCTATCTTGGGAAATTGTAGCAATCTCACAACCATCAATTTGT
CCATAAACAAGTTAACAGGAGGTATACCCCAGGAGCTAGGGAATCTTGAAGAGCTTC
GTTCTTTGATTCTTTTCAAGAACAATTTGGTTGGTCCTCTACCTCCTCAGCTTTCAAAG
TGTACCAAAATGGATAAGTTTGACGTGGGATCAAATTTGTTGAATGGTTCCATTCCATC
GAGTTTGAGAAGTTGGACAGATTTATCAACACTGATTTTAAGTGACAATAGTTTTACTG
GAGAAATTCCACGTTTCTTCACAGAGTTTGAAAAACTTATAGAGCTACGACTTGGTGG
AAATTTGTTTGCAGGTGCGATTCCCTCATCAATCGGAGCATTGGTTAGTCTATCATAT
GCATTGAATCTTAGCAATAATGCATTGACAGGTCGGATTCCTTCAGAGCTTGGGAAGT
TGACAAGCCTACAACAACTCGATCTATCTCATAACAATTTGACAGGGACTTTAAAAGC
TCTTGATCATATGATTTCATTGACTGAGGTGGATGTTTCAGACAATAACTTCACAGGTT
CAGTACCAGAGACGTTTATGAAGCTGTTGAACTCATCATCATTATCATTTTTGGGCAAT
CCCTACCTATGTGTCAGTTACCTTCCATTATGTGGCTCAACTTGTGGAAGAAACAACA
GTTTTAAGCTGTGCAACCGTCAATTAAGCAATCATAAAGGCCTTAGTAAAGTAGAAATT
GCATTTACAGCCCTGGGGTCTTCATTATTCGTTGTTTTTGTGCTTTATGGACTGGTTTA
TATGTTCCTCTTACGCAAAAAGACCAAGCAGGAACTTGAGGTCTCTGCTCAAGACAG
GCTGTCTTCCCTACTCAAGGAAGTAATGGAGGCCACAGAAAATCTTAATGATCAATAT
ATCATTGGGAAAGGAGCCCATGGAACTGTCTATAAGGCCTTTTTGGCTCCAGACAAA
GATTATGCCGTCAAGAAGCTTGTATTTGCAGGGCATGAAGGAACGCGTTCAAGCATG
GTTAGAGAAATTCAAACCCTGGGGACGATAAGGCATCGAAATCTAGTTAAATTGGAAG
ACTTCTGGTTGAGAAAGGACCATGGTTTAATCTTGTACAGGTACATGGAAAATGGGAG
CCTTCATGATGCTCTACATGAAATTAAACCCCCACCAACTCTTGAGTGGATTGTCCGC
TACAGGATAGCACTTGGAACTGCATACGGTTTGGAGTATCTCCATTTTGATTGTGACC
CCCGTATAGTTCATCGAGATGTAAAACCAATGAACATTCTCTTAGACTCTGATATGGA
GCCTCATGTCGCTGATTTTGGTATTGCTAAACTTTTGGATCAGTCTTCTGCATCAACG
GCGTCCGCTGCAGTTGTGGGTACAACTGGATATATTGCACCAGAAAATGCATCTAGA
CCATCAACGAGTGTGGAATCTGATGTGTACAGTTATGGGGTTGTTTTACTTGAGCTGA
TAACTAGGAAGAAGGCATTGGATCCAGCATTTGGGGAGCAAACTGACATTGTAGGAT
GGGCTAGGTCAGCGTGGAGCAACACAGAAGACATTGATCAGATCGTTGATTCAAGCC
TTAAGGAGGAACTCCCGCATTCAAATATAATCGACCAAGTIGTGGATGTGCTTATGGT
GGCTTTCAGATGTACTGATAAAAATCCTAGAAAGAGACCGACGATGAGAGATGTTATC
CAGCAATTATTAGATGCAAATCCCCAAGTGAGAAAAAGAAGGATATTGAATCCTCGTA
ATGGTTTGTCCTAA
SEQ ID NO: 78-SIPEPR1-LIKE1 (RER-LIKE1) Peach Amino Acid Sequence (PpRER-
LIKE1)
MQLYLFNFLLLLCLSVSISTVSSLNSDGVALLSLSKHWTSVPASISSSWNASDSTPCQWV
GIECDNDHNVVSLKLTGYGISGQLGPEISRFRYLKILDLSVNKFSGKIPTELANCSLLENLD
LYENGFSGEIPESFFAIPALAYVHLYSNRLNGSIPGNVGNLSELVHLDLYENQFSGVIPSSV
GNCSKLEDLYLAENQLIGELPKSLNKLENLVYLDVANNSLEGSIPLGSGTCKNLIYLDFSYN
KFSGGIPPGLGNCSNLTQFSAVGSNLEGTIPSSFGQLKYLSILYLPLNHLSGKIPPELGKCE
SLKELHLYTNQLVGEIPGELGMLTQLQDLKLFENRLTGEIPVSIWKIQSLQHILVYNNSLTG
ELPVVMTELKQLKNISLFNNLFFGVIPQTLGINSSLWLLDFTNNKFTGKIPPSLCRGKQLWK
LNMGFNRIQGTIPSDVGNCSSLSRLKLGHNNLTGVLPQFAKNSRLLYMDISNNEISGEIPSI
LGNCSNLTTINLSINKLTGGIPQELGNLEELRSLILFKNNLVGPLPPQLSKCTKMDKFDVGS
NLLNGSIPSSLRSWTDLSTLILSDNSFTGEIPRFFTEFEKLIELRLGGNLFAGAIPSSIGALVS
LSYALNLSNNALTGRIPSELGKLTSLQQLDLSHNNLTGTLKALDHMISLTEVDVSDNNFTG
SVPETFMKLLNSSSLSFLGNPYLCVSYLPLCGSTCGRNNSFKLCNRQLSNHKGLSKVEIA
FTALGSSLFVVFVLYGLVYMFLLRKKTKQELEVSAQDRLSSLLKEVMEATENLNDQYIIGK
GAHGTVYKAFLAPDKDYAVKKLVFAGHEGTRSSMVREIQTLGTIRHRNLVKLEDFWLRKD
HGLILYRYMENGSLHDALHEIKPPPTLEWIVRYRIALGTAYGLEYLHFDCDPRIVHRDVKP
MNILLDSDMEPHVADFGIAKLLDQSSASTASAAVVGTTGYIAPENASRPSTSVESDVYSY
GVVLLELITRKKALDPAFGEQTDIVGWARSAWSNTEDIDQIVDSSLKEELPHSNIIDQVVDV
LMVAFRCTDKNPRKRPTMRDVIQQLLDANPQVRKRRILNPRNGLS
SEQ ID NO: 89-SIPEP (REF1) Barley Amino Acid Sequence (HvREF1)
AVRRPRPPGNPREGRGGGGGHHN
SEQ ID NO: 90-SIPEP (REF1) Barley DNA Sequence (HvREF1)
CGGTGAGGAGGCCGCGGCCGCCCGGCAACCCGAGGGAAGGGCGCGGCGGCGGCG
GAGGACACCACAAC
SEQ ID NO: 91-PROSIPEP (PROREF1)-(PRR) Barley Amino Acid Sequence
(HvPRR)
MDQHQVKEGGTMDQHKEEERQQEEGEPTDQRREEEVAGKPDEPTDRRKEEEEEPEEG
DNLNEPTGQRKEEDKEDKSGELTDQRKADAVAEELTDQRKVEVEEGEGEKETERSSGE
ESMSASSLSLFVHPCSLLLRYLFRAYAWCLGMADSFGGGTRPSAVPAASTNSSREEPGE
AADIGTREISKTTTDRGFYMREVIMRVRAVRRPRPPGNPREGRGGGGGHHN*
SEQ ID NO: 92-PROSIPEP (PROREF1)-(PRR) Barley DNA Sequence (HvPRR)
ATGGATCAGCACCAGGTGAAGGAGGGAGGGACAATGGATCAGCACAAGGAGGAGGA
GCGTCAGCAGGAGGAGGGAGAGCCGACGGATCAGCGCAGGGAGGAGGAGGTGGC
GGGCAAGCCCGACGAGCCGACGGATCGGCGCAAGGAGGAGGAGGAGGAGCCGGA
GGAGGGAGACAACCTCAACGAGCCAACGGGTCAGCGCAAGGAGGAAGACAAGGAG
GACAAGTCTGGAGAGCTCACGGACCAGCGCAAGGCGGATGCGGTGGCTGAAGAGCT
CACGGACCAGCGCAAGGTGGAGGTGGAGGAGGGGGAGGGGGAGAAGGAGACGGA
GAGGAGCTCCGGCGAAGAGTCCATGTCGGCCTCGTCGCTCTCGCTCTTCGTGCACC
CGTGCTCGCTCCTGCTGCGGTACCTCTTCCGCGCCTACGCGTGGTGCCTGGGCATG
GCCGACTCCTTCGGCGGCGGCACAAGGCCGAGCGCCGTCCCCGCTGCATCTACCAA
CTCATCCCGGGAAGAACCAGGCGAGGCGGCGGACATCGGGACCAGGGAGATCTCC
AAGACCACCACTGACAGAGGGTTCTACATGCGGGAGGTGATCATGCGCGTGAGGGC
GGTGAGGAGGCCGCGGCCGCCCGGCAACCCGAGGGAAGGGCGCGGCGGCGGCGG
AGGACACCACAACTAG
SEQ ID NO: 93-SIPEPR1 (RER) Barley Amino Acid Sequence (HvRER)
MGLISWHRLLVFFNLVSLCCGLSSDGHALLALSRRLILPDIISSNWSSSDTTPCGWKGVQC
EMNIVVHLNLSYSEVSGSIGPEVGRLKYLRQLDLSSNNISGPIPHELGNCVLLDLLDLSGN
SLSGGIPASLVNLKKLSQLGLYSNSLSGEIPEGLFKNRFLERVYLQDNELSGSIPSSVGEM
KSLKYFTLDGNMLSGALPDSIGNCTKLEILYLYDNKLNGSLPRSLSNIKGLVLFDASNNSFT
GDISFRFRRCKLEVLVLSSNQISGEIPGWLGNCSSLTTLAFLHNRLSGQIPTSLGLLKKLSF
LILTQNSLSGVIPPEIGSCRSLVWLQLGTNQLEGTVPKQLSNLSKLRRLFLFENRLTGEFP
RDIWGIQGLEYILLYNNSLSGVLPPMSAELKHLQFVKLMDNLFTGVIPPGFGGNSPLVEIDF
TNNGFVGGIPPNICLGKRLKVWNLGHNFLNGTIPSTVANCPSLERVRLHNNRLNGQVPQF
RDCANLRYIDLSDNSLSGHIPASLGRCANITTINWSKNKLGGPIPHELGQLVKLESLDLSHN
SLEGAIPAQISSCSKLHLFDLSFNFLNGSALTTVCKLEFMLNLRLQGNRLSGGIPDCILQLH
GLVELQLGGNVLGGNLPSSLGALKRLSTALNLSSNGLEGSIPSELRYLVDLASLDLSGNNL
SGDLAPLGSLRALYTLNLSNNRFSGPVPENLIQFINSTPSPFSGNSGLCVSCHDGDSSCK
GANVLEPCSSLRKRGVHGRVKIAMICLGSVFVGAFLVLCIFLKYRGSKTKPEGELNPFFGE
SSSKLNEVLESTENFDDKYIIGTGGQGTVYKATLNSGEVYAVKKLVGHAHKILHGSMIREM
NTLGQIRHRNLVKLKDVLFKREYGLILYEFMDNGSLYDVLHGTEAAPNLEWRIRYDIALGT
AHGLAYLHNDCHPAIIHRDIKPKNILLDKDMVPHISDFGIAKLINLSPADSQTTGIVGTVGYM
APEMAFSTRSTIEFDVYSYGVVLLELITRKMALDPSLPEDLDLVSWVSSTLNEGNVIESVC
DPALVREVCGTAELEEVCSVLSIALRCTAEDARHRPSMMDVVKELTHARRDVVSLPKQGI
SGSSSSCQNLST
SEQ ID NO: 94-SIPEPR1 (RER) Barley DNA Sequence (HvRER)
ATGGGGCTGATTTCATGGCATCGTTTACTTGTCTTCTTCAACCTGGTCTCGCTCTGTT
GCGGTTTGAGTTCAGATGGCCATGCCCTTCTGGCTCTCTCCAGGAGGCTCATACTAC
CCGACATCATAAGCTCGAACTGGAGTTCTTCTGATACGACTCCCTGTGGGTGGAAGG
GAGTTCAGTGCGAGATGAACATTGTAGTTCACCTCAACCTATCATACTCCGAAGTTTC
TGGTTCCATTGGTCCTGAGGTAGGGCGTCTGAAGTACCTGCGGCAACTCGATTTGTC
AAGCAACAACATCTCCGGTCCGATCCCTCATGAATTGGGCAACTGTGTTCTGCTTGAT
CTGCTGGATCTGTCAGGTAACAGCCTTTCCGGTGGGATCCCGGCATCCCTCGTGAAT
CTGAAGAAGCTATCGCAGCTCGGTTTGTACAGCAACTCCCTCAGTGGAGAGATACCC
GAGGGCCTGTTCAAGAACCGGTTTCTGGAGCGGGTGTATCTCCAAGACAATGAACTC
AGTGGTTCTATCCCTTCATCGGTTGGTGAGATGAAGAGTCTTAAATACTTTACGTTGG
ATGGGAACATGTTGTCTGGAGCTCTGCCGGATTCGATTGGCAACTGCACAAAGTTGG
AGATTCTATATCTATATGATAATAAACTGAATGGGAGTCTTCCAAGATCATTGAGCAAC
ATCAAAGGGCTCGTGCTCTTCGATGCCAGCAACAACAGTTTTACAGGTGACATCTCTT
TCAGATTCAGGAGATGCAAACTTGAAGTGTTAGTGCTATCTTCAAATCAGATCAGCGG
GGAAATCCCTGGGTGGCTGGGAAATTGTAGCAGCTTAACCACGCTTGCATTTCTCCA
CAACCGTCTCTCTGGCCAGATACCGACTTCACTGGGTTTATTGAAAAAGCTATCATTC
CTTATACTTACTCAGAATTCTTTGTCTGGGGTAATCCCTCCTGAGATCGGTAGCTGCC
GGTCGCTGGTGTGGCTGCAGCTGGGCACAAACCAGCTTGAGGGCACTGTTCCGAAA
CAGCTGTCTAATCTGAGTAAGTTGCGGCGGCTATTTCTGTTTGAGAATCGCCTCACAG
GGGAGTTCCCTCGGGATATTTGGGGTATCCAAGGCCTCGAATACATCCTTCTGTACA
ACAACAGCCTATCTGGGGTACTACCTCCAATGTCAGCCGAGTTGAAACACCTACAGTT
CGTCAAACTTATGGATAATCTGTTCACTGGAGTCATACCACCAGGATTTGGGGGTAAT
AGCCCTTTAGTAGAGATTGACTTCACAAATAACGGATTTGTTGGTGGAATCCCGCCGA
ACATTTGTTTGGGTAAAAGATTGAAAGTTTGGAACTTGGGGCATAATTTTCTTAATGGT
ACCATCCCGTCCACTGTTGCTAACTGCCCAAGTTTGGAACGAGTTCGTCTCCATAACA
ACCGTCTCAATGGGCAAGTTCCGCAATTCCGAGACTGTGCAAATCTGCGGTACATAG
ATTTGAGTGACAATTCCTTAAGTGGCCATATTCCTGCAAGTTTGGGCAGATGTGCTAA
CATTACAACGATAAACTGGTCTAAAAACAAGCTTGGTGGACCAATACCGCACGAACTT
GGACAATTGGTGAAGCTGGAAAGTCTTGACCTCTCCCACAACAGCCTAGAGGGGGCA
ATTCCTGCACAGATTTCCAGTTGCTCGAAGTTGCACTTGTTTGATTTGAGTTTCAACTT
TTTGAATGGTTCTGCGCTCACAACAGTATGCAAGCTGGAGTTTATGTTAAATCTGCGG
CTACAGGGGAATAGATTAAGTGGAGGCATTCCAGATTGCATCTTGCAGTTACATGGG
CTAGTCGAGCTGCAACTTGGTGGCAATGTTCTTGGGGGTAATCTCCCTTCATCATTAG
GAGCTTTGAAAAGATTGAGTACTGCACTAAACCTTAGCAGCAACGGGCTGGAGGGCA
GCATTCCATCTGAATTACGTTACTTGGTGGATCTTGCAAGCTTAGATTTGTCTGGTAAT
AATCTCAGTGGGGATCTTGCTCCGTTAGGAAGTCTACGTGCATTGTATACCTTGAATC
TTTCCAATAACAGATTCAGCGGGCCAGTGCCAGAGAATCTTATACAGTTTATAAATTC
CACACCAAGTCCATTCAGTGGAAATTCAGGTCTCTGTGTGTCTTGCCATGATGGTGAT
TCTTCTTGCAAGGGAGCTAATGTTTTGGAACCTTGTAGTTCATTGAGGAAAAGAGGAG
TACATGGCCGAGTCAAGATAGCTATGATATGTCTTGGTTCGGTTTTTGTTGGTGCATT
TCTGGTACTCTGTATCTTTCTGAAGTACAGAGGTTCCAAGACTAAACCTGAGGGAGAG
TTAAATCCATTTTTTGGGGAGTCATCTTCTAAATTAAATGAGGTTTTGGAATCAACTGA
AAACTTTGATGACAAGTACATCATCGGCACAGGTGGCCAAGGAACTGTATACAAGGC
AACACTGAATTCAGGAGAAGTATATGCCGTGAAGAAGCTTGTGGGTCATGCACACAA
GATTTTGCATGGAAGCATGATAAGGGAGATGAATACACTTGGTCAAATTAGGCACAG
GAACTTAGTAAAACTAAAGGATGTTTTGTTCAAGCGTGAGTATGGGTTGATCCTCTAT
GAATTTATGGACAATGGTAGCCTTTATGATGTTCTCCATGGGACCGAGGCAGCTCCAA
ATCTAGAGTGGAGGATACGGTATGACATAGCTCTTGGTACAGCACATGGTTTAGCTTA
TCTCCACAACGACTGCCACCCAGCCATTATTCACCGTGACATTAAACCCAAAAATATA
CTGTTGGATAAGGACATGGTGCCACATATTTCAGACTTTGGCATTGCGAAACTTATCA
ACCTGTCTCCTGCTGATTCACAGACTACTGGAATTGTTGGCACTGTTGGATATATGGC
CCCAGAGATGGCATTTTCAACCAGGAGTACCATAGAGTTCGACGTGTACAGCTACGG
AGTGGTATTACTTGAACTGATTACCAGAAAAATGGCCCTGGACCCCTCATTGCCTGAA
GATCTGGACCTAGTCAGTTGGGTGTCCTCCACCCTGAATGAGGGCAACGTAATCGAA
TCCGTGTGCGACCCTGCTCTAGTGCGTGAGGTATGTGGCACTGCTGAACTGGAGGAA
GTATGCAGTGTGCTGTCGATAGCCCTTAGATGCACCGCGGAGGACGCAAGACACAG
GCCTTCCATGATGGACGTTGTGAAAGAGCTGACGCATGCTAGGCGTGATGTTGTATC
GCTACCCAAGCAGGGGATATCTGGTTCCAGCAGTTCCTGTCAAAACCTGTCAACTTG
A

1. A tissue regeneration factor comprising an amino acid sequence as defined in SEQ ID NO: 2 or a functional variant or homologue thereof, wherein the homologue is selected from SEQ ID NO: 4, 6, 8, 10, 12, 14, 16, 18, 89 or 20 or a functional variant or homologue thereof of any of SEQ ID NOs 2, 4, 6, 8, 10, 12, 14, 16, 18, 89 or 20.

2. The tissue regeneration factor of claim 1, wherein the homologue or functional variant comprises a conserved motif, where the sequence of the conserved motif is selected from (SEQ ID NO: 79) GXPPXXNN, (SEQ ID NO: 80) SSGXXGXXN and (SEQ ID NO: 81) SGGXGGXHX, where X is any amino acid.

3. A plant tissue culture medium comprising the tissue regeneration factor of claim 1.

4. The plant tissue culture medium of claim 3, wherein the tissue culture medium is a callus-inducing medium, a shoot-inducing medium or a root-inducing medium.

5. The plant tissue culture medium of claim 3, wherein the plant tissue culture medium further comprises auxin and/or cytokinin.

6. The plant tissue culture medium of claim 3, wherein the plant tissue culture medium comprises the tissue regeneration factor at a concentration between 0.00001 and 100 nM or at a concentration of around 0.1 nM, 1 nM, 10 nM, 50 nM or 100 nM.

7. (canceled)

8. A nucleic acid construct comprising a nucleic acid sequence encoding at least one of a tissue regeneration factor, a tissue regeneration factor precursor and a tissue regeneration factor receptor;
wherein the tissue regeneration factor is selected from SEQ ID NO: 2 or functional variant or homologue thereof, wherein the homologue is selected from SEQ ID NO: 4, 6, 8, 10, 12, 14, 16, 18, 89 or 20 or a functional variant or homologue thereof; and
wherein the tissue regeneration factor precursor is selected from SEQ ID NO: 22 or a functional variant or homologue thereof, wherein the homologue is selected from SEQ ID NO: 24, 26, 28, 30, 32, 34, 36, 38, 91 or 40 or a functional variant or homologue thereof; and
wherein the tissue regeneration factor receptor is selected from SEQ ID NO: 42 or a functional variant or homologue thereof, wherein the homologue is selected from SEQ ID NO: 44, 46, 48, 50, 52, 54, 56, 58, 60, 62, 64, 66, 68, 70, 72, 74, 76, 93 or 78 or a functional variant or homologue thereof.

9. The nucleic acid construct of claim 8, wherein the homologue or functional variant of the tissue regeneration factor or tissue regeneration factor precursor comprises a conserved motif, where the sequence of the conserved motif is selected from (SEQ ID NO: 79) GXPPXXNN, (SEQ ID NO: 80) SSGXXGXXN and (SEQ ID NO: 81) SGGXGGXHX, where X is any amino acid.

10. The nucleic acid construct of claim 8, wherein the nucleic acid construct comprises a nucleic acid sequence encoding a tissue regeneration factor precursor and/or a tissue regeneration factor receptor.

11. (canceled)

12. The nucleic acid construct of claim 8, wherein the nucleic acid sequence encoding at least one of a tissue regeneration factor, a tissue regeneration factor precursor and a tissue regeneration factor receptor is operably linked to one or more regulatory sequences wherein the regulatory sequence is an constitutive or strong promoter or an inducible promoter, wherein the inducible promoter is the nopaline synthase promoter.

13. (canceled)

14. A host cell comprising the nucleic acid construct of claim 8.

15. A genetically altered plant, plant part thereof, or plant cell, wherein said plant, part thereof or plant cell is characterized by increased expression and/or levels at least one of
a tissue regeneration factor; and/or
a tissue regeneration factor precursor; and/or
iii a tissue regeneration factor receptor
wherein the tissue regeneration factor is selected from SEQ ID NO: 2 or functional variant or homologue thereof, wherein the homologue is selected from SEQ ID NO: 4, 6, 8, 10, 12, 14, 16, 18, 89 or 20 or a functional variant or homologue thereof; and
wherein the tissue regeneration factor precursor is selected from SEQ ID NO: 22 or a functional variant or homologue thereof, wherein the homologue is selected from SEQ ID NO: 24, 26, 28, 30, 32, 34, 36, 38, 91 or 40 or a functional variant or homologue thereof; and
wherein the tissue regeneration factor receptor is selected from SEQ ID NO: 42 or a functional variant or homologue thereof, wherein the homologue is selected from SEQ ID NO: 44, 46, 48, 50, 52, 54, 56, 58, 60, 62, 64, 66, 68, 70, 72, 74, 76, 93 or 78 or a functional variant or homologue thereof; or
wherein said plant is characterized by one or more mutation in the plant genome, where the mutation is the insertion of at least one additional copy of a nucleic sequence encoding a tissue regeneration factor precursor and/or at least one additional copy of a nucleic acid sequence encoding a tissue regeneration factor receptor, such that said nucleic acid sequence(s) is operably linked to a regulatory sequence, and wherein preferably the mutation is introduced using targeted genome editing;
wherein the tissue regeneration factor precursor is selected from SEQ ID NO: 22 or a functional variant or homologue thereof, wherein the homologue is selected from SEQ ID NO: 24, 26, 28, 30, 32, 34, 36, 38, 91 or 40 or a functional variant or homologue thereof; and
wherein the tissue regeneration factor receptor is selected from SEQ ID NO: 42 or a functional variant or homologue thereof, wherein the homologue is selected from SEQ ID NO: 44, 46, 48, 50, 52, 54, 56, 58, 60, 62, 64, 66, 68, 70, 72, 74, 76, 93 or 78 or a functional variant or homologue thereof.

16. The genetically altered plant of claim 15, wherein the homologue or functional variant of the tissue regeneration factor or tissue regeneration factor precursor comprises a conserved motif, where the sequence of the conserved motif is selected from (SEQ ID NO: 79) GXPPXXNN, (SEQ ID NO: 80) SSGXXGXXN and (SEQ ID NO: 81) SGGXGGXHX, where X is any amino acid.

17. A genetically altered plant, part thereof or plant cell, wherein the plant, part thereof or plant cell comprises the nucleic acid construct of claim 8.

18. (canceled)

19. (canceled)

20. (canceled)

21. (canceled)

22. A method of increasing the regenerative efficiency of a plant, plant part thereof or one or more plant cells, the method comprising
administering the tissue regeneration factor of claim 1 to the plant, part thereof or one or more plant cells.

23. A method of producing a plant, plant part thereof or one or more plant cells with increased regeneration efficiency, the method comprising
introducing and expressing in the plant, part thereof or one or more plant cells, the nucleic acid construct of claim 8.

24. The method of claim 23, the method further comprising producing a plant from the one or more plant cells.

25. The method of claim 22, wherein 0.1-500 nmol/L of the tissue regeneration factor is administered exogenously to the plant, part thereof or plant cell.

26. (canceled)

27. The method of claim 22, wherein the method increases one or more of callus formation and shoot regeneration.

28. The method of claim 27, wherein the plant part is an explant, wherein preferably the explant is selected from a nodal segment, apical meristem, root, cotyledon, embryo, leaf disc, leaf blade, pedicle, petiole, pollen, microspore, anther, ovary, hypocotyl.

29. (canceled)

30. (canceled)
